SEQUENCE LISTING (86 sequences)

SEQ ID NO: 1 - protein, 217 aa, Artificial Sequence
  Synthetic Polypeptide
APEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGK

SEQ ID NO: 2 - protein, 217 aa, Artificial Sequence
  Synthetic Polypeptide
APEFLGGPSVFLFPPKPKDQLMISRTPEVTCVVVDVSQEDPEVQFNWYVDGVEVHNAKTKPREEQFNSTYRVVSVLTTPHSDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVLHEALHNHYTQKSLSLSLGK

SEQ ID NO: 3 - protein, 217 aa, Artificial Sequence
  Synthetic Polypeptide
APEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 4 - protein, 11 aa, Artificial Sequence
  Synthetic Polypeptide
SKYGPPCPSCP

SEQ ID NO: 5 - protein, 11 aa, Artificial Sequence
  Synthetic Polypeptide
SKYGPPCPPCP

SEQ ID NO: 6 - protein, 6 aa, Artificial Sequence
  Synthetic Polypeptide
SKYGPP

SEQ ID NO: 7 - protein, 6 aa, Artificial Sequence
  Synthetic Polypeptide
IEGRMD

SEQ ID NO: 8 - protein, 9 aa, Artificial Sequence
  Synthetic Polypeptide
GGGVPRDCG

SEQ ID NO: 9 - protein, 15 aa, Artificial Sequence
  Synthetic Polypeptide
IEGRMDGGGGAGGGG

SEQ ID NO: 10 - protein, 8 aa, Artificial Sequence
  Synthetic Polypeptide
GGGSGGGS

SEQ ID NO: 11 - protein, 12 aa, Artificial Sequence
  Synthetic Polypeptide
GGGSGGGGSGGG

SEQ ID NO: 12 - protein, 14 aa, Artificial Sequence
  Synthetic Polypeptide
EGKSSGSGSESKST

SEQ ID NO: 13 - protein, 4 aa, Artificial Sequence
  Synthetic Polypeptide
GGSG

SEQ ID NO: 14 - protein, 12 aa, Artificial Sequence
  Synthetic Polypeptide
GGSGGGSGGGSG

SEQ ID NO: 15 - protein, 15 aa, Artificial Sequence
  Synthetic Polypeptide
EAAAKEAAAKEAAAK

SEQ ID NO: 16 - protein, 20 aa, Artificial Sequence
  Synthetic Polypeptide
EAAAREAAAREAAAREAAAR

SEQ ID NO: 17 - protein, 17 aa, Artificial Sequence
  Synthetic Polypeptide
GGGGSGGGGSGGGGSAS

SEQ ID NO: 18 - protein, 9 aa, Artificial Sequence
  Synthetic Polypeptide
GGGGAGGGG

SEQ ID NO: 19 - skipped

SEQ ID NO: 20 - protein, 6 aa, Artificial Sequence
  Synthetic Polypeptide
GSGSGS

SEQ ID NO: 21 - protein, 10 aa, Artificial Sequence
  Synthetic Polypeptide
GSGSGSGSGS

SEQ ID NO: 22 - protein, 7 aa, Artificial Sequence
  Synthetic Polypeptide
GGGGSAS

SEQ ID NO: 23 - protein, 20 aa, Artificial Sequence
  Synthetic Polypeptide
APAPAPAPAPAPAPAPAPAP

SEQ ID NO: 24 - protein, 4 aa, Artificial Sequence
  Synthetic Polypeptide
CPPC

SEQ ID NO: 25 - protein, 5 aa, Artificial Sequence
  Synthetic Polypeptide
GGGGS

SEQ ID NO: 26 - protein, 10 aa, Artificial Sequence
  Synthetic Polypeptide
GGGGSGGGGS

SEQ ID NO: 27 - protein, 15 aa, Artificial Sequence
  Synthetic Polypeptide
GGGGSGGGGSGGGGS

SEQ ID NO: 28 - protein, 20 aa, Artificial Sequence
  Synthetic Polypeptide
GGGGSGGGGSGGGGSGGGGS

SEQ ID NO: 29 - protein, 25 aa, Artificial Sequence
  Synthetic Polypeptide
GGGGSGGGGSGGGGSGGGGSGGGGS

SEQ ID NO: 30 - protein, 30 aa, Artificial Sequence
  Synthetic Polypeptide
GGGGSGGGGSGGGGSGGGGSGGGGSGGGGS

SEQ ID NO: 31 - protein, 35 aa, Artificial Sequence
  Synthetic Polypeptide
GGGGSGGGGSGGGGSGGGGSGGGGSGGGGSGGGGS

SEQ ID NO: 32 - protein, 40 aa, Artificial Sequence
  Synthetic Polypeptide
GGGGSGGGGSGGGGSGGGGSGGGGSGGGGSGGGGSGGGGS

SEQ ID NO: 33 - protein, 16 aa, Artificial Sequence
  Synthetic Polypeptide
GGSGGSGGGGSGGGGS

SEQ ID NO: 34 - protein, 8 aa, Artificial Sequence
  Synthetic Polypeptide
GGGGGGGG

SEQ ID NO: 35 - protein, 6 aa, Artificial Sequence
  Synthetic Polypeptide
GGGGGG

SEQ ID NO: 36 - protein, 5 aa, Artificial Sequence
  Synthetic Polypeptide
EAAAK

SEQ ID NO: 37 - protein, 10 aa, Artificial Sequence
  Synthetic Polypeptide
EAAAKEAAAK

SEQ ID NO: 38 - protein, 15 aa, Artificial Sequence
  Synthetic Polypeptide
EAAAKEAAAKEAAAK

SEQ ID NO: 39 - protein, 12 aa, Artificial Sequence
  Synthetic Polypeptide
AEAAAKEAAAKA

SEQ ID NO: 40 - protein, 17 aa, Artificial Sequence
  Synthetic Polypeptide
AEAAAKEAAAKEAAAKA

SEQ ID NO: 41 - protein, 22 aa, Artificial Sequence
  Synthetic Polypeptide
AEAAAKEAAAKEAAAKEAAAKA

SEQ ID NO: 42 - protein, 27 aa, Artificial Sequence
  Synthetic Polypeptide
AEAAAKEAAAKEAAAKEAAAKEAAAKA

SEQ ID NO: 43 - protein, 46 aa, Artificial Sequence
  Synthetic Polypeptide
AEAAAKEAAAKEAAAKEAAAKALEAEAAAKEAAAKEAAAKEAAAKA

SEQ ID NO: 44 - protein, 5 aa, Artificial Sequence
  Synthetic Polypeptide
PAPAP

SEQ ID NO: 45 - protein, 18 aa, Artificial Sequence
  Synthetic Polypeptide
KESGSVSSEQLAQFRSLD

SEQ ID NO: 46 - protein, 12 aa, Artificial Sequence
  Synthetic Polypeptide
GSAGSAAGSGEF

SEQ ID NO: 47 - protein, 5 aa, Artificial Sequence
  Synthetic Polypeptide
GGGSE

SEQ ID NO: 48 - protein, 5 aa, Artificial Sequence
  Synthetic Polypeptide
GSESG

SEQ ID NO: 49 - protein, 5 aa, Artificial Sequence
  Synthetic Polypeptide
GSEGS

SEQ ID NO: 50 - protein, 35 aa, Artificial Sequence
  Synthetic Polypeptide
GEGGSGEGSSGEGSSSEGGGSEGGGSEGGGSEGGS

SEQ ID NO: 51 - protein, 234 aa, Artificial Sequence
  Synthetic Polypeptide
SKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGKIEGRMD

SEQ ID NO: 52 - protein, 234 aa, Artificial Sequence
  Synthetic Polypeptide
SKYGPPCPSCPAPEFLGGPSVFLFPPKPKDQLMISRTPEVTCVVVDVSQEDPEVQFNWYVDGVEVHNAKTKPREEQFNSTYRVVSVLTTPHSDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVLHEALHNHYTQKSLSLSLGKIEGRMD

SEQ ID NO: 53 - protein, 234 aa, Artificial Sequence
  Synthetic Polypeptide
SKYGPPCPSCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMD

SEQ ID NO: 54 - protein, 234 aa, Artificial Sequence
  Synthetic Polypeptide
SKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGKIEGRMD

SEQ ID NO: 55 - protein, 234 aa, Artificial Sequence
  Synthetic Polypeptide
SKYGPPCPPCPAPEFLGGPSVFLFPPKPKDQLMISRTPEVTCVVVDVSQEDPEVQFNWYVDGVEVHNAKTKPREEQFNSTYRVVSVLTTPHSDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVLHEALHNHYTQKSLSLSLGKIEGRMD

SEQ ID NO: 56 - protein, 234 aa, Artificial Sequence
  Synthetic Polypeptide
SKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMD

SEQ ID NO: 57 - protein, 140 aa, Artificial Sequence
  Synthetic Polypeptide
PSTLIQRHNNSSLNTRTQKARHCGHCPEEWITYSNSCYYIGKERRTWEESLLACTSKNSSLLSIDNEEEMKFLSIISPSSWIGVFRNSSHHPWVTMNGLAFKHEIKDSDNAELNCAVLQVNRLKSAQCGSSIIYHCKHKL

SEQ ID NO: 58 - protein, 150 aa, Artificial Sequence
  Synthetic Polypeptide
TPYTEAKAQINSSMTRTHRDINYTLSSAQPCPHCPKEWISYSHNCYFIGMERKSWNDSLVSCISKNCSLLYIDSEEEQDFLQSLSLISWTGILRKGRGQPWVWKEDSIFKPKIAEILHDECNCAMMSASGLTADNCTTLHPYLCKCKFPI

SEQ ID NO: 59 - protein, 208 aa, Artificial Sequence [sequence omitted]

SEQ ID NO: 60 - protein, 209 aa, Artificial Sequence
  Synthetic Polypeptide
VDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKN

SEQ ID NO: 61 - protein, 582 aa, Artificial Sequence
  Synthetic Polypeptide
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDPSTLIQRHNNSSLNTRTQKARHCGHCPEEWITYSNSCYYIGKERRTWEESLLACTSKNSSLLSIDNEEEMKFLSIISPSSWIGVFRNSSHHPWVTMNGLAFKHEIKDSDNAELNCAVLQVNRLKSAQCGSSIIYHCKHKL

SEQ ID NO: 62 - protein, 592 aa, Artificial Sequence
  Synthetic Polypeptide
VDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGIIEGRMDTPYTEAKAQINSSMTRTHRDINYTLSSAQPCPHCPKEWISYSHNCYFIGMERKSWNDSLVSCISKNCSLLYIDSEEEQDFLQSLSLISWTGILRKGRGQPWVWKEDSIFKPKIAEILHDECNCAMMSASGLTADNCTTLHPYLCKCKFPI

SEQ ID NO: 63 - protein, 224 aa, Artificial Sequence
  Synthetic Polypeptide
APLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIP

SEQ ID NO: 64 - protein, 221 aa, Artificial Sequence
  Synthetic Polypeptide
VSVETQAYFNGTAYLPCPFTKAQNISLSELVVFWQDQQKLVLYEHYLGTEKLDSVNAKYLGRTSFDRNNWTLRLHNVQIKDMGSYDCFIQKKPPTGSIILQQTLTELSVIANFSEPEIKLAQNVTGNSGINLTCTSKQGHPKPKKMYFLITNSTNEYGDNMQISQDNVTELFSISNSLSLSFPDGVWHMTVVCVLETESMKISSKPLNFTQEFPSPQTYWK

SEQ ID NO: 65 - protein, 598 aa, Artificial Sequence
  Synthetic Polypeptide
APLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDPSTLIQRHNNSSLNTRTQKARHCGHCPEEWITYSNSCYYIGKERRTWEESLLACTSKNSSLLSIDNEEEMKFLSIISPSSWIGVFRNSSHHPWVTMNGLAFKHEIKDSDNAELNCAVLQVNRLKSAQCGSSIIYHCKHKL

SEQ ID NO: 66 - protein, 604 aa, Artificial Sequence
  Synthetic Polypeptide
VSVETQAYFNGTAYLPCPFTKAQNISLSELVVFWQDQQKLVLYEHYLGTEKLDSVNAKYLGRTSFDRNNWTLRLHNVQIKDMGSYDCFIQKKPPTGSIILQQTLTELSVIANFSEPEIKLAQNVTGNSGINLTCTSKQGHPKPKKMYFLITNSTNEYGDNMQISQDNVTELFSISNSLSLSFPDGVWHMTVVCVLETESMKISSKPLNFTQEFPSPQTYWKVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGIIEGRMDTPYTEAKAQINSSMTRTHRDINYTLSSAQPCPHCPKEWISYSHNCYFIGMERKSWNDSLVSCISKNCSLLYIDSEEEQDFLQSLSLISWTGILRKGRGQPWVWKEDSIFKPKIAEILHDECNCAMMSASGLTADNCTTLHPYLCKCKFPI

SEQ ID NO: 67 - protein, 187 aa, Artificial Sequence
  Synthetic Polypeptide
FSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHR

SEQ ID NO: 68 - protein, 561 aa, Artificial Sequence
  Synthetic Polypeptide
FSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDPSTLIQRHNNSSLNTRTQKARHCGHCPEEWITYSNSCYYIGKERRTWEESLLACTSKNSSLLSIDNEEEMKFLSIISPSSWIGVFRNSSHHPWVTMNGLAFKHEIKDSDNAELNCAVLQVNRLKSAQCGSSIIYHCKHKL

SEQ ID NO: 69 - protein, 143 aa, Artificial Sequence
  Synthetic Polypeptide
LDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQ

SEQ ID NO: 70 - protein, 149 aa, Artificial Sequence
  Synthetic Polypeptide
SGWLLEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQGM

SEQ ID NO: 71 - protein, 517 aa, Artificial Sequence
  Synthetic Polypeptide
LDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDPSTLIQRHNNSSLNTRTQKARHCGHCPEEWITYSNSCYYIGKERRTWEESLLACTSKNSSLLSIDNEEEMKFLSIISPSSWIGVFRNSSHHPWVTMNGLAFKHEIKDSDNAELNCAVLQVNRLKSAQCGSSIIYHCKHKL

SEQ ID NO: 72 - protein, 493 aa, Artificial Sequence
  Synthetic Polypeptide
SEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQGMVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGIIEGRMDTPYTEAKAQINSSMTRTHRDINYTLSSAQPCPHCPKEWISYSHNCYFIGMERKSWNDSLVSCISKNCSLLYIDSEEEQDFLQSLSLISWTGILRKGRGQPWVWKEDSIFKPKIAEILHDECNCAMMSASGLTADNCTTLHPYLCKCKFPI

SEQ ID NO: 73 - protein, 205 aa, Artificial Sequence
  Synthetic Polypeptide
QSSLTPLMVNGILGESVTLPLEFPAGEKVNFITWLFNETSLAFIVPHETKSPEIHVTNPKQGKRLNFTQSYSLQLSNLKMEDTGSYRAQISTKTSAKLSSYTLRILRQLRNIQVTNHSQLFQNMTCELHLTCSVEDADDNVSFRWEALGNTLSSQPNLTVSWDPRISSEQDYTCIAENAVSNLSFSVSAQKLCEDVKIQYTDTKM

SEQ ID NO: 74 - protein, 209 aa, Artificial Sequence
  Synthetic Polypeptide
EVSQSSSDPQLMNGVLGESAVLPLKLPAGKIANIIIWNYEWEASQVTALVINLSNPESPQIMNTDVKKRLNITQSYSLQISNLTMADTGSYTAQITTKDSEVITFKYILRVFERLGNLETTNYTLLLENGTCQIHLACVLKNQSQTVSVEWQATGNISLGGPNVTIFWDPRNSGDQTYVCRAKNAVSNLSVSVSTQSLCKGVLTNPPWN

SEQ ID NO: 75 - protein, 579 aa, Artificial Sequence
  Synthetic Polypeptide
QSSLTPLMVNGILGESVTLPLEFPAGEKVNFITWLFNETSLAFIVPHETKSPEIHVTNPKQGKRLNFTQSYSLQLSNLKMEDTGSYRAQISTKTSAKLSSYTLRILRQLRNIQVTNHSQLFQNMTCELHLTCSVEDADDNVSFRWEALGNTLSSQPNLTVSWDPRISSEQDYTCIAENAVSNLSFSVSAQKLCEDVKIQYTDTKMSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDPSTLIQRHNNSSLNTRTQKARHCGHCPEEWITYSNSCYYIGKERRTWEESLLACTSKNSSLLSIDNEEEMKFLSIISPSSWIGVFRNSSHHPWVTMNGLAFKHEIKDSDNAELNCAVLQVNRLKSAQCGSSIIYHCKHKL

SEQ ID NO: 76 - protein, 591 aa, Artificial Sequence
  Synthetic Polypeptide
VSQSSSDPQLMNGVLGESAVLPLKLPAGKIANIIIWNYEWEASQVTALVINLSNPESPQIMNTDVKKRLNITQSYSLQISNLTMADTGSYTAQITTKDSEVITFKYILRVFERLGNLETTNYTLLLENGTCQIHLACVLKNQSQTVSVEWQATGNISLGGPNVTIFWDPRNSGDQTYVCRAKNAVSNLSVSVSTQSLCKGVLTNPPWNVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGIIEGRMDTPYTEAKAQINSSMTRTHRDINYTLSSAQPCPHCPKEWISYSHNCYFIGMERKSWNDSLVSCISKNCSLLYIDSEEEQDFLQSLSLISWTGILRKGRGQPWVWKEDSIFKPKIAEILHDECNCAMMSASGLTADNCTTLHPYLCKCKFPI

SEQ ID NO: 77 - protein, 144 aa, Artificial Sequence
  Synthetic Polypeptide
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 78 - protein, 161 aa, Artificial Sequence
  Synthetic Polypeptide
IPPHVPKSDVEMEAQKDASIHLSCNRTIHPLKHFNSDVMASDNGGAVKLPQLCKFCDVRLSTCDNQKSCMSNCSITAICEKPHEVCVAVWRKNDKNITLETVCHDPKLTYHGFTLEDAASPKCVMKEKKRAGETFFMCACNMEECNDYIIFSEEYTTSSPD

SEQ ID NO: 79 - protein, 518 aa, Artificial Sequence
  Synthetic Polypeptide
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDPSTLIQRHNNSSLNTRTQKARHCGHCPEEWITYSNSCYYIGKERRTWEESLLACTSKNSSLLSIDNEEEMKFLSIISPSSWIGVFRNSSHHPWVTMNGLAFKHEIKDSDNAELNCAVLQVNRLKSAQCGSSIIYHCKHKL

SEQ ID NO: 80 - protein, 544 aa, Artificial Sequence
  Synthetic Polypeptide
IPPHVPKSDVEMEAQKDASIHLSCNRTIHPLKHFNSDVMASDNGGAVKLPQLCKFCDVRLSTCDNQKSCMSNCSITAICEKPHEVCVAVWRKNDKNITLETVCHDPKLTYHGFTLEDAASPKCVMKEKKRAGETFFMCACNMEECNDYIIFSEEYTTSSPDVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGIIEGRMDTPYTEAKAQINSSMTRTHRDINYTLSSAQPCPHCPKEWISYSHNCYFIGMERKSWNDSLVSCISKNCSLLYIDSEEEQDFLQSLSLISWTGILRKGRGQPWVWKEDSIFKPKIAEILHDECNCAMMSASGLTADNCTTLHPYLCKCKFPI

SEQ ID NO: 81 - protein, 193 aa, Artificial Sequence
  Synthetic Polypeptide
QGHLVHMTVVSGSNVTLNISESLPENYKQLTWFYTFDQKIVEWDSRKSKYFESKFKGRVRLDPQSGALYISKVQKEDNSTYIMRVLKKTGNEQEWKIKLQVLDPVPKPVIKIEKIEDMDDNCYLKLSCVIPGESVNYTWYGDKRPFPKELQNSVLETTLMPHNYSRCYTCQVSNSVSSKNGTVCLSPPCTLAR

SEQ ID NO: 82 - protein, 195 aa, Artificial Sequence
  Synthetic Polypeptide
FQGHSIPDINATTGSNVTLKIHKDPLGPYKRITWLHTKNQKILEYNYNSTKTIFESEFKGRVYLEENNGALHISNVRKEDKGTYYMRVLRETENELKITLEVFDPVPKPSIEINKTEASTDSCHLRLSCEVKDQHVDYTWYESSGPFPKKSPGYVLDLIVTPQNKSTFYTCQVSNPVSSKNDTVYFTLPCDLARS

SEQ ID NO: 83 - protein, 568 aa, Artificial Sequence
  Synthetic Polypeptide
QGHLVHMTVVSGSNVTLNISESLPENYKQLTWFYTFDQKIVEWDSRKSKYFESKFKGRVRLDPQSGALYISKVQKEDNSTYIMRVLKKTGNEQEWKIKLQVLDPVPKPVIKIEKIEDMDDNCYLKLSCVIPGESVNYTWYGDKRPFPKELQNSVLETTLMPHNYSRCYTCQVSNSVSSKNGTVCLSPPCTLARSSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDPSTLIQRHNNSSLNTRTQKARHCGHCPEEWITYSNSCYYIGKERRTWEESLLACTSKNSSLLSIDNEEEMKFLSIISPSSWIGVFRNSSHHPWVTMNGLAFKHEIKDSDNAELNCAVLQVNRLKSAQCGSSIIYHCKHKL

SEQ ID NO: 84 - protein, 578 aa, Artificial Sequence
  Synthetic Polypeptide
FQGHSIPDINATTGSNVTLKIHKDPLGPYKRITWLHTKNQKILEYNYNSTKTIFESEFKGRVYLEENNGALHISNVRKEDKGTYYMRVLRETENELKITLEVFDPVPKPSIEINKTEASTDSCHLRLSCEVKDQHVDYTWYESSGPFPKKSPGYVLDLIVTPQNKSTFYTCQVSNPVSSKNDTVYFTLPCDLARSVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGIIEGRMDTPYTEAKAQINSSMTRTHRDINYTLSSAQPCPHCPKEWISYSHNCYFIGMERKSWNDSLVSCISKNCSLLYIDSEEEQDFLQSLSLISWTGILRKGRGQPWVWKEDSIFKPKIAEILHDECNCAMMSASGLTADNCTTLHPYLCKCKFPI

SEQ ID NO: 85 - protein, 343 aa, Artificial Sequence
  Synthetic Polypeptide
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIY

SEQ ID NO: 86 - protein, 717 aa, Artificial Sequence
  Synthetic polypeptide
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDPSTLIQRHNNSSLNTRTQKARHCGHCPEEWITYSNSCYYIGKERRTWEESLLACTSKNSSLLSIDNEEEMKFLSIISPSSWIGVFRNSSHHPWVTMNGLAFKHEIKDSDNAELNCAVLQVNRLKSAQCGSSIIYHCKHKL

(19) United States
(12) Patent Application Publication
(10) Pub. No.: US 2023/0045794 A1
(43) Pub. Date: Feb. 16, 2023

(54) NK CELL-DIRECTED CHIMERIC PROTEINS

(71) Applicant: Shattuck Labs, Inc., Austin, TX (US)

(72) Inventors: Taylor SCHREIBER, Austin, TX (US); Casey SHUPTRINE, Austin, TX (US); George FROMM, Austin, TX (US); Suresh DE SILVA, Austin, TX (US)

(21) Appl. No.: 17/720,771

(22) Filed: Apr. 14, 2022

Related U.S. Application Data
(63) Continuation of application No. 17/017,117, filed on Sep. 10, 2020, now abandoned.
Application No. 17/017,117 is a continuation of application No. PCT/US20/33169, filed on May 15, 2020.
(60) Provisional application No. 62/848,915, filed on May 16, 2019.

(51) Int. Cl. C07K 14/705; A61P 35/00; A61K 38/00; C07K 14/74

(52) U.S. Cl. CPC C07K 14/70532; A61P 35/00; A61K 38/00; C07K 14/70539; C07K 2319/30

(57) ABSTRACT

The present invention relates, inter alia, to compositions and methods, including chimeric proteins comprising an extracellular domain of a Type I transmembrane protein or a portion of a membrane-anchored extracellular protein and a portion of the extracellular domain of a Type II transmembrane protein, wherein the Type II transmembrane protein is naturally expressed on the surface of a Natural Killer (NK) cell that find use in the treatment of disease, such as cancer and viral infections.

### PRIORITY

This application claims the benefit of, and priority to, U.S. application No. 62/848,915, filed May 16, 2019, which is hereby incorporated by reference in its entirety.

### TECHNICAL FIELD

The present invention relates to, inter alia, compositions and methods, including chimeric proteins that find use in the treatment of disease, such as immunotherapies for cancer and viral infection.

### DESCRIPTION OF THE TEXT FILE SUBMITTED ELECTRONICALLY

This application contains a sequence listing. It has been submitted electronically via EFS-Web as an ASCII text file entitled “SHK-016PC_SequenceListing_ST25”. The sequence listing is 128,123 bytes in size, and was prepared on or about May 15, 2020. The sequence listing is hereby incorporated by reference in its entirety.

### BACKGROUND

The immune system is central to the body's response to foreign entities that can cause disease and to the body's response to cancer cells. However, many cancers and virally infected cells have developed mechanisms to avoid the immune system by, for instance, delivering immune inhibitory signals to Natural killer (NK) cells. NK cells are lymphocytes that can mediate lysis of certain tumor cells and virus-infected cells without previous activation. There remains an unmet need for therapeutics that block immune inhibitory signals originating from a cancer cell or a virus-infected cell and/or prevent reception of inhibitory signals by an NK cell.

### SUMMARY

Accordingly, in various aspects, the present invention provides for compositions and methods that are useful for cancer and antiviral immunotherapy. For instance, the present invention, in part, relates to specific chimeric proteins that simultaneously block immune inhibitory signals originating from a cancer cell or a virus-infected cell and preventing reception of inhibitory signals by an NK cell. Accordingly, the present compositions and methods overcome various deficiencies in the field of cancer and antiviral immunotherapy.

The present invention is based, in part, on the discovery that chimeric proteins can be engineered comprising a portion of the extracellular domain of a Type I transmembrane protein or a portion of a membrane-anchored extracellular protein and the extracellular domain of a Type II transmembrane protein in which the Type II transmembrane protein is naturally expressed on the surface of a Natural Killer (NK) cell. These chimeric proteins can inhibit transmission and/or reception of immunosuppressive signals. In some instances, the Type I transmembrane protein end of a chimeric protein disrupts, blocks, reduces, inhibits and/or sequesters the transmission of immune inhibitory signals, e.g., originating from a cancer cell or a virus-infected cell that is attempting to avoid its detection and/or destruction. In other instances, the Type I transmembrane protein may provide an immune-stimulatory signal that increases the activity of another immune cell. The Type II transmembrane protein end of a chimeric protein disrupts, blocks, reduces, inhibits and/or sequesters the inhibitory signals thereby preventing their reception by an NK cell. Together, these two actions allow for an anti-cancer attack or an attack of a virus-infected cell by the NK cell.

An aspect of the present invention is a chimeric protein of a general structure of: N terminus-(a)-(b)-(c)-C terminus, where (a) is a first domain comprising a portion of the extracellular domain of a Type I transmembrane protein or a portion of a membrane-anchored extracellular protein, (b) is a linker adjoining the first and second domains, and (c) is a second domain comprising a portion of the extracellular domain of a Type II transmembrane protein, wherein the Type II transmembrane protein is naturally expressed on the surface of a Natural Killer (NK) cell.

Another aspect of the present invention is a chimeric protein of a general structure of: N terminus-(a)-(b)-(c)-C terminus, where (c) comprises a portion of NKG2A that is capable of binding an NKG2A ligand.

In an aspect, the present invention provides a chimeric protein of a general structure of: N terminus-(a)-(b)-(c)-C terminus, where (a) is a first domain comprising a portion of the extracellular domain of a Type I transmembrane protein or a portion of a membrane-anchored extracellular protein, (b) is a linker adjoining the first and second domains, and (c) is a second domain comprising a portion of NKG2A that is capable of binding an NKG2A ligand.

Another aspect of the present invention is a chimeric protein comprising: (a) a first domain comprising a portion of CD80 that is capable of binding a CD80 ligand/receptor, (b) a second domain comprising a portion of NKG2A that is capable of binding an NKG2A ligand, and (c) a linker linking the first domain and the second domain and comprising a hinge-CH2-CH3 Fc domain.

Yet another aspect of the present invention is a chimeric protein comprising: (a) a first domain comprising a portion of CD86 that is capable of binding a CD86 ligand/receptor, (b) a second domain comprising a portion of NKG2A that is capable of binding an NKG2A ligand, and (c) a linker linking the first domain and the second domain and comprising a hinge-CH2-CH3 Fc domain.

In an aspect, the present invention provides a chimeric protein comprising: (a) a first domain comprising a portion of CD48 that is capable of binding a CD48 ligand/receptor, (b) a second domain comprising a portion of NKG2A that is capable of binding an NKG2A ligand, and (c) a linker linking the first domain and the second domain and comprising a hinge-CH2-CH3 Fc domain.

In another aspect, the present invention provides a chimeric protein comprising: (a) a first domain comprising a portion of CD58 that is capable of binding a CD58 ligand/receptor, (b) a second domain comprising a portion of NKG2A that is capable of binding an NKG2A ligand, and (c) a linker linking the first domain and the second domain and comprising a hinge-CH2-CH3 Fc domain.

In yet another aspect, the present invention provides a chimeric protein comprising: (a) a first domain comprising a portion of PD-1 that is capable of binding a PD-1 ligand, (b) a second domain comprising a portion of NKG2A that is capable of binding an NKG2A ligand, and (c) a linker linking the first domain and the second domain and comprising a hinge-CH2-CH3 Fc domain.

An aspect of the present invention is a chimeric protein comprising: (a) a first domain comprising a portion of SLAMF6 that is capable of binding a SLAMF6 ligand/receptor, (b) a second domain comprising a portion of NKG2A that is capable of binding an NKG2A ligand, and (c) a linker linking the first domain and the second domain and comprising a hinge-CH2-CH3 Fc domain.

An aspect of the present invention is a chimeric protein comprising: (a) a first domain comprising a portion of SIRPα that is capable of binding a SIRPα ligand/receptor, (b) a second domain comprising a portion of NKG2A that is capable of binding an NKG2A ligand, and (c) a linker linking the first domain and the second domain and comprising a hinge-CH2-CH3 Fc domain.

Another aspect of the present invention is a chimeric protein comprising: (a) a first domain comprising a portion of TGFBR2 that is capable of binding a TGFBR2 ligand, (b) a second domain comprising a portion of NKG2A that is capable of binding an NKG2A ligand, and (c) a linker linking the first domain and the second domain and comprising a hinge-CH2-CH3 Fc domain.

An aspect of the present invention is the use of a herein-disclosed chimeric protein as a medicament in the treatment of a cancer or a viral infection.

Another aspect of the present invention is the use of a herein-disclosed chimeric protein, in the manufacture of a medicament.

Yet another aspect of the present invention is an expression vector comprising a nucleic acid that encodes a herein-disclosed chimeric protein.

In an aspect, the present invention provides a host cell comprising an expression vector that comprises a nucleic acid that encodes a herein-disclosed chimeric protein.

In another aspect, the present invention provides a pharmaceutical composition comprising a therapeutically effective amount of a herein-disclosed chimeric protein.

In yet another an aspect, the present invention provides a method of treating a cancer or treating a viral infection, comprising administering to a subject in need thereof an effective amount of a pharmaceutical composition comprising a therapeutically effective amount of a herein-disclosed chimeric protein.

Any aspect or embodiment disclosed herein can be combined with any other aspect or embodiment as disclosed herein.

### BRIEF DESCRIPTION OF THE DRAWINGS

FIG. 1A shows schematic illustrations of Type I transmembrane proteins (left protein) and Type II transmembrane proteins (right protein). FIG. 1B shows two membrane-anchored extracellular proteins, with the curved lines represents the anchoring domains; the left protein has its carboxy terminus anchored to the cell membrane and the right protein has its amino terminus anchored to the cell membrane. FIG. 1C and FIG. 1D show illustrations of chimeric proteins of the present invention; there, linkers connect the two extracellular binding domains.

FIG. 2A is a cartoon showing the structure of an illustrative chimeric protein of the present invention: mouse CD86-Fc-NKG2A (mCD86-Fc-NKG2A). FIG. 2B are Western blots showing characterization of the mCD86-Fc-NKG2A chimeric protein. The Western blots demonstrate the chimeric protein's native state and tendency to form a multimer. Untreated samples (i.e., without a reducing agent or a deglycosylation agent, yet boiled) of the mCD86-Fc-NKG2A chimeric protein, were loaded into lane 2 in all the blots. Samples in lane 3 were treated with a reducing agent, β-mercaptoethanol, and were boiled. Samples in lane 4 were treated with a deglycosylation agent, the reducing agent, and were boiled. Each lane 1 included the protein size ladder. Each individual domain of the chimeric protein was probed using an anti-NKG2A antibody (left blot), an anti-Fc antibody (center blot), or an anti-CD86 antibody (right blot).

FIG. 3A to FIG. 3C show ELISA assays demonstrating the binding affinity of the Fc domain of mCD86-Fc-NKG2A (FIG. 3A), the NKG2A domain mCD86-Fc-NKG2A (FIG. 3B), and the CD86 domain of mCD86-Fc-NKG2A (FIG. 3C) for their respective binding partners.

FIG. 4A is a cartoon showing the structure of an illustrative chimeric protein of the present invention: mouse CD80-Fc-NKG2A (mCD80-Fc-NKG2A). FIG. 4B are Western blots showing characterization of the mCD80-Fc-NKG2A chimeric protein. The Western blots demonstrate the chimeric protein's native state and tendency to form a multimer. Untreated samples (i.e., without a reducing agent or a deglycosylation agent, yet boiled) of the mCD80-Fc-NKG2A chimeric protein, were loaded into lane 2 in all the blots. Samples in lane 3 were treated with a reducing agent, 3-mercaptoethanol, and were boiled. Samples in lane 4 were treated with a deglycosylation agent, the reducing agent, and were boiled. Each lane 1 included the protein size ladder. The two ECD domains of the chimeric protein was probed using an anti-NKG2A antibody (left blot) or an anti-CD80 antibody (right blot).

FIG. 5A to FIG. 5D show ELISA assays demonstrating the binding affinity of the Fc domain of mCD80-Fc-NKG2A (FIG. 5A), the NKG2A domain mCD80-Fc-NKG2A (FIG. 5B), and the CD80 domain of mCD80-Fc-NKG2A (FIG. 5C and FIG. 5D) for their respective binding partners. In FIG. 5C, the CD80 domain of mCD80-Fc-NKG2A chimeric protein was bound to CD28 and in FIG. 5D, the CD80 domain of mCD80-Fc-NKG2A chimeric protein was bound to CTLA-4.

FIG. 6A is a cartoon showing the structure of an illustrative chimeric protein of the present invention: mouse CD48-Fc-NKG2A (mCD48-Fc-NKG2A). FIG. 6B are Western blots showing characterization of the mCD48-Fc-NKG2A chimeric protein. The Western blots demonstrate the chimeric protein's native state and tendency to form a multimer. Untreated samples (i.e., without a reducing agent or a deglycosylation agent, yet boiled) of the mCD48-Fc-NKG2A chimeric protein, were loaded into lane 2 in all the blots. Samples in lane 3 were treated with a reducing agent, 3-mercaptoethanol, and were boiled. Samples in lane 4 were treated with a deglycosylation agent, the reducing agent, and were boiled. Each lane 1 included the protein size ladder. Each individual domain of the chimeric protein was probed using an anti-NKG2A antibody (left blot), an anti-Fc antibody (center blot), or an anti-CD48 antibody (right blot).

FIG. 7A to FIG. 7D show ELISA assays demonstrating the binding affinity of the Fc domain of mCD48-Fc-NKG2A (FIG. 7A), the NKG2A domain mCD48-Fc-NKG2A (FIG. 7B), and the CD48 domain of mCD48-Fc-NKG2A (FIG. 7C and FIG. 7D) for their respective binding partners. In FIG. 7C, the CD48 domain of mCD48-Fc-NKG2A chimeric protein was bound to CD2 and in FIG. 7D, the CD48 domain of mCD48-Fc-NKG2A chimeric protein was bound to 2B4.

FIG. 8A is a cartoon showing the structure of an illustrative chimeric protein of the present invention: human PD-1-Fc-hNKG2A (hPD-1-Fc-hNKG2A). FIG. 8B are Western blots showing characterization of the hPD-1-Fc-hNKG2A chimeric protein. The Western blots demonstrate the chimeric protein's native state and tendency to form a multimer. Untreated samples (i.e., without a reducing agent or a deglycosylation agent, yet boiled) of the hPD-1-Fc-hNKG2A chimeric protein, were loaded into lane 2 in all the blots. Samples in lane 3 were treated with a reducing agent, 3-mercaptoethanol, and were boiled. Samples in lane 4 were treated with a deglycosylation agent, the reducing agent, and were boiled. Each lane 1 included the protein size ladder. The two ECD domains the chimeric protein was probed using an anti-NKG2A antibody (left blot) or an anti-PD-1 antibody (right blot).

FIG. 9A to FIG. 9D show ELISA assays demonstrating the binding affinity of the Fc domain of hPD-1-Fc-hNKG2A (FIG. 9A), the PD-1 domain of hPD-1-Fc-hNKG2A (FIG. 9B), and the hNKG2A domain hPD-1-Fc-hNKG2A (FIG. 9C and FIG. 9D) for their respective binding partners.

FIG. 10A and FIG. 10B shows images of native, non-denaturing (non-SDS) Polyacrylamide gel electrophoresis (PAGE) for illustrative chimeric proteins of the present invention. Proteins in the gel images of FIG. 10A, were not stained with Coomassie Brilliant Blue whereas the proteins in the gel image of FIG. 10B were Coomassie stained.

FIG. 11A to FIG. 11E shows in vivo reductions in tumor volume size resulting from methods of cancer treatments according to the present invention.

FIG. 12A is a cartoon showing the structure of an illustrative chimeric protein of the present invention: human CD86-Fc-hNKG2A (hCD86-Fc-NKG2A). FIG. 12B are Western blots showing characterization of the hCD86-Fc-NKG2A chimeric protein. The Western blots demonstrate the chimeric protein's native state and tendency to form a multimer. Untreated samples (i.e., without a reducing agent or a deglycosylation agent, yet boiled) of the hCD86-Fc-NKG2A chimeric protein, were loaded into the lane marked as NR of each of the blots. Samples in the lane marked as R were treated with a reducing agent, β-mercaptoethanol, and were boiled. Samples in the lane marked as DG were treated with a deglycosylation agent, the reducing agent, and were boiled. The lane marked as L included the protein size ladder. Each individual domain of the chimeric protein was probed using an anti-CD86 antibody (left blot), an anti-Fc antibody (center blot), or an anti-NKG2A antibody (right blot).

FIG. 13A is a cartoon showing the structure of an illustrative chimeric protein of the present invention: human CD48-Fc-hNKG2A (hCD48-Fc-NKG2A). FIG. 13B are Western blots showing characterization of the hCD48-Fc-NKG2A chimeric protein. The Western blots demonstrate the chimeric protein's native state and tendency to form a multimer. Untreated samples (i.e., without a reducing agent or a deglycosylation agent, yet boiled) of the hCD48-Fc-NKG2A chimeric protein, were loaded into the lane marked as NR of each of the blots. Samples in the lane marked as R were treated with a reducing agent, β-mercaptoethanol, and were boiled. Samples in the lane marked as DG were treated with a deglycosylation agent, the reducing agent, and were boiled. The lane marked as L included the protein size ladder. Each individual domain of the chimeric protein was probed using an anti-CD48 antibody (left blot), an anti-Fc antibody (center blot), or an anti-NKG2A antibody (right blot).

FIG. 14A is a cartoon showing the structure of an illustrative chimeric protein of the present invention: human CD58-Fc-hNKG2A (hCD58-Fc-NKG2A). FIG. 14B are Western blots showing characterization of the hCD58-Fc-NKG2A chimeric protein. The Western blots demonstrate the chimeric protein's native state and tendency to form a multimer. Untreated samples (i.e., without a reducing agent or a deglycosylation agent, yet boiled) of the hCD58-Fc-NKG2A chimeric protein, were loaded into the lane marked as NR of each of the blots. Samples in the lane marked as R were treated with a reducing agent, β-mercaptoethanol, and were boiled. Samples in the lane marked as DG were treated with a deglycosylation agent, the reducing agent, and were boiled. The lane marked as L included the protein size ladder. Each individual domain of the chimeric protein was probed using an anti-hCD58 antibody (left blot), an anti-Fc antibody (center blot), or an anti-NKG2A antibody (right blot).

FIG. 15A is a cartoon showing the structure of an illustrative chimeric protein of the present invention: human CD80-Fc-hNKG2A (hCD80-Fc-NKG2A). FIG. 15B are Western blots showing characterization of the hCD80-Fc-NKG2A chimeric protein. The Western blots demonstrate the chimeric protein's native state and tendency to form a multimer. Untreated samples (i.e., without a reducing agent or a deglycosylation agent, yet boiled) of the hCD80-Fc-NKG2A chimeric protein, were loaded into the lane marked as NR of each of the blots. Samples in the lane marked as R were treated with a reducing agent, β-mercaptoethanol, and were boiled. Samples in the lane marked as DG were treated with a deglycosylation agent, the reducing agent, and were boiled. The lane marked as L included the protein size ladder. Each individual domain of the chimeric protein was probed using an anti-CD80 antibody (left blot), an anti-Fc antibody (center blot), or an anti-NKG2A antibody (right blot).

FIG. 16A is a cartoon showing the structure of an illustrative chimeric protein of the present invention: human SLAMF6-Fc-hNKG2A (hSLAMF6-Fc-NKG2A). FIG. 16B are Western blots showing characterization of the hSLAMF6-Fc-NKG2A chimeric protein. The Western blots demonstrate the chimeric protein's native state and tendency to form a multimer. Untreated samples (i.e., without a reducing agent or a deglycosylation agent, yet boiled) of the hSLAMF6-Fc-NKG2A chimeric protein, were loaded into the lane marked as NR of each of the blots. Samples in the lane marked as R were treated with a reducing agent, β-mercaptoethanol, and were boiled. Samples in the lane marked as DG were treated with a deglycosylation agent, the reducing agent, and were boiled. The lane marked as L included the protein size ladder. Each individual domain of the chimeric protein was probed using an anti-hSLAMF6 antibody (left blot), an anti-Fc antibody (center blot), or an anti-NKG2A antibody (right blot).

FIG. 17A is a cartoon showing the structure of an illustrative chimeric protein of the present invention: mouse CD80-Fc-hNKG2A (mCD80-Fc-NKG2A). FIG. 17B are Western blots showing characterization of the mCD80-Fc-NKG2A chimeric protein. The Western blots demonstrate the chimeric protein's native state and tendency to form a multimer. Untreated samples (i.e., without a reducing agent or a deglycosylation agent, yet boiled) of the mCD80-Fc-NKG2A chimeric protein, were loaded into the lane marked as NR of each of the blots. Samples in the lane marked as R were treated with a reducing agent, β-mercaptoethanol, and were boiled. Samples in the lane marked as DG were treated with a deglycosylation agent, the reducing agent, and were boiled. The lane marked as L included the protein size ladder. Each individual domain of the chimeric protein was probed using an anti-mCD80 antibody (left blot), an anti-Fc antibody (center blot), or an anti-NKG2A antibody (right blot).

FIG. 18A is a cartoon showing the structure of an illustrative chimeric protein of the present invention: mouse CD86-Fc-hNKG2A (mCD86-Fc-NKG2A). FIG. 18B are Western blots showing characterization of the mCD86-Fc-NKG2A chimeric protein. The Western blots demonstrate the chimeric protein's native state and tendency to form a multimer. Untreated samples (i.e., without a reducing agent or a deglycosylation agent, yet boiled) of the mCD86-Fc-NKG2A chimeric protein, were loaded into the lane marked as NR of each of the blots. Samples in the lane marked as R were treated with a reducing agent, β-mercaptoethanol, and were boiled. Samples in the lane marked as DG were treated with a deglycosylation agent, the reducing agent, and were boiled. The lane marked as L included the protein size ladder. Each individual domain of the chimeric protein was probed using an anti-mCD86 antibody (left blot), an anti-Fc antibody (center blot), or an anti-NKG2A antibody (right blot).

FIG. 19A is a cartoon showing the structure of an illustrative chimeric protein of the present invention: mouse PD-1-Fc-hNKG2A (mPD-1-Fc-NKG2A). FIG. 19B are Western blots showing characterization of the mPD-1-Fc-NKG2A chimeric protein. The Western blots demonstrate the chimeric protein's native state and tendency to form a multimer. Untreated samples (i.e., without a reducing agent or a deglycosylation agent, yet boiled) of the mPD-1-Fc-NKG2A chimeric protein, were loaded into the lane marked as NR of each of the blots. Samples in the lane marked as R were treated with a reducing agent, β-mercaptoethanol, and were boiled. Samples in the lane marked as DG were treated with a deglycosylation agent, the reducing agent, and were boiled. The lane marked as L included the protein size ladder. Each individual domain of the chimeric protein was probed using an anti-mPD-1 antibody (left blot), an anti-Fc antibody (center blot), or an anti-NKG2A antibody (right blot).

FIG. 20A is a cartoon showing the structure of an illustrative chimeric protein of the present invention: mouse mSIRPα-Fc-hNKG2A (mSIRPα-Fc-NKG2A). FIG. 20B are Western blots showing characterization of the mSIRPα-Fc-NKG2A chimeric protein. The Western blots demonstrate the chimeric protein's native state and tendency to form a multimer. Untreated samples (i.e., without a reducing agent or a deglycosylation agent, yet boiled) of the mSIRPα-Fc-NKG2A chimeric protein, were loaded into the lane marked as NR of each of the blots. Samples in the lane marked as R were treated with a reducing agent, β-mercaptoethanol, and were boiled. Samples in the lane marked as DG were treated with a deglycosylation agent, the reducing agent, and were boiled. The lane marked as L included the protein size ladder. Each individual domain of the chimeric protein was probed using an anti-mSIRPα antibody (left blot), an anti-Fc antibody (center blot), or an anti-NKG2A antibody (right blot).

FIG. 21A is a cartoon showing the structure of an illustrative chimeric protein of the present invention: mouse TGFBR2-Fc-hNKG2A (mTGFBR2-Fc-NKG2A). FIG. 21B are Western blots showing characterization of the mTGFBR2-Fc-NKG2A chimeric protein. The Western blots demonstrate the chimeric protein's native state and tendency to form a multimer. Untreated samples (i.e., without a reducing agent or a deglycosylation agent, yet boiled) of the mTGFBR2-Fc-NKG2A chimeric protein, were loaded into the lane marked as NR of each of the blots. Samples in the lane marked as R were treated with a reducing agent, β-mercaptoethanol, and were boiled. Samples in the lane marked as DG were treated with a deglycosylation agent, the reducing agent, and were boiled. The lane marked as L included the protein size ladder. Each individual domain of the chimeric protein was probed using an anti-mSIRPα antibody (left blot), an anti-Fc antibody (center blot), or an anti-mTGFBR2 antibody (right blot).

FIG. 22 shows the results of ELISA assays demonstrating the dose-dependent binding of the Fc domain of the mSLAMF6-Fc-NKG2A and mPD-1-Fc-NKG2A chimeric proteins to anti-Fc antibody. Anti-mFc was coated on plates and increasing amounts of the indicated chimeric proteins were added to the plates. The binding was detected using an anti-mFc HRP. mFc IgG was used as a positive control.

FIG. 23 shows the results of ELISA assays demonstrating the dose-dependent binding of the Fc domain of the hCD86-Fc-NKG2A and hTGFBR2-Fc-NKG2A chimeric proteins to anti-human Fc antibody. Anti-human IgG was coated on plates and increasing amounts of the indicated chimeric proteins were added to the plates. hIgG was used as a positive control, and mCD80-Fc-NKG2A was used as a negative control. The binding was detected using an anti-human Fcgamma HRP.

FIG. 24 shows the results of ELISA assays demonstrating the dose-dependent binding of the Fc domain of the hCD48-Fc-NKG2A, hCD58-Fc-NKG2A, and hCD80-Fc-NKG2A chimeric proteins to anti-human Fc antibody. Anti-human IgG was coated on plates and increasing amounts of the indicated chimeric proteins were added to the plates. hIgG was used as a positive control. The binding was detected using an anti-human Fcgamma HRP.

FIG. 25 shows the results of ELISA assays demonstrating the dose-dependent binding of the hCD48-Fc-NKG2A, hCD58-Fc-NKG2A, and hCD80-Fc-NKG2A chimeric proteins to HLA-E. HLA-E was coated on plates and increasing amounts of the indicated chimeric proteins were added to the plates. The binding was detected using an anti-human Fcgamma HRP.

FIG. 26 shows the results of ELISA assays demonstrating the dose-dependent binding of the hCD86-Fc-NKG2A chimeric protein to HLA-E. HLA-E was coated on plates and increasing amounts of hCD86-Fc-NKG2A were added to the plates. The binding was detected using an anti-human Fcgamma HRP.

FIG. 27 shows the results of ELISA assays demonstrating the dose-dependent binding of the hCD48-Fc-NKG2A, hCD58-Fc-NKG2A, and hCD80-Fc-NKG2A chimeric proteins to HLA-E. Increasing amounts of the indicated chimeric proteins were coated on plates and detected using an anti-human HLA-E-His. A chimeric protein comprising ECD of a type II transmembrane protein other than NKG2A was used as a negative control.

FIG. 28 shows the results of ELISA assays demonstrating the dose-dependent binding of the hTGFBR2-Fc-NKG2A and hSLAMF6-Fc-NKG2A chimeric proteins to HLA-E. Increasing amounts of the indicated chimeric proteins were coated on plates and detected using an anti-human HLA-E-His. A chimeric protein comprising ECD of a type II transmembrane protein other than NKG2A was used as a negative control.

FIG. 29 shows the results of ELISA assays demonstrating the dose-dependent binding of the hCD48-Fc-NKG2A chimeric protein to h2B4. h2B4 or hCD28-His, which was used as a negative control, were coated on plates and detected using the hCD48-Fc-NKG2A chimeric protein chimeric protein.

FIG. 30 shows the results of ELISA assays demonstrating the dose-dependent binding of the mTGFBR2-Fc-NKG2A chimeric protein to mTGFβ1. mTGFβ1 was coated on plates and detected using mTGFBR2-Fc-NKG2A, or mCD80-Fc-NKG2A, which was used as a negative control.

FIG. 31 shows the results of ELISA assays demonstrating ELISA assays demonstrating the dose-dependent binding of the hCD48-Fc-NKG2A and hCD58-Fc-NKG2A bind chimeric proteins to hCD2. Recombinant human CD2 (rhCD2) protein was coated on plates. Increasing amounts of the indicated chimeric proteins or Recombinant human CD58-Fc fusion protein (rhCD58-Fc) were added and detected using an anti-mFc-HRP (for mCD80-Fc-NKG2A) or anti-hFc-HRP (for rest of the proteins).

FIG. 32 shows the results of ELISA assays demonstrating the dose-dependent binding of the hCD48-Fc-NKG2A chimeric protein to recombinant human 2B4 protein (rh2B4). rh2B4-His was coated on plates. Increasing amounts of the indicated chimeric proteins or rhCD48-Fc were added and detected using an anti-mFc-HRP (for mCD80-Fc-NKG2A) or anti-hFc-HRP (for rest of the proteins).

FIG. 33 shows the results of ELISA assays demonstrating the dose-dependent binding of the mCD86-Fc-NKG2A chimeric protein to mCD28. mCD28-Fc was coated on plates. Increasing amounts of the mCD86-Fc-NKG2A chimeric protein was added and detected using an anti-mFc-HRP.

FIG. 34 shows the results of ELISA assays demonstrating the dose-dependent binding of the hCD80-Fc-NKG2A, hCD86-Fc-NKG2A chimeric proteins to recombinant human CD28 (rhCD28). rhCD28-His was coated on plates. Increasing amounts of the indicated chimeric proteins or hCD86-Fc (a positive control) were added and detected using an anti-mFc-HRP. mCD48-Fc-NKG2A was used a negative control.

FIG. 35 shows the results of ELISA assays demonstrating the dose-dependent binding of the mPD1-Fc-NKG2A, chimeric protein mPD-L1. Increasing amounts of mPD1-Fc-NKG2A were coated on plates, and detected using mPD-L1-His.

FIG. 36 shows the results of ELISA assays demonstrating that the mCD48-Fc-NKG2A chimeric protein binds to both Qa1 and anti-CD48 simultaneously in a dose dependent manner. An anti-mouse Qa1 antibody was coated on plates. Recombinant Qa1 protein, and increasing amounts of the mCD48-Fc-NKG2A chimeric protein was added in that order, and detected using an anti-mCD48 antibody.

FIG. 37 shows the results of ELISA assays demonstrating that the mCD86-Fc-NKG2A chimeric protein binds to both HLA-E and anti-CD86 simultaneously in a dose dependent manner. HLA-E-His was coated on plates. Increasing amounts of the mCD86-Fc-NKG2A chimeric protein was added and detected using an anti-mCD86. A chimeric protein comprising ECD of a type II transmembrane protein other than NKG2A was used as a negative control.

FIG. 38 shows the results of ELISA assays demonstrating that the mSIRPα-Fc-NKG2A chimeric protein binds to both anti-NKG2A and mCD47 simultaneously in a dose dependent manner. anti-NKG2A was coated on plates. Increasing amounts of the mSIRPα-Fc-NKG2A chimeric protein was added and detected using mCD47-His. The CD86-Fc-NKG2A chimeric protein was used as a negative control.

FIG. 39 shows the results of ELISA assays demonstrating that the hPD-1-Fc-NKG2A chimeric protein binds to both hPD-L1 and HLA-E simultaneously in a dose dependent manner. hPD-L1-Fc was coated on plates. Increasing amounts of the hPD-1-Fc-NKG2A chimeric protein was added and detected using HLA-E-His. A chimeric protein comprising ECD of a type II transmembrane protein other than NKG2A was used as a negative control.

FIG. 40 shows the results of ELISA assays demonstrating that the hCD80-Fc-NKG2A chimeric protein binds to both rhCD28 and HLA-E simultaneously in a dose dependent manner. rhCD28-Fc was coated on plates. Increasing amounts of the hCD80-Fc-NKG2A chimeric protein was added and detected using HLA-E-His.

FIG. 41 shows the results of ELISA assays demonstrating that the hCD86-Fc-NKG2A chimeric protein binds to both rhCD28 and HLA-E simultaneously in a dose dependent manner. rhCD28-Fc was coated on plates. Increasing amounts of the hCD86-Fc-NKG2A chimeric protein was added and detected using HLA-E-His. A chimeric protein comprising ECD of a type II transmembrane protein other than NKG2A was used as a negative control.

FIG. 42 shows the results of ELISA assays demonstrating that the hSLAMF6-Fc-NKG2A chimeric protein binds to both recombinant human SLAMF6 (rhSLAMF6) and HLA-E simultaneously in a dose dependent manner. rhSLAMF6-Fc was coated on plates. Increasing amounts of the hSLAMF6-Fc-NKG2A chimeric protein was added and detected using HLA-E-His. A chimeric protein comprising ECD of a type II transmembrane protein other than NKG2A was used as a negative control.

FIG. 43 shows the results of ELISA assays demonstrating that the mPD-1-Fc-NKG2A chimeric protein binds to both recombinant mouse PD-L1 (rmPD-L1) and HLA-E simultaneously in a dose dependent manner. rmPD-L1-Fc was coated on plates. Increasing amounts of the mPD-1-Fc-NKG2A chimeric protein was added and detected using HLA-E-His. A chimeric protein comprising ECD of a type II transmembrane protein other than NKG2A was used as a negative control.

FIG. 44 shows the results of ELISA assays demonstrating simultaneous binding to two ligands by the chimeric proteins disclosed herein. (i) recombinant human 2B4-Fc fusion protein (rh2B4-Fc) was coated on plates. Increasing amounts of the hCD48-Fc-NKG2A or hCD86-Fc-NKG2A chimeric proteins were added and detected using HLA-E-His. The hCD86-Fc-NKG2A chimeric protein was used as a negative control (Negative control #1). These data demonstrate that the hCD48-Fc-NKG2A chimeric protein binds to both rh2B4 and HLA-E simultaneously in a dose dependent manner. (ii) recombinant human CD2-Fc fusion protein (rhCD2-Fc) was coated on plates. Increasing amounts of the hCD58-Fc-NKG2A or hCD86-Fc-NKG2A chimeric proteins were added and detected using HLA-E-His. The hCD86-Fc-NKG2A chimeric protein was used as a negative control (Negative control #2). These data demonstrate that the hCD58-Fc-NKG2A chimeric protein binds to both human CD2 and HLA-E simultaneously in a dose dependent manner.

FIG. 45 shows the flow cytometry analysis showing the generation of the CHO-K1 cells expressing h2B4 (the binding partner of CD48). A positive clone was stained with an anti-2B4 antibody. A control clone was stained with isotype control antibody.

FIG. 46A and FIG. 46B show the binding of the hCD48-Fc-NKG2A chimeric protein to Wild type (WT) CHO-K1 cells (FIG. 46A) or the CHO-K1/h2B4 cells (FIG. 46B) as measured by flow cytometry. The dose dependent shifts in the CHO-K1/h2B4 cells, but not WT CHO-K1 cells illustrate dose dependent binding of the hCD48-Fc-NKG2A chimeric protein to h2B4 expressed by the CHO-K1/h2B4 cells.

FIG. 47 shows the quantitation of the binding of the hCD48-Fc-NKG2A chimeric protein to the CHO-K1/h2B4 cells in comparison to WT CHO-K1 cells as measured by flow cytometry.

FIG. 48 shows the flow cytometry analysis showing the generation of the CHO-K1 cells expressing m2B4 (the binding partner of CD48). A positive clone was stained with an anti-2B4 antibody. A control was stained with isotype control antibody. An unstained control was also analyzed.

FIG. 49A and FIG. 49B show the binding of the mCD48-Fc-NKG2A chimeric protein to WT CHO-K1 cells (FIG. 49A) or the CHO-K1/m2B4 cells (FIG. 49B) as measured by flow cytometry. The dose dependent shifts in the CHO-K1/m2B4 cells, but not WT CHO-K1 cells illustrate dose dependent binding of the mCD48-Fc-NKG2A chimeric protein to m2B4 expressed by the CHO-K1/m2B4 cells.

FIG. 50 shows the quantitation of the binding of the mCD48-Fc-NKG2A chimeric protein to the CHO-K1/m2B4 cells in comparison to WT CHO-K1 cells as measured by flow cytometry.

FIG. 51 shows the flow cytometry analysis showing the generation of the CHO-K1 cells expressing hPD-L1. A positive clone was stained with an anti-hPD-L1 antibody. Unstained cells were used as the negative control.

FIG. 52A and FIG. 52B show the binding of the hPD-1-Fc-NKG2A chimeric protein to WT CHO-K1 cells (FIG. 52A) or the CHO-K1/hPD-L1 cells (FIG. 52B) as measured by flow cytometry. The dose dependent shifts in the CHO-K1/hPD-L1 cells, but not WT CHO-K1 cells illustrate dose dependent binding of the hPD-1-Fc-NKG2A chimeric protein to hPD-L1 expressed by the CHO-K1/hPD-L1 cells.

FIG. 53 shows the quantitation of the binding of the hPD-1-Fc-NKG2A chimeric protein to the CHO-K1/hPD-L1 cells in comparison to WT CHO-K1 cells as measured by flow cytometry.

FIG. 54 shows the flow cytometry analysis showing the generation of the CHO-K1 cells expressing mPD-L1. A positive clone was stained with an anti-mPD-L1 antibody. Unstained cells were used as the negative control.

FIG. 55A and FIG. 55B show the binding of the mPD-1-Fc-NKG2A chimeric protein to WT CHO-K1 cells (FIG. 55A) or the CHO-K1/mPD-L1 cells (FIG. 55B) as measured by flow cytometry. The greater dose dependent shifts in the CHO-K1/mPD-L1 cells, compared to WT CHO-K1 cells illustrate dose dependent binding of the mPD-1-Fc-NKG2A chimeric protein to mPD-L1 expressed by the CHO-K1/mPD-L1 cells.

FIG. 56 shows the quantitation of the binding of the mPD-1-Fc-NKG2A chimeric protein to the CHO-K1/mPD-L1 cells in comparison to WT CHO-K1 cells as measured by flow cytometry.

FIG. 57 shows the flow cytometry analysis showing the generation of the CHO-K1 cells expressing mQa1 (the binding partner of CD48). A positive clone was stained with an anti-mQa1 antibody. A control was stained with isotype control antibody. An unstained control was also analyzed.

FIG. 58A and FIG. 58B show the binding of the mCD48-Fc-NKG2A chimeric protein to WT CHO-K1 cells (FIG. 58A) or the CHO-K1/mQa1 cells (FIG. 58B) as measured by flow cytometry. The dose dependent shifts in the CHO-K1/mQa1 cells, but not WT CHO-K1 cells illustrate dose dependent binding of the mCD48-Fc-NKG2A chimeric protein to mQa1 expressed by the CHO-K1/mQa1 cells.

FIG. 59 shows the quantitation of the binding of the mCD48-Fc-NKG2A chimeric protein to the CHO-K1/mQa1 cells in comparison to WT CHO-K1 cells as measured by flow cytometry.

FIG. 60 shows the flow cytometry analysis showing the generation of the CHO-K1 cells expressing hCD2. A positive clone was stained with an anti-hCD2 antibody. Unstained cells and isotype control-stained cells were used as the negative controls.

FIG. 61A and FIG. 61B show the binding of the hCD58-Fc-NKG2A chimeric protein to WT CHO-K1 cells (FIG. 61A) or the CHO-K1/hCD2 cells (FIG. 61B) as measured by flow cytometry. The dose dependent shifts in the CHO-K1/hCD2 cells, but not WT CHO-K1 cells illustrate dose dependent binding of the hCD58-Fc-NKG2A chimeric protein to hCD2 expressed by the CHO-K1/hCD2 cells.

FIG. 62 shows the quantitation of the binding of the hCD58-Fc-NKG2A chimeric protein to the CHO-K1/hCD2 cells in comparison to WT CHO-K1 cells as measured by flow cytometry.

FIG. 63 shows the flow cytometry analysis showing the generation of the CHO-K1 cells expressing hCD28. Two positive clones were stained with an anti-hCD28 antibody. Unstained cells and isotype control-stained cells were used as the negative controls.

FIG. 64A and FIG. 64B show the binding of the hCD86-Fc-NKG2A chimeric protein to WT CHO-K1 cells (FIG. 64A) or the CHO-K1/hCD28 cells (FIG. 64B) as measured by flow cytometry. The dose dependent shifts in the CHO-K1/hCD28 cells, but not WT CHO-K1 cells illustrate dose dependent binding of the hCD86-Fc-NKG2A chimeric protein to hCD28 expressed by the CHO-K1/hCD28 cells.

FIG. 65 shows the quantitation of the binding of the hCD86-Fc-NKG2A chimeric protein to the CHO-K1/hCD28 cells in comparison to WT CHO-K1 cells as measured by flow cytometry.

FIG. 66 shows the flow cytometry analysis showing the generation of the CHO-K1 cells expressing mCD28. Two positive clones were stained with an anti-mCD28 antibody. Unstained cells and isotype control-stained cells were used as the negative controls.

FIG. 67A and FIG. 67B show the binding of the mCD80-Fc-NKG2A chimeric protein to WT CHO-K1 cells (FIG. 67A) or the CHO-K1/mCD28 cells (FIG. 67B) as measured by flow cytometry. The dose dependent shifts in the CHO-K1/mCD28 cells, but not WT CHO-K1 cells illustrate dose dependent binding of the mCD80-Fc-NKG2A chimeric protein to mCD28 expressed by the CHO-K1/mCD28 cells.

FIG. 68 shows the quantitation of the binding of the mCD80-Fc-NKG2A chimeric protein to the CHO-K1/mCD28 cells in comparison to WT CHO-K1 cells as measured by flow cytometry.

FIG. 69A and FIG. 69B show the binding of the mCD86-Fc-NKG2A chimeric protein to WT CHO-K1 cells (FIG. 69A) or the CHO-K1/mCD28 cells (FIG. 69B) as measured by flow cytometry. The dose dependent shifts in the CHO-K1/mCD28 cells, compared to WT CHO-K1 cells illustrate dose dependent binding of the mCD86-Fc-NKG2A chimeric protein to mCD28 expressed by the CHO-K1/mCD28 cells.

FIG. 70 shows the quantitation of the binding of the mCD86-Fc-NKG2A chimeric protein to the CHO-K1/mCD28 cells in comparison to WT CHO-K1 cells as measured by flow cytometry.

FIG. 71 shows the results of luciferase assays illustrating that the mCD48-Fc-NKG2A chimeric protein activates 2B4 signaling in a dose dependent manner. The CHO-K1/m2B4 cells used in these assays harbor an NFκB-luciferase reporter that is sensitive to the binding of a ligand to the m2B4 protein that the cells express. WT CHO-K1 cells were used as a negative control. Increasing amounts of the mCD48-Fc-NKG2A chimeric protein was incubated with the CHO-K1/m2B4 cells, or WT CHO-K1 cells, and the activation of the m2B4 was measured by a luciferase assay.

FIG. 72 shows the results of luciferase assays illustrating that the mCD48-Fc-NKG2A chimeric protein activates m2B4 and mCD2 signaling in a dose dependent manner. The CHO-K1/m2B4 and CHO-K1/mCD2 cells used in these assays harbor an NFκB-luciferase reporter that is sensitive to the binding of a ligand to the m2B4 and mCD2 protein, respectively, that the cells express. WT CHO-K1 cells were used as a negative control. Increasing amounts of the mCD48-Fc-NKG2A chimeric protein was incubated with the CHO-K1/m2B4 cells, CHO-K1/mCD2 cells or WT CHO-K1 cells, and the activation of the m2B4 cells or mCD2 was measured by a luciferase assay.

FIG. 73 shows the results of luciferase assays illustrating that the mSLAMF6-Fc-NKG2A chimeric protein activates mSLAMF6 signaling in a dose dependent manner. The CHO-K1/mSLAMF6 cells used in these assays harbor an NFκB-luciferase reporter that is sensitive to the binding of a ligand to the mSLAMF6 protein that the cells express. WT CHO-K1 cells were used as a negative control. Increasing amounts of the mSLAMF6-Fc-NKG2A chimeric protein was incubated with the CHO-K1/mSLAMF6 cells or WT CHO-K1 cells, and the activation of the CHO-K1/mSLAMF6 cells was measured by a luciferase assay.

FIG. 74 shows the results of luciferase assays illustrating that the hCD80-Fc-NKG2A chimeric protein activates CD28 signaling in a dose dependent manner. The CHO-K1/CD28 cells used in these assays harbor an NFκB-luciferase reporter that is sensitive to the binding of a ligand to the CD28 protein that the cells express. WT CHO-K1 cells were used as a negative control. Increasing amounts of the hCD80-Fc-NKG2A chimeric protein was incubated with the CHO-K1/CD28 cells, or WT CHO-K1 cells, and the activation of the CD28 was measured by a luciferase assay.

FIG. 75 shows the results of luciferase assays illustrating that the hCD86-Fc-NKG2A chimeric protein activates hCD28 signaling in a dose dependent manner. The CHO-K1/hCD28 cells used in these assays harbor an NFκB-luciferase reporter that is sensitive to the binding of a ligand to the hCD28 protein that the cells express. WT CHO-K1 cells were used as a negative control. Increasing amounts of the hCD86-Fc-NKG2A chimeric protein was incubated with the CHO-K1/hCD28 cells, or WT CHO-K1 cells, and the activation of the hCD28 was measured by a luciferase assay.

FIG. 76 shows the results of luciferase assays illustrating that the hPD-1-Fc-NKG2A chimeric protein activates HLA-E signaling in a dose dependent manner. The CHO-K1/HLA-E cells used in these assays harbor an NFκB-luciferase reporter that is sensitive to the binding of a ligand to the HLA-E protein that the cells express. WT CHO-K1 cells were used as a negative control. Increasing amounts of the hPD-1-Fc-NKG2A chimeric protein was incubated with the CHO-K1/HLA-E cells, or WT CHO-K1 cells, and the activation of the HLA-E was measured by a luciferase assay.

FIG. 77 shows the results of luciferase assays illustrating that the mCD80-Fc-NKG2A chimeric protein activates Qa1 signaling in a dose dependent manner. The CHO-K1/Qa1 cells used in these assays harbor an NFκB-luciferase reporter that is sensitive to the binding of a ligand to the Qa1 protein that the cells express. WT CHO-K1 cells were used as a negative control. Increasing amounts of the mCD80-Fc-NKG2A chimeric protein was incubated with the CHO-K1/Qa1 cells, or WT CHO-K1 cells, and the activation of the Qa1 was measured by a luciferase assay.

FIG. 78 shows the results of luciferase assays illustrating that the mPD-1-Fc-NKG2A chimeric protein activates Qa1 signaling in a dose dependent manner. The CHO-K1/Qa1 cells used in these assays harbor an NFκB-luciferase reporter that is sensitive to the binding of a ligand to the Qa1 protein that the cells express. WT CHO-K1 cells were used as a negative control. Increasing amounts of the mPD-1-Fc-NKG2A chimeric protein was incubated with the CHO-K1/Qa1 cells, or WT CHO-K1 cells, and the activation of the Qa1 was measured by a luciferase assay.

FIG. 79 shows the results of luciferase assays illustrating that the TGFBR2-Fc-NKG2A chimeric protein activates Qa1 signaling in a dose dependent manner. The CHO-K1/Qa1 cells used in these assays harbor an NFκB-luciferase reporter that is sensitive to the binding of a ligand to the Qa1 protein that the cells express. WT CHO-K1 cells were used as a negative control. Increasing amounts of the TGFBR2-Fc-NKG2A chimeric protein was incubated with the CHO-K1/Qa1 cells, or WT CHO-K1 cells, and the activation of the Qa1 was measured by a luciferase assay.

FIG. 80 shows the results of luciferase assays illustrating that the hCD48-Fc-NKG2A chimeric protein activates h2B4 signaling in a dose dependent manner. The CHO-K1/h2B4 cells used in these assays harbor an NFκB-luciferase reporter that is sensitive to the binding of a ligand to the h2B4 protein that the cells express. WT CHO-K1 cells were used as a negative control. Increasing amounts of the hCD48-Fc-NKG2A chimeric protein was incubated with the CHO-K1/h2B4 cells, or WT CHO-K1 cells, and the activation of the h2B4 was measured by a luciferase assay.

FIG. 81 shows the results of luciferase assays illustrating that the hCD58-Fc-NKG2A chimeric protein activates hCD2 signaling in a dose dependent manner. The CHO-K1/hCD2 cells used in these assays harbor an NFκB-luciferase reporter that is sensitive to the binding of a ligand to the hCD2 protein that the cells express. WT CHO-K1 cells were used as a negative control. Increasing amounts of the hCD58-Fc-NKG2A chimeric protein was incubated with the CHO-K1/hCD2 cells, or WT CHO-K1 cells, and the activation of the hCD2 was measured by a luciferase assay.

FIG. 82 shows the binding of the hCD86-Fc-NKG2A chimeric protein to NK92-CD16V cells as measured by flow cytometry. The dose dependent shifts demonstrate the dose dependent binding of the hCD86-Fc-NKG2A chimeric protein to NK92-CD16V cells.

FIG. 83A and FIG. 83B show the binding of the hCD48-Fc-NKG2A chimeric protein to NK92-CD16V cells. FIG. 83A results of a flow cytometry-based binding assay. FIG. 83B shows the quantitation of the binding of the hCD48-Fc-NKG2A chimeric protein to NK92-CD16V effector cells. The dose dependent binding demonstrate that the hCD48-Fc-NKG2A chimeric protein effectively bind to NK92-CD16V cells.

FIG. 84 shows the binding of the hCD80-Fc-NKG2A chimeric protein to NK92-CD16V cells as measured by flow cytometry. The dose dependent shifts demonstrate the dose dependent binding of the hCD80-Fc-NKG2A chimeric protein to NK92-CD16V cells.

FIG. 85 shows the binding of the hPD-1-Fc-NKG2A chimeric protein to NK92-CD16V cells as measured by flow cytometry. The dose dependent shifts demonstrate the dose dependent binding of the hPD-1-Fc-NKG2A chimeric protein to NK92-CD16V cells.

FIG. 86 demonstrates that the mCD86-Fc-NKG2A chimeric protein induces apoptosis of antigen positive (OVA+) target cells mediated by the antigen activated T cells (OT-1 naïve T cells) in a dose-dependent manner. Increasing amounts of the mCD86-Fc-NKG2A chimeric protein was incubated with OT-1 naïve T cells (effector cells) and OVA+ cells (target cells) at the effector cells: target cell ration of 5:1. Apoptosis was assessed by measuring caspase 3/7 activity.

FIG. 87 demonstrates that the hCD86-Fc-NKG2A chimeric protein, in combination with an anti-EGFR antibody, induces NK cell-mediated antibody dependent cellular cytotoxicity in the EGFR-positive A431 human non-small cell lung cancer (NSCLC) cells (target cells) in a dose-dependent manner. Increasing amounts of the hCD86-Fc-NKG2A chimeric protein and 1 μg/ml Cetuximab NK92-CD16V cells (effector cells) and A431 cells (target cells) at the effector cells: target cell ration of 5:1. Apoptosis was assessed by measuring annexin V.

FIG. 88 demonstrates that the hCD86-Fc-NKG2A chimeric protein, in combination with an anti-EGFR antibody, induces NK cell-mediated antibody dependent cellular cytotoxicity in the EGFR-positive A549 human lung carcinoma cells (target cells) in a dose-dependent manner. Increasing amounts of the hCD86-Fc-NKG2A chimeric protein and 10 μg/ml Cetuximab were incubated with NK92-CD16V cells (effector cells) and A549 cells (target cells) at the effector cells: target cell ration of 5:1. Apoptosis was assessed by measuring annexin V.

FIG. 89A and FIG. 89B demonstrate that the mCD86-Fc-NKG2A chimeric protein induces apoptosis mediated by freshly isolated splenocytes in the murine reticulum cell sarcoma A20 cells in a dose-dependent manner. Increasing amounts of the mCD86-Fc-NKG2A chimeric protein was incubated with freshly isolated splenocytes (effector cells) and A20 cells (target cells). Negative controls included splenocytes only (without target cells) and A20 cells only (without effector cells). Apoptosis was assessed by measuring caspase 3/7 activity. FIG. 89A shows the caspase 3/7 activity as function of time. FIG. 89B shows a bar graph showing the caspase 3/7 activity at 3.5 hr. note that no apoatosis was observed without splenocytes (the black bar).

FIG. 90A and FIG. 90B demonstrate the efficacy of the CD86-Fc-NKG2A chimeric protein against the murine colorectal carcinoma cell line CT26 allografts. Mice were inoculated with CT26 cells. After the tumors were established, the mice were randomly assigned to the following treatment groups: (1) untreated, (2) 100 μg/mouse of an anti-Qa1 antibody (Bioxcell Clone 4C2.4A7.5H11), and (3) 300 μg/mouse of the CD86-Fc-NKG2A chimeric protein. The mice were treated on days 5, 8, 11, 14, 16, and 18 days post-inoculation. Tumor volumes were measured. FIG. 90A shows the tumor volumes plotted as a function of time. FIG. 90B shows the tumor volumes on day 18. ** denotes p≤0.01, and *** denotes p≤0.001 between the indicated groups.

FIG. 91A and FIG. 91B demonstrate the efficacy of the CD80-Fc-NKG2A chimeric protein against the murine colorectal carcinoma cell line CT26 allografts. Mice were inoculated with CT26 cells. After the tumors were established, the mice were randomly assigned to the following treatment groups: (1) untreated, (2) 100 μg/mouse of an anti-Qa1 antibody (Bioxcell Clone 4C2.4A7.5H11), and (3) 300 μg/mouse of the CD80-Fc-NKG2A chimeric protein. The mice were treated on 5, 8, 11, 14, 16, and 18 post-inoculation. Tumor volumes were measured. FIG. 91A shows the tumor volumes plotted as a function of time. FIG. 91B shows the tumor volumes on day 18. ** denotes p≤0.01 between the indicated groups.

FIG. 92A and FIG. 92B demonstrate the efficacy of the CD48-Fc-NKG2A chimeric protein against the murine colorectal carcinoma cell line CT26 allografts. Mice were inoculated with CT26 cells. After the tumors were established, the mice were randomly assigned to the following treatment groups: (1) untreated, (2) 100 μg/mouse of an anti-Qa1 antibody (Bioxcell Clone 4C2.4A7.5H11), and (3) 300 μg/mouse of the CD48-Fc-NKG2A chimeric protein. The mice were treated on days 5, 8, 11, 14, 16, and 18 post-inoculation. Tumor volumes were measured. FIG. 92A shows the tumor volumes plotted as a function of time. FIG. 92B shows the tumor volumes on day 18. ** denotes p≤0.01 between the indicated groups.

FIG. 93A and FIG. 93B demonstrate the efficacy of the PD-1-Fc-NKG2A chimeric protein against murine colorectal carcinoma cell line CT26 allografts. Mice were inoculated with CT26 cells. After the tumors were established, the mice were randomly assigned to the following treatment groups: (1) untreated, (2) 100 μg/mouse of an anti-Qa1 antibody (Bioxcell Clone 4C2.4A7.5H11), and (3) 300 μg/mouse of the PD-1-Fc-NKG2A chimeric protein. The mice were treated on days 5, 8, 11, and 14 days post-inoculation. Tumor volumes were measured. FIG. 93A shows the tumor volumes plotted as a function of time. FIG. 93B shows the tumor volumes on day 11. * denotes p≤0.05 between the indicated groups.

FIG. 94A and FIG. 94B demonstrate the efficacy of the CD86-Fc-NKG2A chimeric protein against allografts of the murine lymphoma cell line EG7, which has been engineered to express the novel antigen OVA (EG7-OVA). Mice were inoculated with EG7-OVA cells, and infused with CD4 and CD8 OVA specific T cells. Mice were randomly assigned to one of four treatment groups: (1) untreated, (2) 100 μg/mouse anti-PD1 antibody (Bioxcell clone RMP1-14), (3) 100 μg/mouse anti-NKG2A (BioXcell clone 20D5), and (4) 300 μg/mouse of the CD86-Fc-NKG2A chimeric protein. The mice were treated on days 0, 2, 4, 6, 8 and 10 post-inoculation. The mice were treated on days 0, 2, 4, 6, 8 and 10 post-inoculation. Tumor volumes were measured on indicated days. FIG. 94A shows the tumor volumes plotted as a function of time. FIG. 94B shows the tumor volumes on day 7. denotes p≤0.01 between the indicated groups.

FIG. 95 shows the effect of the CD86-Fc-NKG2A chimeric protein effector memory T cells (T_{EM} cells) against tumor allograft. Tail blood from was drawn from the mice described in the description of FIG. 94A and FIG. 94B and analyzed the immune makeup in the peripheral system on the indicated days. The fraction of effector memory T cells (T_{EM} cells) on days 0 and 3 are plotted. Cells from OT-I:GFP from transgenic to OT-1 mice were used to measure OT-1 cells.

FIG. 96A and FIG. 96B demonstrate the efficacy of the CD86-Fc-NKG2A chimeric protein against allografts of the murine myelomonocytic leukemia cell line WEHI-3. Balb/c mice were inoculated with WEHI-3 cells. After the tumors were established, the mice were randomly assigned to the following treatment groups: (1) untreated, (2) 100 μg/mouse of an anti-PD1 antibody (Bioxcell clone RMP1-14), (3) 100 μg/mouse of an anti-NKG2A antibody (BioXcell clone 20D5) and (4) 300 μg/mouse of the CD86-Fc-NKG2A chimeric protein. The mice were treated on days 0, 2, 4, 6, 8 and 10 post-inoculation. The mice were treated six times, two days apart. Tumor volumes were measured on indicated days. FIG. 96A shows the tumor volumes plotted as a function of time. FIG. 96B shows the tumor volumes on day 18. * denotes p≤0.05 between the indicated groups, and *** denotes p≤0.001 between the indicated groups.

FIG. 97A and FIG. 97B demonstrate the efficacy of the SIRPα-Fc-NKG2A chimeric protein against allografts of the murine myelomonocytic leukemia cell line WEHI-3. Balb/c mice were inoculated with WEHI-3 cells. After the tumors were established, the mice were randomly assigned to the following treatment groups: (1) untreated, (2) 100 μg/mouse of an anti-PD1 antibody (Bioxcell clone RMP1-14), (3) 100 μg/mouse of an anti-NKG2A antibody (BioXcell clone 20D5) and (4) 300 μg/mouse of the SIRPα-Fc-NKG2A chimeric protein. The mice were treated on days 0, 2, 4, 6, 8 and 10 post-inoculation. The mice were treated six times, two days apart. Tumor volumes were measured on indicated days. FIG. 97A shows the tumor volumes plotted as a function of time. FIG. 97B shows the tumor volumes on day 18. * denotes p≤0.05 between the indicated groups, and *** denotes p≤0.001 between the indicated groups.

FIG. 98A and FIG. 98B demonstrate the efficacy of the CD48-Fc-NKG2A chimeric protein against allografts of the murine myelomonocytic leukemia cell line WEHI-3. Balb/c mice were inoculated with WEHI-3 cells. After the tumors were established, the mice were randomly assigned to the following treatment groups: (1) untreated, (2) 100 μg/mouse of an anti-PD1 antibody (Bioxcell clone RMP1-14), (3) 100 μg/mouse of an anti-NKG2A antibody (BioXcell clone 20D5) and (4) 300 μg/mouse of the CD48-Fc-NKG2A chimeric protein. The mice were treated on days 0, 2, 4, 6, 8 and 10 post-inoculation. The mice were treated six times, two days apart. Tumor volumes were measured on indicated days. FIG. 98A shows the tumor volumes plotted as a function of time. FIG. 98B shows the tumor volumes on day 18. * denotes p≤0.05 between the indicated groups, and ** denotes p≤0.01 between the indicated groups.

FIG. 99A and FIG. 99B demonstrate the efficacy of the TGFBR2-Fc-NKG2A chimeric protein against allografts of the murine myelomonocytic leukemia cell line WEHI-3. Balb/c mice were inoculated with WEHI-3 cells. After the tumors were established, the mice were randomly assigned to the following treatment groups: (1) untreated, (2) 100 μg/mouse of an anti-PD1 antibody (Bioxcell clone RMP1-14), (3) 100 μg/mouse of an anti-NKG2A antibody (BioXcell clone 20D5) and (4) 300 μg/mouse of the TGFBR2-Fc-NKG2A chimeric protein. The mice were treated on days 0, 2, 4, 6, 8 and 10 post-inoculation. The mice were treated six times, two days apart. Tumor volumes were measured on indicated days. FIG. 99A shows the tumor volumes plotted as a function of time. FIG. 99B shows the tumor volumes on day 18. * denotes p≤0.05 between the indicated groups, and ** denotes p≤0.01 between the indicated groups.

FIG. 100A and FIG. 100B demonstrate CD8 T cells are required for the anti-tumor effect of the CD86-Fc-NKG2A chimeric protein against allografts of the murine myelomonocytic leukemia cell line WEHI-3. Balb/c mice were inoculated with WEHI-3 cells. After the tumors were established, the mice were randomly assigned to the following treatment groups: (1) 250 μg/mouse of an anti-CD8a antibody (Bioxcell clone 2.43), (2) 300 μg/mouse of the CD86-Fc-NKG2A chimeric protein, and (3) 250 μg/mouse of the anti-CD8a antibody (Bioxcell clone 2.43)+300 μg/mouse of the CD86-Fc-NKG2A chimeric protein. Tumor volumes were measured on indicated days. FIG. 100A shows the tumor volumes plotted as a function of time. FIG. 100B shows the tumor volumes on day 18. * denotes p≤0.05 between the indicated groups.

FIG. 101A to FIG. 101C demonstrate that CD86-Fc-NKG2A induced the growth of cytokine secretory cells in the spleen compared to both untreated and anti-NKG2A antibody therapy. Balb/c mice were inoculated with WEHI-3 cells. After the tumors were established, the mice were randomly assigned to the following treatment groups: (1) untreated, (2) 100 μg/mouse of an anti-NKG2A antibody (BioXcell clone 20D5) and (3) 300 μg/mouse of the CD86-Fc-NKG2A chimeric protein. The mice were treated on days 1, 3, 5, and 7 post-inoculation. At day 8 post treatment, the mice were sacrificed, their spleens were isolated, digested and analyzed for the amounts of the different immune cells. FIG. 101A shows the CD3- CD11 b+CD27+ cells in the spleens of the mice from indicated treatment groups. FIG. 101B shows the CD3- NKP46+CD11b+CD27+ cells in the spleens of the mice from indicated treatment groups. FIG. 101C shows the CD3- KLRG1+CD11b+CD27+ cells in the spleens of the mice from indicated treatment groups. Statistical analyses were performed using a Students T test and *p<0.05, **p<0.01, and ***p<0.001.

FIG. 102A and FIG. 102B demonstrate that CD86-Fc-NKG2A induced the growth of activated cytotoxic T cells and CD107+cell (a marker for enhanced cytotoxicity) in the spleen of treated mice compared to untreated mice. FIG. 102A shows the PD-1+ cytotoxic T lymphocytes (CTLs) in the spleens of the mice from indicated treatment groups.

FIG. 102B shows the CD107+ cells in the spleens of the mice from indicated treatment groups. Statistical analyses were performed using a Students T test and *p<0.05, and **p<0.01.

FIG. 103 demonstrates that CD86-Fc-NKG2A induced the enhanced infiltration of immune cells into the tumor with the cytolytic marker granzyme B in the tumors of treated mice compared to untreated. Balb/c mice were inoculated with WEHI-3 cells. Statistical analyses were performed using a Students T test and *p<0.05.

FIG. 104A to FIG. 104C demonstrate that CD86-Fc-NKG2A enhanced infiltration of immune cells into the tumor that express potent activation markers (CD137, IFN-γ, and PD1). FIG. 104A shows the CD137+ cells in the tumors of the mice from indicated treatment groups. FIG. 104B shows the IFNγ+ cells in the tumors of the mice from indicated treatment groups. FIG. 104C shows the PD-1+ cytotoxic T lymphocytes (CTLs) in the tumors of the mice from indicated treatment groups. Statistical analyses were performed using a Students T test and *p<0.05.

FIG. 105A to FIG. 105C demonstrate that CD86-Fc-NKG2A induced enhanced infiltration of immune cells in the draining lymph node of effector memory T cells, central memory t cells, and NKG2A+CD8+ T cells indicating mCD86-Fc-NKG2A was able to induce the infiltration and proliferation of these important effector immune cells. FIG. 105A shows the effector memory T cells in the lymph nodes of the mice from indicated treatment groups. FIG. 105B shows the central memory T cells in the lymph nodes of the mice from indicated treatment groups. FIG. 105C shows the NKG2a+ cytotoxic T lymphocytes (CTLs) in the lymph nodes of the mice from indicated treatment groups. Statistical analyses were performed using a Students T test and *p<0.05, **p<0.01, and ***p<0.001.

### DETAILED DESCRIPTION

The present invention is based, in part, on the development of chimeric proteins that block immune inhibitory signals originating from a cancer cell or a virus-infected cell and/or prevent reception of inhibitory signals by a Natural killer (NK) cell.

NK cells are lymphocytes that can mediate lysis of certain tumor cells and virus-infected cells without previous activation; they can also regulate specific humoral and cell-mediated immunity. NK cells express C-Type Lectin receptors which receive an inhibitory signal when bound to non-classical MHC Class I Proteins, e.g., HLA-E (in humans) and Qa1 (in mice). HLA-E/Qa1 are expressed by virtually all cells and are often upregulated by cancer cells. An example of C-Type Lectin receptors expressed by NK cells are the members of the NKG2 family of receptors, which includes NKG2A, NKG2B, NKG2C, NKG2D, NKG2E, NKG2F, and NKG2H. NKG2A, for example, dimerizes with CD94 (KLRD1), which stabilizes NKG2A at the surface and allows the NKG2A/CD94 heterodimer to recognize and bind HLA-E, thereby receiving an immune inhibitory signal. NKG2A is also expressed by cytotoxic T cells, Gamma delta (γδ) T cells, and Natural killer T (NKT) cells. Blocking NKG2A's reception of an immune inhibitory signal, from a cancer cell or a virus-infected cell, will prevent inhibition of the NK cell and will allow it to kill the cancer cell or the virus-infected cell.

The chimeric proteins of the present invention can be engineered comprising a portion of the extracellular domain of a Type I transmembrane protein or a portion of a membrane-anchored extracellular protein and the extracellular domain of a Type II transmembrane protein in which the Type II transmembrane protein is naturally expressed on the surface of a Natural Killer (NK) cell. These chimeric proteins can inhibit transmission and/or reception of immunosuppressive signals. In some instances, the Type I transmembrane protein end of a chimeric protein disrupts, blocks, reduces, inhibits and/or sequesters the transmission of immune inhibitory signals, e.g., originating from a cancer cell or a virus-infected cell that is attempting to avoid its detection and/or destruction. In other instances, the Type I transmembrane protein may provide an immune-stimulatory signal that increases the activity of another immune cell. The Type II transmembrane protein end of a chimeric protein disrupts, blocks, reduces, inhibits and/or sequesters the inhibitory signals thereby preventing their reception by an NK cell. Together, these two actions allow for an anti-cancer attack or an attack of a virus-infected cell by the NK cell.

In embodiments, an extracellular domain refers to a portion of a transmembrane protein which is capable of interacting with the extracellular environment. In embodiments, an extracellular domain refers to a portion of a transmembrane protein which is sufficient for binding to a ligand or receptor and is effective in transmitting a signal to a cell. In embodiments, an extracellular domain is the entire amino acid sequence of a transmembrane protein which is normally present at the exterior of a cell or of the cell membrane. In embodiments, an extracellular domain is that portion of an amino acid sequence of a transmembrane protein which is external of a cell or of the cell membrane and is needed for signal transduction and/or ligand binding as may be assayed using methods know in the art (e.g., in vitro ligand binding and/or cellular activation assays).

Transmembrane proteins typically consist of an extracellular domain, one or a series of transmembrane domains, and an intracellular domain. Without wishing to be bound by theory, the extracellular domain of a transmembrane protein is responsible for interacting with a soluble receptor or ligand or membrane-bound receptor or ligand (i.e., a membrane of an adjacent cell). Without wishing to be bound by theory, the trans-membrane domain(s) is responsible for localizing the transmembrane protein to the plasma membrane. Without wishing to be bound by theory, the intracellular domain of a transmembrane protein is responsible for coordinating interactions with cellular signaling molecules to coordinate intracellular responses with the extracellular environment (or visa-versa).

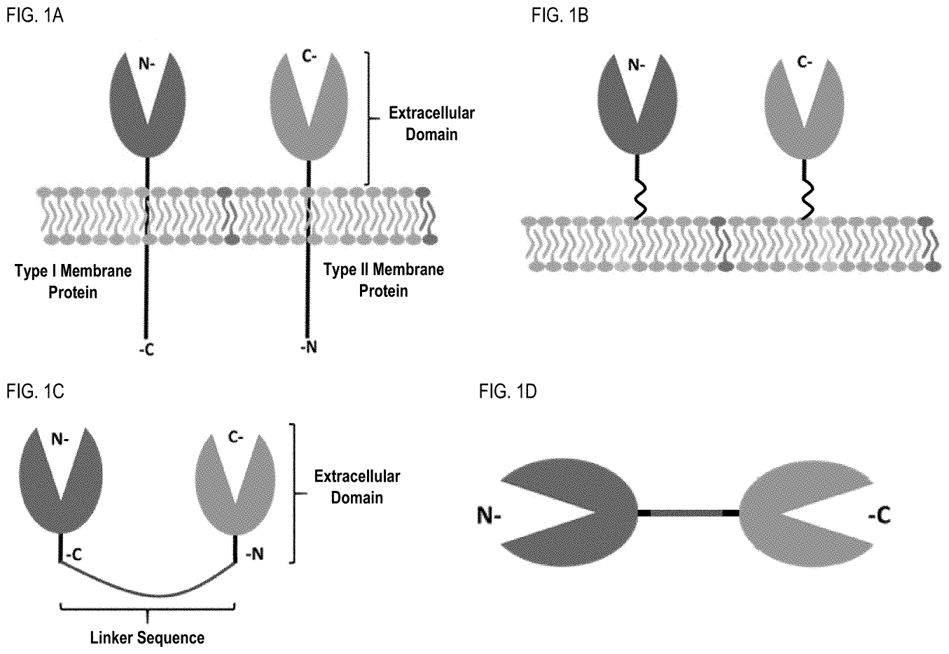

There are generally two types of single-pass transmembrane proteins: Type I transmembrane proteins which have an extracellular amino terminus and an intracellular carboxy terminus (see, FIG. 1A, left protein) and Type II transmembrane proteins which have an extracellular carboxy terminus and an intracellular amino terminus (see, FIG. 1A, right protein). Type I and Type II transmembrane proteins can be either receptors or ligands. For Type I transmembrane proteins the amino terminus of the protein faces outside the cell, and therefore contains the functional domains that are responsible for interacting with other binding partners (either ligands or receptors) in the extracellular environment. For Type II transmembrane proteins, the carboxy terminus of the protein faces outside the cell, and therefore contains the functional domains that are responsible for interacting with other binding partners (either ligands or receptors) in the extracellular environment. Thus, these two types of transmembrane proteins have opposite orientations to each other relative to the cell membrane, with the amino terminus of a Type I transmembrane protein is orientated away from the cell membrane whereas the amino terminus of a Type II transmembrane protein is orientated towards from the cell membrane.

In embodiments, a chimeric protein comprises a portion of a membrane-anchored extracellular protein. Generally, membrane-anchored extracellular protein resides in and interacts with the extracellular environment. In embodiments, the portion of the membrane-anchored extracellular protein is sufficient for binding to a ligand or receptor. In embodiments, the portion is the entire amino acid sequence of the membrane-anchored extracellular protein. Determining whether the portion of the membrane-anchored extracellular protein is capable ligand/receptor binding may be assayed using methods know in the art (e.g., in vitro ligand binding and/or cellular activation assays). FIG. 1B shows two membrane-anchored extracellular proteins, with the curved lines represents the anchoring domains; the left protein has its carboxy terminus anchored to the cell membrane and the right protein has its amino terminus anchored to the cell membrane.

In chimeric proteins of the present invention, a Type I transmembrane protein and a Type II transmembrane protein may be engineered such that their transmembrane and intracellular domains are omitted and the transmembrane proteins' extracellular domains are adjoined using a linker sequence to generate a single chimeric protein. Alternately, two membrane-anchored extracellular proteins may be engineered such that a portion of their extracellular domains are adjoined using a linker sequence to generate a single chimeric protein. Finally, one membrane-anchored extracellular protein and one transmembrane protein (lacking its transmembrane and intracellular domains) may be adjoined using a linker sequence to generate a single chimeric protein. As shown in FIG. 1C and FIG. 1D, the extracellular domain of a Type I transmembrane protein or a carboxy-terminus anchored extracellular protein and the extracellular domain of a Type II transmembrane protein or an amino-anchored extracellular protein are combined into a single chimeric protein. FIG. 1C depicts the linkage of a liberated Type I transmembrane protein (from its transmembrane and intracellular domains) or a liberated carboxy-terminus anchored extracellular protein (from its anchoring domain) and a liberated Type II transmembrane protein (from its transmembrane and intracellular domains) or a liberated amino-terminus anchored extracellular protein (from its anchoring domain) have been adjoined by a linker sequence. The extracellular domains in this depiction may include the entire amino acid sequence of the Type I protein's extracellular domain or the entire amino acid sequence of the carboxy-anchored extracellular protein, or a fraction thereof, wherein the fraction retains the ability to bind the intended ligand/receptor. Likewise, the extracellular domains in this depiction may include the entire amino acid sequence of the Type II protein's extracellular domain or the entire amino acid sequence of the amino-anchored extracellular protein, or a fraction thereof, wherein the fraction retains the ability to bind the intended ligand/receptor. Moreover, the chimeric protein comprises sufficient overall flexibility and/or physical distance between domains such that a first extracellular domain (shown at the left end of the chimeric protein in FIG. 1C and FIG. 1D) is sterically capable of binding its receptor/ligand and/or a second extracellular domain (shown at the right end of the chimeric protein in FIG. 1C and FIG. 1D) is sterically capable of binding its receptor/ligand. FIG. 1D depicts adjoined extracellular domains in a linear chimeric protein wherein each extracellular domain of the chimeric protein is facing “outward”.

Importantly, since a chimeric protein of the present invention disrupts, blocks, reduces, inhibits, and/or sequesters the transmission of immunosuppressive signals with one domain, and also either (i) the reception of immunosuppressive signals or (ii) provide an immune stimulatory signal with the other domain, it can provide an anti-tumor effect and/or an antiviral effect by two distinct pathways; this dual-action is more likely to provide a therapeutic effect in a patient and/or to provide an enhanced therapeutic effect in a patient. Furthermore, since such chimeric proteins can act via two distinct pathways, they can be efficacious, at least, in patients who respond poorly to treatments that target one of the two pathways. Thus, a patient who is a poor responder to treatments acting via one of the two pathways can receive a therapeutic benefit by targeting the other pathway.

The present chimeric proteins provide advantages including, without limitation, ease of use and ease of production. This is because two distinct immunotherapy agents are combined into a single product which may allow for a single manufacturing process instead of two independent manufacturing processes. In addition, administration of a single agent instead of two separate agents allows for easier administration and greater patient compliance. Further, in contrast to, for example, monoclonal antibodies, which are large multimeric proteins containing numerous disulfide bonds and post-translational modifications such as glycosylation, the present chimeric proteins are easier and more cost effective to manufacture.

### Chimeric Proteins

The chimeric proteins of the present invention comprise a portion of the extracellular domain of a Type I transmembrane protein or a portion of a membrane-anchored extracellular protein and the extracellular domain of a Type II transmembrane protein in which the Type II transmembrane protein is naturally expressed on the surface of a Natural Killer (NK) cell, e.g., an NKG2 family of receptors. These chimeric proteins can, at least, block transmission of immune inhibitory signals to an NK cell which allow for an anti-tumor attack or an attack of a virus-infected cell.

An aspect of the present invention is a chimeric protein of a general structure of: N terminus-(a)-(b)-(c)-C terminus, where (a) is a first domain comprising a portion of the extracellular domain of a Type I transmembrane protein or a portion of a membrane-anchored extracellular protein, (b) is a linker adjoining the first and second domains, and (c) is a second domain comprising a portion of the extracellular domain of a Type II transmembrane protein, wherein the Type II transmembrane protein is naturally expressed on the surface of a Natural Killer (NK) cell.

In embodiments, the Type II transmembrane protein is a member of the NKG2 family of receptors.

In embodiments, the member of the NKG2 family of receptors is selected from NKG2A, NKG2B, NKG2C, NKG2D, NKG2E, NKG2F, and NKG2H, e.g., the member of the NKG2 family of receptors is NKG2A. In embodiments, the second domain is capable of binding an NKG2A ligand, e.g., HLA-E (in humans) or Qa1 (in mice). In embodiments, the second domain comprises substantially all the extracellular domain of NKG2A. In embodiments, binding the NKG2A ligand blocks transmission of an immune inhibitory signal to an NK cell.

In embodiments, the Type I transmembrane protein is selected from CD80, CD86, CD58, PD-1, SLAMF6, SIRPα and TGFBR2. In embodiments, the first domain is capable of binding the ligand/receptor for the Type I transmembrane protein. In embodiments, the Type I transmembrane protein is CD80. In embodiments, the ligand/receptor is CTLA-4 or CD28. In embodiments, the Type I transmembrane protein is CD86. In embodiments, the ligand/receptor is CTLA-4 or CD28. In embodiments, the Type I transmembrane protein is CD58. In embodiments, the ligand/receptor is CD2. In embodiments, the Type I transmembrane protein is PD-1. In embodiments, the ligand/receptor is PD-L1 or PD-L2. In embodiments, the Type I transmembrane protein is SLAMF6. In embodiments, the ligand/receptor is SAP or EAT2. In embodiments, the Type I transmembrane protein is SIRPα. In embodiments, the ligand/receptor is CD47. In embodiments, the Type I transmembrane protein is TGFBR2. In embodiments, the ligand TGFβ3 or TGFβ1. In embodiments, the first domain comprises substantially all the extracellular domain the Type I transmembrane protein. In embodiments, binding the first domain to its ligand/receptor inhibits an immunosuppressive signal. In embodiments, binding the first domain to its ligand/receptor activates an immunosuppressive signal.

In embodiments, the membrane-anchored extracellular protein is CD48. In embodiments, the ligand/receptor is CD2. In embodiments, the ligand/receptor is 2B4. In embodiments, the first domain comprises substantially all of the mature CD48 polypeptide. In embodiments, binding the first domain to its ligand/receptor inhibits an immunosuppressive signal. In embodiments, binding the first domain to its ligand/receptor activates an immunosuppressive signal.

In embodiments, the chimeric protein is capable of forming a stable synapse between cells, e.g., an NK cell and a tumor cell or an NK cell and a virus-infected cell. In embodiments, the stable synapse between cells provides spatial orientation that favors tumor reduction or killing of virus-infected cells by the NK cell. In embodiments, the spatial orientation positions NK cells to attack target cells selected from tumor cells and virus-infected cells and/or sterically prevents the target cells from delivering negative signals, including negative signals beyond those masked by the chimeric protein of the invention.

In embodiments, binding of either or both of the first domain and the second domains to its ligand/receptor occurs with slow off rates (K_{off}), which provides a long interaction of a receptor and its ligand. In embodiments, the long interaction provides sustained negative signal masking effect, sustained inhibition of an immunosuppressive signal, and/or sustained activation of an immunosuppressive signal. In embodiments, the long interaction provides for NK cell proliferation and/or allows for anti-tumor attack or attack of a virus-infected cell. In embodiments, the long interaction allows sufficient signal transmission to provide release of a stimulatory signal, e.g., a cytokine.

In embodiments, the chimeric protein is capable of providing a sustained immunomodulatory effect.

In embodiments, wherein the linker is a polypeptide selected from a flexible amino acid sequence, an IgG hinge region, and an antibody sequence.

In embodiments, the linker comprises at least one cysteine residue capable of forming a disulfide bond and/or comprises a hinge-CH2-CH3 Fc domain, e.g., derived from IgG, IgA, IgD, or IgE. In embodiments, the IgG is selected from IgG1, IgG2, IgG3, and IgG4 and the IgA is selected from IgA1 and IgA2. In embodiments, the IgG is IgG4, e.g., a human IgG4. In embodiments, the linker comprises an amino acid sequence that is at least 95% identical to the amino acid sequence of SEQ ID NO: 1, SEQ ID NO: 2, or SEQ ID NO: 3.

Another aspect of the present invention is a chimeric protein comprising: (a) a first domain comprising a portion of CD80 that is capable of binding a CD80 ligand/receptor, (b) a second domain comprising a portion of NKG2A that is capable of binding an NKG2A ligand, and (c) a linker linking the first domain and the second domain and comprising a hinge-CH2-CH3 Fc domain. In embodiments, the hinge-CH2-CH3 Fc domain is derived from IgG, IgA, IgD, or IgE. In embodiments, the IgG is selected from IgG1, IgG2, IgG3, and IgG4 and the IgA is selected from IgA1 and IgA2. In embodiments, the IgG is IgG4, e.g., a human IgG4. In embodiments, the linker comprises an amino acid sequence that is at least 95% identical to the amino acid sequence of SEQ ID NO: 1, SEQ ID NO: 2, or SEQ ID NO: 3.

Yet another aspect of the present invention is a chimeric protein comprising: (a) a first domain comprising a portion of CD86 that is capable of binding a CD86 ligand/receptor, (b) a second domain comprising a portion of NKG2A that is capable of binding an NKG2A ligand, and (c) a linker linking the first domain and the second domain and comprising a hinge-CH2-CH3 Fc domain. In embodiments, the hinge-CH2-CH3 Fc domain is derived from IgG, IgA, IgD, or IgE. In embodiments, the IgG is selected from IgG1, IgG2, IgG3, and IgG4 and the IgA is selected from IgA1 and IgA2. In embodiments, the IgG is IgG4, e.g., a human IgG4. In embodiments, the linker comprises an amino acid sequence that is at least 95% identical to the amino acid sequence of SEQ ID NO: 1, SEQ ID NO: 2, or SEQ ID NO: 3.

In an aspect, the present invention provides a chimeric protein comprising: (a) a first domain comprising a portion of CD48 that is capable of binding a CD48 ligand/receptor, (b) a second domain comprising a portion of NKG2A that is capable of binding an NKG2A ligand, and (c) a linker linking the first domain and the second domain and comprising a hinge-CH2-CH3 Fc domain. In embodiments, the hinge-CH2-CH3 Fc domain is derived from IgG, IgA, IgD, or IgE. In embodiments, the IgG is selected from IgG1, IgG2, IgG3, and IgG4 and the IgA is selected from IgA1 and IgA2. In embodiments, the IgG is IgG4, e.g., a human IgG4. In embodiments, the linker comprises an amino acid sequence that is at least 95% identical to the amino acid sequence of SEQ ID NO: 1, SEQ ID NO: 2, or SEQ ID NO: 3.

In another aspect, the present invention provides a chimeric protein comprising: (a) a first domain comprising a portion of CD58 that is capable of binding a CD58 ligand/receptor, (b) a second domain comprising a portion of NKG2A that is capable of binding an NKG2A ligand, and (c) a linker linking the first domain and the second domain and comprising a hinge-CH2-CH3 Fc domain. In embodiments, the hinge-CH2-CH3 Fc domain is derived from IgG, IgA, IgD, or IgE. In embodiments, the IgG is selected from IgG1, IgG2, IgG3, and IgG4 and the IgA is selected from IgA1 and IgA2. In embodiments, the IgG is IgG4, e.g., a human IgG4. In embodiments, the linker comprises an amino acid sequence that is at least 95% identical to the amino acid sequence of SEQ ID NO: 1, SEQ ID NO: 2, or SEQ ID NO: 3.

In yet another aspect, the present invention provides a chimeric protein comprising: (a) a first domain comprising a portion of PD-1 that is capable of binding a PD-1 ligand, (b) a second domain comprising a portion of NKG2A that is capable of binding an NKG2A ligand, and (c) a linker linking the first domain and the second domain and comprising a hinge-CH2-CH3 Fc domain. In embodiments, the hinge-CH2-CH3 Fc domain is derived from IgG, IgA, IgD, or IgE. In embodiments, the IgG is selected from IgG1, IgG2, IgG3, and IgG4 and the IgA is selected from IgA1 and IgA2. In embodiments, the IgG is IgG4, e.g., a human IgG4. In embodiments, the linker comprises an amino acid sequence that is at least 95% identical to the amino acid sequence of SEQ ID NO: 1, SEQ ID NO: 2, or SEQ ID NO: 3.

An aspect of the present invention is a chimeric protein comprising: (a) a first domain comprising a portion of SLAMF6 that is capable of binding a SLAMF6 ligand/receptor, (b) a second domain comprising a portion of NKG2A that is capable of binding an NKG2A ligand, and (c) a linker linking the first domain and the second domain and comprising a hinge-CH2-CH3 Fc domain. In embodiments, the hinge-CH2-CH3 Fc domain is derived from IgG, IgA, IgD, or IgE. In embodiments, the IgG is selected from IgG1, IgG2, IgG3, and IgG4 and the IgA is selected from IgA1 and IgA2. In embodiments, the IgG is IgG4, e.g., a human IgG4. In embodiments, the linker comprises an amino acid sequence that is at least 95% identical to the amino acid sequence of SEQ ID NO: 1, SEQ ID NO: 2, or SEQ ID NO: 3.

An aspect of the present invention is a chimeric protein comprising: (a) a first domain comprising a portion of SIRPα that is capable of binding a SIRPα ligand/receptor, (b) a second domain comprising a portion of NKG2A that is capable of binding an NKG2A ligand, and (c) a linker linking the first domain and the second domain and comprising a hinge-CH2-CH3 Fc domain. In embodiments, the hinge-CH2-CH3 Fc domain is derived from IgG, IgA, IgD, or IgE. In embodiments, the IgG is selected from IgG1, IgG2, IgG3, and IgG4 and the IgA is selected from IgA1 and IgA2. In embodiments, the IgG is IgG4, e.g., a human IgG4. In embodiments, the linker comprises an amino acid sequence that is at least 95% identical to the amino acid sequence of SEQ ID NO: 1, SEQ ID NO: 2, or SEQ ID NO: 3.

Another aspect of the present invention is a chimeric protein comprising: (a) a first domain comprising a portion of TGFBR2 that is capable of binding a TGFBR2 ligand, (b) a second domain comprising a portion of NKG2A that is capable of binding an NKG2A ligand, and (c) a linker linking the first domain and the second domain and comprising a hinge-CH2-CH3 Fc domain. In embodiments, the hinge-CH2-CH3 Fc domain is derived from IgG, IgA, IgD, or IgE. In embodiments, the IgG is selected from IgG1, IgG2, IgG3, and IgG4 and the IgA is selected from IgA1 and IgA2. In embodiments, the IgG is IgG4, e.g., a human IgG4. In embodiments, the linker comprises an amino acid sequence that is at least 95% identical to the amino acid sequence of SEQ ID NO: 1, SEQ ID NO: 2, or SEQ ID NO: 3.

In embodiments, the hinge-CH2-CH3 Fc domain is derived from IgG, IgA, IgD, or IgE. In embodiments, the IgG is selected from IgG1, IgG2, IgG3, and IgG4 and the IgA is selected from IgA1 and IgA2. In embodiments, the IgG is IgG4, e.g., a human IgG4. In embodiments, the linker comprises an amino acid sequence that is at least 95% identical to the amino acid sequence of SEQ ID NO: 1, SEQ ID NO: 2, or SEQ ID NO: 3.

In a chimeric protein of the present invention, the chimeric protein is a recombinant fusion protein, e.g., a single polypeptide having the extracellular domains disclosed herein. For example, in embodiments, the chimeric protein is translated as a single unit in a prokaryotic cell, a eukaryotic cell, or a cell-free expression system.

In embodiments, the present chimeric protein is producible in a mammalian host cell as a secretable and fully functional single polypeptide chain.

In embodiments, chimeric protein refers to a recombinant protein of multiple polypeptides, e.g., multiple extracellular domains disclosed herein, that are combined (via covalent or no-covalent bonding) to yield a single unit, e.g., in vitro (e.g., with one or more synthetic linkers disclosed herein).

In embodiments, the chimeric protein is chemically synthesized as one polypeptide or each domain may be chemically synthesized separately and then combined. In embodiments, a portion of the chimeric protein is translated and a portion is chemically synthesized.

Other configurations of first and second domains are envisioned, e.g., the first domain is outward facing and the second domain is inward facing, the first domain is inward facing and the second domain is outward facing, and the first and second domains are both inward facing. When both domains are “inward facing”, the chimeric protein would have an amino-terminal to carboxy-terminal configuration comprising a portion of the extracellular domain of the Type II protein or the portion of the amino-anchored extracellular protein, a linker, and a portion of the extracellular domain of a Type I protein or the portion of the carboxy-anchored extracellular protein. In such configurations, it may be necessary for the chimeric protein to include extra “slack”, as described elsewhere herein, to permit binding domains of the chimeric protein to one or both of its receptors/ligands.

Chimeric proteins of the present invention have a first domain which is sterically capable of binding its ligand/receptor and/or a second domain which is sterically capable of binding its ligand/receptor. This means that there is sufficient overall flexibility in the chimeric protein and/or physical distance between an extracellular domain (or portion thereof) and the rest of the chimeric protein such that the ligand/receptor binding domain of the extracellular domain is not sterically hindered from binding its ligand/receptor. This flexibility and/or physical distance (which is herein referred to as “slack”) may be normally present in the extracellular domain(s), normally present in the linker, and/or normally present in the chimeric protein (as a whole). Alternately, or additionally, the chimeric protein may be modified by including one or more additional amino acid sequences (e.g., the joining linkers described below) or synthetic linkers (e.g., a polyethylene glycol (PEG) linker) which provide additional slack needed to avoid steric hindrance.

The NKG2A protein belongs to the killer cell lectin-like receptor family, also called the NKG2 family, which is a group of transmembrane proteins preferentially expressed in Natural killer (NK) cells. This family of proteins is characterized by the Type II membrane orientation and the presence of a C-type lectin domain. This protein forms a complex with another family member, KLRD1/CD94, and has been implicated in the recognition of the MHC class I HLA-E molecules in NK cells.

In embodiments, the chimeric proteins of the present invention comprise variants of the extracellular domain of NKG2A. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of NKG2A, e.g., human NKG2A.

In embodiments, the extracellular domain of human NKG2A has the following amino acid sequence:

(SEQ ID NO: 57)
PSTLIQRHNNSSLNTRTQKARHCGHCPEEWITYSNSCYYIGKERRTWEES
LLACTSKNSSLLSIDNEEEMKFLSIISPSSWIGVFRNSSHHPWVTMNGLA
FKHEIKDSDNAELNCAVLQVNRLKSAQCGSSIIYHCKHKL.

In embodiments, a chimeric protein comprises a variant of the extracellular domain of NKG2A. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 57.

In embodiments, the second domain of a chimeric protein comprises an amino acid sequence that is at least 95% identical to the amino acid sequence of SEQ ID NO: 57.

In embodiments, the portion of the extracellular domain of NKG2A is a truncation of SEQ ID NO: 57. However, the truncation retains the ability to bind an NKG2A ligand, e.g., HLA-E.

One of ordinary skill may select variants of the known amino acid sequence of NKG2A by consulting the literature, e.g. Houchins et al., “DNA sequence analysis of NKG2, a family of related cDNA clones encoding type II integral membrane proteins on human natural killer cells” J. Exp. Med. 173 (4), 1017-1020 (1991); Sullivan et al., “The heterodimeric assembly of the CD94-NKG2 receptor family and implications for human leukocyte antigen-E recognition” Immunity 27 (6), 900-911 (2007); Petrie et al., “CD94-NKG2A recognition of human leukocyte antigen (HLA)-E bound to an HLA class I leader sequence.” J. Exp. Med. 205 (3), 725-735 (2008); and Kaiser et al., “Structural basis for NKG2A/CD94 recognition of HLA-E.” Proc. Natl. Acad. Sci. U.S.A. 105 (18), 6696-6701 (2008), each of which is incorporated by reference in its entirety.

CD80 is a membrane receptor that is activated by the binding of CD28 or cytotoxic T-lymphocyte-associated protein 4 (CTLA-4). The activated protein induces T-cell proliferation and cytokine production. CD80 can act as a receptor for adenovirus subgroup B and may play a role in lupus neuropathy.

In embodiments, the chimeric proteins of the present invention comprise variants of the extracellular domain of CD80. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of CD80, e.g., human CD80.

In embodiments, the extracellular domain of human CD80 has the following amino acid sequence:

(SEQ ID NO: 59)
VIHVTKEVKEVATLSCGHNVSVEELAQTRIYWQKEKKMVLTMMSGDMNIW
PEYKNRTIFDITNNLSIVILALRPSDEGTYECVVLKYEKDAFKREHLAEV
TLSVKADFPTPSISDFEIPTSNIRRIICSTSGGFPEPHLSWLENGEELNA
INTTVSQDPETELYAVSSKLDFNMTTNHSFMCLIKYGHLRVNQTFNWNTT
KQEHFPDN.

In embodiments, a chimeric protein comprises a variant of the extracellular domain of CD80. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 59.

In embodiments, the first domain of a chimeric protein comprises an amino acid sequence that is at least 95% identical to the amino acid sequence of SEQ ID NO: 59.

In embodiments, the portion of the extracellular domain of CD80 is a truncation of SEQ ID NO: 59. However, the truncation retains the ability to bind a CD80 ligand/receptor, e.g., CTLA-4 and CD28.

One of ordinary skill may select variants of the known amino acid sequence of CD80 by consulting the literature, e.g., Freeman et al., “B7, a new member of the Ig superfamily with unique expression on activated and neoplastic B cells.” J. Immunol. 143 (8), 2714-2722 (1989); Freeman et al., “Structure, expression, and T cell costimulatory activity of the murine homologue of the human B lymphocyte activation antigen B7.” J. Exp. Med. 174 (3), 625-631 (1991); Lanier et al, “CD80 (B7) and CD86 (B70) provide similar costimulatory signals for T cell proliferation, cytokine production, and generation of CTL.” J. Immunol. 154 (1), 97-105 (1995); Vandenborre et al., “Interaction of CTLA-4 (CD152) with CD80 or CD86 inhibits human T-cell activation.” Immunology 98 (3), 413-421 (1999); Ikemizu et al., Structure and dimerization of a soluble form of B7-1.” Immunity 12 (1), 51-60 (2000); and Stamper et al., “Crystal structure of the B7-1/CTLA-4 complex that inhibits human immune responses.” Nature 410 (6828), 608-611 (2001), each of which is incorporated by reference in its entirety.

In embodiments, a chimeric protein of the present invention comprises: (1) a first domain comprising the amino acid sequence of SEQ ID NO: 59, (b) a second domain comprises the amino acid sequence of SEQ ID NO: 57, and (c) a linker comprises an amino acid sequence that is at least 95% identical to SEQ ID NO: 1, SEQ ID NO: 2, or SEQ ID NO: 3.

In embodiments, a CD80-Fc-NKG2A chimeric protein of the present invention may comprise the following amino acid sequence:

(SEQ ID NO: 61)
VIHVTKEVKEVATLSCGHNVSVEELAQTRIYWQKEKKMVLTMMSGDMNIW
PEYKNRTIFDITNNLSIVILALRPSDEGTYECVVLKYEKDAFKREHLAEV
TLSVKADFPTPSISDFEIPTSNIRRIICSTSGGFPEPHLSWLENGEELNA
INTTVSQDPETELYAVSSKLDFNMTTNHSFMCLIKYGHLRVNQTFNWNTT
KQEHFPDNSKYGPPCPPCPAPEFLGGPSVFLFPPKPKDQLMISRTPEVTC
VWVDVSQEDPEVQFNWYVDGVEVHNAKTKPREEQFNSTYRVVSVLTVLHQ
DWLSGKEYKCKVSSKGLPSSIEKTISNATGQPREPQVYTLPPSQEEMTKN
QVSLTCLVKGFYPSDIAVEWESNGQPENNYKTTPPVLDSDGSFFLYSRLT
VDKSRWQEGNVFSCSVLHEALHNHYTQKSLSLSLGKIEGRMDPSTLIQRH
NNSSLNTRTQKARHCGHCPEEWITYSNSCYYIGKERRTWEESLLACTSKN
SSLLSIDNEEEMKFLSIISPSSWIGVFRNSSHHPWVTMNGLAFKHEIKDS
DNAELNCAVLQVNRLKSAQCGSSIIYHCKHKL.

In embodiments, a chimeric protein comprises a variant of a CD80-Fc-NKG2A chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 61.

CD86 is a Type I membrane protein that is a member of the immunoglobulin superfamily. This protein is expressed by antigen-presenting cells, and it is the ligand for two proteins at the cell surface of T cells, CD28 antigen and cytotoxic T-lymphocyte-associated protein 4 (CTLA-4). Binding of CD86 with CD28 antigen is a costimulatory signal for activation of the T-cell. Binding of CD86 with CTLA-4 negatively regulates T-cell activation and diminishes the immune response.

In embodiments, the chimeric proteins of the present invention comprise variants of the extracellular domain of CD86. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of CD86, e.g., human CD86.

In embodiments, the extracellular domain of human CD86 has the following amino acid sequence:

(SEQ ID NO: 63)
APLKIQAYFNETADLPCQFANSQNQSLSELVVFWQDQENLVLNEVYLGKE
KFDSVHSKYMGRTSFDSDSWTLRLHNLQIKDKGLYQCIIHHKKPTGMIRI
HQMNSELSVLANFSQPEIVPISNITENVYINLTCSSIHGYPEPKKMSVLL
RTKNSTIEYDGVMQKSQDNVTELYDVSISLSVSFPDVTSNMTIFCILETD
KTRLLSSPFSIELEDPQPPPDHIP.

In embodiments, a chimeric protein comprises a variant of the extracellular domain of CD86. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 63.

In embodiments, the first domain of a chimeric protein comprises an amino acid sequence that is at least 95% identical to the amino acid sequence of SEQ ID NO: 63.

In embodiments, the portion of the extracellular domain of CD86 is a truncation of SEQ ID NO: 63. However, the truncation retains the ability to bind a CD86 ligand/receptor, e.g., CTLA-4 and CD28.

One of ordinary skill may select variants of the known amino acid sequence of CD86 by consulting the literature, e.g., Azuma et al., “B70 antigen is a second ligand for CTLA-4 and CD28” Nature 366 (6450), 76-79 (1993); Freeman et al., “Cloning of B7-2: a CTLA-4 counter-receptor that costimulates human T cell proliferation.” Science 262 (5135), 909-911 (1993); Lanier et al., “CD80 (B7) and CD86 (B70) provide similar costimulatory signals for T cell proliferation, cytokine production, and generation of CTL.” J. Immunol. 154 (1), 97-105 (1995); Engel et al., “The B7-2 (B70) costimulatory molecule expressed by monocytes and activated B lymphocytes is the CD86 differentiation antigen.” Blood 84 (5), 1402-1407 (1994); Schwartz et al., “Structural basis for co-stimulation by the human CTLA-4/B7-2 complex.” Nature 410 (6828), 604-608 (2001); and Zhang et al., “Crystal structure of the receptor-binding domain of human B7-2: insights into organization and signaling.” Proc. Natl. Acad. Sci. U.S.A. 100 (5), 2586-2591 (2003), each of which is incorporated by reference in its entirety.

In embodiments, a chimeric protein of the present invention comprises: (1) a first domain comprising the amino acid sequence of SEQ ID NO: 63, (b) a second domain comprises the amino acid sequence of SEQ ID NO: 57, and (c) a linker comprises an amino acid sequence that is at least 95% identical to SEQ ID NO: 1, SEQ ID NO: 2, or SEQ ID NO: 3.

In embodiments, a CD86-Fc-NKG2A chimeric protein of the present invention may comprise the following amino acid sequence:

(SEQ ID NO: 65)
APLKIQAYFNETADLPCQFANSQNQSLSELVVFWQDQENLVLNEVYLGKE
KFDSVHSKYMGRTSFDSDSWTLRLHNLQIKDKGLYQCIIHHKKPTGMIRI
HQMNSELSVLANFSQPEIVPISNITENVYINLTCSSIHGYPEPKKMSVLL
RTKNSTIEYDGVMQKSQDNVTELYDVSISLSVSFPDVTSNMTIFCILETD
KTRLLSSPFSIELEDPQPPPDHIPSKYGPPCPPCPAPEFLGGPSVFLFPP
KPKDQLMISRTPEVTCWVDVSQEDPEVQFNWYVDGVEVHNAKTKPREEQF
NSTYRWVSVLTVLHQDWLSGKEYKCKVSSKGLPSSIEKTISNATGQPREP
QVYTLPPSQEEMTKNQVSLTCLVKGFYPSDIAVEWESNGQPENNYKTTPP
VLDSDGSFFLYSRLTVDKSRWQEGNVFSCSVLHEALHNHYTQKSLSLSLG
KIEGRMDPSTLIQRHNNSSLNTRTQKARHCGHCPEEWITYSNSCYYIGKE
RRTWEESLLACTSKNSSLLSIDNEEEMKFLSIISPSSWIGVFRNSSHHPW
VTMNGLAFKHEIKDSDNAELNCAVLQVNRLKSAQCGSSIIYHCKHKL.

In embodiments, a chimeric protein comprises a variant of a CD86-Fc-NKG2A chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 65.

CD58 is a member of the immunoglobulin superfamily. The encoded protein is a ligand of the T lymphocyte CD2 protein, and functions in adhesion and activation of T lymphocytes.

In embodiments, the chimeric proteins of the present invention comprise variants of the extracellular domain of CD58. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of CD58, e.g., human CD58.

In embodiments, the extracellular domain of human CD58 has the following amino acid sequence:

(SEQ ID NO: 67)
FSQQIYGVVYGNVTFHVPSNVPLKEVLWKKQKDKVAELENSEFRAFSSFK
NRVYLDTVSGSLTIYNLTSSDEDEYEMESPNITDTMKFFLYVLESLPSPT
LTCALTNGSIEVQCMIPEHYNSHRGLIMYSWDCPMEQCKRNSTSIYFKME
NDLPQKIQCTLSNPLFNTTSSIILTTCIPSSGHSRHR.

In embodiments, a chimeric protein comprises a variant of the extracellular domain of CD58. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 67.

In embodiments, the first domain of a chimeric protein comprises an amino acid sequence that is at least 95% identical to the amino acid sequence of SEQ ID NO: 67.

In embodiments, the portion of the extracellular domain of CD58 is a truncation of SEQ ID NO: 67. However, the truncation retains the ability to bind a CD58 ligand/receptor, e.g., CD2.

One of ordinary skill may select variants of the known amino acid sequence of CD58 by consulting the literature, e.g., Wallner et al., “Primary structure of lymphocyte function-associated antigen 3 (LFA-3). The ligand of the T lymphocyte CD2 glycoprotein.” J. Exp. Med. 166 (4), 923-932 (1987); Omaetxebarria et al., “Computational approach for identification and characterization of GPI-anchored peptides in proteomics experiments.” Proteomics 7 (12), 1951-1960 (2007); Wang et al., “Structure of a heterophilic adhesion complex between the human CD2 and CD58 (LFA-3) counterreceptors.” Cell 97 (6), 791-803 (1999); and Ikemizu et al., “Crystal structure of the CD2-binding domain of CD58 (lymphocyte function-associated antigen 3) at 1.8-A resolution.” Proc. Natl. Acad. Sci. U.S.A. 96 (8), 4289-4294 (1999), each of which is incorporated by reference in its entirety.

In embodiments, a chimeric protein of the present invention comprises: (1) a first domain comprising the amino acid sequence of SEQ ID NO: 67, (b) a second domain comprises the amino acid sequence of SEQ ID NO: 57, and (c) a linker comprises an amino acid sequence that is at least 95% identical to SEQ ID NO: 1, SEQ ID NO: 2, or SEQ ID NO: 3.

In embodiments, a CD58-Fc-NKG2A chimeric protein of the present invention may comprise the following amino acid sequence:

(SEQ ID NO: 68)
FSQQIYGVVYGNVTFHVPSNVPLKEVLWKKQKDKVAELENSEFRAFSSFK
NRVYLDTVSGSLTIYNLTSSDEDEYEMESPNITDTMKFFLYVLESLPSPT
LTCALTNGSIEVQCMIPEHYNSHRGLIMYSWDCPMEQCKRNSTSIYFKME
NDLPQKIQCTLSNPLFNTTSSIILTTCIPSSGHSRHRSKYGPPCPPCPAP
EFLGGPSVFLFPPKPKDQLMISRTPEVTCVVDVSQEDPEVQFNWYVDGVE
VHNAKTKPREEQFNSTYRVVSVLTVLHQDWLSGKEYKCKVSSKGLPSSIE
KTISNATGQPREPQVYTLPPSQEEMTKNQVSLTCLVKGFYPSDIAVEWES
NGQPENNYKTTPPVLDSDGSFFLYSRLTVDKSRWQEGNVFSCSVLHEALH
NHYTQKSLSLSLGKIEGRMDPSTLIQRHNNSSLNTRTQKARHCGHCPEEW
ITYSNSCYYIGKERRTWEESLLACTSKNSSLLSIDNEEEMKFLSIISPSS
WIGVFRNSSHHPWVTMNGLAFKHEIKDSDNAELNCAVLQVNRLKSAQCGS
SHYHCKHKL.

In embodiments, a chimeric protein comprises a variant of a CD58-Fc-NKG2A chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 68.

PD-1 is a cell surface membrane protein of the immunoglobulin superfamily. This protein is expressed in pro-B-cells and is thought to play a role in their differentiation. In mice, PD-1 expression is induced in the thymus when anti-CD3 antibodies are injected and large numbers of thymocytes undergo apoptosis. Mice deficient for its gene bred on a BALB/c background developed dilated cardiomyopathy and died from congestive heart failure. These studies suggest that PD-1 may also be important in T cell function and contribute to the prevention of autoimmune diseases.

In embodiments, the chimeric proteins of the present invention comprise variants of the extracellular domain of PD-1. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of PD-1, e.g., human PD-1.

In embodiments, the extracellular domain of human PD-1 has the following amino acid sequence:

(SEQ ID NO: 69)
LDSPDRPWNPPTFSPALLVVTEGDNATFTCSFSNTSESFVLNWYRMSPS
NQTDKLAAFPEDRSQPGQDCRFRVTQLPNGRDFHMSVVRARRNDSGTYL
CGAISLAPKAQIKESLRAELRVTERRAEVPTAHPSPSPRPAGQFQ.

In embodiments, a chimeric protein comprises a variant of the extracellular domain of PD-1. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 69.

In embodiments, the first domain of a chimeric protein comprises an amino acid sequence that is at least 95% identical to the amino acid sequence of SEQ ID NO: 69.

In embodiments, the portion of the extracellular domain of PD-1 is a truncation of SEQ ID NO: 69. However, the truncation retains the ability to bind a PD-1 ligand, e.g., PD-L1 and PD-L2.

One of ordinary skill may select variants of the known amino acid sequence of PD-1 by consulting the literature, e.g., Zhang et al “Structural and Functional Analysis of the Costimulatory Receptor Programmed Death-1” Immunity. 2004 March; 20(3):337-47; Lin et al “The PD-1/PD-L1 complex resembles the antigen-binding Fv domains of antibodies and T cell receptors”, Proc Natl Acad Sci USA. 2008 Feb. 26; 105(8):3011-6; Zak et al “Structure of the Complex of Human Programmed Death 1, PD-1, and Its Ligand PD-L1”, Structure. 2015 Dec. 1; 23(12):2341-2348; and Cheng et al “Structure and Interactions of the Human Programmed Cell Death 1 Receptor”, J Biol Chem. 2013 Apr. 26; 288(17):11771-85, each of which is incorporated by reference in its entirety.

In embodiments, a chimeric protein of the present invention comprises: (1) a first domain comprising the amino acid sequence of SEQ ID NO: 69, (b) a second domain comprises the amino acid sequence of SEQ ID NO: 57, and (c) a linker comprises an amino acid sequence that is at least 95% identical to SEQ ID NO: 1, SEQ ID NO: 2, or SEQ ID NO: 3.

In embodiments, a PD-1-Fc-NKG2A chimeric protein of the present invention may comprise the following amino acid sequence:

(SEQ ID NO: 71)
LDSPDRPWNPPTFSPALLVVTEGDNATFTCSFSNTSESFVLNWYRMSPSN
QTDKLAAFPEDRSQPGQDCRFRVTQLPNGRDFHMSVVRARRNDSGTYLCG
AISLAPKAQIKESLRAELRVTERRAEVPTAHPSPSPRPAGQFQSKYGPPC
PPCPAPEFLGGPSVFLFPPKPKDQLMISRTPEVTCVVVDVSQEDPEVQFN
WYVDGVEVHNAKTKPREEQFNSTYRWSVLTVLHQDWLSGKEYKCKVSSKG
LPSSIEKTISNATGQPREPQVYTLPPSQEEMTKNQVSLTCLVKGFYPSDI
AVEWESNGQPENNYKTTPPVLDSDGSFFLYSRLTVDKSRWQEGNVFSCSV
LHEALHNHYTQKSLSLSLGKIEGRMDPSTLIQRHNNSSLNTRTQKARHCG
HCPEEWITYSNSCYYIGKERRTWEESLLACTSKNSSLLSIDNEEEMKFLS
IISPSSWIGVFRNSSHHPWVTMNGLAFKHEIKDSDNAELNCAVLQVNRLK
SAQCGSSIIYHCKHKL.

In embodiments, a chimeric protein comprises a variant of a PD-1-Fc-NKG2A chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 71.

SLAMF6 is a type I transmembrane protein, belonging to the CD2 subfamily of the immunoglobulin superfamily. It is expressed on Natural killer (NK), T, and B lymphocytes. It undergoes tyrosine phosphorylation and associates with the Src homology 2 domain-containing protein (SH2D1A) as well as with SH2 domain-containing phosphatases (SHPs). SLAMF6 functions as a coreceptor in the process of NK cell activation. It can also mediate inhibitory signals in NK cells from X-linked lymphoproliferative patients.

In embodiments, the chimeric proteins of the present invention comprise variants of the extracellular domain of SLAMF6. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of SLAMF6, e.g., human SLAMF6.

In embodiments, the extracellular domain of human SLAMF6 has the following amino acid sequence:

(SEQ ID NO: 73)
QSSLTPLMVNGILGESVTLPLEFPAGEKVNFITWLFNETSLAFIVPHETK
SPEIHVTNPKQGKRLNFTQSYSLQLSNLKMEDTGSYRAQISTKTSAKLSS
YTLRILRQLRNIQVTNHSQLFQNMTCELHLTCSVEDADDNVSFRWEALGN
TLSSQPNLTVSWDPRISSEQDYTCIAENAVSNLSFSVSAQKLCEDVKIQY
TDTKM.

In embodiments, a chimeric protein comprises a variant of the extracellular domain of SLAMF6. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 73.

In embodiments, the first domain of a chimeric protein comprises an amino acid sequence that is at least 95% identical to the amino acid sequence of SEQ ID NO: 73.

In embodiments, the portion of the extracellular domain of SLAMF6 is a truncation of SEQ ID NO: 73. However, the truncation retains the ability to bind a SLAMF6 ligand, e.g., SAP and EAT2.

One of ordinary skill may select variants of the known amino acid sequence of SLAMF6 by consulting the literature, e.g., Bottino et al., “NTB-A [correction of GNTB-A], a novel SH2D1A-associated surface molecule contributing to the inability of natural killer cells to kill Epstein-Barr virus-infected B cells in X-linked lymphoproliferative disease.” J. Exp. Med. 194 (3), 235-246 (2001); Eissmann et al., “Molecular analysis of NTB-A signaling: a role for EAT-2 in NTB-A-mediated activation of human NK cells.” J. Immunol. 177 (5), 3170-3177 (2006); Mayya et al., “Quantitative phosphoproteomic analysis of T cell receptor signaling reveals system-wide modulation of protein-protein interactions.” Sci Signal 2 (84), ra46 (2009); and Cao et al., “NTB-A receptor crystal structure: insights into homophilic interactions in the signaling lymphocytic activation molecule receptor family.” Immunity 25 (4), 559-570 (2006), each of which is incorporated by reference in its entirety.

In embodiments, a chimeric protein of the present invention comprises: (1) a first domain comprising the amino acid sequence of SEQ ID NO: 73, (b) a second domain comprises the amino acid sequence of SEQ ID NO: 57, and (c) a linker comprises an amino acid sequence that is at least 95% identical to SEQ ID NO: 1, SEQ ID NO: 2, or SEQ ID NO: 3.

In embodiments, a SLAMF6-Fc-NKG2A chimeric protein of the present invention may comprise the following amino acid sequence:

(SEQ ID NO: 75)
QSSLTPLMVNGILGESVTLPLEFPAGEKVNFITWLFNETSLAFIVPHETK
SPEIHVTNPKQGKRLNFTQSYSLQLSNLKMEDTGSYRAQISTKTSAKLS
SYTLRILRQLRNIQVTNHSQLFQNMTCELHLTCSVEDADDNVSFRWEALG
NTLSSQPNLTVSWDPRISSEQDYTCIAENAVSNLSFSVSAQKLCEDVKIQ
YTDTKMSKYGPPCPPCPAPEFLGGPSVFLFPPKPKDQLMISRTPEVTCV
VVDVSQEDPEVQFNWYVDGVEVHNAKTKPREEQFNSTYRWVVSVLTVLHQ
DWLSGKEYKCKVSSKGLPSSIEKTISNATGQPREPQVYTLPPSQEEMTKN
QVSLTCLVKGFYPSDIAVEWESNGQPENNYKTTPPVLDSDGSFFLYSRLT
VDKSRWQEGNVFSCSVLHEALHNHYTQKSLSLSLGKIEGRMDPSTLIQRH
NNSSLNTRTQKARHCGHCPEEWITYSNSCYYIGKERRTWEESLLACTSKN
SSLLSIDNEEEMKFLSIISPSSWIGVFRNSSHHPWVTMNGLAFKHEIKDS
DNAELNCAVLQVNRLKSAQCGSSIIYHCKHKL.

In embodiments, a chimeric protein comprises a variant of a SLAMF6-Fc-NKG2A chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 75.

Signal regulatory protein a (SIRPα), also known as CD172a or SHP substrate 1 (SHPS-1), is a is a type I transmembrane protein. SIRPα is established as an immunoreceptor harboring an IgV domain. It is expressed on Natural killer (NK), monocytes, granulocytes, dendritic cells and hematopoietic stem cells, myeloid stem cells and neurons. SIRPα is a negative regulator of the phosphatidylinositol 3-kinase signaling and mitogen-activated protein kinase pathways. CD47, which is a membrane protein expressed in nearly all cell types, modulates the function of SIRPα. The binding of Ig domain of CD47 and the N-terminal Ig domain of SIRPα is believed to be sufficient for mediating transcellular bidirectional signaling. The binding of SIRPα to CD47 promotes tyrosine phosphorylation of the cytoplasmic region of SIRPα. The protein tyrosine phosphatase, SHP-2 (also known as tyrosine-protein phosphatase non-receptor type 11), then binds to the cytoplasmic region of SIRPα to mediate the negative signaling regulatory functions of SIRPα by dephosphorylating its substrates. Functional CD47/SIRPα interaction is required for optimal human T- and natural killer- (NK) cell homeostasis.

In embodiments, the chimeric proteins of the present invention comprise variants of the extracellular domain of SIRPα. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of SIRPα, e.g., human SIRPα.

In embodiments, the extracellular domain of human SIRPα has the following amino acid sequence:

(SEQ ID NO: 85)
EEELQVIQPDKSVLVAAGETATLRCTATSLIPVGPIQWFRGAGPGRELIY
NQKEGHFPRVTTVSDLTKRNNMDFSIRIGNITPADAGTYYCVKFRKGSPD
DVEFKSGAGTELSVRAKPSAPVVSGPAARATPQHTVSFTCESHGFSPRDI
TLKWFKNGNELSDFQTNVDPVGESVSYSIHSTAKVVLTREDVHSQVICEV
AHVTLQGDPLRGTANLSETIRVPPTLEVTQQPVRAENQVNVTCQVRKFYP
QRLQLTWLENGNVSRTETASTVTENKDGTYNWMSWLLVNVSAHRDDVKLT
CQVEHDGQPAVSKSHDLKVSAHPKEQGSNTAAENTGSNERNIY.

In embodiments, a chimeric protein comprises a variant of the extracellular domain of SIRPα. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 73.

In embodiments, the first domain of a chimeric protein comprises an amino acid sequence that is at least 95% identical to the amino acid sequence of SEQ ID NO: 85.

In embodiments, the portion of the extracellular domain of SIRPα is a truncation of SEQ ID NO: 85. However, the truncation retains the ability to bind a SIRPα ligand, e.g., CD47.

One of ordinary skill may select variants of the known amino acid sequence of SIRPα by consulting the literature, e.g., Sano et al., “Gene structure of mouse BIT/SHPS-1.” Biochem J. 344 Pt 3:667-75 (1999); Ho et al., “Velcro′ engineering of high affinity CD47 ectodomain as signal regulatory protein a (SIRPα) antagonists that enhance antibody-dependent cellular phagocytosis.” J Biol Chem. 290(20):12650-63. (2015); Wong et al., “Polymorphism in the innate immune receptor SIRPα controls CD47 binding and autoimmunity in the nonobese diabetic mouse.” J Immunol. 193(10):4833-44 (2014); and Pan et al., “Studying the mechanism of CD47-SIRPα interactions on red blood cells by single molecule force spectroscopy.” Nanoscale 6(17):9951-4 (2014); Hatherley et al., “Structure of signal-regulatory protein alpha: a link to antigen receptor evolution.” J Biol Chem. 284(39):26613-9 (2009), each of which is incorporated by reference in its entirety.

In embodiments, a chimeric protein of the present invention comprises: (1) a first domain comprising the amino acid sequence of SEQ ID NO: 85, (b) a second domain comprises the amino acid sequence of SEQ ID NO: 57, and (c) a linker comprises an amino acid sequence that is at least 95% identical to SEQ ID NO: 1, SEQ ID NO: 2, or SEQ ID NO: 3.

In embodiments, a SIRPα-Fc-NKG2A chimeric protein of the present invention may comprise the following amino acid sequence:

(SEQ ID NO: 86)
EEELQVIQPDKSVLVAAGETATLRCTATSLIPVGPIQWFRGAGPGRELIY
NQKEGHFPRVTTVSDLTKRNNMDFSIRIGNITPADAGTYYCVKFRKGSPD
DVEFKSGAGTELSVRAKPSAPWSGPAARATPQHTVSFTCESHGFSPRDIT
LKWFKNGNELSDFQTNVDPVGESVSYSIHSTAKVVLTREDVHSQVICEVA
HVTLQGDPLRGTANLSETIRVPPTLEVTQQPVRAENQVNVTCQVRKFYPQ
RLQLTWLENGNVSRTETASTVTENKDGTYNWMSWLLVNVSAHRDDVKLTC
QVEHDGQPAVSKSHDLKVSAHPKEQGSNTAAENTGSNERNIYSKYGPPCP
PCPAPEFLGGPSVFLFPPKPKDQLMISRTPEVTCVVVDVSQEDPEVQFNW
YVDGVEVHNAKTKPREEQFNSTYRVVSVLTVLHQDWLSGKEYKCKVSSKG
LPSSIEKTISNATGQPREPQVYTLPPSQEEMTKNQVSLTCLVKGFYPSDI
AVEWESNGQPENNYKTTPPVLDSDGSFFLYSRLTVDKSRWQEGNVFSCSV
LHEALHNHYTQKSLSLSLGKIEGRMDPSTLIQRHNNSSLNTRTQKARHCG
HCPEEWITYSNSCYYIGKERRTWEESLLACTSKNSSLLSIDNEEEMKFLS
IISPSSWIGVFRNSSHHPWTMNGLAFKHEIKDSDNAELNCAVLQVNRLKS
AQCGSSIIYHCKHKL.

In embodiments, a chimeric protein comprises a variant of a SIRPα-Fc-NKG2A chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 86.

TGFBR2 is a transmembrane protein that has a protein kinase domain, forms a heterodimeric complex with TGF-beta receptor type-1, and binds TGF-beta. This receptor/ligand complex phosphorylates proteins, which then enter the nucleus and regulate the transcription of genes related to cell proliferation, cell cycle arrest, wound healing, immunosuppression, and tumorigenesis. Mutations in the gene encoding TGFBR2 have been associated with Marfan Syndrome, Loeys-Deitz Aortic Aneurysm Syndrome, and the development of various types of tumors.

In embodiments, the chimeric proteins of the present invention comprise variants of the extracellular domain of TGFBR2. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of TGFBR2, e.g., human TGFBR2.

In embodiments, the extracellular domain of human TGFBR2 has the following amino acid sequence:

(SEQ ID NO: 77)
TIPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKCI
MKEKKKPGETFFMCSCSSDECNDNIIFSEEYNTSNPDLLLVIFQ.

In embodiments, a chimeric protein comprises a variant of the extracellular domain of TGFBR2. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 77.

In embodiments, the first domain of a chimeric protein comprises an amino acid sequence that is at least 95% identical to the amino acid sequence of SEQ ID NO: 77.

In embodiments, the portion of the extracellular domain of TGFBR2 is a truncation of SEQ ID NO: 77. However, the truncation retains the ability to bind a TGFBR2 ligand, e.g., TGFβ3 or TGFβ1.

One of ordinary skill may select variants of the known amino acid sequence of TGFBR2 by consulting the literature, e.g., Lin et al., “Expression cloning of the TGF-beta type II receptor, a functional transmembrane serine/threonine kinase.” Cell 68 (4), 775-785 (1992); Daub et al., “Kinase-selective enrichment enables quantitative phosphoproteomics of the kinome across the cell cycle.” Mol. Cell 31 (3), 438-448 (2008); Hart et al., “Crystal structure of the human TbetaR2 ectodomain—TGF-beta3 complex.” Nat. Struct. Biol. 9 (3), 203-208 (2002); Boesen et al., “The 1.1 A crystal structure of human TGF-beta type II receptor ligand binding domain.” Structure 10 (7), 913-919 (2002); Deep et al., “Solution structure and backbone dynamics of the TGFbeta type II receptor extracellular domain.” Biochemistry 42 (34), 10126-10139 (2003); Groppe et al., “Cooperative assembly of TGF-beta superfamily signaling complexes is mediated by two disparate mechanisms and distinct modes of receptor binding.” Mol. Cell 29 (2), 157-168 (2008); and Radaev et al., “Ternary complex of transforming growth factor-beta1 reveals isoform-specific ligand recognition and receptor recruitment in the superfamily.” J. Biol. Chem. 285 (19), 14806-14814 (2010), each of which is incorporated by reference in its entirety.

In embodiments, a chimeric protein of the present invention comprises: (1) a first domain comprising the amino acid sequence of SEQ ID NO: 77, (b) a second domain comprises the amino acid sequence of SEQ ID NO: 57, and (c) a linker comprises an amino acid sequence that is at least 95% identical to SEQ ID NO: 1, SEQ ID NO: 2, or SEQ ID NO: 3.

In embodiments, a TGFBR2-Fc-NKG2A chimeric protein of the present invention may comprise the following amino acid sequence:

(SEQ ID NO: 79)
TIPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDLLLVIFQSKYGP
PCPPCPAPEFLGGPSVFLFPPKPKDQLMISRTPEVTCVVVDVSQEDPEVQ
FNWYVDGVEVHNAKTKPREEQFNSTYRVVSVLTVLHQDWLSGKEYKCKVS
SKGLPSSIEKTISNATGQPREPQVYTLPPSQEEMTKNQVSLTCLVKGFYP
SDIAVEWESNGQPENNYKTTPPVLDSDGSFFLYSRLTVDKSRWQEGNVFS
CSVLHEALHNHYTQKSLSLSLGKIEGRMDPSTLIQRHNNSSLNTRTQKAR
HCGHCPEEWITYSNSCYYIGKERRTWEESLLACTSKNSSLLSIDNEEEMK
FLSIISPSSWIGVFRNSSHHPWVTMNGLAFKHEIKDSDNAELNCAVLQVN
RLKSAQCGSSIIYHCKHKL.

In embodiments, a chimeric protein comprises a variant of a TGFBR2-Fc-NKG2A chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 79.

CD48 is a member of the CD2 subfamily of immunoglobulin-like receptors which includes SLAM (signaling lymphocyte activation molecules) proteins. CD48 is found on the surface of lymphocytes and other immune cells, dendritic cells and endothelial cells, and participates in activation and differentiation pathways in these cells. CD48 does not have a transmembrane domain, however, but is held at the cell surface by a GPI anchor via a C-terminal domain which maybe cleaved to yield a soluble form of the receptor.

In embodiments, the chimeric proteins of the present invention comprise variants of the portion of the membrane-anchored extracellular protein CD48. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of CD48, e.g., human CD48.

In embodiments, the membrane-anchored extracellular protein human CD48 has the following amino acid sequence:

(SEQ ID NO: 81)
QGHLVHMTVVSGSNVTLNISESLPENYKQLTWFYTFDQKIVEWDSRKSKY
FESKFKGRVRLDPQSGALYISKVQKEDNSTYIMRVLKKTGNEQEWKIKLQ
VLDPVPKPVIKIEKIEDMDDNCYLKLSCVIPGESVNYTWYGDKRPFPKE
LQNSVLETTLMPHNYSRCYTCQVSNSVSSKNGTVCLSPPCTLAR.

In embodiments, a chimeric protein comprises a variant of CD48. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 81.

In embodiments, the first domain of a chimeric protein comprises an amino acid sequence that is at least 95% identical to the amino acid sequence of SEQ ID NO: 81.

In embodiments, the portion of CD48 is a truncation of SEQ ID NO: 81. However, the truncation retains the ability to bind a CD48 ligand/receptor, e.g., CD2 and 2B4.

One of ordinary skill may select variants of the known amino acid sequence of CD48 by consulting the literature, e.g., Korinek et al., “The human leucocyte antigen CD48 (MEM-102) is closely related to the activation marker Blast-1” Immunogenetics 33 (2), 108-112 (1991); Del Porto et al., “TCT.1, a target molecule for gamma/delta T cells, is encoded by an immunoglobulin superfamily gene (Blast-1) located in the CD1 region of human chromosome 1.” J. Exp. Med. 173 (6), 1339-1344 (1991); Wollscheid et al., “Mass-spectrometric identification and relative quantification of N-linked cell surface glycoproteins.” Nat. Biotechnol. 27 (4), 378-386 (2009); and RIKEN structural genomics initiative (RSGI) Submitted August 2007, each of which is incorporated by reference in its entirety.

In embodiments, a chimeric protein of the present invention comprises: (1) a first domain comprising the amino acid sequence of SEQ ID NO: 81, (b) a second domain comprises the amino acid sequence of SEQ ID NO: 57, and (c) a linker comprises an amino acid sequence that is at least 95% identical to SEQ ID NO: 1, SEQ ID NO: 2, or SEQ ID NO: 3.

In embodiments, a CD48-Fc-NKG2A chimeric protein of the present invention may comprise the following amino acid sequence:

(SEQ ID NO: 83)
QGHLVHMTVVSGSNVTLNISESLPENYKQLTWFYTFDQKIVEWDSRKSKY
FESKFKGRVRLDPQSGALYISKVQKEDNSTYIMRVLKKTGNEQEWKIKLQ
VLDPVPKPVIKIEKIEDMDDNCYLKLSCVIPGESVNYTWYGDKRPFPKEL
QNSVLETTLMPHNYSRCYTCQVSNSVSSKNGTVCLSPPCTLARSSKYGPP
CPPCPAPEFLGGPSVFLFPPKPKDQLMISRTPEVTCVWDVSQEDPEVQFN
WYVDGVEVHNAKTKPREEQFNSTYRVVSVLTVLHQDWLSGKEYKCKVSSK
GLPSSIEKTISNATGQPREPQVYTLPPSQEEMTKNQVSLTCLVKGFYPSD
IAVEWESNGQPENNYKTTPPVLDSDGSFFLYSRLTVDKSRWQEGNVFSCS
VLHEALHNHYTQKSLSLSLGKIEGRMDPSTLIQRHNNSSLNTRTQKARHC
GHCPEEWITYSNSCYYIGKERRTWEESLLACTSKNSSLLSIDNEEEMKFL
SIISPSSWIGVFRNSSHHPWVTMNGLAFKHEIKDSDNAELNCAVLQVNRL
KSAQCGSSIIYHCKHKL.

In embodiments, a chimeric protein comprises a variant of a CD48-Fc-NKG2A chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 83.

In any herein-disclosed aspect and embodiment, the chimeric protein may comprise an amino acid sequence having one or more amino acid mutations relative to any of the protein sequences disclosed herein. In embodiments, the one or more amino acid mutations may be independently selected from substitutions, insertions, deletions, and truncations.

In embodiments, the amino acid mutations are amino acid substitutions, and may include conservative and/or non-conservative substitutions. “Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe. As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt a-helices. As used herein, “non-conservative substitutions” are defined as exchanges of an amino acid by another amino acid listed in a different group of the six standard amino acid groups (1) to (6) shown above.

In embodiments, the substitutions may also include non-classical amino acids (e.g., selenocysteine, pyrrolysine, N-formylmethionine β-alanine, GABA and δ-Aminolevulinic acid, 4-aminobenzoic acid (PABA), D-isomers of the common amino acids, 2,4-diaminobutyric acid, a-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general).

Mutations may also be made to the nucleotide sequences of the chimeric proteins by reference to the genetic code, including taking into account codon degeneracy.

In embodiments, a chimeric protein is capable of binding murine ligand(s)/receptor(s).

In embodiments, a chimeric protein is capable of binding human ligand(s)/receptor(s).

In embodiments, each extracellular domain (or variant thereof) of the chimeric protein binds to its cognate receptor or ligand with a K_{D} of about 1 nM to about 5 nM, for example, about 1 nM, about 1.5 nM, about 2 nM, about 2.5 nM, about 3 nM, about 3.5 nM, about 4 nM, about 4.5 nM, or about 5 nM. In embodiments, the chimeric protein binds to a cognate receptor or ligand with a K_{D} of about 5 nM to about 15 nM, for example, about 5 nM, about 5.5 nM, about 6 nM, about 6.5 nM, about 7 nM, about 7.5 nM, about 8 nM, about 8.5 nM, about 9 nM, about 9.5 nM, about 10 nM, about 10.5 nM, about 11 nM, about 11.5 nM, about 12 nM, about 12.5 nM, about 13 nM, about 13.5 nM, about 14 nM, about 14.5 nM, or about 15 nM.

In embodiments, each extracellular domain (or variant thereof) of the chimeric protein binds to its cognate receptor or ligand with a K_{D} of less than about 1 μM, about 900 nM, about 800 nM, about 700 nM, about 600 nM, about 500 nM, about 400 nM, about 300 nM, about 200 nM, about 150 nM, about 130 nM, about 100 nM, about 90 nM, about 80 nM, about 70 nM, about 60 nM, about 55 nM, about 50 nM, about 45 nM, about 40 nM, about 35 nM, about 30 nM, about 25 nM, about 20 nM, about 15 nM, about 10 nM, or about 5 nM, or about 1 nM (as measured, for example, by surface plasmon resonance or biolayer interferometry). In embodiments, the chimeric protein binds to human CSF1 with a K_{D} of less than about 1 nM, about 900 pM, about 800 pM, about 700 pM, about 600 pM, about 500 pM, about 400 pM, about 300 pM, about 200 pM, about 100 pM, about 90 pM, about 80 pM, about 70 pM, about 60 pM about 55 pM about 50 pM about 45 pM, about 40 pM, about 35 pM, about 30 pM, about 25 pM, about 20 pM, about 15 pM, or about 10 pM, or about 1 pM (as measured, for example, by surface plasmon resonance or biolayer interferometry).

As used herein, a variant of an extracellular domain is capable of binding the receptor/ligand of a native extracellular domain. For example, a variant may include one or more mutations in an extracellular domain which do not affect its binding affinity to its receptor/ligand; alternately, the one or more mutations in an extracellular domain may improve binding affinity for the receptor/ligand; or the one or more mutations in an extracellular domain may reduce binding affinity for the receptor/ligand, yet not eliminate binding altogether. In embodiments, the one or more mutations are located outside the binding pocket where the extracellular domain interacts with its receptor/ligand. In embodiments, the one or more mutations are located inside the binding pocket where the extracellular domain interacts with its receptor/ligand, as long as the mutations do not eliminate binding altogether. Based on the skilled artisan's knowledge and the knowledge in the art regarding receptor-ligand binding, s/he would know which mutations would permit binding and which would eliminate binding.

In embodiments, the chimeric protein exhibits enhanced stability and protein half-life.

A chimeric protein of the present invention may comprise more than two extracellular domains. For example, the chimeric protein may comprise three, four, five, six, seven, eight, nine, ten, or more extracellular domains. A second extracellular domain may be separated from a third extracellular domain via a linker, as disclosed herein.

Alternately, a second extracellular domain may be directly linked (e.g., via a peptide bond) to a third extracellular domain. In embodiments, a chimeric protein includes extracellular domains that are directly linked and extracellular domains that are indirectly linked via a linker, as disclosed herein.

### Linkers

In embodiments, the chimeric protein comprises a linker.

In embodiments, the linker comprising at least one cysteine residue capable of forming a disulfide bond. The at least one cysteine residue is capable of forming a disulfide bond between a pair (or more) of chimeric proteins. Without wishing to be bound by theory, such disulfide bond forming is responsible for maintaining a useful multimeric state of chimeric proteins. This allows for efficient production of the chimeric proteins; it allows for desired activity in vitro and in vivo.

In a chimeric protein of the present invention, the linker is a polypeptide selected from a flexible amino acid sequence, an IgG hinge region, or an antibody sequence.

In embodiments, the linker is derived from naturally-occurring multi-domain proteins or is an empirical linker as described, for example, in Chichili et al., (2013), Protein Sci. 22(2):153-167, Chen et al., (2013), Adv Drug Deliv Rev. 65(10):1357-1369, the entire contents of which are hereby incorporated by reference. In embodiments, the linker may be designed using linker designing databases and computer programs such as those described in Chen et al., (2013), Adv Drug Deliv Rev. 65(10):1357-1369 and Crasto et. al., (2000), Protein Eng. 13(5):309-312, the entire contents of which are hereby incorporated by reference.

In embodiments, the linker comprises a polypeptide. In embodiments, the polypeptide is less than about 500 amino acids long, about 450 amino acids long, about 400 amino acids long, about 350 amino acids long, about 300 amino acids long, about 250 amino acids long, about 200 amino acids long, about 150 amino acids long, or about 100 amino acids long. For example, the linker may be less than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long.

In embodiments, the linker is flexible.

In embodiments, the linker is rigid.

In embodiments, the linker is substantially comprised of glycine and serine residues (e.g., about 30%, or about 40%, or about 50%, or about 60%, or about 70%, or about 80%, or about 90%, or about 95%, or about 97%, or about 98%, or about 99%, or about 100% glycines and serines).

In embodiments, the linker comprises a hinge region of an antibody (e.g., of IgG, IgA, IgD, and IgE, inclusive of subclasses (e.g., IgG1, IgG2, IgG3, and IgG4, and IgA1, and IgA2)). The hinge region, found in IgG, IgA, IgD, and IgE class antibodies, acts as a flexible spacer, allowing the Fab portion to move freely in space. In contrast to the constant regions, the hinge domains are structurally diverse, varying in both sequence and length among immunoglobulin classes and subclasses. For example, the length and flexibility of the hinge region varies among the IgG subclasses. The hinge region of IgG1 encompasses amino acids 216-231 and, because it is freely flexible, the Fab fragments can rotate about their axes of symmetry and move within a sphere centered at the first of two inter-heavy chain disulfide bridges. IgG2 has a shorter hinge than IgG1, with 12 amino acid residues and four disulfide bridges. The hinge region of IgG2 lacks a glycine residue, is relatively short, and contains a rigid poly-proline double helix, stabilized by extra inter-heavy chain disulfide bridges. These properties restrict the flexibility of the IgG2 molecule. IgG3 differs from the other subclasses by its unique extended hinge region (about four times as long as the IgG1 hinge), containing 62 amino acids (including 21 prolines and 11 cysteines), forming an inflexible poly-proline double helix. In IgG3, the Fab fragments are relatively far away from the Fc fragment, giving the molecule a greater flexibility. The elongated hinge in IgG3 is also responsible for its higher molecular weight compared to the other subclasses. The hinge region of IgG4 is shorter than that of IgG1 and its flexibility is intermediate between that of IgG1 and IgG2. The flexibility of the hinge regions reportedly decreases in the order IgG3>IgG1>IgG4>IgG2. In embodiments, the linker may be derived from human IgG4 and contain one or more mutations to enhance dimerization (including S228P) or FcRn binding.

According to crystallographic studies, the immunoglobulin hinge region can be further subdivided functionally into three regions: the upper hinge region, the core region, and the lower hinge region. See Shin et al., 1992 Immunological Reviews 130:87. The upper hinge region includes amino acids from the carboxyl end of CH1 to the first residue in the hinge that restricts motion, generally the first cysteine residue that forms an interchain disulfide bond between the two heavy chains. The length of the upper hinge region correlates with the segmental flexibility of the antibody. The core hinge region contains the inter-heavy chain disulfide bridges, and the lower hinge region joins the amino terminal end of the CH2 domain and includes residues in CH2. Id. The core hinge region of wild-type human IgG1 contains the sequence CPPC (SEQ ID NO: 24) which, when dimerized by disulfide bond formation, results in a cyclic octapeptide believed to act as a pivot, thus conferring flexibility. In embodiments, the present linker comprises, one, or two, or three of the upper hinge region, the core region, and the lower hinge region of any antibody (e.g., of IgG, IgA, IgD, and IgE, inclusive of subclasses (e.g., IgG1, IgG2, IgG3, and IgG4, and IgA1 and IgA2)). The hinge region may also contain one or more glycosylation sites, which include a number of structurally distinct types of sites for carbohydrate attachment. For example, IgA1 contains five glycosylation sites within a 17-amino-acid segment of the hinge region, conferring resistance of the hinge region polypeptide to intestinal proteases, considered an advantageous property for a secretory immunoglobulin. In embodiments, the linker of the present invention comprises one or more glycosylation sites.

In embodiments, the linker comprises an Fc domain of an antibody (e.g., of IgG, IgA, IgD, and IgE, inclusive of subclasses (e.g., IgG1, IgG2, IgG3, and IgG4, and IgA1 and IgA2)).

In a chimeric protein of the present invention, the linker comprises a hinge-CH2-CH3 Fc domain derived from IgG4. In embodiments, the linker comprises a hinge-CH2-CH3 Fc domain derived from a human IgG4. In embodiments, the linker comprises an amino acid sequence that is at least 95% identical to the amino acid sequence of any one of SEQ ID NO: 1 to SEQ ID NO: 3, e.g., at least 95% identical to the amino acid sequence of SEQ ID NO: 2. In embodiments, the linker comprises one or more joining linkers, such joining linkers independently selected from SEQ ID NOs: 4-50 (or a variant thereof). In embodiments, the linker comprises two or more joining linkers each joining linker independently selected from SEQ ID NOs: 4-50 (or a variant thereof); wherein one joining linker is N terminal to the hinge-CH2-CH3 Fc domain and another joining linker is C terminal to the hinge-CH2-CH3 Fc domain.

In embodiments, the linker comprises a hinge-CH2-CH3 Fc domain derived from a human IgG1 antibody. In embodiments, the Fc domain exhibits increased affinity for and enhanced binding to the neonatal Fc receptor (FcRn). In embodiments, the Fc domain includes one or more mutations that increases the affinity and enhances binding to FcRn. Without wishing to be bound by theory, it is believed that increased affinity and enhanced binding to FcRn increases the in vivo half-life of the present chimeric proteins.

In embodiments, the Fc domain in a linker contains one or more amino acid substitutions at amino acid residue 250, 252, 254, 256, 308, 309, 311, 416, 428, 433 or 434 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference), or equivalents thereof. In embodiments, the amino acid substitution at amino acid residue 250 is a substitution with glutamine. In embodiments, the amino acid substitution at amino acid residue 252 is a substitution with tyrosine, phenylalanine, tryptophan or threonine. In embodiments, the amino acid substitution at amino acid residue 254 is a substitution with threonine. In embodiments, the amino acid substitution at amino acid residue 256 is a substitution with serine, arginine, glutamine, glutamic acid, aspartic acid, or threonine. In embodiments, the amino acid substitution at amino acid residue 308 is a substitution with threonine. In embodiments, the amino acid substitution at amino acid residue 309 is a substitution with proline. In embodiments, the amino acid substitution at amino acid residue 311 is a substitution with serine. In embodiments, the amino acid substitution at amino acid residue 385 is a substitution with arginine, aspartic acid, serine, threonine, histidine, lysine, alanine or glycine. In embodiments, the amino acid substitution at amino acid residue 386 is a substitution with threonine, proline, aspartic acid, serine, lysine, arginine, isoleucine, or methionine. In embodiments, the amino acid substitution at amino acid residue 387 is a substitution with arginine, proline, histidine, serine, threonine, or alanine. In embodiments, the amino acid substitution at amino acid residue 389 is a substitution with proline, serine or asparagine. In embodiments, the amino acid substitution at amino acid residue 416 is a substitution with serine. In embodiments, the amino acid substitution at amino acid residue 428 is a substitution with leucine. In embodiments, the amino acid substitution at amino acid residue 433 is a substitution with arginine, serine, isoleucine, proline, or glutamine. In embodiments, the amino acid substitution at amino acid residue 434 is a substitution with histidine, phenylalanine, or tyrosine.

In embodiments, the Fc domain linker (e.g., comprising an IgG constant region) comprises one or more mutations such as substitutions at amino acid residue 252, 254, 256, 433, 434, or 436 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference). In embodiments, the IgG constant region includes a triple M252Y/S254T/T256E mutation or YTE mutation. In embodiments, the IgG constant region includes a triple H433K/N434F/Y436H mutation or KFH mutation. In embodiments, the IgG constant region includes an YTE and KFH mutation in combination.

In embodiments, the linker comprises an IgG constant region that contains one or more mutations at amino acid residues 250, 253, 307, 310, 380, 428, 433, 434, and 435 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference). Illustrative mutations include T250Q, M428L, T307A, E380A, I253A, H310A, M428L, H433K, N434A, N434F, N434S, and H435A. In embodiments, the IgG constant region comprises a M428L/N434S mutation or LS mutation. In embodiments, the IgG constant region comprises a T250Q/M428L mutation or QL mutation. In embodiments, the IgG constant region comprises an N434A mutation. In embodiments, the IgG constant region comprises a T307A/E380A/N434A mutation or MA mutation. In embodiments, the IgG constant region comprises an I253A/H310A/H435A mutation or IHH mutation. In embodiments, the IgG constant region comprises a H433K/N434F mutation. In embodiments, the IgG constant region comprises a M252Y/S254T/T256E and a H433K/N434F mutation in combination.

Additional exemplary mutations in the IgG constant region are described, for example, in Robbie, et al., Antimicrobial Agents and Chemotherapy (2013), 57(12):6147-6153, Dall'Acqua et al., JBC (2006), 281(33):23514-24, Dall'Acqua et al., Journal of Immunology (2002), 169:5171-80, Ko et al. Nature (2014) 514:642-645, Grevys et al. Journal of Immunology. (2015), 194(11):5497-508, and U.S. Pat. No. 7,083,784, the entire contents of which are hereby incorporated by reference.

An illustrative Fc stabilizing mutant is S228P. Illustrative Fc half-life extending mutants are T250Q, M428L, V308T, L309P, and Q311S and the present linkers may comprise 1, or 2, or 3, or 4, or 5 of these mutants.

In embodiments, the chimeric protein binds to FcRn with high affinity. In embodiments, the chimeric protein may bind to FcRn with a K_{D} of about 1 nM to about 80 nM. For example, the chimeric protein may bind to FcRn with a K_{D} of about 1 nM, about 2 nM, about 3 nM, about 4 nM, about 5 nM, about 6 nM, about 7 nM, about 8 nM, about 9 nM, about 10 nM, about 15 nM, about 20 nM, about 25 nM, about 30 nM, about 35 nM, about 40 nM, about 45 nM, about 50 nM, about 55 nM, about 60 nM, about 65 nM, about 70 nM, about 71 nM, about 72 nM, about 73 nM, about 74 nM, about 75 nM, about 76 nM, about 77 nM, about 78 nM, about 79 nM, or about 80 nM. In embodiments, the chimeric protein may bind to FcRn with a K_{D} of about 9 nM. In embodiments, the chimeric protein does not substantially bind to other Fc receptors (i.e. other than FcRn) with effector function.

In embodiments, the Fc domain in a linker has the amino acid sequence of SEQ ID NO: 1 (see Table 1, below), or at least 90%, or 93%, or 95%, or 97%, or 98%, or 99% identity thereto. In embodiments, mutations are made to SEQ ID NO: 1 to increase stability and/or half-life. For instance, in embodiments, the Fc domain in a linker comprises the amino acid sequence of SEQ ID NO: 2 (see Table 1, below), or at least 90%, or 93%, or 95%, or 97%, or 98%, or 99% identity thereto. For instance, in embodiments, the Fc domain in a linker comprises the amino acid sequence of SEQ ID NO: 3 (see Table 1, below), or at least 90%, or 93%, or 95%, or 97%, or 98%, or 99% identity thereto.

Further, one or more joining linkers may be employed to connect an Fc domain in a linker (e.g., one of SEQ ID NO: 1, SEQ ID NO: 2, SEQ ID NO: 3 or at least 90%, or 93%, or 95%, or 97%, or 98%, or 99% identity thereto) and the extracellular domains. For example, any one of SEQ ID NO: 4, SEQ ID NO: 5, SEQ ID NO: 6, SEQ ID NO: 7, SEQ ID NO: 8, SEQ ID NO: 9, or variants thereof may connect an extracellular domain as disclosed herein and an Fc domain in a linker as disclosed herein. Optionally, any one of SEQ ID NOs: 4 to 50, or variants thereof are located between an extracellular domain as disclosed herein and an Fc domain as disclosed herein.

In embodiments, the present chimeric proteins may comprise variants of the joining linkers disclosed in Table 1, below. For instance, a linker may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the amino acid sequence of any one of SEQ ID NOs: 4 to 50.

In embodiments, the first and second joining linkers may be different or they may be the same.

Without wishing to be bound by theory, including a linker comprising at least a part of an Fc domain in a chimeric protein, helps avoid formation of insoluble and, likely, non-functional protein concatamers and/or aggregates. This is in part due to the presence of cysteines in the Fc domain which are capable of forming disulfide bonds between chimeric proteins.

In embodiments, a chimeric protein may comprise one or more joining linkers, as disclosed herein, and lack an Fc domain linker, as disclosed herein.

In embodiments, the first and/or second joining linkers are independently selected from the amino acid sequences of SEQ ID NOs: 4 to 50 and are provided in Table 1 below:

TABLE 1
Illustrative linkers (Fc domain linkers and joining linkers)

| SEQ | |
|---|---|
| ID | |
| NO. | Sequence |
| 1 | APEFLGGPSVFLFPPKPKDTLMISRTPEVTCVVVDVSQEDPEVQFNWYVDGVEVHNAKTKPREEQFNS |
| | TYRVVSVLTVLHQDWLSGKEYKCKVSSKGLPSSIEKTISNATGQPREPQVYTLPPSQEEMTKNQVSLT |
| | CLVKGFYPSDIAVEWESNGQPENNYKTTPPVLDSDGSFFLYSRLTVDKSSWQEGNVFSCSVMHEALHN |
| | HYTQKSLSLSLGK |
| 2 | APEFLGGPSVFLFPPKPKDQLMISRTPEVTCVVVDVSQEDPEVQFNWYVDGVEVHNAKTKPREEQFNS |
| | TYRVVSVLTTPHSDWLSGKEYKCKVSSKGLPSSIEKTISNATGQPREPQVYTLPPSQEEMTKNQVSLT |
| | CLVKGFYPSDIAVEWESNGQPENNYKTTPPVLDSDGSFFLYSRLTVDKSSWQEGNVFSCSVLHEALHN |
| | HYTQKSLSLSLGK |
| 3 | APEFLGGPSVFLFPPKPKDQLMISRTPEVTCVVVDVSQEDPEVQFNWYVDGVEVHNAKTKPREEQFNS |
| | TYRVVSVLTVLHQDWLSGKEYKCKVSSKGLPSSIEKTISNATGQPREPQVYTLPPSQEEMTKNQVSLT |
| | CLVKGFYPSDIAVEWESNGQPENNYKTTPPVLDSDGSFFLYSRLTVDKSRWQEGNVFSCSVLHEALHN |
| | HYTQKSLSLSLGK |
| 4 | SKYGPPCPSCP |
| 5 | SKYGPPCPPCP |
| 6 | SKYGPP |
| 7 | IEGRMD |
| 8 | GGGVPRDCG |
| 9 | IEGRMDGGGGAGGGG |
| 10 | GGGSGGGS |
| 11 | GGGSGGGGSGGG |
| 12 | EGKSSGSGSESKST |
| 13 | GGSG |
| 14 | GGSGGGSGGGSG |
| 15 | EAAAKEAAAKEAAAK |
| 16 | EAAAREAAAREAAAREAAAR |
| 17 | GGGGSGGGGSGGGGSAS |
| 18 | GGGGAGGGG |
| 19 | GS or GGS or LE |
| 20 | GSGSGS |
| 21 | GSGSGSGSGS |
| 22 | GGGGSAS |
| 23 | APAPAPAPAPAPAPAPAPAP |
| 24 | OPPO |
| 25 | GGGGS |
| 26 | GGGGSGGGGS |
| 27 | GGGGSGGGGSGGGGS |
| 28 | GGGGSGGGGSGGGGSGGGGS |
| 29 | GGGGSGGGGSGGGGSGGGGSGGGGS |
| 30 | GGGGSGGGGSGGGGSGGGGSGGGGSGGGGS |
| 31 | GGGGSGGGGSGGGGSGGGGSGGGGSGGGGSGGGGS |
| 32 | GGGGSGGGGSGGGGSGGGGSGGGGSGGGGSGGGGSGGGGS |
| 33 | GGSGGSGGGGSGGGGS |
| 34 | GGGGGGGG |
| 35 | GGGGGG |
| 36 | EAAAK |
| 37 | EAAAKEAAAK |
| 38 | EAAAKEAAAKEAAAK |
| 39 | AEAAAKEAAAKA |
| 40 | AEAAAKEAAAKEAAAKA |
| 41 | AEAAAKEAAAKEAAAKEAAAKA |
| 42 | AEAAAK EAAAK EAAAK EAAAK EAAAKA |
| 43 | AEAAAKEAAAK EAAAK EAAAKALEAEAAAK EAAAK EAAAK EAAAKA |
| 44 | PAPAP |
| 45 | KESGSVSSEQLAQFRSLD |
| 46 | GSAGSAAGSGEF |
| 47 | GGGSE |
| 48 | GSESG |
| 49 | GSEGS |
| 50 | GEGGSGEGSSGEGSSSEGGGSEGGGSEGGGSEGGS |

In embodiments, the joining linker substantially comprises glycine and serine residues (e.g., about 30%, or about 40%, or about 50%, or about 60%, or about 70%, or about 80%, or about 90%, or about 95%, or about 97%, or about 98%, or about 99%, or about 100% glycines and serines). For example, in embodiments, the joining linker is (Gly₄Ser)ₙ, where n is from about 1 to about 8, e.g., 1, 2, 3, 4, 5, 6, 7, or 8 (SEQ ID NO: 25 to SEQ ID NO: 32, respectively). In embodiments, the joining linker sequence is GGSGGSGGGGSGGGGS (SEQ ID NO: 33). Additional illustrative joining linkers include, but are not limited to, linkers having the sequence LE, (EAAAK)ₙ (n=1-3) (SEQ ID NO: 36 to SEQ ID NO: 38), A(EAAAK)ₙA (n=2-5) (SEQ ID NO: 39 to SEQ ID NO: 42), A(EAAAK)₄ALEA(EAAAK)₄A (SEQ ID NO: 43), PAPAP (SEQ ID NO: 44), KESGSVSSEQLAQFRSLD (SEQ ID NO: 45), GSAGSAAGSGEF (SEQ ID NO: 46), and (XP)ₙ, with X designating any amino acid, e.g., Ala, Lys, or Glu. In embodiments, the joining linker is GGS. In embodiments, a joining linker has the sequence (Gly)ₙ where n is any number from 1 to 100, for example: (Gly)₈ (SEQ ID NO: 34) and (Gly)₆ (SEQ ID NO: 35).

In embodiments, the joining linker is one or more of GGGSE (SEQ ID NO: 47), GSESG (SEQ ID NO: 48), GSEGS (SEQ ID NO: 49), GEGGSGEGSSGEGSSSEGGGSEGGGSEGGGSEGGS (SEQ ID NO: 50), and a joining linker of randomly placed G, S, and E every 4 amino acid intervals.

In embodiments, where a chimeric protein comprises an extracellular domain (ECD) of CD80, one joining linker preceding an Fc domain, a second joining linker following the Fc domain, and an ECD of NKG2A, the chimeric protein may comprise the following structure:
ECD of CD80—Joining Linker 1—Fc Domain—Joining Linker 2—ECD of NKG2A

An example of this chimeric protein comprises the sequence of SEQ ID NO: 61.

In embodiments, where a chimeric protein comprises an extracellular domain (ECD) of CD86, one joining linker preceding an Fc domain, a second joining linker following the Fc domain, and an ECD of NKG2A, the chimeric protein may comprise the following structure:
ECD of CD86—Joining Linker 1—Fc Domain—Joining Linker 2—ECD of NKG2A

An example of this chimeric protein comprises the sequence of SEQ ID NO: 65.

In embodiments, where a chimeric protein comprises an extracellular domain (ECD) of CD58, one joining linker preceding an Fc domain, a second joining linker following the Fc domain, and an ECD of NKG2A, the chimeric protein may comprise the following structure:

ECD of CD58—Joining Linker 1—Fc Domain—Joining Linker 2—ECD of NKG2A

An example of this chimeric protein comprises the sequence of SEQ ID NO: 68.

In embodiments, where a chimeric protein comprises an extracellular domain (ECD) of PD-1, one joining linker preceding an Fc domain, a second joining linker following the Fc domain, and an ECD of NKG2A, the chimeric protein may comprise the following structure:

ECD of PD-1—Joining Linker 1—Fc Domain—Joining Linker 2—ECD of NKG2A

An example of this chimeric protein comprises the sequence of SEQ ID NO: 71.

In embodiments, where a chimeric protein comprises an extracellular domain (ECD) of SLAMF6, one joining linker preceding an Fc domain, a second joining linker following the Fc domain, and an ECD of NKG2A, the chimeric protein may comprise the following structure:

ECD of SLAMF6—Joining Linker 1—Fc Domain—Joining Linker 2—ECD of NKG2A

An example of this chimeric protein comprises the sequence of SEQ ID NO: 75.

In embodiments, where a chimeric protein comprises an extracellular domain (ECD) of SIRPα, one joining linker preceding an Fc domain, a second joining linker following the Fc domain, and an ECD of NKG2A, the chimeric protein may comprise the following structure:

ECD of SIRPα—Joining Linker 1—Fc Domain—Joining Linker 2—ECD of NKG2A

An example of this chimeric protein comprises the sequence of SEQ ID NO: 86.

In embodiments, where a chimeric protein comprises an extracellular domain (ECD) of TGFBR2, one joining linker preceding an Fc domain, a second joining linker following the Fc domain, and an ECD of NKG2A, the chimeric protein may comprise the following structure:

ECD of TGFBR2—Joining Linker 1—Fc Domain—Joining Linker 2—ECD of NKG2A

An example of this chimeric protein comprises the sequence of SEQ ID NO: 79.

In embodiments, where a chimeric protein comprises a portion of the membrane-anchored extracellular protein CD48, one joining linker preceding an Fc domain, a second joining linker following the Fc domain, and an ECD of NKG2A, the chimeric protein may comprise the following structure:

Portion of CD48—Joining Linker 1—Fc Domain—Joining Linker 2—ECD of NKG2A

An example of this chimeric protein comprises the sequence of SEQ ID NO: 83.

The combination of a first joining linker, an Fc Domain linker, and a second joining linker is referend to herein as a “modular linker”. In embodiments, a chimeric protein comprises a modular linker as shown in Table 2:

TABLE 2
Illustrative modular linkers

| | | Joining | Modular Linker = Joining Linker |
|---|---|---|---|
| Joining Linker 1 | Fc | Linker 2 | 1 + Fc + Joining Linker 2 |
| SKYGPPCPSCP | APEFLGGPSVFLFPPKPKDTLMIS | IEGRMD | SKYGPPCPSCPAPEFLGGPSVFL |
| (SEQ ID NO: 4) | RTPEVTCVWDVSQEDPEVQFN | (SEQ ID NO: 7) | FPPKPKDTLMISRTPEVTCVWDV |
| | WYVDGVEVHNAKTKPREEQFNS | | SQEDPEVQFNWYVDGVEVHNAK |
| | TYRWSVLTVLHQDWLSGKEYKC | | TKPREEQFNSTYRWSVLTVLHQ |
| | KVSSKGLPSSIEKTISNATGQPRE | | DWLSGKEYKCKVSSKGLPSSIEK |
| | PQVYTLPPSQEEMTKNQVSLTCL | | TISNATGQPREPQVYTLPPSQEE |
| | VKGFYPSDIAVEWESNGQPENNY | | MTKNQVSLTCLVKGFYPSDIAVE |
| | KTTPPVLDSDGSFFLYSRLTVDKS | | WESNGQPENNYKTTPPVLDSDG |
| | SWQEGNVFSCSVMHEALHNHYT | | SFFLYSRLTVDKSSWQEGNVFSC |
| | QKSLSLSLGK (SEQ ID NO: 1) | | SVMHEALHNHYTQKSLSLSLGKIE |
| | | | GRMD (SEQ ID NO: 51) |
| SKYGPPCPSCP | APEFLGGPSVFLFPPKPKDQLMIS | IEGRMD | SKYGPPCPSCPAPEFLGGPSVFL |
| (SEQ ID NO: 4) | RTPEVTCVWDVSQEDPEVQFN | (SEQ ID NO: 7) | FPPKPKDQLMISRTPEVTCVWD |
| | WYVDGVEVHNAKTKPREEQFNS | | VSQEDPEVQFNWYVDGVEVHNA |
| | TYRWSVLTTPHSDWLSGKEYKC | | KTKPREEQFNSTYRVVSVLTTPH |
| | KVSSKGLPSSIEKTISNATGQPRE | | SDWLSGKEYKCKVSSKGLPSSIE |
| | PQVYTLPPSQEEMTKNQVSLTCL | | KTISNATGQPREPQVYTLPPSQE |
| | VKGFYPSDIAVEWESNGQPENNY | | EMTKNQVSLTCLVKGFYPSDIAV |
| | KTTPPVLDSDGSFFLYSRLTVDKS | | EWESNGQPENNYKTTPPVLDSD |
| | SWQEGNVFSCSVLHEALHNHYT | | GSFFLYSRLTVDKSSWQEGNVFS |
| | QKSLSLSLGK (SEQ ID NO: 2) | | CSVLHEALHNHYTQKSLSLSLGKI |
| | | | EGRMD (SEQ ID NO: 52) |
| SKYGPPCPSCP | APEFLGGPSVFLFPPKPKDQLMIS | IEGRMD | SKYGPPCPSCPAPEFLGGPSVFL |
| (SEQ ID NO: 4) | RTPEVTCVWDVSQEDPEVQFN | (SEQ ID NO: 7) | FPPKPKDQLMISRTPEVTCVWD |
| | WYVDGVEVHNAKTKPREEQFNS | | VSQEDPEVQFNWYVDGVEVHNA |
| | TYRWSVLTVLHQDWLSGKEYKC | | KTKPREEQFNSTYRVVSVLTVLH |
| | KVSSKGLPSSIEKTISNATGQPRE | | QDWLSGKEYKCKVSSKGLPSSIE |
| | PQVYTLPPSQEEMTKNQVSLTCL | | KTISNATGQPREPQVYTLPPSQE |
| | VKGFYPSDIAVEWESNGQPENNY | | EMTKNQVSLTCLVKGFYPSDIAV |
| | KTTPPVLDSDGSFFLYSRLTVDKS | | EWESNGQPENNYKTTPPVLDSD |
| | RWQEGNVFSCSVLHEALHNHYT | | GSFFLYSRLTVDKSRWQEGNVFS |
| | QKSLSLSLGK (SEQ ID NO: 3) | | CSVLHEALHNHYTQKSLSLSLGKI |
| | | | EGRMD (SEQ ID NO: 53) |
| SKYGPPCPPCP | APEFLGGPSVFLFPPKPKDTLMIS | IEGRMD | SKYGPPCPPCPAPEFLGGPSVFL |
| (SEQ ID NO: 5) | RTPEVTCVWDVSQEDPEVQFN | (SEQ ID NO: 7) | FPPKPKDTLMISRTPEVTCVWDV |
| | WYVDGVEVHNAKTKPREEQFNS | | SQEDPEVQFNWYVDGVEVHNAK |
| | TYRWSVLTVLHQDWLSGKEYKC | | TKPREEQFNSTYRWSVLTVLHQ |
| | KVSSKGLPSSIEKTISNATGQPRE | | DWLSGKEYKCKVSSKGLPSSIEK |
| | PQVYTLPPSQEEMTKNQVSLTCL | | TISNATGQPREPQVYTLPPSQEE |
| | VKGFYPSDIAVEWESNGQPENNY | | MTKNQVSLTCLVKGFYPSDIAVE |
| | KTTPPVLDSDGSFFLYSRLTVDKS | | WESNGQPENNYKTTPPVLDSDG |
| | SWQEGNVFSCSVMHEALHNHYT | | SFFLYSRLTVDKSSWQEGNVFSC |
| | QKSLSLSLGK (SEQ ID NO: 1) | | SVMHEALHNHYTQKSLSLSLGKIE |
| | | | GRMD (SEQ ID NO: 54) |
| SKYGPPCPPCP | APEFLGGPSVFLFPPKPKDQLMIS | IEGRMD | SKYGPPCPPCPAPEFLGGPSVFL |
| (SEQ ID NO: 5) | RTPEVTCVWDVSQEDPEVQFN | (SEQ ID NO: 7) | FPPKPKDQLMISRTPEVTCVWD |
| | WYVDGVEVHNAKTKPREEQFNS | | VSQEDPEVQFNWYVDGVEVHNA |
| | TYRWSVLTTPHSDWLSGKEYKC | | KTKPREEQFNSTYRVVSVLTTPH |
| | KVSSKGLPSSIEKTISNATGQPRE | | SDWLSGKEYKCKVSSKGLPSSIE |
| | PQVYTLPPSQEEMTKNQVSLTCL | | KTISNATGQPREPQVYTLPPSQE |
| | VKGFYPSDIAVEWESNGQPENNY | | EMTKNQVSLTCLVKGFYPSDIAV |
| | KTTPPVLDSDGSFFLYSRLTVDKS | | EWESNGQPENNYKTTPPVLDSD |
| | SWQEGNVFSCSVLHEALHNHYT | | GSFFLYSRLTVDKSSWQEGNVFS |
| | QKSLSLSLGK (SEQ ID NO: 2) | | CSVLHEALHNHYTQKSLSLSLGKI |
| | | | EGRMD (SEQ ID NO: 55) |
| SKYGPPCPPCP | APEFLGGPSVFLFPPKPKDQLMIS | IEGRMD | SKYGPPCPPCPAPEFLGGPSVFL |
| (SEQ ID NO: 5) | RTPEVTCVWDVSQEDPEVQFN | (SEQ ID NO: 7) | FPPKPKDQLMISRTPEVTCVWD |
| | WYVDGVEVHNAKTKPREEQFNS | | VSQEDPEVQFNWYVDGVEVHNA |
| | TYRWSVLTVLHQDWLSGKEYKC | | KTKPREEQFNSTYRVVSVLTVLH |
| | KVSSKGLPSSIEKTISNATGQPRE | | QDWLSGKEYKCKVSSKGLPSSIE |
| | PQVYTLPPSQEEMTKNQVSLTCL | | KTISNATGQPREPQVYTLPPSQE |
| | VKGFYPSDIAVEWESNGQPENNY | | EMTKNQVSLTCLVKGFYPSDIAV |
| | KTTPPVLDSDGSFFLYSRLTVDKS | | EWESNGQPENNYKTTPPVLDSD |
| | RWQEG NVFSCSVLH EALH N HYT | | GSFFLYSRLTVDKSRWQEGNVFS |
| | QKSLSLSLGK (SEQ ID NO: 3) | | CSVLHEALHNHYTQKSLSLSLGKI |
| | | | EGRMD (SEQ ID NO: 56) |

In embodiments, the present chimeric proteins may comprise variants of the modular linkers disclosed in Table 2, above. For instance, a linker may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the amino acid sequence of any one of SEQ ID NOs: 51 to 56.

In embodiments, the linker may be flexible, including without limitation highly flexible. In embodiments, the linker may be rigid, including without limitation a rigid alpha helix. Characteristics of illustrative joining linkers is shown below in Table 3:

TABLE 3
Characteristics of illustrative joining linkers

| Joining Linker Sequence | Characteristics |
|---|---|
| SKYGPPCPPCP (SEQ ID NO: 5) | IgG4 Hinge Region |
| IEGRMD (SEQ ID NO: 7) | Linker |
| GGGVPRDCG (SEQ ID NO: 8) | Flexible |
| GGGSGGGS (SEQ ID NO: 10) | Flexible |
| GGGSGGGGSGGG (SEQ ID NO: 11) | Flexible |
| EGKSSGSGSESKST (SEQ ID NO: 12) | Flexible + soluble |
| GGSG (SEQ ID NO: 13) | Flexible |
| GGSGGGSGGGSG (SEQ ID NO: 14) | Flexible |
| EAAAKEAAAKEAAAK (SEQ ID NO: 15) | Rigid Alpha Helix |
| EAAAREAAAREAAAREAAAR | Rigid Alpha Helix |
| (SEQ ID NO: 16) | |
| GGGGSGGGGSGGGGSAS | Flexible |
| (SEQ ID NO: 17) | |
| GGGGAGGGG (SEQ ID NO: 18) | Flexible |
| GS (SEQ ID NO: 19) | Highly flexible |
| GSGSGS (SEQ ID NO: 20) | Highly flexible |
| GSGSGSGSGS (SEQ ID NO: 21) | Highly flexible |
| GGGGSAS (SEQ ID NO: 22) | Flexible |
| APAPAPAPAPAPAPAPAPAP | Rigid |
| (SEQ ID NO: 23) | |

In embodiments, the linker may be functional. For example, without limitation, the linker may function to improve the folding and/or stability, improve the expression, improve the pharmacokinetics, and/or improve the bioactivity of the present chimeric protein. In another example, the linker may function to target the chimeric protein to a particular cell type or location.

In embodiments, a chimeric protein comprises only one joining linkers.

In embodiments, a chimeric protein lacks joining linkers.

In embodiments, the linker is a synthetic linker such as polyethylene glycol (PEG).

In embodiments, a chimeric protein has a first domain which is sterically capable of binding its ligand/receptor and/or the second domain which is sterically capable of binding its ligand/receptor. Thus, there is enough overall flexibility in the chimeric protein and/or physical distance between an extracellular domain (or portion thereof) and the rest of the chimeric protein such that the ligand/receptor binding domain of the extracellular domain is not sterically hindered from binding its ligand/receptor. This flexibility and/or physical distance (which is referred to as “slack”) may be normally present in the extracellular domain(s), normally present in the linker, and/or normally present in the chimeric protein (as a whole). Alternately, or additionally, an amino acid sequence (for example) may be added to one or more extracellular domains and/or to the linker to provide the slack needed to avoid steric hindrance. Any amino acid sequence that provides slack may be added. In embodiments, the added amino acid sequence comprises the sequence (Gly)ₙ where n is any number from 1 to 100. Additional examples of addable amino acid sequence include the joining linkers described in Table 1 and Table 3. In embodiments, a polyethylene glycol (PEG) linker may be added between an extracellular domain and a linker to provide the slack needed to avoid steric hindrance. Such PEG linkers are well known in the art.

In embodiments, a chimeric protein of the present invention comprises the extracellular domain of CD80 (or a variant thereof), a linker, and the extracellular domain of NKG2A (or a variant thereof). In embodiments, the linker comprises a hinge-CH2-CH3 Fc domain, e.g., from an IgG1 or from IgG4, including human IgG1 or IgG4. Thus, in embodiments, a chimeric protein of the present invention comprises the extracellular domain of CD80 (or a variant thereof), linker comprising a hinge-CH2-CH3 Fc domain, and the extracellular domain of NKG2A (or a variant thereof). Such a chimeric protein may be referred to herein as “CD80-Fc-NKG2A”.

In embodiments, a chimeric protein of the present invention comprises the extracellular domain of CD86 (or a variant thereof), a linker, and the extracellular domain of NKG2A (or a variant thereof). In embodiments, the linker comprises a hinge-CH2-CH3 Fc domain, e.g., from an IgG1 or from IgG4, including human IgG1 or IgG4. Thus, in embodiments, a chimeric protein of the present invention comprises the extracellular domain of CD86 (or a variant thereof), linker comprising a hinge-CH2-CH3 Fc domain, and the extracellular domain of NKG2A (or a variant thereof). Such a chimeric protein may be referred to herein as “CD86-Fc-NKG2A”.

In embodiments, a chimeric protein of the present invention comprises the extracellular domain of CD58 (or a variant thereof), a linker, and the extracellular domain of NKG2A (or a variant thereof). In embodiments, the linker comprises a hinge-CH2-CH3 Fc domain, e.g., from an IgG1 or from IgG4, including human IgG1 or IgG4. Thus, in embodiments, a chimeric protein of the present invention comprises the extracellular domain of CD58 (or a variant thereof), linker comprising a hinge-CH2-CH3 Fc domain, and the extracellular domain of NKG2A (or a variant thereof). Such a chimeric protein may be referred to herein as “CD58-Fc-NKG2A”.

In embodiments, a chimeric protein of the present invention comprises the extracellular domain of PD-1 (or a variant thereof), a linker, and the extracellular domain of NKG2A (or a variant thereof). In embodiments, the linker comprises a hinge-CH2-CH3 Fc domain, e.g., from an IgG1 or from IgG4, including human IgG1 or IgG4. Thus, in embodiments, a chimeric protein of the present invention comprises the extracellular domain of PD-1 (or a variant thereof), linker comprising a hinge-CH2-CH3 Fc domain, and the extracellular domain of NKG2A (or a variant thereof). Such a chimeric protein may be referred to herein as “PD-1-Fc-NKG2A”.

In embodiments, a chimeric protein of the present invention comprises the extracellular domain of SLAMF6 (or a variant thereof), a linker, and the extracellular domain of NKG2A (or a variant thereof). In embodiments, the linker comprises a hinge-CH2-CH3 Fc domain, e.g., from an IgG1 or from IgG4, including human IgG1 or IgG4. Thus, in embodiments, a chimeric protein of the present invention comprises the extracellular domain of SLAMF6 (or a variant thereof), linker comprising a hinge-CH2-CH3 Fc domain, and the extracellular domain of NKG2A (or a variant thereof). Such a chimeric protein may be referred to herein as “SLAMF6-Fc-NKG2A”.

In embodiments, a chimeric protein of the present invention comprises the extracellular domain of SIRPα (or a variant thereof), a linker, and the extracellular domain of NKG2A (or a variant thereof). In embodiments, the linker comprises a hinge-CH2-CH3 Fc domain, e.g., from an IgG1 or from IgG4, including human IgG1 or IgG4. Thus, in embodiments, a chimeric protein of the present invention comprises the extracellular domain of SIRPα (or a variant thereof), linker comprising a hinge-CH2-CH3 Fc domain, and the extracellular domain of NKG2A (or a variant thereof). Such a chimeric protein may be referred to herein as “SIRPα-Fc-NKG2A”.

In embodiments, a chimeric protein of the present invention comprises the extracellular domain of TGFBR2 (or a variant thereof), a linker, and the extracellular domain of NKG2A (or a variant thereof). In embodiments, the linker comprises a hinge-CH2-CH3 Fc domain, e.g., from an IgG1 or from IgG4, including human IgG1 or IgG4. Thus, in embodiments, a chimeric protein of the present invention comprises the extracellular domain of TGFBR2 (or a variant thereof), linker comprising a hinge-CH2-CH3 Fc domain, and the extracellular domain of NKG2A (or a variant thereof). Such a chimeric protein may be referred to herein as “TGFBR2-Fc-NKG2A”.

In embodiments, a chimeric protein of the present invention comprises a portion of the membrane-anchored extracellular protein CD48 (or a variant thereof), a linker, and the extracellular domain of NKG2A (or a variant thereof). In embodiments, the linker comprises a hinge-CH2-CH3 Fc domain, e.g., from an IgG1 or from IgG4, including human IgG1 or IgG4. Thus, in embodiments, a chimeric protein of the present invention comprises the portion of the membrane-anchored extracellular protein CD48 (or a variant thereof), linker comprising a hinge-CH2-CH3 Fc domain, and the extracellular domain of NKG2A (or a variant thereof). Such a chimeric protein may be referred to herein as “CD48-Fc-NKG2A”.

Non-limiting examples of chimeric proteins of the present disclosure are is shown below in Table 4:

| The Extracellular | | |
|---|---|---|
| Domain (ECD) of a | | |
| Type Transmembrane | | |
| Protein or a | The ECD Of A | |
| Membrane-Anchored | TypeII | Chimeric Protein of a General Structure of: |
| Extracellular | Transmembrane | N terminus-(a)-(b)-(c)-C terminus, Where |
| Protein (a) | Protein (c) | (b) is a Liker Adjoining (a) and (b) |
| Human CD80 ECD: | Human NKG2A | Human CD80-Fc-NKG2A chimeric protein: |
| VIHVTKEVKEVATLSCG | ECD: | VIHVTKEVKEVATLSCGHNVSVEELAQTRIYWQKEKK |
| HNVSVEELAQTRIYWQ | PSTLIQRHNNSS | MVLTMMSGDMNIWPEYKNRTIFDITNNLSIVILALRPS |
| KEKKMVLTMMSGDMNI | LNTRTQKARHC | DEGTYECVVLKYEKDAFKREHLAEVTLSVKADFPTPS |
| WPEYKNRTIFDITNNLSI | GHCPEEWITYS | ISDFEIPTSNIRRIICSTSGGFPEPHLSWLENGEELNAI |
| VILALRPSDEGTYECVV | NSCYYIGKERR | NTTVSQDPETELYAVSSKLDFNMTTNHSFMCLIKYGH |
| LKYEKDAFKREHLAEVT | TWEESLLACTS | LRVNQTFNWNTTKQEHFPDNSKYGPPCPPCPAPEFL |
| LSVKADFPTPSISDFEIP | KNSSLLSIDNEE | GGPSVFLFPPKPKDQLMISRTPEVTCVVVDVSQEDP |
| TSNIRRIICSTSGGFPEP | EMKFLSIISPSS | EVQFNWYVDGVEVHNAKTKPREEQFNSTYRWSVLT |
| HLSWLENGEELNAINTT | WIGVFRNSSHH | VLHQDWLSGKEYKCKVSSKGLPSSIEKTISNATGQPR |
| VSQDPETELYAVSSKL | PWVTMNGLAFK | EPQVYTLPPSQEEMTKNQVSLTCLVKGFYPSDIAVE |
| DFNMTTNHSFMCLIKY | HEIKDSDNAELN | WESNGQPENNYKTTPPVLDSDGSFFLYSRLTVDKSR |
| GHLRVNQTFNWNTTKQ | CAVLQVNRLKS | WQEGNVFSCSVLHEALHNHYTQKSLSLSLGKIEGRM |
| EHFPDN (SEQ ID NO: | AQCGSSHYHCK | DPSTLIQRHNNSSLNTRTQKARHCGHCPEEWITYSN |
| 59) | HKL (SEQ ID | SCYYIGKERRTWEESLLACTSKNSSLLSIDNEEEMKF |
| | NO: 57) | LSIISPSSWIGVFRNSSHHPWTMNGLAFKHEIKDSD |
| | | NAELNCAVLQVNRLKSAQCGSSIIYHCKHKL (SEQ ID |
| | | NO: 61) |
| Mouse CD80 ECD: | Mouse NKG2A | Mouse CD80-Fc-NKG2A |
| VDEQLSKSVKDKVLLPCRY | ECD: | VDEQLSKSVKDKVLLPCRYNSPHEDESEDRIYWQKHDK |
| NSPHEDESEDRIYWQKHD | TPYTEAKAQINS | VVLSVIAGKLKVWPEYKNRTLYDNTTYSLIILGLVLSDRGT |
| KVVLSVIAGKLKVWPEYKN | SMTRTHRDINY | YSCWQKKERGTYEVKHLALVKLSIKADFSTPNITESGNP |
| RTLYDNTTYSLIILGLVLSD | TLSSAQPCPHC | SADTKRITCFASGGFPKPRFSWLENGRELPGINTTISQDP |
| RGTYSCWQKKERGTYEV | PKEWISYSHNC | ESELYTISSQLDFNTTRNHTIKCLIKYGDAHVSEDFTWEK |
| KHLALVKLSIKADFSTPNIT | YFIGMERKSWN | PPEDPPDSKNVPRDCGCKPCICTVPEVSSVFIFPPKPKD |
| ESGNPSADTKRITCFASGG | DSLVSCISKNCS | VLTITLTPKVTCWVDISKDDPEVQFSWFVDDVEVHTAQT |
| FPKPRFSWLENGRELPGIN | LLYIDSEEEQDF | QPREEQFNSTFRSVSELPIMHQDWLNGKEFKCRVNSAA |
| TTISQDPESELYTISSQLDF | LQSLSLISWTGI | FPAPIEKTISKTKGRPKAPQVYTIPPPKEQMAKDKVSLTC |
| NTTRNHTIKCLIKYGDAHV | LRKGRGQPWV | MITDFFPEDITVEWQWNGQPAENYKNTQPIMDTDGSYF |
| SEDFTWEKPPEDPPDSKN | WKEDSIFKPKIA | VYSKLNVQKSNWEAGNTFTCSVLHEGLHNHHTEKSLSH |
| (SEQ ID NO: 60) | EILHDECNCAM | SPGIIEGRMDTPYTEAKAQINSSMTRTHRDINYTLSSAQP |
| | MSASGLTADNC | CPHCPKEWISYSHNCYFIGMERKSWNDSLVSCISKNCSL |
| | TTLHPYLCKCKF | LYIDSEEEQDFLQSLSLISWTGILRKGRGQPWVWKEDSIF |
| | PI (SEQ ID NO: | KPKIAEILHDECNCAMMSASGLTADNCTTLHPYLCKCKF |
| | 58) | PI |
| | | (SEQ ID NO: 62) |
| Human CD86 ECD: | Human NKG2A | Human CD86-Fc-NKG2A chimeric protein: |
| APLKIQAYFNETADLPC | ECD: | APLKIQAYFNETADLPCQFANSQNQSLSELVVFWQD |
| QFANSQNQSLSELVVF | PSTLIQRHNNSS | QENLVLNEVYLGKEKFDSVHSKYMGRTSFDSDSWTL |
| WQDQENLVLNEVYLGK | LNTRTQKARHC | RLHNLQIKDKGLYQCIIHHKKPTGMIRIHQMNSELSVL |
| EKFDSVHSKYMGRTSF | GHCPEEWITYS | ANFSQPEIVPISNITENVYINLTCSSIHGYPEPKKMSVL |
| DSDSWTLRLHNLQIKD | NSCYYIGKERR | LRTKNSTIEYDGVMQKSQDNVTELYDVSISLSVSFPD |
| KGLYQCIIHHKKPTGMI | TWEESLLACTS | VTSNMTIFCILETDKTRLLSSPFSIELEDPQPPPDHIPS |
| RIHQMNSELSVLANFS | KNSSLLSIDNEE | KYGPPCPPCPAPEFLGGPSVFLFPPKPKDQLMISRTP |
| QPEIVPISNITENVYINLT | EMKFLSIISPSS | EVTCVWDVSQEDPEVQFNWYVDGVEVHNAKTKPR |
| CSSIHGYPEPKKMSVLL | WIGVFRNSSHH | EEQFNSTYRVVSVLTVLHQDWLSGKEYKCKVSSKGL |
| RTKNSTIEYDGVMQKS | PWVTMNGLAFK | PSSIEKTISNATGQPREPQVYTLPPSQEEMTKNQVSL |
| QDNVTELYDVSISLSVS | HEIKDSDNAELN | TCLVKGFYPSDIAVEWESNGQPENNYKTTPPVLDSD |
| FPDVTSNMTIFCILETDK | CAVLQVNRLKS | GSFFLYSRLTVDKSRWQEGNVFSCSVLHEALHNHYT |
| TRLLSSPFSIELEDPQP | AQCGSSHYHCK | QKSLSLSLGKIEGRMDPSTLIQRHNNSSLNTRTQKAR |
| PPDHIP (SEQ ID NO: | HKL (SEQ ID | HCGHCPEEWITYSNSCYYIGKERRTWEESLLACTSK |
| 63) | NO: 57) | NSSLLSIDNEEEMKFLSIISPSSWIGVFRNSSHHPWWT |
| | | MNGLAFKHEIKDSDNAELNCAVLQVNRLKSAQCGSSI |
| | | IYHCKHKL (SEQ ID NO: 65) |
| Mouse CD86 ECD: | Mouse NKG2A | Mouse CD86-Fc-NKG2A chimeric protein: |
| VSVETQAYFNGTAYLP | ECD: | VSVETQAYFNGTAYLPCPFTKAQNISLSELVVFWQDQ |
| CPFTKAQNISLSELWF | TPYTEAKAQINS | QKLVLYEHYLGTEKLDSVNAKYLGRTSFDRNNWTLR |
| WQDQQKLVLYEHYLGT | SMTRTHRDINY | LHNVQIKDMGSYDCFIQKKPPTGSIILQQTLTELSVIAN |
| EKLDSVNAKYLGRTSF | TLSSAQPCPHC | FSEPEIKLAQNVTGNSGINLTCTSKQGHPKPKKMYFLI |
| DRNNWTLRLHNVQIKD | PKEWISYSHNC | TNSTNEYGDNMQISQDNVTELFSISNSLSLSFPDGVW |
| MGSYDCFIQKKPPTGSI | YFIGMERKSWN | HMTVVCVLETESMKISSKPLNFTQEFPSPQTYWKVP |
| ILQQTLTELSVIANFSEP | DSLVSCISKNCS | RDCGCKPCICTVPEVSSVFIFPPKPKDVLTITLTPKVT |
| EIKLAQNVTGNSGINLT | LLYIDSEEEQDF | CVVVDISKDDPEVQFSWFVDDVEVHTAQTQPREEQF |
| CTSKQGHPKPKKMYFLI | LQSLSLISWTGI | NSTFRSVSELPIMHQDWLNGKEFKCRVNSAAFPAPIE |
| TNSTNEYGDNMQISQD | LRKGRGQPWV | KTISKTKGRPKAPQVYTIPPPKEQMAKDKVSLTCMITD |
| NVTELFSISNSLSLSFP | WKEDSIFKPKIA | FFPEDITVEWQWNGQPAENYKNTQPIMDTDGSYFVY |
| DGVWHMTVVCVLETES | EILHDECNCAM | SKLNVQKSNWEAGNTFTCSVLHEGLHNHHTEKSLSH |
| MKISSKPLNFTQEFPSP | MSASGLTADNC | SPGIIEGRMDTPYTEAKAQINSSMTRTHRDINYTLSSA |
| QTYWK (SEQ ID NO: | TTLHPYLCKCKF | QPCPHCPKEWISYSHNCYFIGMERKSWNDSLVSCIS |
| 64) | PI (SEQ ID NO: | KNCSLLYIDSEEEQDFLQSLSLISWTGILRKGRGQPW |
| | 58) | VWKEDSIFKPKIAEILHDECNCAMMSASGLTADNCTT |
| | | LHPYLCKCKFPI (SEQ ID NO: 66) |
| Human CD58 ECD: | Human NKG2A | Human CD58-Fc-NKG2A chimeric protein: |
| FSQQIYGVVYGNVTFH | ECD: | FSQQIYGVVYGNVTFHVPSNVPLKEVLWKKQKDKVA |
| VPSNVPLKEVLWKKQK | PSTLIQRHNNSS | ELENSEFRAFSSFKNRVYLDTVSGSLTIYNLTSSDED |
| DKVAELENSEFRAFSSF | LNTRTQKARHC | EYEMESPNITDTMKFFLYVLESLPSPTLTCALTNGSIE |
| KNRVYLDTVSGSLTIYN | GHCPEEWITYS | VQCMIPEHYNSHRGLIMYSWDCPMEQCKRNSTSIYF |
| LTSSDEDEYEMESPNIT | NSCYYIGKERR | KMENDLPQKIQCTLSNPLFNTTSSIILTTCIPSSGHSRH |
| DTMKFFLYVLESLPSPT | TWEESLLACTS | RSKYGPPCPPCPAPEFLGGPSVFLFPPKPKDQLMISR |
| LTCALTNGSIEVQCMIP | KNSSLLSIDNEE | TPEVTCVVVDVSQEDPEVQFNWYVDGVEVHNAKTK |
| EHYNSHRGLIMYSWDC | EMKFLSIISPSS | PREEQFNSTYRVVSVLTVLHQDWLSGKEYKCKVSSK |
| PMEQCKRNSTSIYFKM | WIGVFRNSSHH | GLPSSIEKTISNATGQPREPQVYTLPPSQEEMTKNQV |
| ENDLPQKIQCTLSNPLF | PWVTMNGLAFK | SLTCLVKGFYPSDIAVEWESNGQPENNYKTTPPVLDS |
| NTTSSIILTTCIPSSGHS | HEIKDSDNAELN | DGSFFLYSRLTVDKSRWQEGNVFSCSVLHEALHNHY |
| RHR (SEQ ID NO: 67) | CAVLQVNRLKS | TQKSLSLSLGKIEGRMDPSTLIQRHNNSSLNTRTQKA |
| | AQCGSSIIYHCK | RHCGHCPEEWITYSNSCYYIGKERRTWEESLLACTS |
| | HKL (SEQ ID | KNSSLLSIDNEEEMKFLSIISPSSWIGVFRNSSHHPWV |
| | NO: 57) | TMNGLAFKHEIKDSDNAELNCAVLQVNRLKSAQCGS |
| | | SHYHCKHKL (SEQ ID NO: 68) |
| Human PD-1 ECD: | Human NKG2A | Human PD-1-Fc-NKG2A chimeric protein: |
| LDSPDRPWNPPTFSPA | ECD: | LDSPDRPWNPPTFSPALLVVTEGDNATFTCSFSNTS |
| LLVVTEGDNATFTCSFS | PSTLIQRHNNSS | ESFVLNWYRMSPSNQTDKLAAFPEDRSQPGQDCRF |
| NTSESFVLNWYRMSPS | LNTRTQKARHC | RVTQLPNGRDFHMSWRARRNDSGTYLCGAISLAPK |
| NQTDKLAAFPEDRSQP | GHCPEEWITYS | AQIKESLRAELRVTERRAEVPTAHPSPSPRPAGQFQS |
| GQDCRFRVTQLPNGR | NSCYYIGKERR | KYGPPCPPCPAPEFLGGPSVFLFPPKPKDQLMISRTP |
| DFHMSVVRARRNDSGT | TWEESLLACTS | EVTCVVVDVSQEDPEVQFNWYVDGVEVHNAKTKPR |
| YLCGAISLAPKAQIKESL | KNSSLLSIDNEE | EEQFNSTYRVVSVLTVLHQDWLSGKEYKCKVSSKGL |
| RAELRVTERRAEVPTA | EMKFLSIISPSS | PSSIEKTISNATGQPREPQVYTLPPSQEEMTKNQVSL |
| HPSPSPRPAGQFQ | WIGVFRNSSHH | TCLVKGFYPSDIAVEWESNGQPENNYKTTPPVLDSD |
| (SEQ ID NO: 69) | PWVTMNGLAFK | GSFFLYSRLTVDKSRWQEGNVFSCSVLHEALHNHYT |
| | HEIKDSDNAELN | QKSLSLSLGKIEGRMDPSTLIQRHNNSSLNTRTQKAR |
| | CAVLQVNRLKS | HCGHCPEEWITYSNSCYYIGKERRTWEESLLACTSK |
| | AQCGSSIIYHCK | NSSLLSIDNEEEMKFLSIISPSSWIGVFRNSSHHPWVT |
| | HKL (SEQ ID | MNGLAFKHEIKDSDNAELNCAVLQVNRLKSAQCGSSI |
| | NO: 57) | IYHCKHKL (SEQ ID NO: 71) |
| Mouse PD-1 ECD: | Mouse NKG2A | Mouse PD-1-Fc-NKG2A chimeric protein: |
| SGWLLEVPNGPWRSLT | ECD: | SEDLMLNWNRLSPSNQTEKQAAFCNGLSQPVQDAR |
| FYPAWLTVSEGANATF | TPYTEAKAQINS | FQIIQLPNRHDFHMNILDTRRNDSGIYLCGAISLHPKA |
| TCSLSNWSEDLMLNW | SMTRTHRDINY | KIEESPGAELVVTERILETSTRYPSPSPKPEGRFQGM |
| NRLSPSNQTEKQAAFC | TLSSAQPCPHC | VPRDCGCKPCICTVPEVSSVFIFPPKPKDVLTITLTPK |
| NGLSQPVQDARFQIIQL | PKEWISYSHNC | VTCWVDISKDDPEVQFSWFVDDVEVHTAQTQPREE |
| PNRHDFHMNILDTRRN | YFIGMERKSWN | QFNSTFRSVSELPIMHQDWLNGKEFKCRVNSAAFPA |
| DSGIYLCGAISLHPKAKI | DSLVSCISKNCS | PIEKTISKTKGRPKAPQVYTIPPPKEQMAKDKVSLTCM |
| EESPGAELVVTERILET | LLYIDSEEEQDF | ITDFFPEDITVEWQWNGQPAENYKNTQPIMDTDGSY |
| STRYPSPSPKPEGRFQ | LQSLSLISWTGI | FVYSKLNVQKSNWEAGNTFTCSVLHEGLHNHHTEKS |
| GM (SEQ ID NO: 70) | LRKGRGQPWV | LSHSPGIIEGRMDTPYTEAKAQINSSMTRTHRDINYTL |
| | WKEDSIFKPKIA | SSAQPCPHCPKEWISYSHNCYFIGMERKSWNDSLVS |
| | EILHDECNCAM | CISKNCSLLYIDSEEEQDFLQSLSLISWTGILRKGRGQ |
| | MSASGLTADNC | PWVWKEDSIFKPKIAEILHDECNCAMMSASGLTADNC |
| | TTLHPYLCKCKF | TTLHPYLCKCKFPI (SEQ ID NO: 72) |
| | PI (SEQ ID NO: | |
| | 58) | |
| Human SLAMF6 ECD: | Human NKG2A | Human SLAMF6-Fc-NKG2A chimeric protein: |
| QSSLTPLMVNGILGESV | ECD: | QSSLTPLMVNGILGESVTLPLEFPAGEKVNFITWLFNE |
| TLPLEFPAGEKVNFITW | PSTLIQRHNNSS | TSLAFIVPHETKSPEIHVTNPKQGKRLNFTQSYSLQLS |
| LFNETSLAFIVPHETKS | LNTRTQKARHC | NLKMEDTGSYRAQISTKTSAKLSSYTLRILRQLRNIQV |
| PEIHVTNPKQGKRLNFT | GHCPEEWITYS | TNHSQLFQNMTCELHLTCSVEDADDNVSFRWEALG |
| QSYSLQLSNLKMEDTG | NSCYYIGKERR | NTLSSQPNLTVSWDPRISSEQDYTCIAENAVSNLSFS |
| SYRAQISTKTSAKLSSY | TWEESLLACTS | VSAQKLCEDVKIQYTDTKMSKYGPPCPPCPAPEFLG |
| TLRILRQLRNIQVTNHS | KNSSLLSIDNEE | GPSVFLFPPKPKDQLMISRTPEVTCVVVDVSQEDPEV |
| QLFQNMTCELHLTCSV | EMKFLSIISPSS | QFNWYVDGVEVHNAKTKPREEQFNSTYRVVSVLTVL |
| EDADDNVSFRWEALGN | WIGVFRNSSHH | HQDWLSGKEYKCKVSSKGLPSSIEKTISNATGQPREP |
| TLSSQPNLTVSWDPRIS | PWWVTMNGLAFK | QVYTLPPSQEEMTKNQVSLTCLVKGFYPSDIAVEWE |
| SEQDYTCIAENAVSNLS | HEIKDSDNAELN | SNGQPENNYKTTPPVLDSDGSFFLYSRLTVDKSRWQ |
| FSVSAQKLCEDVKIQYT | CAVLQVNRLKS | EGNVFSCSVLHEALHNHYTQKSLSLSLGKIEGRMDPS |
| DTKM (SEQ ID NO: 73) | AQCGSSIIYHCK | TLIQRHNNSSLNTRTQKARHCGHCPEEWITYSNSCY |
| | HKL (SEQ ID | YIGKERRTWEESLLACTSKNSSLLSIDNEEEMKFLSIIS |
| | NO: 57) | PSSWIGVFRNSSHHPWVTMNGLAFKHEIKDSDNAEL |
| | | NCAVLQVNRLKSAQCGSSIIYHCKHKL (SEQ ID NO: |
| | | 75) |
| Mouse SLAMF6 ECD: | Mouse NKG2A | Mouse SLAMF6-Fc-NKG2A chimeric protein: |
| EVSQSSSDPQLMNGVL | ECD: | VSQSSSDPQLMNGVLGESAVLPLKLPAGKIANIIIWNY |
| GESAVLPLKLPAGKIANI | TPYTEAKAQINS | EWEASQVTALVINLSNPESPQIMNTDVKKRLNITQSY |
| IIWNYEWEASQVTALVI | SMTRTHRDINY | SLQISNLTMADTGSYTAQITTKDSEVITFKYILRVFERL |
| NLSNPESPQIMNTDVK | TLSSAQPCPHC | GNLETTNYTLLLENGTCQIHLACVLKNQSQTVSVEWQ |
| KRLNITQSYSLQISNLT | PKEWISYSHNC | ATGNISLGGPNVTIFWDPRNSGDQTYVCRAKNAVSN |
| MADTGSYTAQITTKDSE | YFIGMERKSWN | LSVSVSTQSLCKGVLTNPPWNVPRDCGCKPCICTVP |
| VITFKYILRVFERLGNLE | DSLVSCISKNCS | EVSSVFIFPPKPKDVLTITLTPKVTCVVVDISKDDPEVQ |
| TTNYTLLLENGTCQIHL | LLYIDSEEEQDF | FSWFVDDVEVHTAQTQPREEQFNSTFRSVSELPIMH |
| ACVLKNQSQTVSVEWQ | LQSLSLISWTGI | QDWLNGKEFKCRVNSAAFPAPIEKTISKTKGRPKAPQ |
| ATGNISLGGPNVTIFWD | LRKGRGQPWV | VYTIPPPKEQMAKDKVSLTCMITDFFPEDITVEWQWN |
| PRNSGDQTYVCRAKNA | WKEDSIFKPKIA | GQPAENYKNTQPIMDTDGSYFVYSKLNVQKSNWEA |
| VSNLSVSVSTQSLCKG | EILHDECNCAM | GNTFTCSVLHEGLHNHHTEKSLSHSPGIIEGRMDTPY |
| VLTNPPWN (SEQ ID | MSASGLTADNC | TEAKAQINSSMTRTHRDINYTLSSAQPCPHCPKEWIS |
| NO: 74) | TTLHPYLCKCKF | YSHNCYFIGMERKSWNDSLVSCISKNCSLLYIDSEEE |
| | PI (SEQ ID NO: | QDFLQSLSLISWTGILRKGRGQPWVWKEDSIFKPKIA |
| | 58) | EILHDECNCAMMSASGLTADNCTTLHPYLCKCKFPI |
| | | (SEQ ID NO: 76) |
| Human TGFBR2 ECD: | Human NKG2A | Human TGFBR2-Fc-NKG2A chimeric protein: |
| TIPPHVQKSVNNDMIVT | ECD: | TIPPHVQKSVNNDMIVTDNNGAVKFPQLCKFCDVRFS |
| DNNGAVKFPQLCKFCD | PSTLIQRHNNSS | TCDNQKSCMSNCSITSICEKPQEVCVAVWRKNDENIT |
| VRFSTCDNQKSCMSNC | LNTRTQKARHC | LETVCHDPKLPYHDFILEDAASPKCIMKEKKKPGETFF |
| SITSICEKPQEVCVAVW | GHCPEEWITYS | MCSCSSDECNDNIIFSEEYNTSNPDLLLVIFQSKYGPP |
| RKNDENITLETVCHDPK | NSCYYIGKERR | CPPCPAPEFLGGPSVFLFPPKPKDQLMISRTPEVTCV |
| LPYHDFILEDAASPKCI | TWEESLLACTS | VVDVSQEDPEVQFNWYVDGVEVHNAKTKPREEQFN |
| MKEKKKPGETFFMCSC | KNSSLLSIDNEE | STYRVVSVLTVLHQDWLSGKEYKCKVSSKGLPSSIEK |
| SSDECNDNHFSEEYNT | EMKFLSIISPSS | TISNATGQPREPQVYTLPPSQEEMTKNQVSLTCLVK |
| SNPDLLLVIFQ (SEQ ID | WIGVFRNSSHH | GFYPSDIAVEWESNGQPENNYKTTPPVLDSDGSFFL |
| NO: 77) | PWVTMNGLAFK | YSRLTVDKSRWQEGNVFSCSVLHEALHNHYTQKSLS |
| | HEIKDSDNAELN | LSLGKIEGRMDPSTLIQRHNNSSLNTRTQKARHCGH |
| | CAVLQVNRLKS | CPEEWITYSNSCYYIGKERRTWEESLLACTSKNSSLL |
| | AQCGSSIIYHCK | SIDNEEEMKFLSIISPSSWIGVFRNSSHHPWVTMNGL |
| | HKL (SEQ ID | AFKHEIKDSDNAELNCAVLQVNRLKSAQCGSSIIYHC |
| | NO: 57) | KHKL (SEQ ID NO: 79) |
| Mouse TGFBR2 ECD: | Mouse NKG2A | Mouse TGFBR2-Fc-NKG2A chimeric protein: |
| IPPHVPKSDVEMEAQK | ECD: | IPPHVPKSDVEMEAQKDASIHLSCNRTIHPLKHFNSD |
| DASIHLSCNRTIHPLKH | TPYTEAKAQINS | VMASDNGGAVKLPQLCKFCDVRLSTCDNQKSCMSN |
| FNSDVMASDNGGAVKL | SMTRTHRDINY | CSITAICEKPHEVCVAVWRKNDKNITLETVCHDPKLTY |
| PQLCKFCDVRLSTCDN | TLSSAQPCPHC | HGFTLEDAASPKCVMKEKKRAGETFFMCACNMEEC |
| QKSCMSNCSITAICEKP | PKEWISYSHNC | NDYIIFSEEYTTSSPDVPRDCGCKPCICTVPEVSSVFI |
| HEVCVAVWRKNDKNIT | YFIGMERKSWN | FPPKPKDVLTITLTPKVTCVVVDISKDDPEVQFSWFVD |
| LETVCHDPKLTYHGFTL | DSLVSCISKNCS | DVEVHTAQTQPREEQFNSTFRSVSELPIMHQDWLNG |
| EDAASPKCVMKEKKRA | LLYIDSEEEQDF | KEFKCRVNSAAFPAPIEKTISKTKGRPKAPQVYTIPPP |
| GETFFMCACNMEECN | LQSLSLISWTGI | KEQMAKDKVSLTCMITDFFPEDITVEWQWNGQPAEN |
| DYIIFSEEYTTSSPD | LRKGRGQPWV | YKNTQPIMDTDGSYFVYSKLNVQKSNWEAGNTFTCS |
| (SEQ ID NO: 78) | WKEDSIFKPKIA | VLHEGLHNHHTEKSLSHSPGIIEGRMDTPYTEAKAQI |
| | EILHDECNCAM | NSSMTRTHRDINYTLSSAQPCPHCPKEWISYSHNCY |
| | MSASGLTADNC | FIGMERKSWNDSLVSCISKNCSLLYIDSEEEQDFLQS |
| | TTLHPYLCKCKF | LSLISWTGILRKGRGQPWVWKEDSIFKPKIAEILHDEC |
| | PI (SEQ ID NO: | NCAMMSASGLTADNCTTLHPYLCKCKFPI (SEQ ID |
| | 58) | NO: 80) |
| Human CD48 ECD: | Human NKG2A | Human CD48-Fc-NKG2A chimeric protein: |
| QGHLVHMTVVSGSNVT | ECD: | QGHLVHMTVVSGSNVTLNISESLPENYKQLTWFYTF |
| LNISESLPENYKQLTWF | PSTLIQRHNNSS | DQKIVEWDSRKSKYFESKFKGRVRLDPQSGALYISKV |
| YTFDQKIVEWDSRKSK | LNTRTQKARHC | QKEDNSTYIMRVLKKTGNEQEWKIKLQVLDPVPKPVI |
| YFESKFKGRVRLDPQS | GHCPEEWITYS | KIEKIEDMDDNCYLKLSCVIPGESVNYTWYGDKRPFP |
| GALYISKVQKEDNSTYI | NSCYYIGKERR | KELQNSVLETTLMPHNYSRCYTCQVSNSVSSKNGTV |
| MRVLKKTGNEQEWKIK | TWEESLLACTS | CLSPPCTLARSSKYGPPCPPCPAPEFLGGPSVFLFPP |
| LQVLDPVPKPVIKIEKIE | KNSSLLSIDNEE | KPKDQLMISRTPEVTCVVVDVSQEDPEVQFNWYVDG |
| DMDDNCYLKLSCVIPG | EMKFLSIISPSS | VEVHNAKTKPREEQFNSTYRWSVLTVLHQDWLSGK |
| ESVNYTWYGDKRPFPK | WIGVFRNSSHH | EYKCKVSSKGLPSSIEKTISNATGQPREPQVYTLPPS |
| ELQNSVLETTLMPHNY | PWWVTMNGLAFK | QEEMTKNQVSLTCLVKGFYPSDIAVEWESNGQPENN |
| SRCYTCQVSNSVSSKN | HEIKDSDNAELN | YKTTPPVLDSDGSFFLYSRLTVDKSRWQEGNVFSCS |
| GTVCLSPPCTLAR | CAVLQVNRLKS | VLHEALHNHYTQKSLSLSLGKIEGRMDPSTLIQRHNN |
| (SEQ ID NO: 81) | AQCGSSIIYHCK | SSLNTRTQKARHCGHCPEEWITYSNSCYYIGKERRT |
| | HKL (SEQ ID | WEESLLACTSKNSSLLSIDNEEEMKFLSIISPSSWIGV |
| | NO: 57) | FRNSSHHPWVTMNGLAFKHEIKDSDNAELNCAVLQV |
| | | NRLKSAQCGSSIIYHCKHKL (SEQ ID NO: 83) |
| Mouse CD48 ECD: | Mouse NKG2A | Mouse CD48-Fc-NKG2A chimeric protein: |
| FQGHSIPDINATTGSNV | ECD: | FQGHSIPDINATTGSNVTLKIHKDPLGPYKRITWLHTK |
| TLKIHKDPLGPYKRITW | TPYTEAKAQINS | NQKILEYNYNSTKTIFESEFKGRVYLEENNGALHISNV |
| LHTKNQKILEYNYNSTK | SMTRTHRDINY | RKEDKGTYYMRVLRETENELKITLEVFDPVPKPSIEIN |
| TIFESEFKGRVYLEENN | TLSSAQPCPHC | KTEASTDSCHLRLSCEVKDQHVDYTWYESSGPFPKK |
| GALHISNVRKEDKGTYY | PKEWISYSHNC | SPGYVLDLIVTPQNKSTFYTCQVSNPVSSKNDTVYFT |
| MRVLRETENELKITLEV | YFIGMERKSWN | LPCDLARSVPRDCGCKPCICTVPEVSSVFIFPPKPKD |
| FDPVPKPSIEINKTEAST | DSLVSCISKNCS | VLTITLTPKVTCVVVDISKDDPEVQFSWFVDDVEVHT |
| DSCHLRLSCEVKDQHV | LLYIDSEEEQDF | AQTQPREEQFNSTFRSVSELPIMHQDWLNGKEFKCR |
| DYTWYESSGPFPKKSP | LQSLSLISWTGI | VNSAAFPAPIEKTISKTKGRPKAPQVYTIPPPKEQMAK |
| GYVLDLIVTPQNKSTFY | LRKGRGQPWV | DKVSLTCMITDFFPEDITVEWQWNGQPAENYKNTQPI |
| TCQVSNPVSSKNDTVY | WKEDSIFKPKIA | MDTDGSYFVYSKLNVQKSNWEAGNTFTCSVLHEGL |
| FTLPCDLARS (SEQ ID | EILHDECNCAM | HNHHTEKSLSHSPGIIEGRMDTPYTEAKAQINSSMTR |
| NO: 82) | MSASGLTADNC | THRDINYTLSSAQPCPHCPKEWISYSHNCYFIGMERK |
| | TTLHPYLCKCKF | SWNDSLVSCISKNCSLLYIDSEEEQDFLQSLSLISWTG |
| | PI (SEQ ID NO: | ILRKGRGQPWVWKEDSIFKPKIAEILHDECNCAMMSA |
| | 58) | SGLTADNCTTLHPYLCKCKFPI (SEQ ID NO: 84) |
| Human SIRPa ECD: | Human NKG2A | Human SIRPa-Fc-NKG2A chimeric protein: |
| EEELQVIQPDKSVLVAA | ECD: | EEELQVIQPDKSVLVAAGETATLRCTATSLIPVGPIQW |
| GETATLRCTATSLIPVG | PSTLIQRHNNSS | FRGAGPGRELIYNQKEGHFPRVTTVSDLTKRNNMDF |
| PIQWFRGAGPGRELIY | LNTRTQKARHC | SIRIGNITPADAGTYYCVKFRKGSPDDVEFKSGAGTE |
| NQKEGHFPRVTTVSDL | GHCPEEWITYS | LSVRAKPSAPVVSGPAARATPQHTVSFTCESHGFSP |
| TKRNNMDFSIRIGNITP | NSCYYIGKERR | RDITLKWFKNGNELSDFQTNVDPVGESVSYSIHSTAK |
| ADAGTYYCVKFRKGSP | TWEESLLACTS | VVLTREDVHSQVICEVAHVTLQGDPLRGTANLSETIR |
| DDVEFKSGAGTELSVR | KNSSLLSIDNEE | VPPTLEVTQQPVRAENQVNVTCQVRKFYPQRLQLTW |
| AKPSAPVVSGPAARAT | EMKFLSIISPSS | LENGNVSRTETASTVTENKDGTYNWMSWLLVNVSA |
| PQHTVSFTCESHGFSP | WIGVFRNSSHH | HRDDVKLTCQVEHDGQPAVSKSHDLKVSAHPKEQG |
| RDITLKWFKNGNELSDF | PWVTMNGLAFK | SNTAAENTGSNERNIYSKYGPPCPPCPAPEFLGGPS |
| QTNVDPVGESVSYSIH | HEIKDSDNAELN | VFLFPPKPKDQLMISRTPEVTCWVDVSQEDPEVQFN |
| STAKWLTREDVHSQVI | CAVLQVNRLKS | WYVDGVEVHNAKTKPREEQFNSTYRVVSVLTVLHQD |
| CEVAHVTLQGDPLRGT | AQCGSSIIYHCK | WLSGKEYKCKVSSKGLPSSIEKTISNATGQPREPQVY |
| ANLSETIRVPPTLEVTQ | HKL (SEQ ID | TLPPSQEEMTKNQVSLTCLVKGFYPSDIAVEWESNG |
| QPVRAENQVNVTCQVR | NO: 57) | QPENNYKTTPPVLDSDGSFFLYSRLTVDKSRWQEGN |
| KFYPQRLQLTWLENGN | | VFSCSVLHEALHNHYTQKSLSLSLGKIEGRMDPSTLI |
| VSRTETASTVTENKDG | | QRHNNSSLNTRTQKARHCGHCPEEWITYSNSCYYIG |
| TYNWMSWLLVNVSAH | | KERRTWEESLLACTSKNSSLLSIDNEEEMKFLSIISPS |
| RDDVKLTCQVEHDGQP | | SWIGVFRNSSHHPWVTMNGLAFKHEIKDSDNAELNC |
| AVSKSHDLKVSAHPKE | | AVLQVNRLKSAQCGSSIIYHCKHKL |
| QGSNTAAENTGSNERN | | (SEQ ID NO: 86) |
| IY (SEQ ID NO: 85) | | |

An aspect of the present invention is the use of a herein-disclosed chimeric protein as a medicament in the treatment of a cancer or a viral infection.

Another aspect of the present invention is the use of a herein-disclosed chimeric protein, in the manufacture of a medicament.

Yet another aspect of the present invention is an expression vector comprising a nucleic acid that encodes a herein-disclosed chimeric protein.

In an aspect, the present invention provides a host cell comprising an expression vector that comprises a nucleic acid that encodes a herein-disclosed chimeric protein.

### Diseases, Methods of Treatment, and Mechanisms of Action

A chimeric protein disclosed herein may be used in the treatment of cancer and/or in the treatment of a viral infection.

Aspects of the present invention provide methods of treating a cancer or treating a viral infection. The methods comprise a step of administering to a subject in need thereof an effective amount of a pharmaceutical composition comprising herein-disclosed chimeric protein, e.g., a therapeutically effective amount of the chimeric protein.

It is often desirable to enhance immune stimulatory signal transmission to boost an immune response, for instance to enhance a patient's anti-tumor immune response.

In embodiments, the present invention pertains to cancers and/or tumors; for example, the treatment or prevention of cancers and/or tumors. As disclosed elsewhere herein, the treatment of cancer involves, in embodiments, modulating the immune system with the present chimeric proteins to favor of increasing or activating immune stimulatory signals. In embodiments, the method reduces the amount or activity of regulatory T cells (Tregs) as compared to untreated subjects or subjects treated with antibodies directed to transmembrane proteins, membrane-bound extracellular proteins, and/or their respective ligands or receptors. In embodiments, the method increases priming of effector T cells in draining lymph nodes of the subject as compared to untreated subjects or subjects treated with antibodies directed to transmembrane proteins, membrane-bound extracellular proteins, and/or their respective ligands or receptors. In embodiments, the method causes an overall decrease in immunosuppressive cells and a shift toward a more inflammatory tumor environment as compared to untreated subjects or subjects treated with antibodies directed to transmembrane proteins, membrane-bound extracellular proteins, and/or their respective ligands or receptors.

In embodiments, the present chimeric proteins are capable of, or can be used in methods comprising, modulating the amplitude of an immune response, e.g., modulating the level of effector output. In embodiments, e.g. when used for the treatment of cancer, the present chimeric proteins alter the extent of immune stimulation as compared to immune inhibition to increase the amplitude of a T cell response, including, without limitation, stimulating increased levels of cytokine production, proliferation or target killing potential. In embodiments, the patient's T cells are activated and/or stimulated by the chimeric protein, with the activated T cells being capable of dividing and/or secreting cytokines.

Cancers or tumors refer to an uncontrolled growth of cells and/or abnormal increased cell survival and/or inhibition of apoptosis which interferes with the normal functioning of the bodily organs and systems. Included are benign and malignant cancers, polyps, hyperplasia, as well as dormant tumors or micrometastases. Also, included are cells having abnormal proliferation that is not impeded by the immune system (e.g., virus-infected cells). The cancer may be a primary cancer or a metastatic cancer. The primary cancer may be an area of cancer cells at an originating site that becomes clinically detectable, and may be a primary tumor. In contrast, the metastatic cancer may be the spread of a disease from one organ or part to another non-adjacent organ or part. The metastatic cancer may be caused by a cancer cell that acquires the ability to penetrate and infiltrate surrounding normal tissues in a local area, forming a new tumor, which may be a local metastasis. The cancer may also be caused by a cancer cell that acquires the ability to penetrate the walls of lymphatic and/or blood vessels, after which the cancer cell is able to circulate through the bloodstream (thereby being a circulating tumor cell) to other sites and tissues in the body. The cancer may be due to a process such as lymphatic or hematogeneous spread. The cancer may also be caused by a tumor cell that comes to rest at another site, re-penetrates through the vessel or walls, continues to multiply, and eventually forms another clinically detectable tumor. The cancer may be this new tumor, which may be a metastatic (or secondary) tumor.

The cancer may be caused by tumor cells that have metastasized, which may be a secondary or metastatic tumor. The cells of the tumor may be like those in the original tumor. As an example, if a breast cancer or colon cancer metastasizes to the liver, the secondary tumor, while present in the liver, is made up of abnormal breast or colon cells, not of abnormal liver cells. The tumor in the liver may thus be a metastatic breast cancer or a metastatic colon cancer, not liver cancer.

The cancer may have an origin from any tissue. The cancer may originate from melanoma, colon, breast, or prostate, and thus may be made up of cells that were originally skin, colon, breast, or prostate, respectively. The cancer may also be a hematological malignancy, which may be leukemia or lymphoma. The cancer may invade a tissue such as liver, lung, bladder, or intestinal.

Representative cancers and/or tumors of the present invention include, but are not limited to, cancer is selected from acute lymphoblastic leukemia (ALL); AIDS-related lymphoma; basal cell carcinoma; biliary tract cancer; bladder cancer; bone cancer; brain and central nervous system cancer; breast cancer; cancer of the digestive system; cancer of the head and neck; cancer of the peritoneum; cancer of the respiratory system; cancer of the urinary system; carcinoma; cervical cancer; choriocarcinoma; chronic lymphocytic leukemia (CLL); chronic myeloblastic leukemia; colon and rectum cancer; connective tissue cancer; edema (e.g., that is associated with brain tumors); endometrial cancer; esophageal cancer; eye cancer; gastric cancer (including gastrointestinal cancer); glioblastoma; Hairy cell leukemia; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma (NHL), as well as B-cell lymphoma (including low grade/follicular NHL, high grade immunoblastic NHL, high grade lymphoblastic NHL, high grade small non-cleaved cell NHL, intermediate grade diffuse NHL; intermediate grade/follicular NHL, bulky disease NHL, and small lymphocytic (SL) NHL); mantle cell lymphoma; Meigs' syndrome; melanoma; myeloma; neuroblastoma; oral cavity cancer (lip, tongue, mouth, and pharynx); ovarian cancer; pancreatic cancer; post-transplant lymphoproliferative disorder (PTLD), as well as abnormal vascular proliferation associated with phakomatoses; prostate cancer; rectal cancer; retinoblastoma; rhabdomyosarcoma; salivary gland carcinoma; sarcoma; skin cancer; squamous cell cancer; stomach cancer; testicular cancer; thyroid cancer; uterine or endometrial cancer; vulval cancer; and Waldenstrom's Macroglobulinemia.

In embodiments, the chimeric protein is used to treat a subject that has a treatment-refractory cancer. In embodiments, the chimeric protein is used to treat a subject that is refractory to one or more immune-modulating agents. For example, in embodiments, the chimeric protein is used to treat a subject that presents no response to treatment, or even progress, after 12 weeks or so of treatment. For instance, in embodiments, the subject is refractory to a PD-1 and/or PD-L1 and/or PD-L2 agent, including, for example, nivolumab (ONO-4538/BMS-936558, MDX1106, OPDIVO, BRISTOL MYERS SQUIBB), pembrolizumab (KEYTRUDA, MERCK), pidilizumab (CT-011, CURE TECH), MK-3475 (MERCK), BMS 936559 (BRISTOL MYERS SQUIBB), Ibrutinib (PHARMACYCLICS/ABBVIE), atezolizumab (TECENTRIQ, GENENTECH), and/or MPDL3280A (ROCHE)-refractory patients. For instance, in embodiments, the subject is refractory to an anti-CTLA-4 agent, e.g., ipilimumab (YERVOY)-refractory patients (e.g., melanoma patients). Accordingly, in embodiments the present invention provides methods of cancer treatment that rescue patients that are non-responsive to various therapies, including monotherapy of one or more immune-modulating agents.

In embodiments, the present invention provides chimeric proteins which target a cell or tissue within the tumor microenviroment. In embodiments, the cell or tissue within the tumor microenvironment expresses one or more targets or binding partners of the chimeric protein. The tumor microenvironment refers to the cellular milieu, including cells, secreted proteins, physiological small molecules, and blood vessels in which the tumor exists. In embodiments, the cells or tissue within the tumor microenvironment are one or more of: tumor vasculature; tumor-infiltrating lymphocytes; fibroblast reticular cells; endothelial progenitor cells (EPC); cancer-associated fibroblasts; pericytes; other stromal cells; components of the extracellular matrix (ECM); dendritic cells; antigen presenting cells; T-cells; regulatory T cells; macrophages; neutrophils; and other immune cells located proximal to a tumor. In embodiments, the present chimeric protein targets a cancer cell. In embodiments, the cancer cell expresses one or more of targets or binding partners of the chimeric protein.

In embodiments, the present methods provide treatment with the chimeric protein in a patient who is refractory to an additional agent, such “additional agents” being disclosed elsewhere herein, inclusive, without limitation, of the various chemotherapeutic agents disclosed herein.

The activation of regulatory T cells is critically influenced by costimulatory and co-inhibitory signals. Two major families of costimulatory molecules include the B7 and the tumor necrosis factor (TNF) families. These molecules bind to receptors on T cells belonging to the CD28 or TNF receptor families, respectively. Many well-defined co-inhibitors and their receptors belong to the B7 and CD28 families.

In embodiments, an immune stimulatory signal refers to a signal that enhances an immune response. For example, in the context of oncology, such signals may enhance antitumor immunity. For instance, without limitation, immune stimulatory signal may be identified by directly stimulating proliferation, cytokine production, killing activity, or phagocytic activity of leukocytes. Specific examples include direct stimulation of TNF superfamily receptors such as OX40, LTbR, 4-1BB or TNFRSF25 using either receptor agonist antibodies or using chimeric proteins encoding the ligands for such receptors (OX40L, LIGHT, 4-1BBL, TL1A, respectively). Stimulation from any one of these receptors may directly stimulate the proliferation and cytokine production of individual T cell subsets. Another example includes direct stimulation of an immune inhibitory cell with through a receptor that inhibits the activity of such an immune suppressor cell. This would include, for example, stimulation of CD4+FoxP3+ regulatory T cells with a GITR agonist antibody or GITRL containing chimeric protein, which would reduce the ability of those regulatory T cells to suppress the proliferation of conventional CD4+ or CD8+ T cells. In another example, this would include stimulation of CD40 on the surface of an antigen presenting cell using a CD40 agonist antibody or a chimeric protein containing CD40L, causing activation of antigen presenting cells including enhanced ability of those cells to present antigen in the context of appropriate native costimulatory molecules, including those in the B7 or TNF superfamily. In another example, this would include stimulation of LTBR on the surface of a lymphoid or stromal cell using a LIGHT containing chimeric protein, causing activation of the lymphoid cell and/or production of pro-inflammatory cytokines or chemokines to further stimulate an immune response, optionally within a tumor.

In embodiments, the present chimeric proteins are capable of, or find use in methods involving, enhancing, restoring, promoting and/or stimulating immune modulation. In embodiments, the present chimeric proteins described herein, restore, promote and/or stimulate the activity or activation of one or more immune cells against tumor cells including, but not limited to: T cells, cytotoxic T lymphocytes, T helper cells, natural killer (NK) cells, natural killer T (NKT) cells, anti-tumor macrophages (e.g. M1 macrophages), B cells, and dendritic cells. In embodiments, the present chimeric proteins enhance, restore, promote and/or stimulate the activity and/or activation of T cells, including, by way of a non-limiting example, activating and/or stimulating one or more T-cell intrinsic signals, including a pro-survival signal; an autocrine or paracrine growth signal; a p38 MAPK-, ERK-, STAT-, JAK-, AKT- or PI3K-mediated signal; an anti-apoptotic signal; and/or a signal promoting and/or necessary for one or more of: pro-inflammatory cytokine production or T cell migration or T cell tumor infiltration.

In embodiments, the present chimeric proteins are capable of, or find use in methods involving, causing an increase of one or more of T cells (including without limitation cytotoxic T lymphocytes, T helper cells, natural killer T (NKT) cells), B cells, natural killer (NK) cells, natural killer T (NKT) cells, dendritic cells, monocytes, and macrophages (e.g., one or more of M1 and M2) into a tumor or the tumor microenvironment. In embodiments, the chimeric protein enhances recognition of tumor antigens by CD8+ T cells, particularly those T cells that have infiltrated into the tumor microenvironment. In embodiments, the present chimeric protein induces CD19 expression and/or increases the number of CD19 positive cells (e.g., CD19 positive B cells). In embodiments, the present chimeric protein induces IL-15Rα expression and/or increases the number of IL-15Rα positive cells (e.g., IL-15Rα positive dendritic cells).

In embodiments, the present chimeric proteins are capable of, or find use in methods involving, inhibiting and/or causing a decrease in immunosuppressive cells (e.g., myeloid-derived suppressor cells (MDSCs), regulatory T cells (Tregs), tumor associated neutrophils (TANs), M2 macrophages, and tumor associated macrophages (TAMs)), and particularly within the tumor and/or tumor microenvironment (TME). In embodiments, the present therapies may alter the ratio of M1 versus M2 macrophages in the tumor site and/or TME to favor M1 macrophages.

In embodiments, the present chimeric proteins are able to increase the serum levels of various cytokines including, but not limited to, one or more of IFNγ, TNFα, IL-2, IL-4, IL-5, IL-6, IL-9, IL-10, IL-13, IL-17A, IL-17F, and IL-22. In embodiments, the present chimeric proteins are capable of enhancing IL-2, IL-4, IL-5, IL-10, IL-13, IL-17A, IL-22, TNFα or IFNγ in the serum of a treated subject. In embodiments, administration of the present chimeric protein is capable of enhancing TNFα secretion. In a specific embodiment, administration of the present chimeric protein is capable of enhancing superantigen mediated TNFα secretion by leukocytes. Detection of such a cytokine response may provide a method to determine the optimal dosing regimen for the indicated chimeric protein.

In a chimeric protein of the present invention, the chimeric protein is capable of increasing or preventing a decrease in a sub-population of CD4+ and/or CD8+ T cells.

In a chimeric protein of the present invention, the chimeric protein is capable of enhancing tumor-killing activity by T cells.

In embodiments, the present chimeric proteins inhibit, block and/or reduce cell death of an anti-tumor CD8+ and/or CD4+ T cell; or stimulate, induce, and/or increase cell death of a pro-tumor T cell. T cell exhaustion is a state of T cell dysfunction characterized by progressive loss of proliferative and effector functions, culminating in clonal deletion. Accordingly, a pro-tumor T cell refers to a state of T cell dysfunction that arises during many chronic infections, inflammatory diseases, and cancer. This dysfunction is defined by poor proliferative and/or effector functions, sustained expression of inhibitory receptors and a transcriptional state distinct from that of functional effector or memory T cells. Exhaustion prevents optimal control of infection and tumors. Illustrative pro-tumor T cells include, but are not limited to, Tregs, CD4+ and/or CD8+ T cells expressing one or more checkpoint inhibitory receptors, Th2 cells and Th17 cells. Checkpoint inhibitory receptors refer to receptors expressed on immune cells that prevent or inhibit uncontrolled immune responses. In contrast, an anti-tumor CD8+ and/or CD4+ T cell refers to T cells that can mount an immune response to a tumor.

In embodiments, the present chimeric proteins are capable of, and can be used in methods comprising, increasing a ratio of effector T cells to regulatory T cells. Illustrative effector T cells include ICOS⁺ effector T cells; cytotoxic T cells (e.g., αβ TCR, CD3⁺, CD8⁺, CD45RO⁺); CD4⁺ effector T cells (e.g., αβ TCR, CD3⁺, CD4⁺, CCR7⁺, CD62Lhi, IL⁻7R/CD127⁺); CD8⁺ effector T cells (e.g., αβ TCR, CD3⁺, CD8⁺, CCR7⁺, CD62Lhi, IL⁻7 R/CD127⁺); effector memory T cells (e.g., CD62Llow, CD44⁺, TCR, CD3⁺, IL⁻7R/CD127⁺, IL-15R⁺, CCR7low); central memory T cells (e.g., CCR7⁺, CD62L⁺, CD27⁺; or CCR7hi, CD44⁺, CD62Lhi, TCR, CD3⁺, IL-7R/CD127⁺, IL-15R⁺); CD62L⁺ effector T cells; CD8⁺ effector memory T cells (TEM) including early effector memory T cells (CD27⁺CD62L⁻) and late effector memory T cells (CD27⁻CD62L⁻) (TemE and TemL, respectively); CD127(+)CD25(low/−) effector T cells; CD127(⁻)CD25(⁻) effector T cells; CD8+ stem cell memory effector cells (TSCM) (e.g., CD44(low)CD62L(high)CD122(high)sca(⁺)); TH1 effector T-cells (e.g., CXCR3⁺, CXCR6⁺ and CCR5⁺; or αβ TCR, CD3⁺, CD4⁺, IL-12R⁺, IFNγR⁺, CXCR3⁺), TH2 effector T cells (e.g., CCR3⁺, CCR4⁺ and CCR8⁺; or αβ TCR, CD3⁺, CD4⁺, IL-4R⁺, IL-33R⁺, CCR4⁺, CRTH2⁺); TH9 effector T cells (e.g., αβ TCR, CD3⁺, CD4⁺); TH17 effector T cells (e.g., αβ TCR, CD3⁺, CD4⁺, IL-23R⁺, CCR6⁺, IL-1R⁺); CD4⁺CD45RO⁺CCR7⁺ effector T cells, CD4⁺CD45RO⁺CCR7(⁻) effector T cells; and effector T cells secreting IL-2, IL-4 and/or IFN-γ. Illustrative regulatory T cells include ICOS⁺ regulatory T cells, CD4⁺CD25⁺FOXP3⁺ regulatory T cells, CD4⁺CD25⁺ regulatory T cells, CD4⁺CD25⁻ regulatory T cells, CD4⁺CD25high regulatory T cells, TIM-3⁺PD-1⁺ regulatory T cells, lymphocyte activation gene-3 (LAG-3)⁺ regulatory T cells, CTLA-4/CD152⁺ regulatory T cells, neuropilin-1 (Nrp-1)⁺ regulatory T cells, CCR4⁺CCR8⁺ regulatory T cells, CD62L (L-selectin)⁺ regulatory T cells, CD45RBlow regulatory T cells, CD127low regulatory T cells, LRRC32/GARP⁺ regulatory T cells, CD39⁺ regulatory T cells, GITR⁺ regulatory T cells, LAP⁺ regulatory T cells, 1B11⁺ regulatory T cells, BTLA⁺ regulatory T cells, type 1 regulatory T cells (Tr1 cells), T helper type 3 (Th3) cells, regulatory cell of natural killer T cell phenotype (NKTregs), CD8⁺ regulatory T cells, CD8⁺CD28⁻ regulatory T cells and/or regulatory T-cells secreting IL-10, IL-35, TGF-6, TNF-α, Galectin-1, IFN-γ and/or MCP1.

In embodiments, the chimeric protein of the invention causes an increase in effector T cells (e.g., CD4+CD25− T cells).

In embodiments, the chimeric protein causes a decrease in regulatory T cells (e.g., CD4+CD25+ T cells).

In embodiments, the chimeric protein generates a memory response which may be capable of preventing relapse or protecting the animal from a recurrence and/or preventing, or reducing the likelihood of, metastasis. Thus, an animal treated with the chimeric protein is later able to attack tumor cells and/or prevent development of tumors when rechallenged after an initial treatment with the chimeric protein. Accordingly, a chimeric protein of the present invention stimulates both active tumor destruction and also immune recognition of tumor antigens, which are essential in programming a memory response capable of preventing relapse.

In embodiments, the chimeric protein is capable of causing activation of antigen presenting cells. In embodiments, the chimeric protein is capable enhancing the ability of antigen presenting cells to present antigen.

In embodiments, the present chimeric proteins are capable of, and can be used in methods comprising, transiently stimulating effector T cells for longer than about 12 hours, about 24 hours, about 48 hours, about 72 hours or about 96 hours or about 1 week or about 2 weeks. In embodiments, the transient stimulation of effector T cells occurs substantially in a patient's bloodstream or in a particular tissue/location including lymphoid tissues such as for example, the bone marrow, lymph-node, spleen, thymus, mucosa-associated lymphoid tissue (MALT), non-lymphoid tissues, or in the tumor microenvironment.

In a chimeric protein of the present invention, the present chimeric protein unexpectedly provides binding of the extracellular domain components to their respective binding partners with slow off rates (Kd or K_{off}). In embodiments, this provides an unexpectedly long interaction of the receptor to ligand and vice versa. Such an effect allows for a longer positive signal effect, e.g., increase in or activation of immune stimulatory signals. For example, the present chimeric protein, e.g., via the long off rate binding allows sufficient signal transmission to provide immune cell proliferation, allow for anti-tumor attack, allows sufficient signal transmission to provide release of stimulatory signals, e.g., cytokines.

In a chimeric protein of the present invention, the chimeric protein is capable of forming a stable synapse between cells. The stable synapse of cells promoted by the chimeric proteins (e.g., between cells bearing negative signals) provides spatial orientation to favor tumor reduction—such as positioning the T cells to attack tumor cells and/or sterically preventing the tumor cell from delivering negative signals, including negative signals beyond those masked by the chimeric protein of the invention. In embodiments, this provides longer on-target (e.g., intra-tumoral) half-life (t_{1/2}) as compared to serum t_{1/2} of the chimeric proteins. Such properties could have the combined advantage of reducing off-target toxicities associated with systemic distribution of the chimeric proteins.

In embodiments, the chimeric protein is capable of providing a sustained immunomodulatory effect.

The present chimeric proteins provide synergistic therapeutic effects (e.g., anti-tumor effects) as it allows for improved site-specific interplay of two immunotherapy agents. In embodiments, the present chimeric proteins provide the potential for reducing off-site and/or systemic toxicity.

In embodiments, the present chimeric protein exhibit enhanced safety profiles. In embodiment, the present chimeric protein exhibit reduced toxicity profiles. For example, administration of the present chimeric proteins may result in reduced side effects such as one or more of diarrhea, inflammation (e.g., of the gut), or weight loss, which occur following administration of antibodies directed to the ligand(s)/receptor(s) targeted by the extracellular domains of the present chimeric proteins. In embodiments, the present chimeric protein provides improved safety, as compared to antibodies directed to the ligand(s)/receptor(s) targeted by the extracellular domains of the present chimeric proteins, yet, without sacrificing efficacy.

In embodiments, the present chimeric proteins provide reduced side-effects, e.g., GI complications, relative to current immunotherapies, e.g., antibodies directed to ligand(s)/receptor(s) targeted by the extracellular domains of the present chimeric proteins. Illustrative GI complications include abdominal pain, appetite loss, autoimmune effects, constipation, cramping, dehydration, diarrhea, eating problems, fatigue, flatulence, fluid in the abdomen or ascites, gastrointestinal (GI) dysbiosis, GI mucositis, inflammatory bowel disease, irritable bowel syndrome (IBS-D and IBS-C), nausea, pain, stool or urine changes, ulcerative colitis, vomiting, weight gain from retaining fluid, and/or weakness.

In some aspects, the present chimeric agents are used to treat one or more infections. In embodiments, the present chimeric proteins are used in methods of treating viral infections (including, for example, HIV and HCV). In embodiments, the infections induce immunosuppression. For example, HIV infections often result in immunosuppression in the infected subjects. Accordingly, as disclosed elsewhere herein, the treatment of such infections may involve, in embodiments, modulating the immune system with the present chimeric proteins to favor immune stimulation over blocking or preventing immune inhibition.

In embodiments, the present invention provides methods of treating viral infections including, without limitation, acute or chronic viral infections, for example, of the respiratory tract, of papilloma virus infections, of herpes simplex virus (HSV) infection, of human immunodeficiency virus (HIV) infection, and of viral infection of internal organs such as infection with hepatitis viruses. In embodiments, the viral infection is caused by a member of Flaviviridae. In embodiments, the member of Flaviviridae is selected from Yellow Fever Virus, West Nile virus, Dengue virus, Japanese Encephalitis Virus, St. Louis Encephalitis Virus, and Hepatitis C Virus. In embodiments, the viral infection is caused by a member of Picornaviridae, e.g., poliovirus, rhinovirus, coxsackievirus. In embodiments, the viral infection is caused by a member of Orthomyxoviridae, e.g., an influenza virus. In embodiments, the viral infection is caused by a member of Retroviridae, e.g., a lentivirus. In embodiments, the viral infection is caused by a member of Paramyxoviridae, e.g., respiratory syncytial virus, a human parainfluenza virus, rubulavirus (e.g., mumps virus), measles virus, and human metapneumovirus. In embodiments, the viral infection is caused by a member of Bunyaviridae, e.g., hantavirus. In embodiments, the viral infection is caused by a member of Reoviridae, e.g., a rotavirus.

### Combination Therapies and Conjugation

In embodiments, the invention provides for chimeric proteins and methods that further comprise administering an additional agent to a subject. In embodiments, the invention pertains to co-administration and/or co-formulation. Any of the compositions disclosed herein may be co-formulated and/or co-administered.

In embodiments, any chimeric protein disclosed herein acts synergistically when co-administered with another agent and is administered at doses that are lower than the doses commonly employed when such agents are used as monotherapy. In embodiments, any agent referenced herein may be used in combination with any of the chimeric proteins disclosed herein.

An aspect of the present invention provides a method for treating a cancer or a viral infection in a subject in need thereof comprising: providing the subject a first pharmaceutical composition comprising a therapeutically effective amount of a herein-disclosed chimeric protein; providing the subject a second pharmaceutical composition comprising an antibody that is capable of binding CTLA-4, or an antibody that is capable of binding PD-1 or a PD-1 ligand and capable of inhibiting the interaction of PD-1 with one or more of its ligands.

Another aspect of the present invention provides a method for treating a cancer or a viral infection in a subject comprising: providing the subject a pharmaceutical composition comprising a herein-disclosed chimeric protein. In this aspect, the subject has undergone or is undergoing treatment with an antibody that is capable of binding CTLA-4, or an antibody that is capable of binding PD-1 or a PD-1 ligand and capable of inhibiting the interaction of PD-1 with one or more of its ligands.

Yet another aspect of the present invention provides a method for treating a cancer in a subject comprising: providing the subject a pharmaceutical composition comprising an antibody that is capable of binding CTLA-4, or an antibody that is capable of binding PD-1 or a PD-1 ligand and capable of inhibiting the interaction of PD-1 with one or more of its ligands. In this aspect, the subject has undergone or is undergoing treatment with a herein-disclosed chimeric protein.

In embodiments of any herein-disclosed aspect, the first pharmaceutical composition and the second pharmaceutical composition are provided simultaneously.

In embodiments of any herein-disclosed aspect, the first pharmaceutical composition is provided after the second pharmaceutical composition is provided.

In embodiments of any herein-disclosed aspect, the first pharmaceutical composition is provided before the second pharmaceutical composition is provided.

In embodiments of any herein-disclosed aspect, the dose of the first pharmaceutical composition is less than the dose of the first pharmaceutical composition provided to a subject who has not undergone or is not undergoing treatment with the second pharmaceutical composition.

In embodiments of any herein-disclosed aspect, the dose of the second pharmaceutical composition provided is less than the dose of the second pharmaceutical composition provided to a subject who has not undergone or is not undergoing treatment with the first pharmaceutical composition.

In embodiments of any herein-disclosed aspect, the dose of the pharmaceutical composition provided to the subject is less than the dose of the pharmaceutical composition that is provided to a subject who has not undergone or is not undergoing treatment with an antibody that is capable of binding PD-1 or a PD-1 ligand, or who has not undergone or is not undergoing treatment with an antibody that is capable of binding CTLA-4.

In embodiments of any herein-disclosed aspect, the subject has an increased chance of survival, a gain in weight, a reduction in tumor size or cancer prevalence, and/or a decrease in viral load/virally-infected cells when compared to a subject who has only undergone or is only undergoing treatment with the first pharmaceutical composition.

In embodiments of any herein-disclosed aspect, the subject has an increased chance of survival, a gain in weight, a reduction in tumor size or cancer prevalence, and/or a decrease in viral load/virally-infected cells when compared to a subject who has only undergone or is only undergoing treatment with the second pharmaceutical composition.

In embodiments of any herein-disclosed aspect, the subject has an increased chance of survival, a gain in weight, a reduction in tumor size or cancer prevalence, and/or a decrease in viral load/virally-infected cells when compared to the subject who has not undergone or is not undergoing treatment with an antibody that is capable of binding PD-1 or binding a PD-1 ligand, or the antibody that is capable of binding CTLA-4.

In embodiments of any herein-disclosed aspect, the subject has a cancer or viral infection that is poorly responsive or is refractory to treatment comprising the antibody that is capable of binding PD-1 or binding a PD-1 ligand, or the antibody that is capable of binding CTLA-4.

In embodiments of any herein-disclosed aspect, the linker is a polypeptide selected from a flexible amino acid sequence, an IgG hinge region, and an antibody sequence. In embodiments of any herein-disclosed aspect, the linker comprises at least one cysteine residue capable of forming a disulfide bond and/or comprises a hinge-CH2-CH3 Fc domain. In embodiments of any herein-disclosed aspect, the linker and/or the region linker comprises a hinge-CH2-CH3 Fc domain derived from IgG4, e.g., human IgG4. In embodiments of any herein-disclosed aspect, the linker comprises an amino acid sequence that is at least 95% identical to the amino acid sequence of SEQ ID NO: 1, SEQ ID NO: 2, or SEQ ID NO: 3.

In embodiments of any herein-disclosed aspect, the heterologous chimeric protein is “CD80-Fc-NKG2A”, “CD86-Fc-NKG2A”, “CD58-Fc-NKG2A”, “PD-1-Fc-NKG2A”, “SLAMF6-Fc-NKG2A”, “SIRPα-Fc-NKG2A, “TGFBR2-Fc-NKG2A”, or “CD48-Fc-NKG2A”, as disclosed herein.

In embodiments of any herein-disclosed aspect, the antibody that is capable of binding PD-1 or a PD-1 ligand is selected from the group consisting of nivolumab (ONO 4538, BMS 936558, MDX1106, OPDIVO (Bristol Myers Squibb)), pembrolizumab (KEYTRUDA/MK 3475, Merck), pidilizumab (CT 011, Cure Tech), RMP1-14, AGEN2034 (Agenus), and cemiplimab ((REGN-2810). Such an antibody is capable of inhibiting the interaction of PD-1 with one or more of its ligands.

In embodiments of any herein-disclosed aspect, the antibody that is capable of binding CTLA-4 is selected from the group consisting of YERVOY (ipilimumab), 9D9, tremelimumab (formerly ticilimumab, CP-675,206; MedImmune), AGEN1884, and RG2077.

In embodiments of any herein-disclosed aspect, the subject has a cancer or viral infection that is poorly responsive or is refractory to treatment comprising an antibody that is capable of binding PD-1 or binding a PD-1 ligand, or an antibody that is capable of binding CTLA-4.

In embodiments of any herein-disclosed aspect, the cancer or viral infection is poorly responsive or is non-responsive to treatment with an antibody that is capable of binding PD-1 or binding a PD-1 ligand, or an antibody that is capable of binding CTLA-4 after 12 weeks or so of such treatment.

In embodiments, inclusive of, without limitation, cancer applications, the present additional agent is one or more immune-modulating agents selected from an agent that blocks, reduces and/or inhibits PD-1 and PD-L1 or PD-L2 and/or the binding of PD-1 with PD-L1 or PD-L2 (by way of non-limiting example, one or more of nivolumab (ONO-4538/BMS-936558, MDX1106, OPDIVO, BRISTOL MYERS SQUIBB), pembrolizumab (KEYTRUDA, Merck), pidilizumab (CT-011, CURE TECH), MK-3475 (MERCK), BMS 936559 (BRISTOL MYERS SQUIBB), atezolizumab (TECENTRIQ, GENENTECH), MPDL3280A (ROCHE)), an agent that increases and/or stimulates CD137 (4-1BB) and/or the binding of CD137 (4-1BB) with one or more of 4-1BB ligand (by way of non-limiting example, urelumab (BMS-663513 and anti-4-1BB antibody), and an agent that blocks, reduces and/or inhibits the activity of CTLA-4 and/or the binding of CTLA-4 with one or more of AP2M1, CD80, CD86, SHP-2, and PPP2R5A and/or the binding of OX40 with OX4OL (by way of non-limiting example GBR 830 (GLENMARK), MED16469 (MEDIMMUNE).

In embodiments of any herein-disclosed aspect, the antibody is capable of mediating antibody dependent cellular cytotoxicity (ADCC). In embodiments, the antibody is selected from the group consisting of cetuximab (Eli Lilly and Co), daratumumab (Genmab), gatipotuzumab (Glycotope), margetuximab (Raven biotechnologies), mogamulizumab (Kyowa Kirin International PLC), MEDI-551 or inebilizumab (MedImmune), M0R208 or tafasitamab (MorphoSys AG), ocaratuzumab (Creative BioLabs), GAZYVA® or obinutuzumab (Roche), R05083945 or GA201 (Creative BioLabs), rituximab (Genentech), trastuzumab (Roche), TrasGEX (Glycotope), tomuzotuximab (Glycotope), and ublituximab (TG Therapeutics).

In embodiments, of any herein-disclosed aspect, the antibody is capable of binding to a tumor antigen. In embodiments, the antibody is selected from the group consisting of cetuximab (Eli Lilly and Co), daratumumab (Genmab), gatipotuzumab (Glycotope), margetuximab (Raven biotechnologies), mogamulizumab (Kyowa Kirin International PLC), MEDI-551 or inebilizumab (MedImmune), M0R208 or tafasitamab (MorphoSys AG), ocaratuzumab (Creative BioLabs), GAZYVA® or obinutuzumab (Roche), R05083945 or GA201 (Creative BioLabs), rituximab (Genentech), trastuzumab (Roche), TrasGEX (Glycotope), tomuzotuximab (Glycotope), and ublituximab (TG Therapeutics).

In embodiments of any herein-disclosed aspect, the subject has a cancer or viral infection that is poorly responsive or is refractory to treatment comprising an antibody that is capable of binding capable of binding to a tumor antigen and/or antibody is capable of mediating antibody dependent cellular cytotoxicity (ADCC).

In embodiments of any herein-disclosed aspect, the cancer or viral infection is poorly responsive or is non-responsive to treatment with an antibody that is capable of binding capable of binding to a tumor antigen and/or antibody is capable of mediating antibody dependent cellular cytotoxicity (ADCC) after 12 weeks or so of such treatment.

In embodiments, the chimeric proteins (and/or additional agents) disclosed herein, include derivatives that are modified, i.e., by the covalent attachment of any type of molecule to the composition such that covalent attachment does not prevent the activity of the composition. For example, but not by way of limitation, derivatives include composition that have been modified by, inter alia, glycosylation, lipidation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to specific chemical cleavage, acetylation, formylation, metabolic synthesis of turicamycin, etc. Additionally, the derivative can contain one or more non-classical amino acids. In still other embodiments, the chimeric proteins (and/or additional agents) disclosed herein further comprise a cytotoxic agent, comprising, in illustrative embodiments, a toxin, a chemotherapeutic agent, a radioisotope, and an agent that causes apoptosis or cell death. Such agents may be conjugated to a composition disclosed herein.

The chimeric proteins (and/or additional agents) disclosed herein may thus be modified post-translationally to add effector moieties such as chemical linkers, detectable moieties such as for example fluorescent dyes, enzymes, substrates, bioluminescent materials, radioactive materials, and chemiluminescent moieties, or functional moieties such as for example streptavidin, avidin, biotin, a cytotoxin, a cytotoxic agent, and radioactive materials.

### Pharmaceutical Composition

Aspects of the present invention include a pharmaceutical composition comprising a therapeutically effective amount of a chimeric protein as disclosed herein.

The chimeric proteins (and/or additional agents) disclosed herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

In embodiments, the compositions disclosed herein are in the form of a pharmaceutically acceptable salt.

Further, any chimeric protein (and/or additional agents) disclosed herein can be administered to a subject as a component of a composition, e.g., pharmaceutical composition, that comprises a pharmaceutically acceptable carrier or vehicle. Such pharmaceutical compositions can optionally comprise a suitable amount of a pharmaceutically acceptable excipient so as to provide the form for proper administration. Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In embodiments, the pharmaceutically acceptable excipients are sterile when administered to a subject. Water is a useful excipient when any agent disclosed herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent disclosed herein, if desired, can also comprise minor amounts of wetting or emulsifying agents, or pH buffering agents.

In embodiments, the compositions, e.g., pharmaceutical compositions, disclosed herein are resuspended in a saline buffer (including, without limitation TBS, PBS, and the like).

In embodiments, the chimeric proteins may by conjugated and/or fused with another agent to extend half-life or otherwise improve pharmacodynamic and pharmacokinetic properties. In embodiments, the chimeric proteins may be fused or conjugated with one or more of PEG, XTEN (e.g., as rPEG), polysialic acid (POLYXEN), albumin (e.g., human serum albumin or HAS), elastin-like protein (ELP), PAS, HAP, GLK, CTP, transferrin, and the like. In embodiments, each of the individual chimeric proteins is fused to one or more of the agents described in BioDrugs (2015) 29:215-239, the entire contents of which are hereby incorporated by reference.

The present invention includes the disclosed chimeric protein (and/or additional agents) in various formulations of pharmaceutical composition. Any chimeric protein (and/or additional agents) disclosed herein can take the form of solutions, suspensions, emulsion, drops, tablets, pills, pellets, capsules, capsules containing liquids, powders, sustained-release formulations, suppositories, emulsions, aerosols, sprays, suspensions, or any other form suitable for use. DNA or RNA constructs encoding the protein sequences may also be used. In embodiments, the composition is in the form of a capsule (see, e.g., U.S. Pat. No. 5,698,155). Other examples of suitable pharmaceutical excipients are described in Remington's Pharmaceutical Sciences 1447-1676 (Alfonso R. Gennaro eds., 19th ed. 1995), incorporated herein by reference.

Where necessary, the pharmaceutical compositions comprising the chimeric protein (and/or additional agents) can also include a solubilizing agent. Also, the agents can be delivered with a suitable vehicle or delivery device as known in the art. Combination therapies outlined herein can be co-delivered in a single delivery vehicle or delivery device. Compositions for administration can optionally include a local anesthetic such as, for example, lignocaine to lessen pain at the site of the injection.

The pharmaceutical compositions comprising the chimeric protein (and/or additional agents) of the present invention may conveniently be presented in unit dosage forms and may be prepared by any of the methods well known in the art of pharmacy. Such methods generally include the step of bringing therapeutic agents into association with a carrier, which constitutes one or more accessory ingredients. Typically, the pharmaceutical compositions are prepared by uniformly and intimately bringing therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In embodiments, any chimeric protein (and/or additional agents) disclosed herein is formulated in accordance with routine procedures as a pharmaceutical composition adapted for a mode of administration disclosed herein.

### Administration, Dosing, and Treatment Regimens

As examples, administration results in the release of chimeric protein (and/or additional agents) disclosed herein into the bloodstream (via enteral or parenteral administration), or alternatively, the chimeric protein (and/or additional agents) is administered directly to the site of active disease.

Dosage forms suitable for parenteral administration include, for example, solutions, suspensions, dispersions, emulsions, and the like. They may also be manufactured in the form of sterile solid compositions (e.g., lyophilized composition), which can be dissolved or suspended in sterile injectable medium immediately before use. They may contain, for example, suspending or dispersing agents known in the art.

The dosage of any chimeric protein (and/or additional agents) disclosed herein as well as the dosing schedule can depend on various parameters, including, but not limited to, the disease being treated, the subject's general health, and the administering physician's discretion.

The dosage of any chimeric protein (and/or additional agents) disclosed herein can depend on several factors including the severity of the condition, whether the condition is to be treated or prevented, and the age, weight, and health of the subject to be treated. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the agents being administered, the time of administration, the route of administration, the nature of the formulation, the rate of excretion, the particular disease being treated, the severity of the disorder, and the anatomical location of the disorder. Some variations in the dosage can be expected.

In another embodiment, delivery can be in a vesicle, in particular a liposome (see Langer, 1990, Science 249:1527-1533; Treat et al., in Liposomes in Therapy of Infectious Disease and Cancer, Lopez-Berestein and Fidler (eds.), Liss, New York, pp. 353-365 (1989).

A chimeric protein (and/or additional agents) disclosed herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105).

In another embodiment, a controlled-release system can be placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

The dosage regimen utilizing any chimeric protein (and/or additional agents) disclosed herein can be selected in accordance with a variety of factors including type, species, age, weight, sex and medical condition of the subject; the severity of the condition to be treated; the route of administration; the renal or hepatic function of the subject; the pharmacogenomic makeup of the individual; and the specific compound of the invention employed. Any chimeric protein (and/or additional agents) disclosed herein can be administered in a single daily dose, or the total daily dosage can be administered in divided doses of two, three or four times daily. Furthermore, any chimeric protein (and/or additional agents) disclosed herein can be administered continuously rather than intermittently throughout the dosage regimen.

### Cells and Nucleic Acids

Aspects of the present invention provide an expression vector comprising a nucleic acid which encodes a chimeric protein as disclosed herein. The expression vector comprises a nucleic acid encoding the chimeric protein disclosed herein. In embodiments, the expression vector comprises DNA or RNA. In embodiments, the expression vector is a mammalian expression vector.

Constructs could be produced by cloning of the nucleic acids encoding the three fragments (the extracellular domain of a Type I transmembrane protein or a portion of a membrane-bound extracellular protein, followed by a linker sequence, followed by the extracellular domain of a Type II transmembrane protein, e.g., NKG2A) into a vector (plasmid, viral or other) wherein the amino terminus of the complete sequence corresponded to the ‘left’ side of the molecule containing the Type I transmembrane protein or the portion of a membrane-bound extracellular protein and the carboxy terminus of the complete sequence corresponded to the ‘right’ side of the molecule containing the extracellular domain of a Type II transmembrane protein, e.g., NKG2A. In embodiments of chimeric proteins having one of the other configurations, as described above, a construct would comprise three nucleic acids such that the translated chimeric protein produced would have the desired configuration, e.g., a dual inward-facing chimeric protein. Accordingly, in embodiments, the present chimeric proteins are engineered as such.

Both prokaryotic and eukaryotic vectors can be used for expression of the chimeric protein. Prokaryotic vectors include constructs based on E. coli sequences (see, e.g., Makrides, Microbiol Rev 1996, 60:512-538). Non-limiting examples of regulatory regions that can be used for expression in E. coli include lac, trp, Ipp, phoA, recA, tac, T3, T7 and AP_{L}. Non-limiting examples of prokaryotic expression vectors may include the Agt vector series such as Δgt11 (Huynh et al., in “DNA Cloning Techniques, Vol. I: A Practical Approach,” 1984, (D. Glover, ed.), pp. 49-78, IRL Press, Oxford), and the pET vector series (Studier et al., Methods Enzymol 1990, 185:60-89). Prokaryotic host-vector systems cannot perform much of the post-translational processing of mammalian cells, however. Thus, eukaryotic host-vector systems may be particularly useful. A variety of regulatory regions can be used for expression of the chimeric proteins in mammalian host cells. For example, the SV40 early and late promoters, the cytomegalovirus (CMV) immediate early promoter, and the Rous sarcoma virus long terminal repeat (RSV-LTR) promoter can be used. Inducible promoters that may be useful in mammalian cells include, without limitation, promoters associated with the metallothionein II gene, mouse mammary tumor virus glucocorticoid responsive long terminal repeats (MMTV-LTR), the β-interferon gene, and the hsp70 gene (see, Williams et al., Cancer Res 1989, 49:2735-42; and Taylor et al., Mol Cell Biol 1990, 10:165-75). Heat shock promoters or stress promoters also may be advantageous for driving expression of the chimeric proteins in recombinant host cells.

In embodiments, expression vectors of the invention comprise a nucleic acid encoding the chimeric proteins, or a complement thereof, operably linked to an expression control region, or complement thereof, that is functional in a mammalian cell. The expression control region is capable of driving expression of the operably linked blocking and/or stimulating agent encoding nucleic acid such that the blocking and/or stimulating agent is produced in a human cell transformed with the expression vector.

Expression control regions are regulatory polynucleotides (sometimes referred to herein as elements), such as promoters and enhancers, that influence expression of an operably linked nucleic acid. An expression control region of an expression vector of the invention is capable of expressing operably linked encoding nucleic acid in a human cell. In embodiments, the cell is a tumor cell. In another embodiment, the cell is a non-tumor cell. In embodiments, the expression control region confers regulatable expression to an operably linked nucleic acid. A signal (sometimes referred to as a stimulus) can increase or decrease expression of a nucleic acid operably linked to such an expression control region. Such expression control regions that increase expression in response to a signal are often referred to as inducible. Such expression control regions that decrease expression in response to a signal are often referred to as repressible. Typically, the amount of increase or decrease conferred by such elements is proportional to the amount of signal present; the greater the amount of signal, the greater the increase or decrease in expression.

In embodiments, the present invention contemplates the use of inducible promoters capable of effecting high level of expression transiently in response to a cue. For example, when in the proximity of a tumor cell, a cell transformed with an expression vector for the chimeric protein (and/or additional agents) comprising such an expression control sequence is induced to transiently produce a high level of the agent by exposing the transformed cell to an appropriate cue. Illustrative inducible expression control regions include those comprising an inducible promoter that is stimulated with a cue such as a small molecule chemical compound. Particular examples can be found, for example, in U.S. Pat. Nos. 5,989,910, 5,935,934, 6,015,709, and 6,004,941, each of which is incorporated herein by reference in its entirety.

Expression control regions and locus control regions include full-length promoter sequences, such as native promoter and enhancer elements, as well as subsequences or polynucleotide variants which retain all or part of full-length or non-variant function. As used herein, the term “functional” and grammatical variants thereof, when used in reference to a nucleic acid sequence, subsequence or fragment, means that the sequence has one or more functions of native nucleic acid sequence (e.g., non-variant or unmodified sequence).

As used herein, “operable linkage” refers to a physical juxtaposition of the components so described as to permit them to function in their intended manner. In the example of an expression control element in operable linkage with a nucleic acid, the relationship is such that the control element modulates expression of the nucleic acid. Typically, an expression control region that modulates transcription is juxtaposed near the 5′ end of the transcribed nucleic acid (i.e., “upstream”). Expression control regions can also be located at the 3′ end of the transcribed sequence (i.e., “downstream”) or within the transcript (e.g., in an intron). Expression control elements can be located at a distance away from the transcribed sequence (e.g., 100 to 500, 500 to 1000, 2000 to 5000, or more nucleotides from the nucleic acid). A specific example of an expression control element is a promoter, which is usually located 5′ of the transcribed sequence. Another example of an expression control element is an enhancer, which can be located 5′ or 3′ of the transcribed sequence, or within the transcribed sequence.

Expression systems functional in human cells are well known in the art, and include viral systems. Generally, a promoter functional in a human cell is any DNA sequence capable of binding mammalian RNA polymerase and initiating the downstream (3′) transcription of a coding sequence into mRNA. A promoter will have a transcription initiating region, which is usually placed proximal to the 5′ end of the coding sequence, and typically a TATA box located 25-30 base pairs upstream of the transcription initiation site. The TATA box is thought to direct RNA polymerase II to begin RNA synthesis at the correct site. A promoter will also typically contain an upstream promoter element (enhancer element), typically located within 100 to 200 base pairs upstream of the TATA box. An upstream promoter element determines the rate at which transcription is initiated and can act in either orientation. Of particular use as promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenylation signals include those derived from SV40. Introns may also be included in expression constructs.

There are varieties of techniques available for introducing nucleic acids into viable cells. Techniques suitable for the transfer of nucleic acid into mammalian cells in vitro include the use of liposomes, electroporation, microinjection, cell fusion, polymer-based systems, DEAE-dextran, viral transduction, the calcium phosphate precipitation method, etc. For in vivo gene transfer, a number of techniques and reagents may also be used, including liposomes; natural polymer-based delivery vehicles, such as chitosan and gelatin; viral vectors are also suitable for in vivo transduction. In some situations, it is desirable to provide a targeting agent, such as an antibody or ligand specific for a tumor cell surface membrane protein. Where liposomes are employed, proteins which bind to a cell surface membrane protein associated with endocytosis may be used for targeting and/or to facilitate uptake, e.g., capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262, 4429-4432 (1987); and Wagner et al., Proc. Natl. Acad. Sci. USA 87, 3410-3414 (1990).

Where appropriate, gene delivery agents such as, e.g., integration sequences can also be employed. Numerous integration sequences are known in the art (see, e.g., Nunes-Duby et al., Nucleic Acids Res. 26:391-406, 1998; Sadwoski, J. Bacteriol., 165:341-357, 1986; Bestor, Cell, 122(3):322-325, 2005; Plasterk et al., TIG 15:326-332, 1999; Kootstra et al., Ann. Rev. Pharm. Toxicol., 43:413-439, 2003). These include recombinases and transposases. Examples include Cre (Sternberg and Hamilton, J. Mol. Biol., 150:467-486, 1981), lambda (Nash, Nature, 247, 543-545, 1974), Flp (Broach, et al., Cell, 29:227-234, 1982), R (Matsuzaki, et al., J. Bacteriology, 172:610-618, 1990), cpC31 (see, e.g., Groth et al., J. Mol. Biol. 335:667-678, 2004), sleeping beauty, transposases of the mariner family (Plasterk et al., supra), and components for integrating viruses such as AAV, retroviruses, and antiviruses having components that provide for virus integration such as the LTR sequences of retroviruses or lentivirus and the ITR sequences of AAV (Kootstra et al., Ann. Rev. Pharm. Toxicol., 43:413-439, 2003). In addition, direct and targeted genetic integration strategies may be used to insert nucleic acid sequences encoding the chimeric fusion proteins including CRISPR/CAS9, zinc finger, TALEN, and meganuclease gene-editing technologies.

In embodiments, the expression vectors for the expression of the chimeric proteins (and/or additional agents) are viral vectors. Many viral vectors useful for gene therapy are known (see, e.g., Lundstrom, Trends Biotechnol., 21: 1 17, 122, 2003. Illustrative viral vectors include those selected from Antiviruses (LV), retroviruses (RV), adenoviruses (AV), adeno-associated viruses (AAV), and a viruses, though other viral vectors may also be used. For in vivo uses, viral vectors that do not integrate into the host genome are suitable for use, such as a viruses and adenoviruses. Illustrative types of a viruses include Sindbis virus, Venezuelan equine encephalitis (VEE) virus, and Semliki Forest virus (SFV). For in vitro uses, viral vectors that integrate into the host genome are suitable, such as retroviruses, AAV, and Antiviruses. In embodiments, the invention provides methods of transducing a human cell in vivo, comprising contacting a solid tumor in vivo with a viral vector of the invention.

Aspects of the present invention include a host cell comprising the expression vector which comprises the chimeric protein disclosed herein.

Expression vectors can be introduced into host cells for producing the present chimeric proteins. Cells may be cultured in vitro or genetically engineered, for example. Useful mammalian host cells include, without limitation, cells derived from humans, monkeys, and rodents (see, for example, Kriegler in “Gene Transfer and Expression: A Laboratory Manual,” 1990, New York, Freeman & Co.). These include monkey kidney cell lines transformed by SV40 (e.g., COS-7, ATCC CRL 1651); human embryonic kidney lines (e.g., 293, 293-EBNA, or 293 cells subcloned for growth in suspension culture, Graham et al., J Gen Virol 1977, 36:59); baby hamster kidney cells (e.g., BHK, ATCC CCL 10); Chinese hamster ovary-cells-DH FR (e.g., CHO, Urlaub and Chasin, Proc Natl Acad Sci USA 1980, 77:4216); DG44 CHO cells, CHO-K1 cells, mouse sertoli cells (Mather, Biol Reprod 1980, 23:243-251); mouse fibroblast cells (e.g., NIH-3T3), monkey kidney cells (e.g., CV1 ATCC CCL 70); African green monkey kidney cells. (e.g., VERO-76, ATCC CRL-1587); human cervical carcinoma cells (e.g., HELA, ATCC CCL 2); canine kidney cells (e.g., MDCK, ATCC CCL 34); buffalo rat liver cells (e.g., BRL 3A, ATCC CRL 1442); human lung cells (e.g., W138, ATCC CCL 75); human liver cells (e.g., Hep G2, HB 8065); and mouse mammary tumor cells (e.g., MMT 060562, ATCC CCL51). Illustrative cancer cell types for expressing the chimeric proteins disclosed herein include mouse fibroblast cell line, NIH3T3, mouse Lewis lung carcinoma cell line, LLC, mouse mastocytoma cell line, P815, mouse lymphoma cell line, EL4 and its ovalbumin transfectant, E.G7, mouse melanoma cell line, B16F10, mouse fibrosarcoma cell line, MC57, and human small cell lung carcinoma cell lines, SCLC #2 and SCLC #7.

Host cells can be obtained from normal or affected subjects, including healthy humans, cancer patients, and patients with an infectious disease, private laboratory deposits, public culture collections such as the American Type Culture Collection (ATCC), or from commercial suppliers.

Cells that can be used for production of the present chimeric proteins in vitro, ex vivo, and/or in vivo include, without limitation, epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells (e.g., as obtained from bone marrow), umbilical cord blood, peripheral blood, and fetal liver. The choice of cell type depends on the type of tumor or infectious disease being treated or prevented, and can be determined by one of skill in the art.

Production and purification of Fc-containing macromolecules (such as monoclonal antibodies) has become a standardized process, with minor modifications between products. For example, many Fc containing macromolecules are produced by human embryonic kidney (HEK) cells (or variants thereof) or Chinese Hamster Ovary (CHO) cells (or variants thereof) or in some cases by bacterial or synthetic methods. Following production, the Fc containing macromolecules that are secreted by HEK or CHO cells are purified through binding to Protein A columns and subsequently ‘polished’ using various methods. Generally speaking, purified Fc containing macromolecules are stored in liquid form for some period of time, frozen for extended periods of time or in some cases lyophilized. In embodiments, production of the chimeric proteins contemplated herein may have unique characteristics as compared to traditional Fc containing macromolecules. In certain examples, the chimeric proteins may be purified using specific chromatography resins, or using chromatography methods that do not depend upon Protein A capture. In embodiments, the chimeric proteins may be purified in an oligomeric state, or in multiple oligomeric states, and enriched for a specific oligomeric state using specific methods. Without being bound by theory, these methods could include treatment with specific buffers including specified salt concentrations, pH and additive compositions. In other examples, such methods could include treatments that favor one oligomeric state over another. The chimeric proteins obtained herein may be additionally ‘polished’ using methods that are specified in the art. In embodiments, the chimeric proteins are highly stable and able to tolerate a wide range of pH exposure (between pH 3-12), are able to tolerate a large number of freeze/thaw stresses (greater than 3 freeze/thaw cycles) and are able to tolerate extended incubation at high temperatures (longer than 2 weeks at 40 degrees C.). In embodiments, the chimeric proteins are shown to remain intact, without evidence of degradation, deamidation, etc. under such stress conditions.

Subjects and/or Animals

In embodiments, the subject and/or animal is a mammal, e.g., a human, mouse, rat, guinea pig, dog, cat, horse, cow, pig, rabbit, sheep, or non-human primate, such as a monkey, chimpanzee, or baboon. In embodiments, the subject and/or animal is a non-mammal, such, for example, a zebrafish. In embodiments, the subject and/or animal may comprise fluorescently-tagged cells (with e.g., GFP). In embodiments, the subject and/or animal is a transgenic animal comprising a fluorescent cell.

In embodiments, the subject and/or animal is a human. In embodiments, the human is a pediatric human. In embodiments, the human is an adult human. In embodiments, the human is a geriatric human. In embodiments, the human may be referred to as a patient.

In certain embodiments, the human has an age in a range of from about 0 months to about 6 months old, from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In embodiments, the subject is a non-human animal, and therefore the invention pertains to veterinary use. In a specific embodiment, the non-human animal is a household pet. In another specific embodiment, the non-human animal is a livestock animal.

### Kits and Medicaments

Aspects of the present invention provide kits that can simplify the administration of any agent disclosed herein.

An illustrative kit of the invention comprises any chimeric protein and/or pharmaceutical composition disclosed herein in unit dosage form. In embodiments, the unit dosage form is a container, such as a pre-filled syringe, which can be sterile, containing any agent disclosed herein and a pharmaceutically acceptable carrier, diluent, excipient, or vehicle. The kit can further comprise a label or printed instructions instructing the use of any agent disclosed herein. The kit may also include a lid speculum, topical anesthetic, and a cleaning agent for the administration location. The kit can also further comprise one or more additional agents disclosed herein. In embodiments, the kit comprises a container containing an effective amount of a composition of the invention and an effective amount of another composition, such those disclosed herein.

Aspects of the present invention include use of a chimeric protein as disclosed herein in the manufacture of a medicament, e.g., a medicament for treatment of cancer and/or treatment of a viral infection.

Another aspect of the present invention is the use of a herein-disclosed chimeric protein, in the manufacture of a medicament.

Any aspect or embodiment disclosed herein can be combined with any other aspect or embodiment as disclosed herein

The invention will be further described in the following examples, which do not limit the scope of the invention described in the claims.

### EXAMPLES

The examples herein are provided to illustrate advantages and benefits of the present technology and to further assist a person of ordinary skill in the art with preparing or using the chimeric proteins of the present technology. The examples herein are also presented in order to more fully illustrate the preferred aspects of the present technology. The examples should in no way be construed as limiting the scope of the present disclosure, as defined by the appended claims. The examples can include or incorporate any of the variations, aspects or embodiments of the present technology described above. The variations, aspects or embodiments described above may also further each include or incorporate the variations of any or all other variations, aspects or embodiments of the present technology.

### Example 1. Construction and Characterization of an Illustrative CD86- and NKG2A-based Chimeric Protein

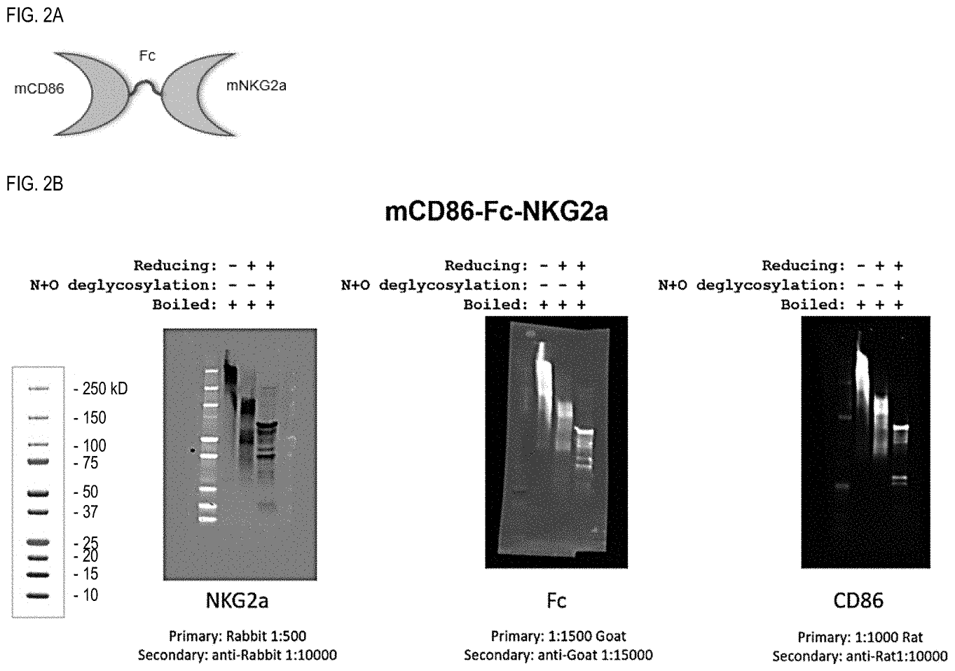

A construct encoding a murine CD86- and NKG2A-based chimeric protein was generated. The “mCD86-Fc-NKG2A” construct included an extracellular domain (ECD) of murine CD86 fused to an ECD of murine NKG2A via a hinge-CH2-CH3 Fc domain derived from IgG1. See, FIG. 2A.

The construct was codon optimized for expression in Chinese Hamster Ovary (CHO) cells, transfected into CHO cells and individual clones were selected for high expression. High expressing clones were then used for small-scale manufacturing in stirred bioreactors in serum-free media and the relevant chimeric fusion proteins were purified with Protein A binding resin columns.

The mCD86-Fc-NKG2A construct was transiently expressed in 293 cells and purified using protein-A affinity chromatography. To understand the native structure of the mCD86-Fc-NKG2A chimeric protein, untreated denatured samples (i.e., boiled in the presence of SDS, without a treatment with a reducing agent or a deglycosylation agent) were compared with (i) reduced samples, which were not deglycosylated (i.e. treated only with β-mercaptoethanol, and boiled in the presence of SDS); and (ii) reduced and deglycosylated samples (i.e. treated both with β-mercaptoethanol and a deglycosylation agent, and boiled in the presence of SDS). In addition, to confirm the presence of each domain of the mCD86-Fc-NKG2A chimeric protein, the gels were run in triplicates and probed using an anti-CD86 antibody (FIG. 2B, left blot), an anti-Fc antibody (FIG. 2B, center blot), or an anti-NKG2A antibody (FIG. 2B, right blot). The Western blots indicated the presence of a dominant dimer band in the non-reduced lanes (FIG. 2B, lane 2 in each blot), which was reduced to a glycosylated monomeric band in the presence of the reducing agent, β-mercaptoethanol (FIG. 2B, lane 3 in each blot). As shown in FIG. 2B, lane 4 in each blot, the chimeric protein ran as a monomer at the predicted molecular weight of about 69 kDa in the presence of both a reducing agent (β-mercaptoethanol) and a deglycosylation agent.

### Example 2. Further Characterization of the Binding Affinity of the Different Domains of the mCD86-Fc-NKG2A Chimeric Protein Using ELISA

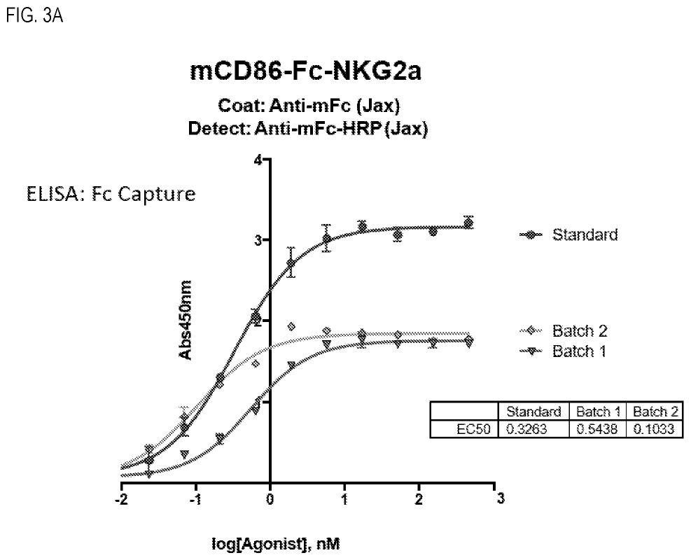

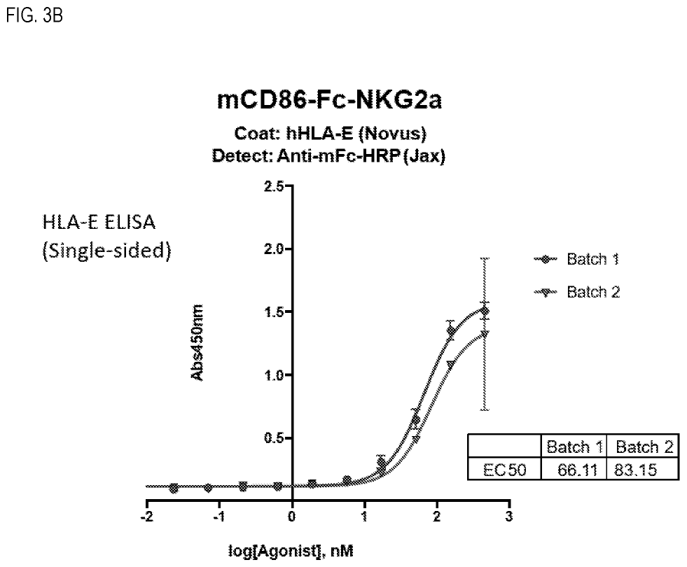

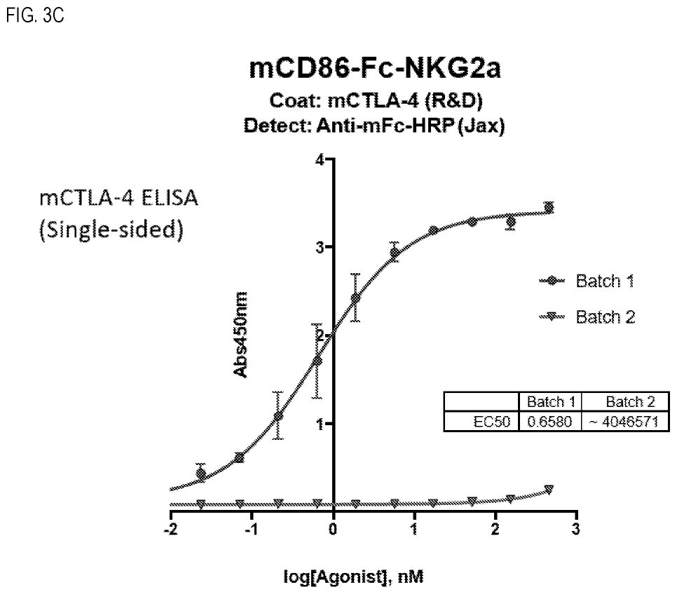

Functional ELISA (enzyme-linked immunosorbent assay) were performed to demonstrate the binding affinity of the different domains of the mCD86-Fc-NKG2A chimeric protein to their respective binding partners. As shown in FIG. 3A, binding of the Fc portion of the mCD86-Fc-NKG2A chimeric protein obtained from two distinct syntheses (Batch 1 and Batch 2) were characterized by capturing the chimeric protein to a plate-bound mouse IgG Fc gamma antibody and detecting via an HRP conjugated anti-mouse Fc antibody. A mouse whole IgG was used to generate a standard curve.

As shown in FIG. 3B, binding of the NKG2A domain of the mCD86-Fc-NKG2A chimeric proteins (Batch 1 and Batch 2) were characterized by capturing to a plate-bound HLA class I histocompatibility antigen, alpha chain E (HLA-E) and detecting via an HRP conjugated anti-mouse Fc antibody. The data shown in FIG. 3B demonstrates that the NKG2A domain of mCD86-Fc-NKG2A effectively interacted with its binding partner in a concentration-dependent manner and with high affinity.

As shown in FIG. 3C, binding of the CD86 domain of the mCD86-Fc-NKG2A chimeric proteins (Batch 1 and Batch 2) were characterized by capturing to a plate-bound mCTLA-4 and detecting via an HRP conjugated anti-mouse Fc antibody. The data shown in FIG. 3C demonstrates that the CD86 domain of mCD86-Fc-NKG2A for Batch 1 effectively interacted with its binding partner in a concentration-dependent manner and with high affinity whereas the Batch 2 interacted less efficiently.

### Example 3. Construction and Characterization of an Illustrative CD80- and NKG2A-Based Chimeric Protein

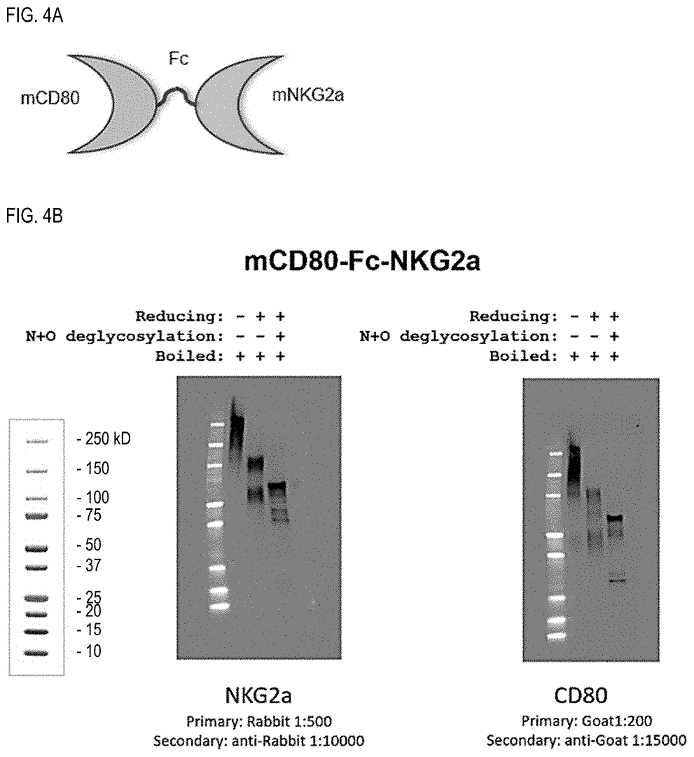

A construct encoding a murine CD80- and NKG2A-based chimeric protein was generated. The “mCD80-Fc-NKG2A” construct included an extracellular domain (ECD) of murine CD80 fused to an ECD of murine NKG2A via a hinge-CH2-CH3 Fc domain derived from IgG1. See, FIG. 4A.

The construct was expressed and purified as described in Example 1.

To understand the native structure of the mCD80-Fc-NKG2A chimeric protein, untreated denatured samples (i.e., boiled in the presence of SDS, without a treatment with a reducing agent or a deglycosylation agent) were compared with (i) reduced samples, which were not deglycosylated (i.e. treated only with β-mercaptoethanol, and boiled in the presence of SDS); and (ii) reduced and deglycosylated samples (i.e. treated both with β-mercaptoethanol and a deglycosylation agent, and boiled in the presence of SDS). In addition, to confirm the presence of each domain of the mCD80-Fc-NKG2A chimeric protein, the gels were run in triplicates and probed using an anti-CD80 antibody (FIG. 4B, left blot), an anti-Fc antibody (FIG. 4B, center blot), or an anti-NKG2A antibody (FIG. 4B, right blot). The Western blots indicated the presence of a dominant dimer band in the non-reduced lanes (FIG. 4B, lane 2 in each blot), which was reduced to a glycosylated monomeric band in the presence of the reducing agent, β-mercaptoethanol (FIG. 4B, lane 3 in each blot). As shown in FIG. 4B, lane 4 in each blot, the chimeric protein ran as a monomer at the predicted molecular weight of about 67 kDa in the presence of both a reducing agent (β-mercaptoethanol) and a deglycosylation agent.

### Example 4. Further Characterization of the Binding Affinity of the Different Domains of the mCD80-Fc-NKG2A Chimeric Protein Using ELISA

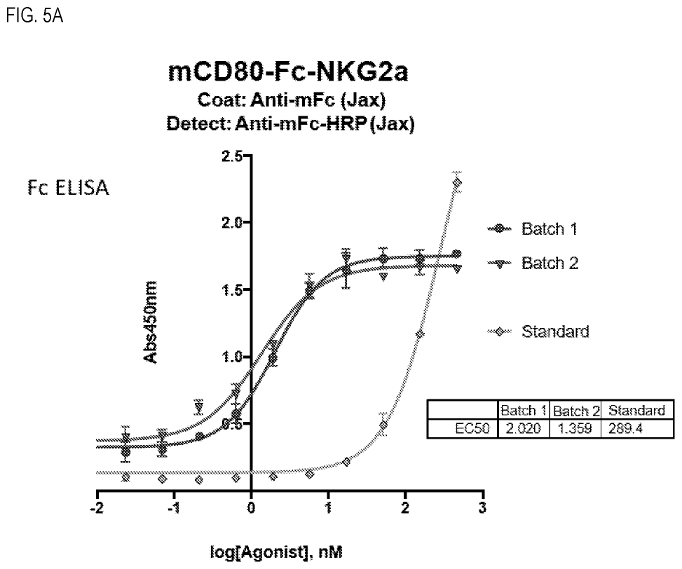

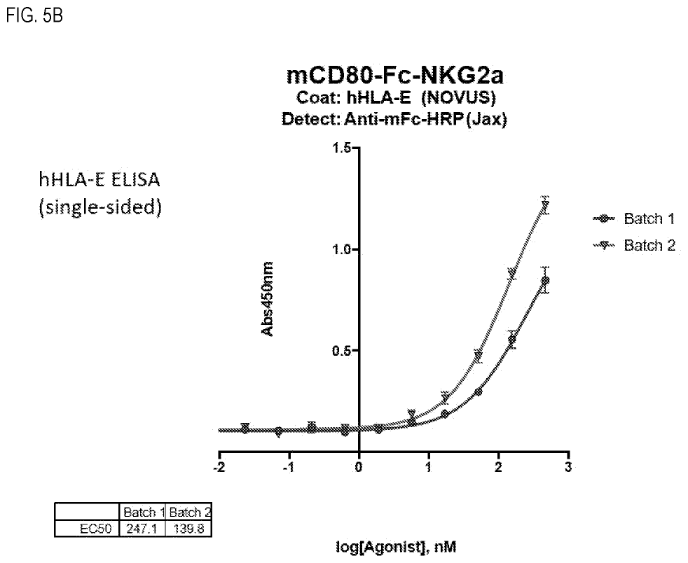

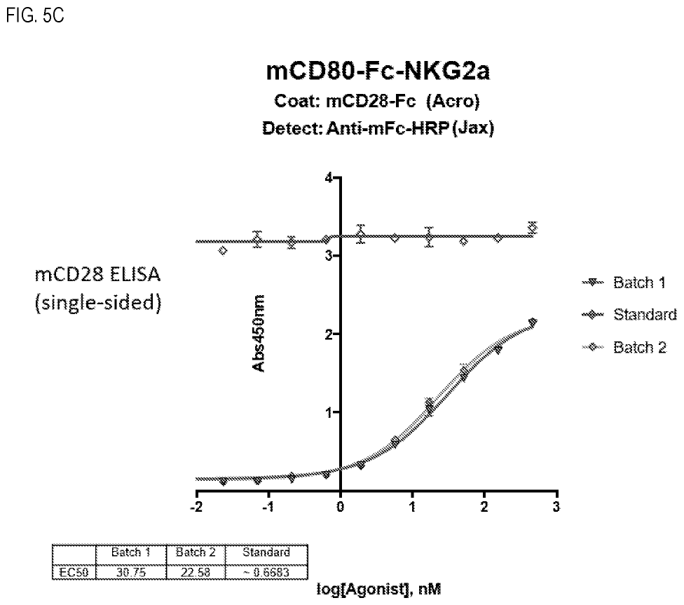

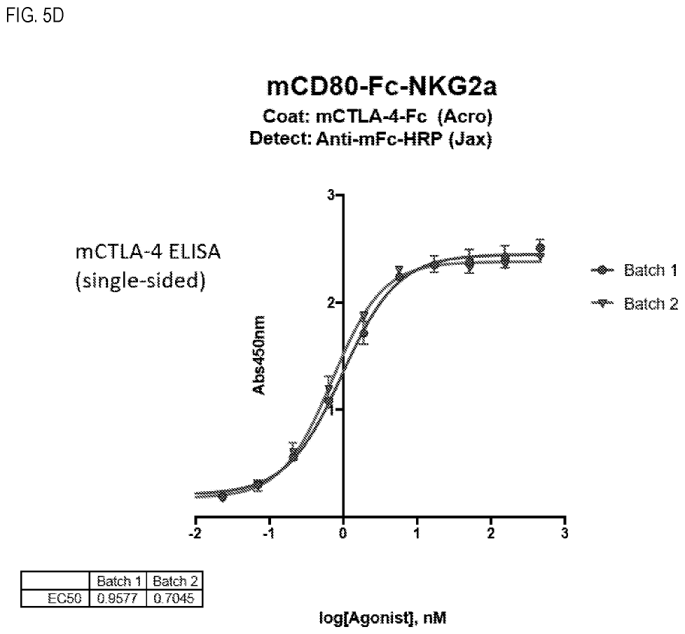

Functional ELISA were performed to demonstrate the binding affinity of the different domains of the mCD80-Fc-NKG2A chimeric protein to their respective binding partners. As shown in FIG. 5A, binding of the Fc portion of the mCD80-Fc-NKG2A chimeric protein obtained from two distinct syntheses (Batch 1 and Batch 2) were characterized by capturing the chimeric protein to a plate-bound mouse IgG Fc gamma antibody and detecting via an HRP conjugated anti-mouse Fc antibody. A mouse whole IgG was used to generate a standard curve.

As shown in FIG. 5B, binding of the NKG2A domain of the mCD80-Fc-NKG2A chimeric proteins (Batch 1 and Batch 2) were characterized by capturing to a plate-bound HLA-E and detecting via an HRP conjugated anti-mouse Fc antibody. The data shown in FIG. 5B demonstrates that the NKG2A domain of mCD80-Fc-NKG2A effectively interacted with its binding partner in a concentration-dependent manner.

Since CD80 is the ligand for, at least, two different proteins on the T cell surface: CD28 (for autoregulation and intercellular association) and CTLA-4 (for attenuation of regulation and cellular disassociation), the affinity of the CD80 domain of the mCD80-Fc-NKG2A chimeric protein was characterized for each of CD28 and CTLA-4. As shown in FIG. 5C and FIG. 5D, binding of the CD80 domain of the mCD80-Fc-NKG2A chimeric proteins (Batch 1 and Batch 2) were characterized by capturing to, respectively, a plate-bound mCD28 (FIG. 5C) and a plate-bound mCTLA-4 (FIG. 5D) and detecting via an HRP conjugated anti-mouse Fc antibody. The data shown in FIG. 5C and FIG. 5D demonstrate that the CD80 domain of mCD80-Fc-NKG2A effectively interacted with its binding partners (CD28 and CTLA-4) in a concentration-dependent manner and with high affinity, with the mCD80-Fc-NKG2A chimeric protein having greater affinity for CTLA-4 than for CD28.

### Example 5. Construction and Characterization of an Illustrative CD48- and NKG2A-based Chimeric Protein

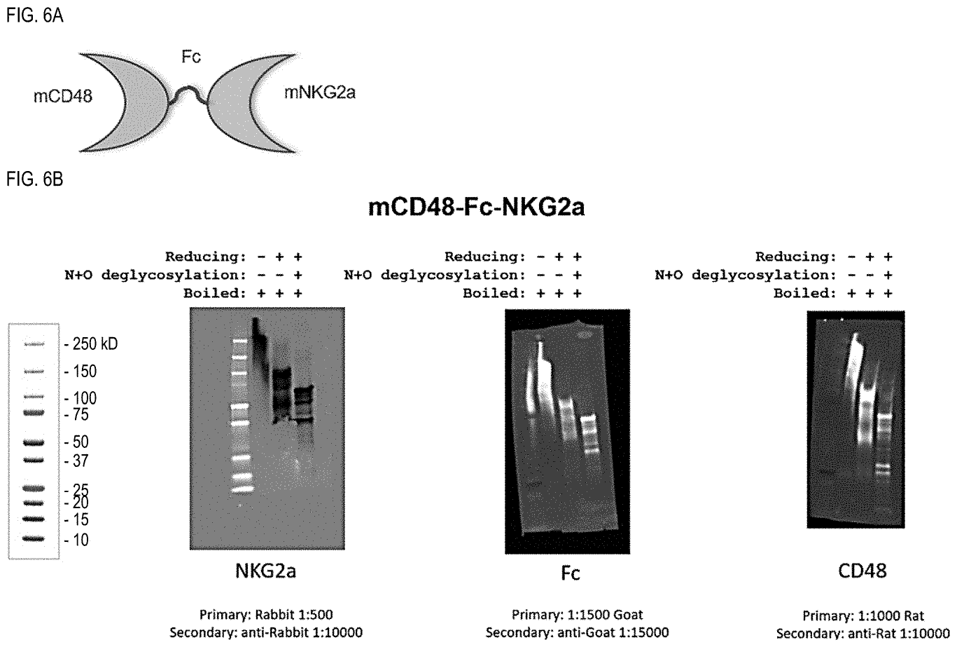

A construct encoding a murine CD48- and NKG2A-based chimeric protein was generated. The “mCD48-Fc-NKG2A” construct included an extracellular domain (ECD) of murine CD48 fused to an ECD of murine NKG2A via a hinge-CH2-CH3 Fc domain derived from IgG1. See, FIG. 6A.

The construct was expressed and purified as described in Example 1.

To understand the native structure of the mCD48-Fc-NKG2A chimeric protein, untreated denatured samples (i.e., boiled in the presence of SDS, without a treatment with a reducing agent or a deglycosylation agent) were compared with (i) reduced samples, which were not deglycosylated (i.e. treated only with β-mercaptoethanol, and boiled in the presence of SDS); and (ii) reduced and deglycosylated samples (i.e. treated both with β-mercaptoethanol and a deglycosylation agent, and boiled in the presence of SDS). In addition, to confirm the presence of each domain of the mCD48-Fc-NKG2A chimeric protein, the gels were run in triplicates and probed using an anti-CD48 antibody (FIG. 6B, left blot), an anti-Fc antibody (FIG. 6B, center blot), or an anti-NKG2A antibody (FIG. 6B, right blot). The Western blots indicated the presence of a dominant dimer band in the non-reduced lanes (FIG. 6B, lane 2 in each blot), which was reduced to a glycosylated monomeric band in the presence of the reducing agent, β-mercaptoethanol (FIG. 6B, lane 3 in each blot). As shown in FIG. 6B, lane 4 in each blot, the chimeric protein ran as a monomer at the predicted molecular weight of about 66 kDa in the presence of both a reducing agent (β-mercaptoethanol) and a deglycosylation agent.

### Example 6. Further Characterization of the Binding Affinity of the Different Domains of the mCD48-Fc-NKG2A Chimeric Protein Using ELISA

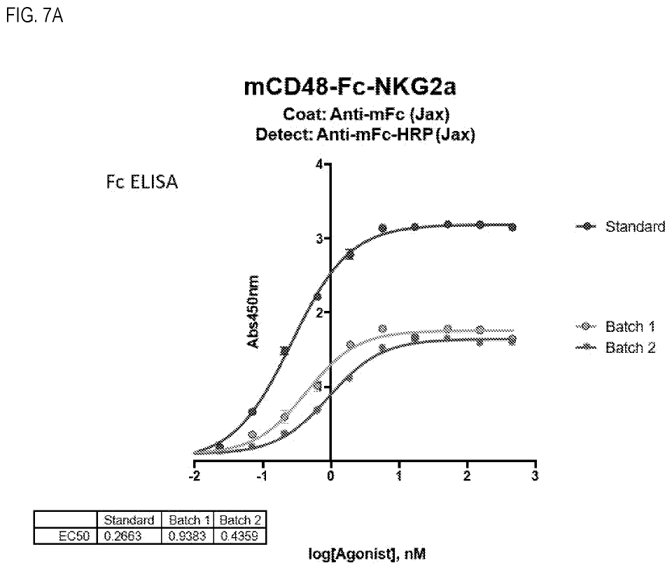

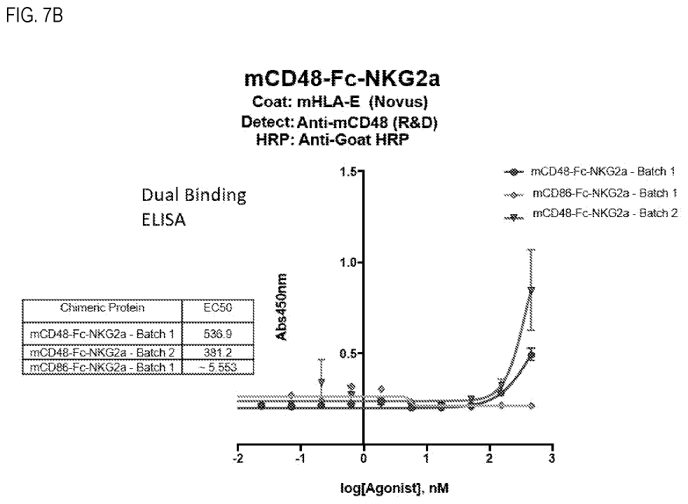

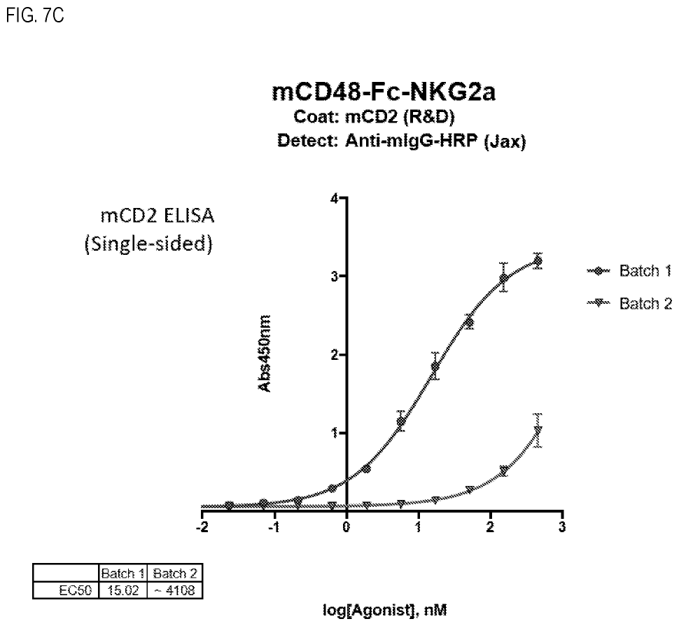

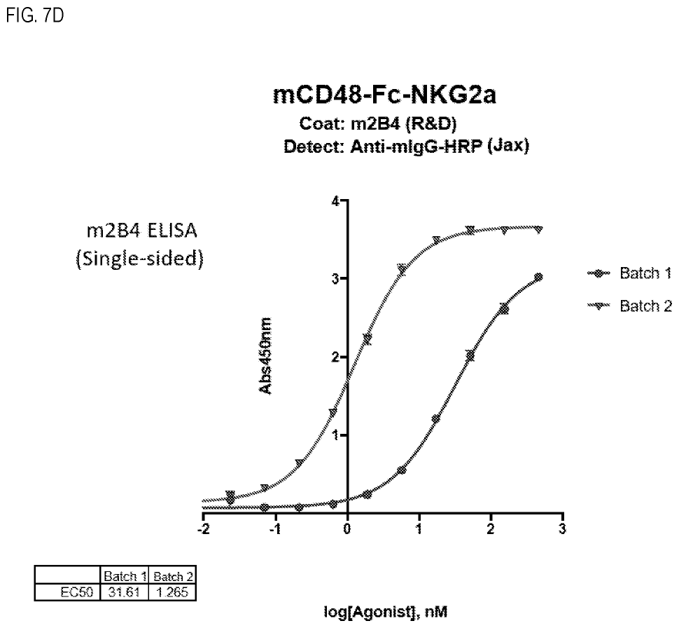

Functional ELISA assays were performed to demonstrate the binding affinity of the different domains of the mCD48-Fc-NKG2A chimeric protein to their respective binding partners. As shown in FIG. 7A, binding of the Fc portion of the mCD48-Fc-NKG2A chimeric protein obtained from two distinct syntheses (Batch 1 and Batch 2) were characterized by capturing the chimeric protein to a plate-bound mouse IgG Fc gamma antibody and detecting via an HRP conjugated anti-mouse Fc antibody. A mouse whole IgG was used to generate a standard curve.

As shown in FIG. 7B, binding of the NKG2A domain of the mCD48-Fc-NKG2A chimeric proteins (Batch 1 and Batch 2) were characterized by capturing to a plate-bound HLA-E and detecting using an anti-mCD48 antibody in conjunction with an anti-goat HRP antibody. The data shown in FIG. 7B demonstrates that the NKG2A domain of mCD48-Fc-NKG2A effectively interacted with its binding partner in a concentration-dependent manner. Also shown in FIG. 7B is binding of the NKG2A domain of mCD86-Fc-NKG2A (Batch 1) to HLA-E.

CD48 had been found to have a low affinity for ligand CD2 and high affinity for ligand 2B4 (CD244), which is also a member of the CD2 subfamily SLAM of IgSF expressed on natural killer cells (NK cells) and other leukocytes. Since CD48 has, at least, two known ligands, the affinity of the CD48 domain of the mCD48-Fc-NKG2A chimeric protein was characterized for each of CD2 and 2B4. As shown in FIG. 7C and FIG. 7D, binding of the CD48 domain of the mCD48-Fc-NKG2A chimeric proteins (Batch 1 and Batch 2) were characterized by capturing to, respectively, a plate-bound mCD2 (FIG. 7C) and a plate-bound m2B4 (FIG. 7D) and detecting via an HRP conjugated anti-mouse Fc antibody. The data shown in FIG. 7C and FIG. 7D demonstrate that the CD48 domain of mCD48-Fc-NKG2A effectively interacted with its binding partners (CD2 and 2B4) in a concentration-dependent manner, with the mCD48-Fc-NKG2A chimeric protein having greater affinity for 2B4 than for CD2, as expected. There was an observed variation in binding ability between batches.

### Example 7. Construction and Characterization of an Illustrative PD-1- and NKG2A-based Chimeric Protein

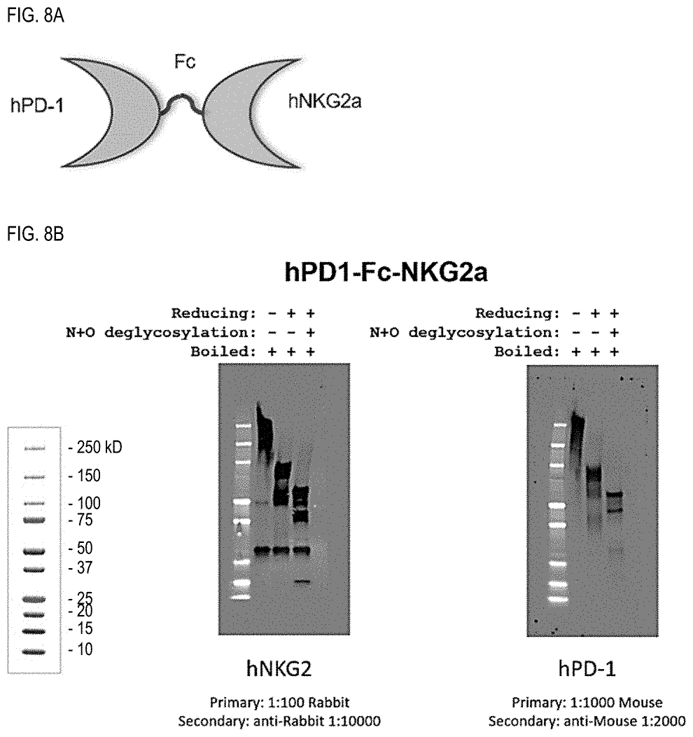

A construct encoding a murine PD-1- and NKG2A-based chimeric protein was generated. The “hPD-1-Fc-hNKG2A” construct included an extracellular domain (ECD) of human programmed cell death protein 1 (PD-1) fused to an ECD of human NKG2A via a hinge-CH2-CH3 Fc domain derived from IgG1. See, FIG. 8A.

The construct was expressed and purified as described in Example 1.

To understand the native structure of the hPD-1-Fc-NKG2A chimeric protein, untreated denatured samples (i.e., boiled in the presence of SDS, without a treatment with a reducing agent or a deglycosylation agent) were compared with (i) reduced samples, which were not deglycosylated (i.e. treated only with β-mercaptoethanol, and boiled in the presence of SDS); and (ii) reduced and deglycosylated samples (i.e. treated both with 3-mercaptoethanol and a deglycosylation agent, and boiled in the presence of SDS). In addition, to confirm the presence of each domain of the hPD-1-Fc-NKG2A chimeric protein, the gels were run in triplicates and probed using an anti-PD-1 antibody (FIG. 8B, left blot), an anti-Fc antibody (FIG. 8B, center blot), or an anti-NKG2A antibody (FIG. 8B, right blot). The Western blots indicated the presence of a dominant dimer band in the non-reduced lanes (FIG. 8B, lane 2 in each blot), which was reduced to a glycosylated monomeric band in the presence of the reducing agent, β-mercaptoethanol (FIG. 8B, lane 3 in each blot). As shown in FIG. 8B, lane 4 in each blot, the chimeric protein ran as a monomer at the predicted molecular weight of about 58 kDa in the presence of both a reducing agent (β-mercaptoethanol) and a deglycosylation agent.

### Example 8: Further Characterization of the Binding Affinity of the Different Domains of the hPD-1-Fc-hNKG2A Chimeric Protein Using ELISA

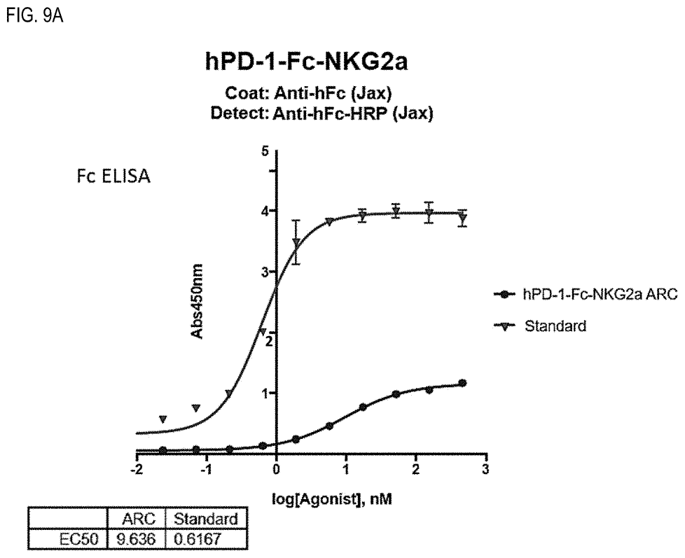

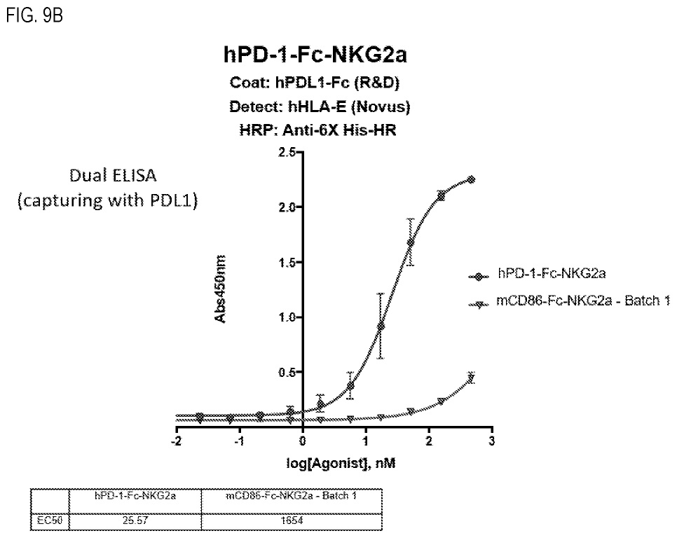

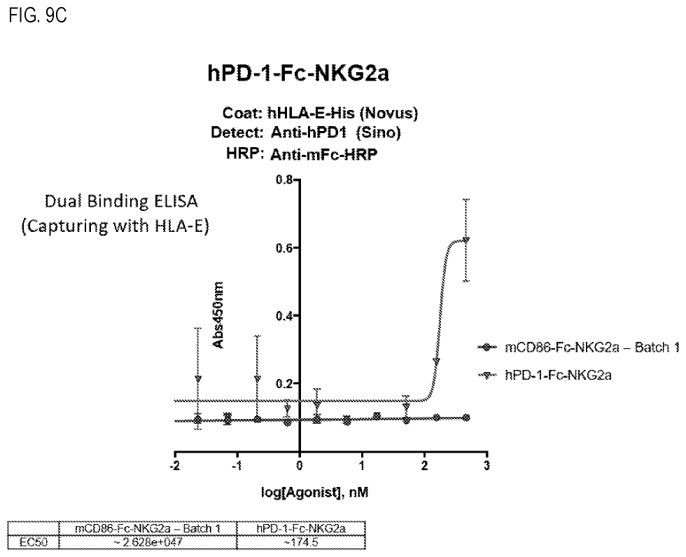

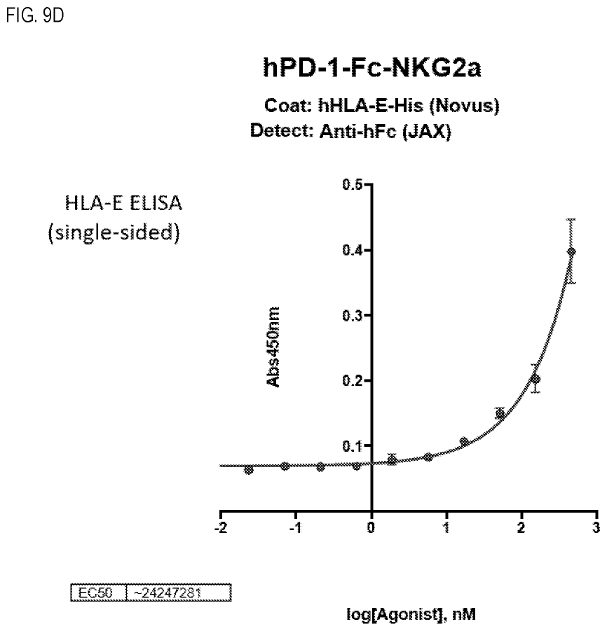

Functional ELISA were performed to demonstrate the binding affinity of the different domains of the hPD-1-Fc-hNKG2A chimeric protein to their respective binding partners. As shown in FIG. 9A, binding of the Fc portion of the hPD-1-Fc-hNKG2A chimeric protein was characterized by capturing the chimeric protein to a plate-bound mouse IgG Fc gamma antibody and detecting via an HRP conjugated anti-mouse Fc antibody. A mouse whole IgG was used to generate a standard curve.

As shown in FIG. 9B, binding of the NKG2A domain of the hPD-1-Fc-hNKG2A chimeric protein was characterized by capturing to a plate-bound PD-L1 and detecting using an anti-human HLA-E antibody in conjunction with an anti-6X His-HRP antibody. The data shown in FIG. 9B demonstrates that the PD-1 domain of hPD-1-Fc-hNKG2A effectively interacted with its binding partner in a concentration-dependent manner.

As shown in FIG. 9C and FIG. 9D, binding of the hNKG2A domain of the hPD-1-Fc-hNKG2A chimeric protein was characterized by capturing to, respectively, a plate-bound HLA-E. In FIG. 9C, the hPD-1-Fc-hNKG2A chimeric protein was detected using an anti-human PD-1 antibody in conjunction with an HRP conjugated anti-mouse Fc antibody. In FIG. 9D, the hPD-1-Fc-hNKG2A chimeric protein was directly detected via an HRP conjugated anti-mouse Fc antibody. The data shown in FIG. 9C and FIG. 9D demonstrate that the hNKG2A domain of hPD-1-Fc-hNKG2A interacted with its binding partner. Also shown in FIG. 9C is an absence of detection of mCD86-Fc-NKG2A (Batch 1) binding using an anti-PD-1 antibody; since mCD86-Fc-NKG2A lacks a PD-1-containing domain, this results was expected.

### Example 9: Characterization of Illustrative Chimeric Proteins Using Non-Denaturing PAGE

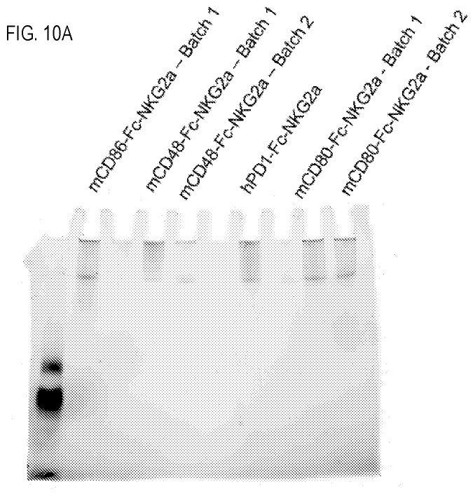

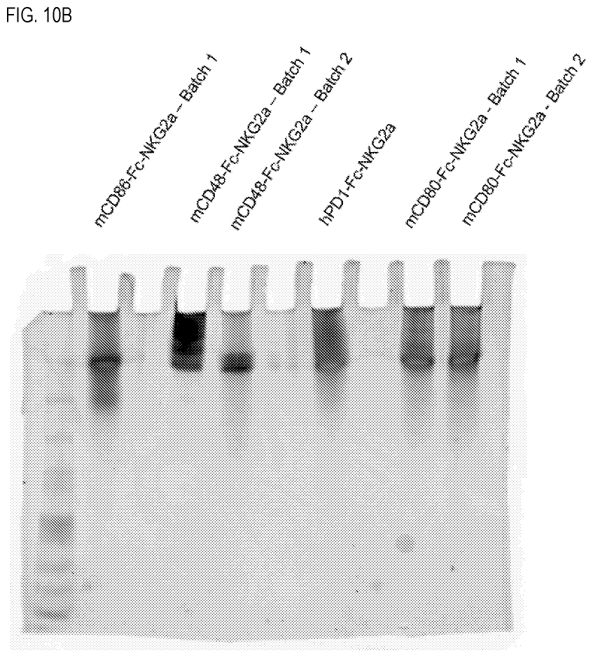

When the chimeric proteins disclosed in Examples 1 to 8 were run on native PAGE, which lacks SDS, and in the absence of a reducing agent the chimeric proteins appear to exist as a dimer. See, FIG. 10A and FIG. 10B. Without wishing to be bound to theory, it appears that the chimeric, proteins which comprise Fc-based linkers, which comprise cysteine residues capable of forming disulfide bonds, are responsible for promoting dimerization of the chimeric proteins. Moreover, since none of the illustrative chimeric proteins in Examples 1 to 8 comprise a TNFRSF ligand, which trimerize, none of the chimeric proteins appeared to exist as a hexamer.

### Example 10: Functional In Vivo Anti-Tumor Activity of Specific Combinations of Antibodies Directed to Immune Checkpoint Molecules and Illustrative Chimeric Proteins

The in vivo ability of specific combinations of antibodies directed to immune checkpoint molecules and chimeric proteins of the present invention to target and reduce tumor volume was determined.

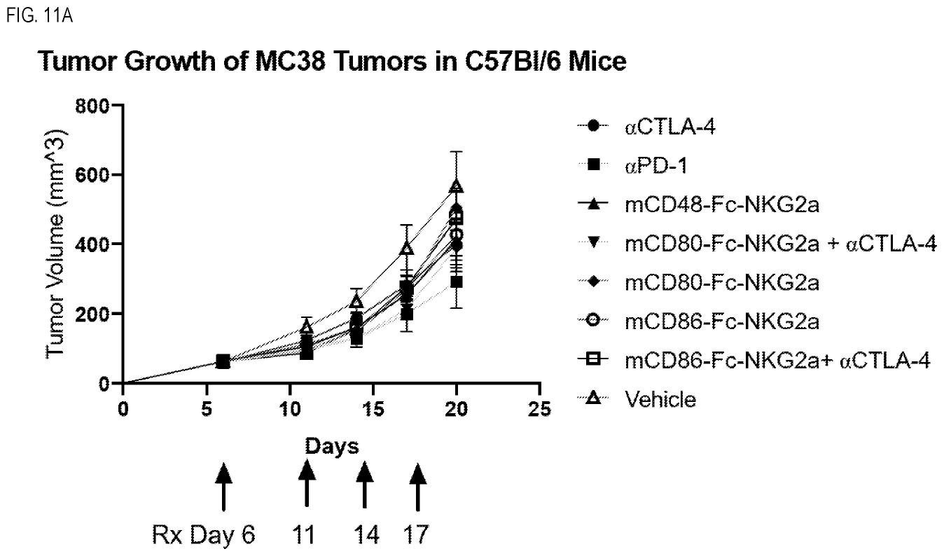

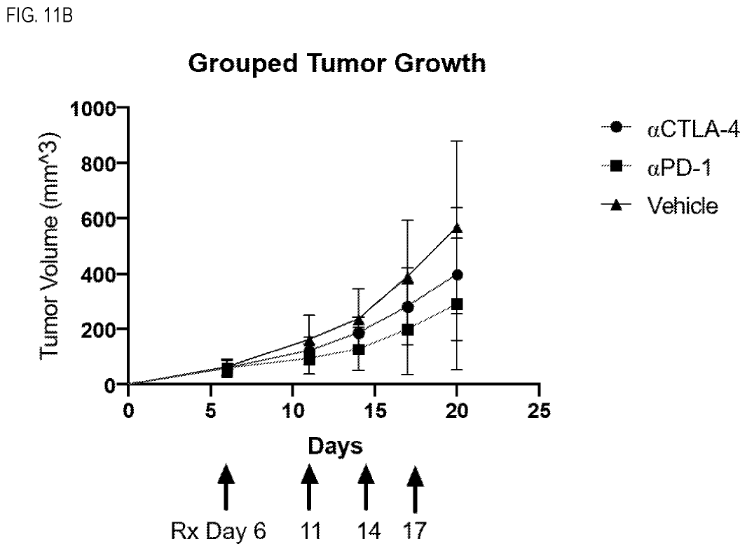

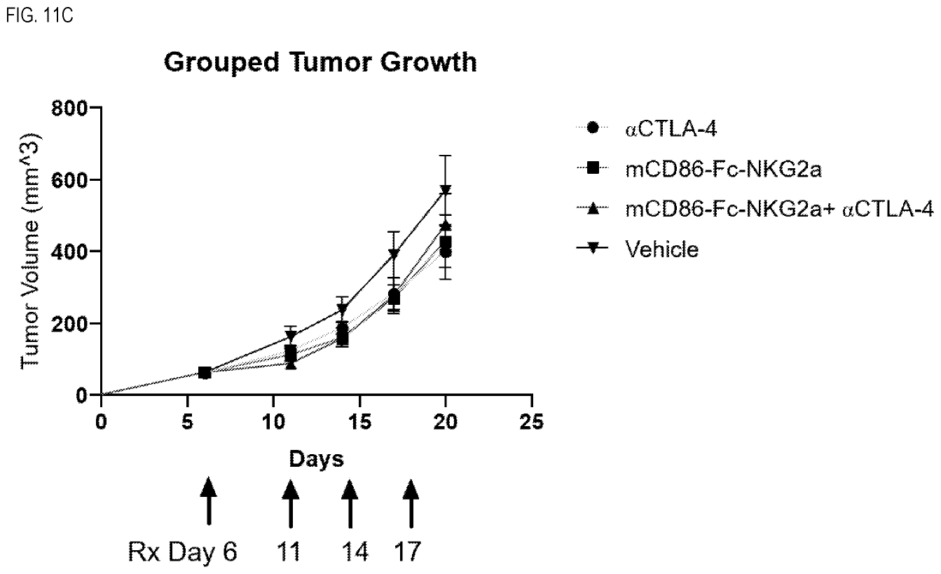

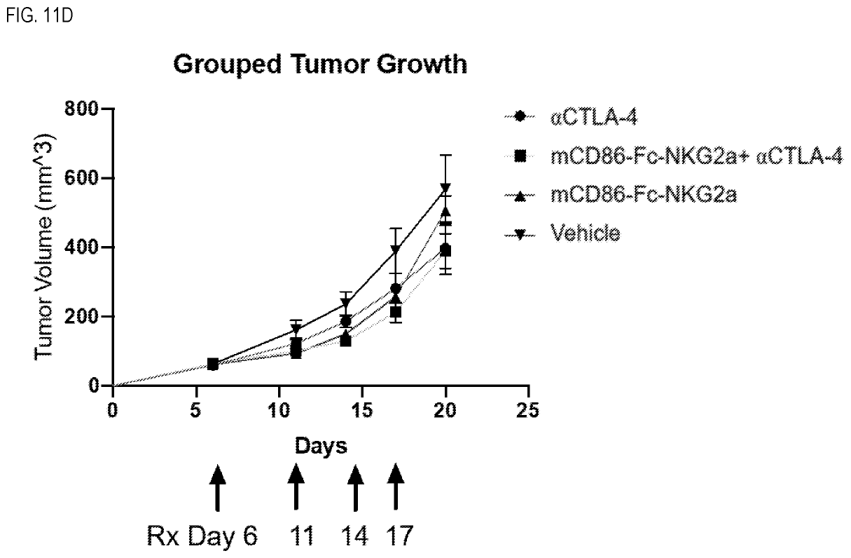

Mice were inoculated with MC38 (colorectal carcinoma) tumors cells. Six days after inoculation, there was no significant difference between starting tumor volumes among the mice, i.e., volumes were approximately 60 mm³. Treatment then began according to the schedule shown in FIG. 11A to FIG. 11E, i.e., on day 6, day 11, day 14, and day 17. Specific combinations were included: an anti-CTLA-4 antibody (9D9); an anti-PD-1 antibody (RMP1-14); the mCD48-Fc-NKG2A chimeric protein; the mCD48-Fc-NKG2A chimeric protein and the anti-CTLA-4 antibody; the mCD80-Fc-NKG2A chimeric protein; the mCD86-Fc-NKG2A chimeric protein; the mCD86-Fc-NKG2A chimeric protein and the anti-CTLA-4 antibody; or a vehicle. Antibodies were administered at the indicated time points as 100 pg intraperitoneal injection (IP) injection and the chimeric proteins were given as a 150 pg IP injection on day 6, day 11, day 14, and day 17. Tumor sizes were assayed every other day until the 20th day after inoculation.

As shown in FIG. 11A to FIG. 11E, the chimeric proteins had anti-tumor efficacy in MC38 cells and at relatively low doses. The data further suggests the possibility of a combinatorial efficacy with the anti-CTLA-4 antibody. The data shown in FIG. 11A consolidates the data shown in FIG. 11B to FIG. 11E.

In a similar set of experiments, mice inoculated with MC38 tumor cells and combination of a chimeric protein with an anti-PD-1 antibody are assayed.

Additionally, mice are inoculated CT26 (colon carcinoma) tumor cells and the anti-cancer effects from treatments with the chimeric proteins with or without an anti-CTLA-4 antibody or an anti-PD-1 antibody are assayed.

### Example 11: Construction and Characterization of an Illustrative Human CD86- and NKG2A-Based Chimeric Protein

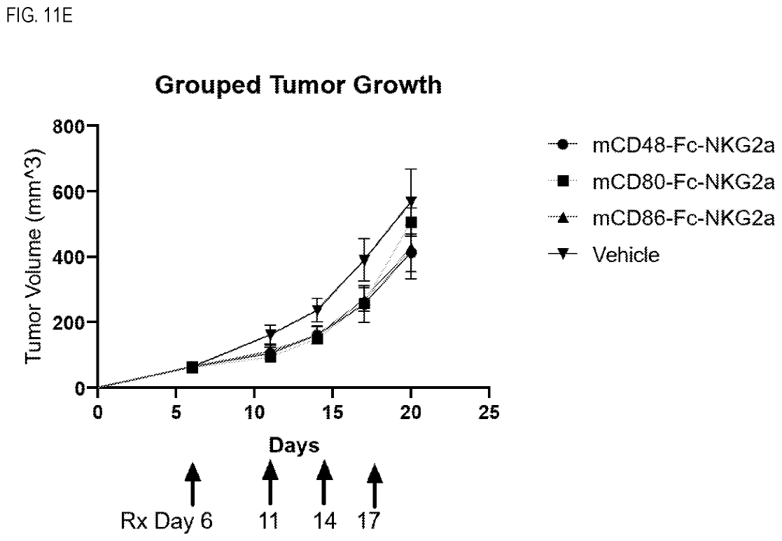

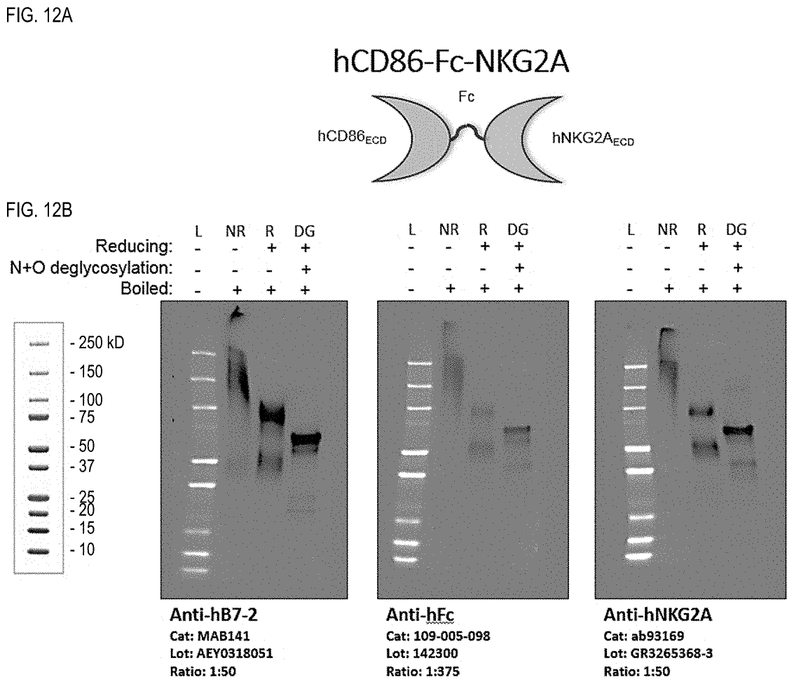

A construct encoding a human CD86- and NKG2A-based chimeric protein was generated. The hCD86-Fc-NKG2A chimeric protein construct included an extracellular domain (ECD) of human CD86 fused to an extracellular domain (ECD) of human NKG2A via a hinge-CH2-CH3 Fc domain derived from IgG1. See, FIG. 12A.

The construct was expressed and purified as described in Example 1.

To understand the native structure of the hCD86-Fc-NKG2A chimeric protein, untreated denatured samples (i.e., boiled in the presence of SDS, without a treatment with a reducing agent or a deglycosylation agent) were compared with (i) reduced samples, which were not deglycosylated (i.e. treated only with β-mercaptoethanol, and boiled in the presence of SDS); and (ii) reduced and deglycosylated samples (i.e. treated both with 3-mercaptoethanol and a deglycosylation agent, and boiled in the presence of SDS). In addition, to confirm the presence of each domain of the hCD86-Fc-NKG2A chimeric protein, the gels were run in triplicates and probed using an anti-CD86 antibody (FIG. 12B, left blot), an anti-Fc antibody (FIG. 12B, center blot), or an anti-NKG2A antibody (FIG. 12B, right blot). Western blot analyses were performed to confirm the presence the two ECD domains and the Fc linker of the hCD86-Fc-NKG2A chimeric protein with antibodies specific to the two ECD domains and the Fc linker (FIG. 12B). As shown in FIG. 12B, the Western blots indicated the presence of a dominant dimer band in the non-reduced lanes (FIG. 12B, lane NR in each blot), which was reduced to a glycosylated monomeric band in the presence of the reducing agent, β-mercaptoethanol (FIG. 12B, lane R in each blot). The chimeric protein ran as a monomer at the predicted molecular weight of about 67 kDa in the presence of both a reducing agent (β-mercaptoethanol) and a deglycosylation agent (FIG. 12B, lane DG in each blot). These results demonstrate the native state and tendency to form a dimer of the hCD86-Fc-NKG2A chimeric protein.

### Example 12: Construction and Characterization of an Illustrative Human CD48- and NKG2A-Based Chimeric Protein

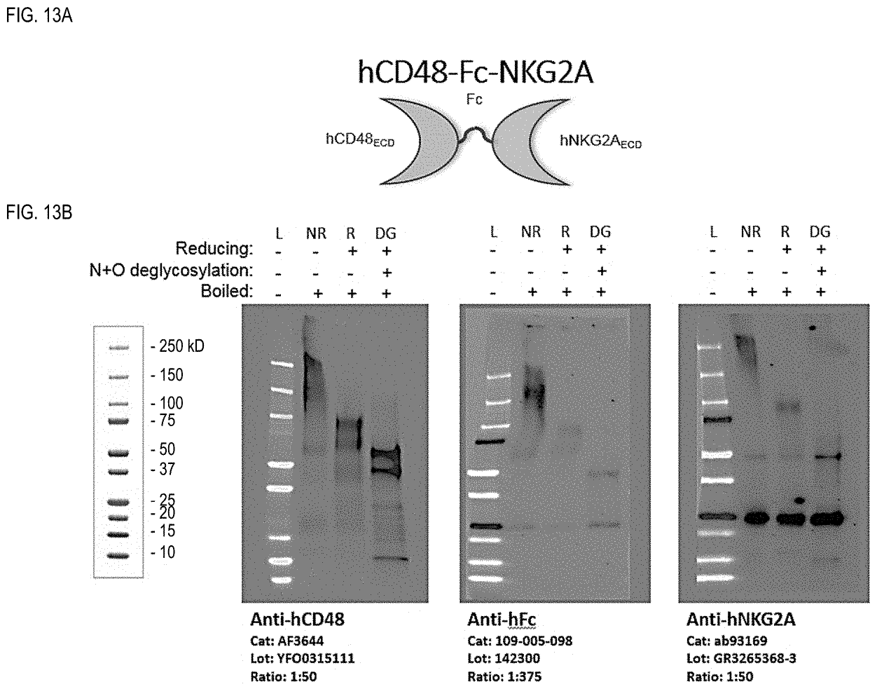

A construct encoding a human CD48- and NKG2A-based chimeric protein was generated. The hCD48-Fc-NKG2A chimeric protein construct included an extracellular domain (ECD) of human CD48 fused to an extracellular domain (ECD) of human NKG2A via a hinge-CH2-CH3 Fc domain derived from IgG1. See, FIG. 13A.

The construct was expressed and purified as described in Example 1.

To understand the native structure of the hCD48-Fc-NKG2A chimeric protein, untreated denatured samples (i.e., boiled in the presence of SDS, without a treatment with a reducing agent or a deglycosylation agent) were compared with (i) reduced samples, which were not deglycosylated (i.e. treated only with β-mercaptoethanol, and boiled in the presence of SDS); and (ii) reduced and deglycosylated samples (i.e. treated both with β-mercaptoethanol and a deglycosylation agent, and boiled in the presence of SDS). In addition, to confirm the presence of each domain of the hCD48-Fc-NKG2A chimeric protein, the gels were run in triplicates and probed using an anti-CD48 antibody (FIG. 13B, left blot), an anti-Fc antibody (FIG. 13B, center blot), or an anti-NKG2A antibody (FIG. 13B, right blot). Western blot analyses were performed to confirm the presence the two ECD domains and the Fc linker of the hCD48-Fc-NKG2A chimeric protein with antibodies specific to the two ECD domains and the Fc linker (FIG. 13B). As shown in FIG. 13B, the Western blots indicated the presence of a dominant dimer band in the non-reduced lanes (FIG. 13B, lane NR in each blot), which was reduced to a glycosylated monomeric band in the presence of the reducing agent, β-mercaptoethanol (FIG. 13B, lane R in each blot). The chimeric protein ran as a monomer at the predicted molecular weight of about 67 kDa in the presence of both a reducing agent (β-mercaptoethanol) and a deglycosylation agent (FIG. 13B, lane DG in each blot). These results demonstrate the native state and tendency to form a dimer of the hCD48-Fc-NKG2A chimeric protein.

### Example 13: Construction and Characterization of an Illustrative Human CD58- and NKG2A-Based Chimeric Protein

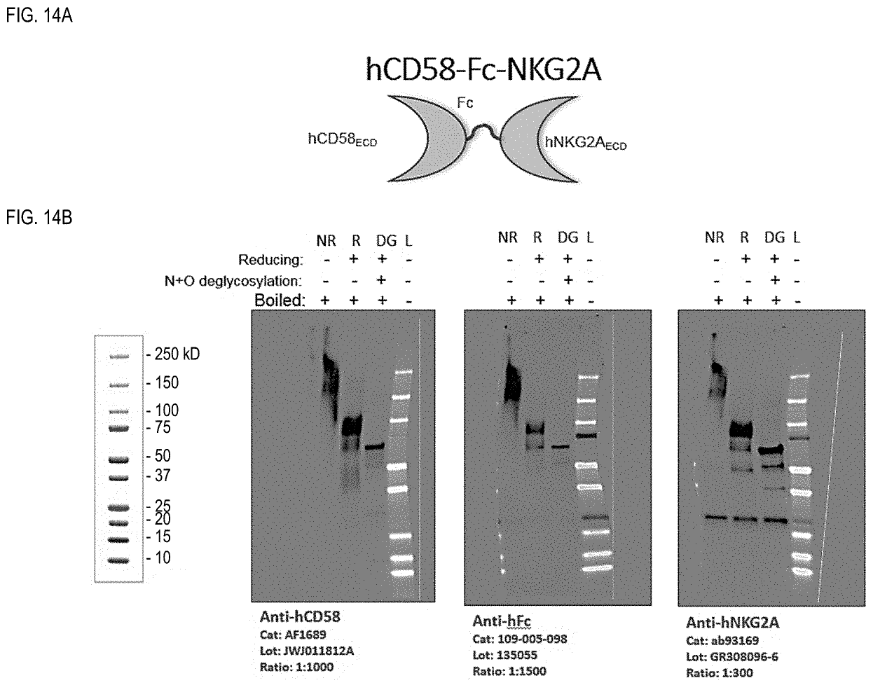

A construct encoding a human CD58- and NKG2A-based chimeric protein was generated. The hCD58-Fc-NKG2A chimeric protein construct included an extracellular domain (ECD) of human CD58 fused to an extracellular domain (ECD) of human NKG2A via a hinge-CH2-CH3 Fc domain derived from IgG1. See, FIG. 14A.

The construct was expressed and purified as described in Example 1.

To understand the native structure of the hCD58-Fc-NKG2A chimeric protein, untreated denatured samples (i.e., boiled in the presence of SDS, without a treatment with a reducing agent or a deglycosylation agent) were compared with (i) reduced samples, which were not deglycosylated (i.e. treated only with 3-mercaptoethanol, and boiled in the presence of SDS); and (ii) reduced and deglycosylated samples (i.e. treated both with β-mercaptoethanol and a deglycosylation agent, and boiled in the presence of SDS). In addition, to confirm the presence of each domain of the hCD58-Fc-NKG2A chimeric protein, the gels were run in triplicates and probed using an anti-CD58 antibody (FIG. 14B, left blot), an anti-Fc antibody (FIG. 14B, center blot), or an anti-NKG2A antibody (FIG. 14B, right blot). Western blot analyses were performed to confirm the presence the two ECD domains and the Fc linker of the hCD58-Fc-NKG2A chimeric protein with antibodies specific to the two ECD domains and the Fc linker (FIG. 14B). As shown in FIG. 14B, the Western blots indicated the presence of a dominant dimer band in the non-reduced lanes (FIG. 14B, lane NR in each blot), which was reduced to a glycosylated monomeric band in the presence of the reducing agent, 3-mercaptoethanol (FIG. 14B, lane R in each blot). The chimeric protein ran as a monomer at the predicted molecular weight of about 67 kDa in the presence of both a reducing agent (3-mercaptoethanol) and a deglycosylation agent (FIG. 14B, lane DG in each blot). These results demonstrate the native state and tendency to form a dimer of the hCD58-Fc-NKG2A chimeric protein.

### Example 14: Construction and Characterization of an Illustrative Human CD80- and NKG2A-Based Chimeric Protein

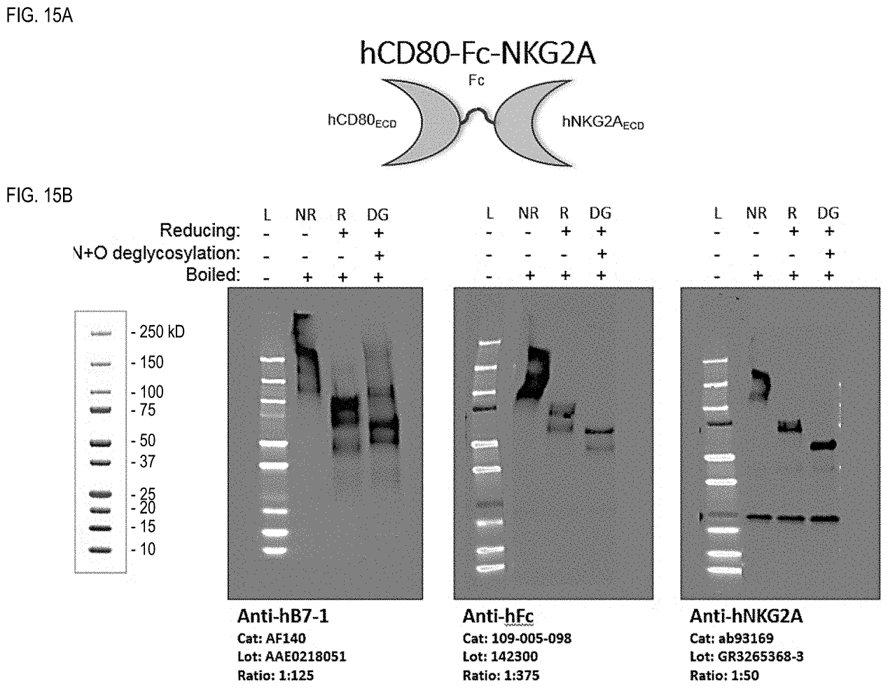

A construct encoding a human CD80- and NKG2A-based chimeric protein was generated. The hCD80-Fc-NKG2A chimeric protein construct included an extracellular domain (ECD) of human CD80 fused to an extracellular domain (ECD) of human NKG2A via a hinge-CH2-CH3 Fc domain derived from IgG1. See, FIG. 15A.

The construct was expressed and purified as described in Example 1.

To understand the native structure of the hCD80-Fc-NKG2A chimeric protein, untreated denatured samples (i.e., boiled in the presence of SDS, without a treatment with a reducing agent or a deglycosylation agent) were compared with (i) reduced samples, which were not deglycosylated (i.e. treated only with 3-mercaptoethanol, and boiled in the presence of SDS); and (ii) reduced and deglycosylated samples (i.e. treated both with 3-mercaptoethanol and a deglycosylation agent, and boiled in the presence of SDS). In addition, to confirm the presence of each domain of the hCD80-Fc-NKG2A chimeric protein, the gels were run in triplicates and probed using an anti-CD80 antibody (FIG. 15B, left blot), an anti-Fc antibody (FIG. 15B, center blot), or an anti-NKG2A antibody (FIG. 15B, right blot). Western blot analyses were performed to confirm the presence the two ECD domains and the Fc linker of the hCD80-Fc-NKG2A chimeric protein with antibodies specific to the two ECD domains and the Fc linker (FIG. 15B). As shown in FIG. 15B, the Western blots indicated the presence of a dominant dimer band in the non-reduced lanes (FIG. 15B, lane NR in each blot), which was reduced to a glycosylated monomeric band in the presence of the reducing agent, β-mercaptoethanol (FIG. 15B, lane R in each blot). The chimeric protein ran as a monomer at the predicted molecular weight of about 67 kDa in the presence of both a reducing agent (β-mercaptoethanol) and a deglycosylation agent (FIG. 15B, lane DG in each blot). These results demonstrate the native state and tendency to form a dimer of the hCD80-Fc-NKG2A chimeric protein.

### Example 15: Construction and Characterization of an Illustrative Human SLAMF6- and NKG2A-Based Chimeric Protein

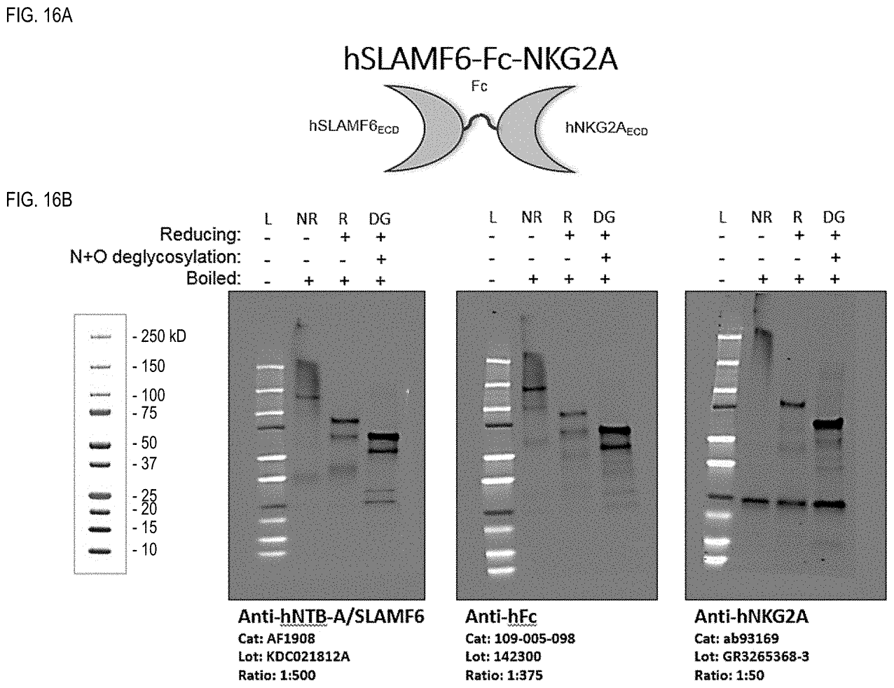

A construct encoding a human SLAMF6- and NKG2A-based chimeric protein was generated. The hSLAMF6-Fc-NKG2A chimeric protein construct included an extracellular domain (ECD) of human SLAMF6 fused to an extracellular domain (ECD) of human NKG2A via a hinge-CH2-CH3 Fc domain derived from IgG1. See, FIG. 16A.

The construct was expressed and purified as described in Example 1.

To understand the native structure of the hSLAMF6-Fc-NKG2A chimeric protein, untreated denatured samples (i.e., boiled in the presence of SDS, without a treatment with a reducing agent or a deglycosylation agent) were compared with (i) reduced samples, which were not deglycosylated (i.e. treated only with β-mercaptoethanol, and boiled in the presence of SDS); and (ii) reduced and deglycosylated samples (i.e. treated both with β-mercaptoethanol and a deglycosylation agent, and boiled in the presence of SDS). In addition, to confirm the presence of each domain of the hSLAMF6-Fc-NKG2A chimeric protein, the gels were run in triplicates and probed using an anti-SLAMF6 antibody (FIG. 16B, left blot), an anti-Fc antibody (FIG. 16B, center blot), or an anti-NKG2A antibody (FIG. 16B, right blot). Western blot analyses were performed to confirm the presence the two ECD domains and the Fc linker of the hSLAMF6-Fc-NKG2A chimeric protein with antibodies specific to the two ECD domains and the Fc linker (FIG. 16B). As shown in FIG. 16B, the Western blots indicated the presence of a dominant dimer band in the non-reduced lanes (FIG. 16B, lane NR in each blot), which was reduced to a glycosylated monomeric band in the presence of the reducing agent, β-mercaptoethanol (FIG. 16B, lane R in each blot). The chimeric protein ran as a monomer at the predicted molecular weight of about 67 kDa in the presence of both a reducing agent (β-mercaptoethanol) and a deglycosylation agent (FIG. 16B, lane DG in each blot). These results demonstrate the native state and tendency to form a dimer of the hSLAMF6-Fc-NKG2A chimeric protein.

### Example 16: Construction and Characterization of an Illustrative Mouse CD80- and NKG2A-Based Chimeric Protein

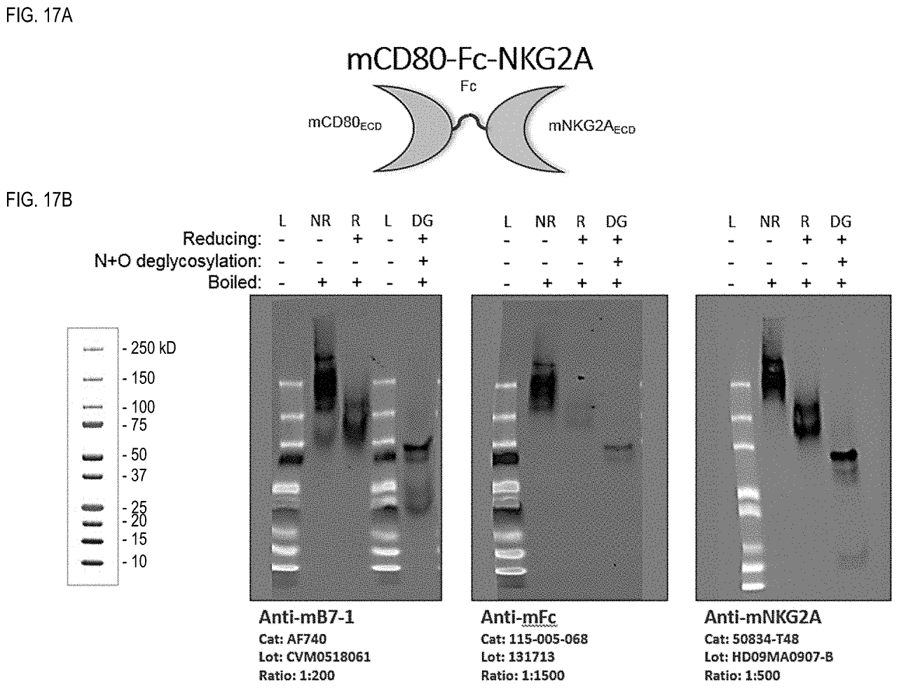

A construct encoding a mouse CD80- and NKG2A-based chimeric protein was generated. The mCD80-Fc-NKG2A chimeric protein construct included an extracellular domain (ECD) of mouse CD80 fused to an extracellular domain (ECD) of mouse NKG2A via a hinge-CH2-CH3 Fc domain derived from IgG1. See, FIG. 17A.

The construct was expressed and purified as described in Example 1.

To understand the native structure of the mCD80-Fc-NKG2A chimeric protein, untreated denatured samples (i.e., boiled in the presence of SDS, without a treatment with a reducing agent or a deglycosylation agent) were compared with (i) reduced samples, which were not deglycosylated (i.e. treated only with 3-mercaptoethanol, and boiled in the presence of SDS); and (ii) reduced and deglycosylated samples (i.e. treated both with β-mercaptoethanol and a deglycosylation agent, and boiled in the presence of SDS). In addition, to confirm the presence of each domain of the mCD80-Fc-NKG2A chimeric protein, the gels were run in triplicates and probed using an anti-CD80 antibody (FIG. 17B, left blot), an anti-Fc antibody (FIG. 17B, center blot), or an anti-NKG2A antibody (FIG. 17B, right blot). Western blot analyses were performed to confirm the presence the two ECD domains and the Fc linker of the mCD80-Fc-NKG2A chimeric protein with antibodies specific to the two ECD domains and the Fc linker (FIG. 17B). As shown in FIG. 17B, the Western blots indicated the presence of a dominant dimer band in the non-reduced lanes (FIG. 17B, lane NR in each blot), which was reduced to a glycosylated monomeric band in the presence of the reducing agent, 3-mercaptoethanol (FIG. 17B, lane R in each blot). The chimeric protein ran as a monomer at the predicted molecular weight of about 67 kDa in the presence of both a reducing agent (3-mercaptoethanol) and a deglycosylation agent (FIG. 17B, lane DG in each blot). These results demonstrate the native state and tendency to form a dimer of the mCD80-Fc-NKG2A chimeric protein.

### Example 17: Construction and Characterization of an Illustrative Mouse CD86- and NKG2A-Based Chimeric Protein

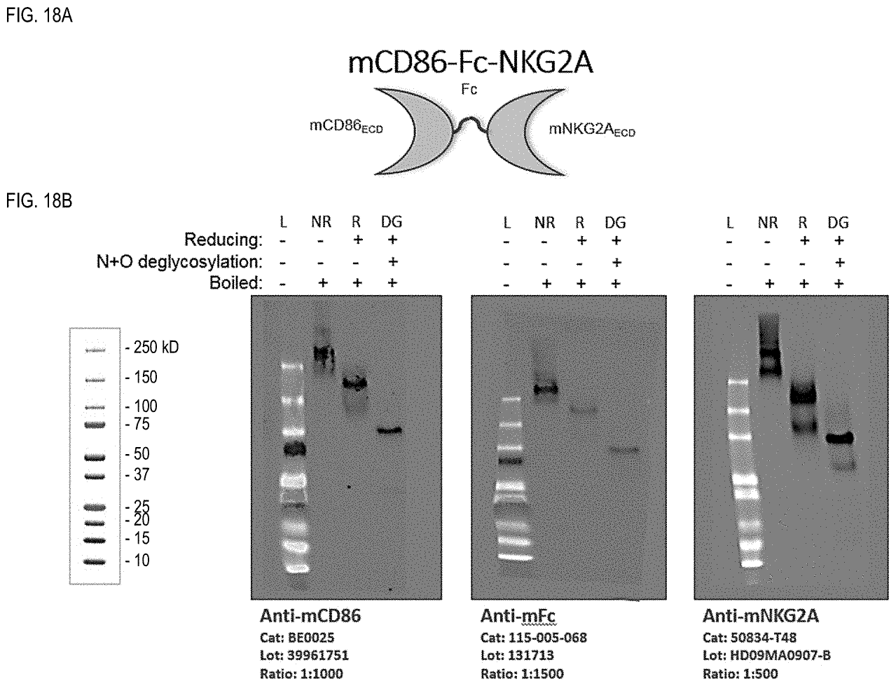

A construct encoding a mouse CD86- and NKG2A-based chimeric protein was generated. The mCD86-Fc-NKG2A chimeric protein construct included an extracellular domain (ECD) of mouse CD86 fused to an extracellular domain (ECD) of Mouse NKG2A via a hinge-CH2-CH3 Fc domain derived from IgG1. See, FIG. 18A.

The construct was expressed and purified as described in Example 1.

To understand the native structure of the mCD86-Fc-NKG2A chimeric protein, untreated denatured samples (i.e., boiled in the presence of SDS, without a treatment with a reducing agent or a deglycosylation agent) were compared with (i) reduced samples, which were not deglycosylated (i.e. treated only with 3-mercaptoethanol, and boiled in the presence of SDS); and (ii) reduced and deglycosylated samples (i.e. treated both with 3-mercaptoethanol and a deglycosylation agent, and boiled in the presence of SDS). In addition, to confirm the presence of each domain of the mCD86-Fc-NKG2A chimeric protein, the gels were run in triplicates and probed using an anti-CD86 antibody (FIG. 18B, left blot), an anti-Fc antibody (FIG. 18B, center blot), or an anti-NKG2A antibody (FIG. 18B, right blot). Western blot analyses were performed to confirm the presence the two ECD domains and the Fc linker of the mCD86-Fc-NKG2A chimeric protein with antibodies specific to the two ECD domains and the Fc linker (FIG. 18B). As shown in FIG. 18B, the Western blots indicated the presence of a dominant dimer band in the non-reduced lanes (FIG. 18B, lane NR in each blot), which was reduced to a glycosylated monomeric band in the presence of the reducing agent, β-mercaptoethanol (FIG. 18B, lane R in each blot). The chimeric protein ran as a monomer at the predicted molecular weight of about 67 kDa in the presence of both a reducing agent (β-mercaptoethanol) and a deglycosylation agent (FIG. 18B, lane DG in each blot). These results demonstrate the native state and tendency to form a dimer of the mCD86-Fc-NKG2A chimeric protein.

### Example 18: Construction and Characterization of an Illustrative Mouse PD-1- and NKG2A-Based Chimeric Protein

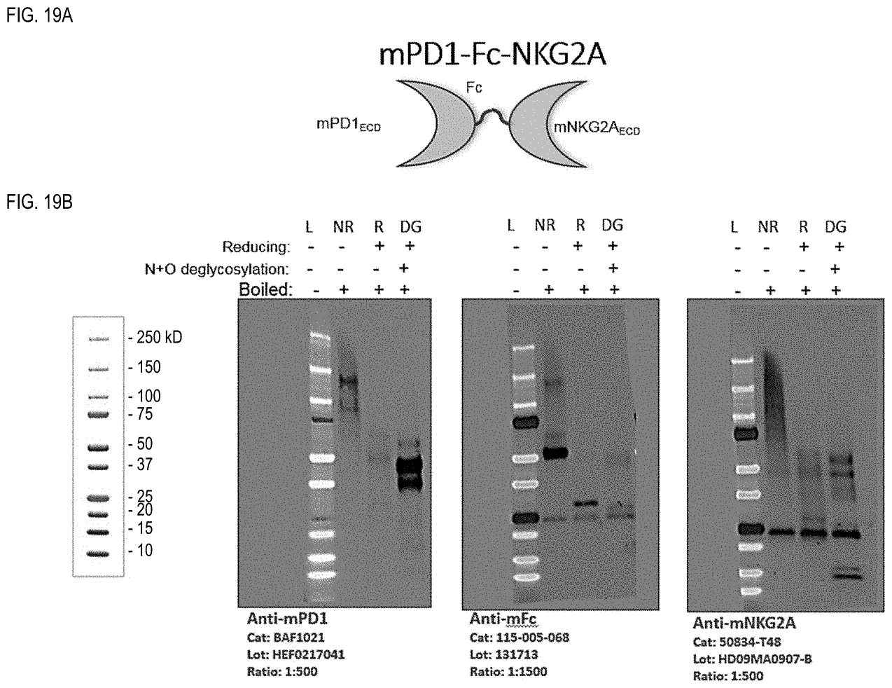

A construct encoding a mouse PD-1- and NKG2A-based chimeric protein was generated. The mPD-1-Fc-NKG2A chimeric protein construct included an extracellular domain (ECD) of mouse PD-1 fused to an extracellular domain (ECD) of Mouse NKG2A via a hinge-CH2-CH3 Fc domain derived from IgG1. See, FIG. 19A.

The construct was expressed and purified as described in Example 1.

To understand the native structure of the mPD-1-Fc-NKG2A chimeric protein, untreated denatured samples (i.e., boiled in the presence of SDS, without a treatment with a reducing agent or a deglycosylation agent) were compared with (i) reduced samples, which were not deglycosylated (i.e. treated only with β-mercaptoethanol, and boiled in the presence of SDS); and (ii) reduced and deglycosylated samples (i.e. treated both with β-mercaptoethanol and a deglycosylation agent, and boiled in the presence of SDS). In addition, to confirm the presence of each domain of the mPD-1-Fc-NKG2A chimeric protein, the gels were run in triplicates and probed using an anti-PD-1 antibody (FIG. 19B, left blot), an anti-Fc antibody (FIG. 19B, center blot), or an anti-NKG2A antibody (FIG. 19B, right blot). Western blot analyses were performed to confirm the presence the two ECD domains and the Fc linker of the mPD-1-Fc-NKG2A chimeric protein with antibodies specific to the two ECD domains and the Fc linker (FIG. 19B). As shown in FIG. 19B, the Western blots indicated the presence of a dominant dimer band in the non-reduced lanes (FIG. 19B, lane NR in each blot), which was reduced to a glycosylated monomeric band in the presence of the reducing agent, β-mercaptoethanol (FIG. 19B, lane R in each blot). The chimeric protein ran as a monomer at the predicted molecular weight of about 67 kDa in the presence of both a reducing agent (β-mercaptoethanol) and a deglycosylation agent (FIG. 19B, lane DG in each blot). These results demonstrate the native state and tendency to form a dimer of the mPD-1-Fc-NKG2A chimeric protein.

### Example 19: Construction and Characterization of an Illustrative SIRPα- and NKG2A-Based Chimeric Protein

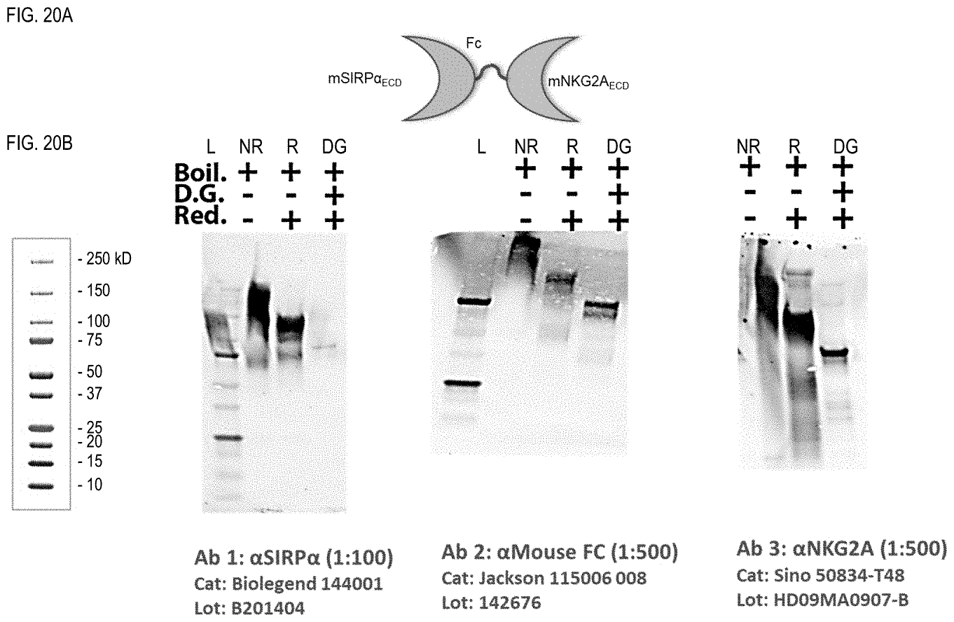

A construct encoding a mouse SIRPα- and NKG2A-based chimeric protein was generated. The SIRPα-Fc-NKG2A chimeric protein construct included an extracellular domain (ECD) of SIRPα fused to an extracellular domain (ECD) of Mouse NKG2A via a hinge-CH2-CH3 Fc domain derived from IgG1. See, FIG. 20A.

The construct was expressed and purified as described in Example 1.

To understand the native structure of the SIRPα-Fc-NKG2A chimeric protein, untreated denatured samples (i.e., boiled in the presence of SDS, without a treatment with a reducing agent or a deglycosylation agent) were compared with (i) reduced samples, which were not deglycosylated (i.e. treated only with 3-mercaptoethanol, and boiled in the presence of SDS); and (ii) reduced and deglycosylated samples (i.e. treated both with 3-mercaptoethanol and a deglycosylation agent, and boiled in the presence of SDS). In addition, to confirm the presence of each domain of the SIRPα-Fc-NKG2A chimeric protein, the gels were run in triplicates and probed using an anti-SIRPα antibody (FIG. 20B, left blot), an anti-Fc antibody (FIG. 20B, center blot), or an anti-NKG2A antibody (FIG. 20B, right blot). Western blot analyses were performed to confirm the presence the two ECD domains and the Fc linker of the SIRPα-Fc-NKG2A chimeric protein with antibodies specific to the two ECD domains and the Fc linker (FIG. 20B). As shown in FIG. 20B, the Western blots indicated the presence of a dominant dimer band in the non-reduced lanes (FIG. 20B, lane NR in each blot), which was reduced to a glycosylated monomeric band in the presence of the reducing agent, 3-mercaptoethanol (FIG. 20B, lane R in each blot). The chimeric protein ran as a monomer at the predicted molecular weight of about 67 kDa in the presence of both a reducing agent (3-mercaptoethanol) and a deglycosylation agent (FIG. 20B, lane DG in each blot). These results demonstrate the native state and tendency to form a dimer of the SIRPα-Fc-NKG2A chimeric protein.

### Example 20: Construction and Characterization of an Illustrative TGFBR2- and NKG2A-Based Chimeric Protein

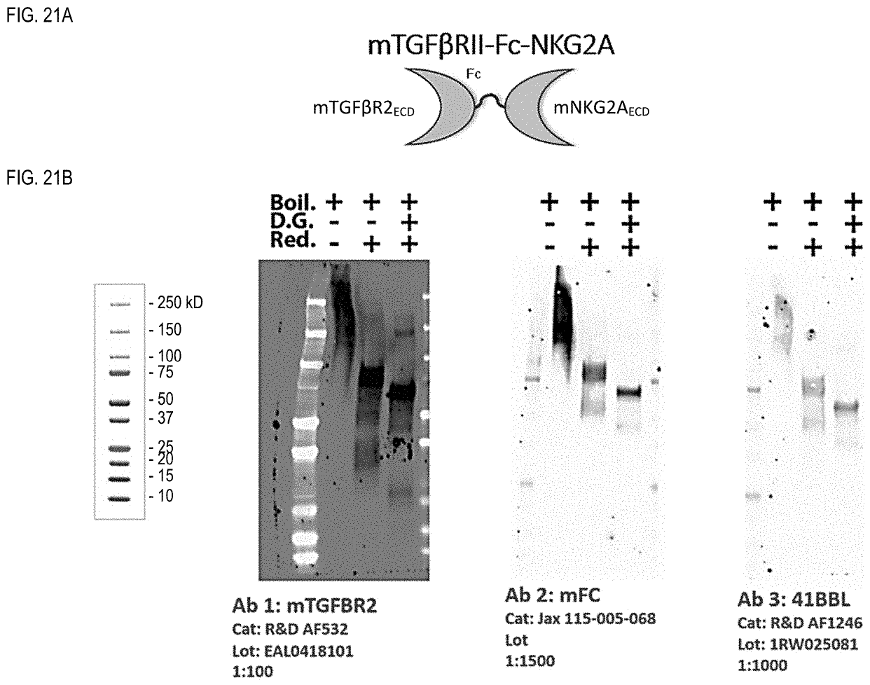

A construct encoding a mouse TGFBR2- and NKG2A-based chimeric protein was generated. The TGFBR2-Fc-NKG2A chimeric protein construct included an extracellular domain (ECD) of mouse TGFBR2 fused to an extracellular domain (ECD) of Mouse NKG2A via a hinge-CH2-CH3 Fc domain derived from IgG1. See, FIG. 21A.

The construct was expressed and purified as described in Example 1.

To understand the native structure of the mTGFBR2-Fc-NKG2A chimeric protein, untreated denatured samples (i.e., boiled in the presence of SDS, without a treatment with a reducing agent or a deglycosylation agent) were compared with (i) reduced samples, which were not deglycosylated (i.e. treated only with 3-mercaptoethanol, and boiled in the presence of SDS); and (ii) reduced and deglycosylated samples (i.e. treated both with 3-mercaptoethanol and a deglycosylation agent, and boiled in the presence of SDS). In addition, to confirm the presence of each domain of the TGFBR2-Fc-NKG2A chimeric protein, the gels were run in triplicates and probed using an anti-TGFBR2 antibody (FIG. 21B, left blot), an anti-Fc antibody (FIG. 21B, center blot), or an anti-NKG2A antibody (FIG. 21B, right blot). Western blot analyses were performed to confirm the presence the two ECD domains and the Fc linker of the TGFBR2-Fc-NKG2A chimeric protein with antibodies specific to the two ECD domains and the Fc linker (FIG. 21B). As shown in FIG. 21B, the Western blots indicated the presence of a dominant dimer band in the non-reduced lanes (FIG. 21B, lane NR in each blot), which was reduced to a glycosylated monomeric band in the presence of the reducing agent, β-mercaptoethanol (FIG. 21B, lane R in each blot). The chimeric protein ran as a monomer at the predicted molecular weight of about 67 kDa in the presence of both a reducing agent (β-mercaptoethanol) and a deglycosylation agent (FIG. 21B, lane DG in each blot). These results demonstrate the native state and tendency to form a dimer of the TGFBR2-Fc-NKG2A chimeric protein.

### Example 21: Detection of Mouse Fc in the Illustrative NKG2A-Based Chimeric Proteins Using ELISA

To understand whether the Fc linkers from the native chimeric proteins can be accessed by anti-mouse Fc antibodies, ELISA-based assays were performed. For these assays, an anti-mFc IgG antibody was coated on plates and increasing amounts of the mPD-1-Fc-NKG2A and mSLAMF6-Fc-NKG2a chimeric proteins were added to the plates for capture by the plate-bound anti-mFc IgG antibody. The binding of the mPD-1-Fc-NKG2A and mSLAMF6-Fc-NKG2a chimeric proteins bound to the anti-mFc IgG antibody were detected using an anti-mFc HRP. An IgG antibody mouse Fc was used to generate a standard curve. mFc IgG was used as a positive control.

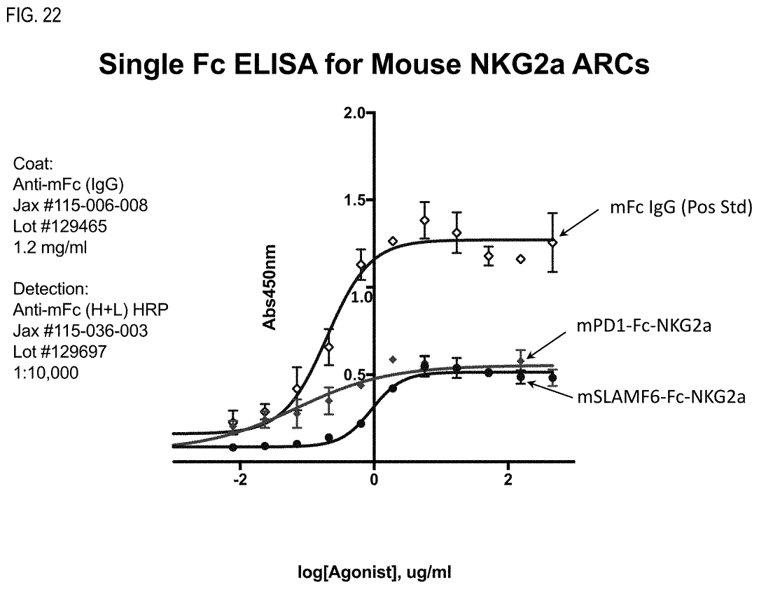

As shown in FIG. 22, the Fc domain of the mSLAMF6-Fc-NKG2A and mPD-1-Fc-NKG2A chimeric proteins bound to the plate-bound anti-mFc IgG antibody in a dose-dependent manner. These results demonstrate the presence of a mouse Fc domain and its accessibility to anti-mFc IgG antibodies.

### Example 22: Detection of Human Fc in the Illustrative NKG2A-Based Chimeric Proteins Using ELISA

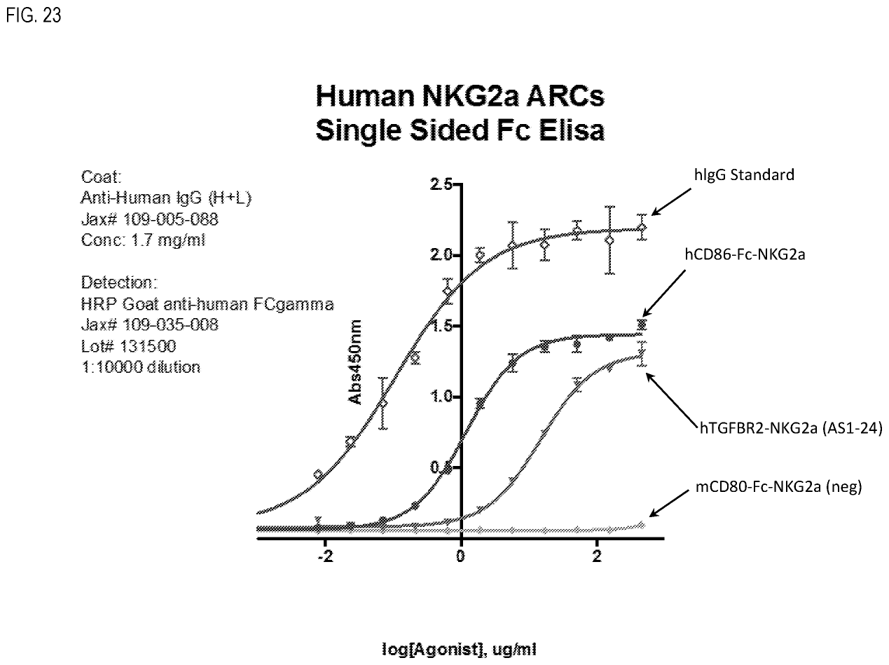

To understand whether the Fc linkers from the native chimeric proteins can be accessed by anti-human antibodies, ELISA-based assays were performed. For these assays, an anti-human IgG antibody was coated on plates and increasing amounts of the hCD86-Fc-NKG2A and hTGFBR2-Fc-NKG2A, mCD80-Fc-NKG2a chimeric proteins, and a human IgG antibody were added to the plates for capture by the plate-bound anti-human antibody. The human IgG antibody was used as a positive control and to generate a standard curve. The mCD80-Fc-NKG2a chimeric protein was included as a negative control as containing a non-human Fc domain. The protein bound to the anti-human IgG antibody was detected using an anti-human Fcgamma HRP. As shown in FIG. 23, the hCD86-Fc-NKG2A and hTGFBR2-Fc-NKG2A chimeric proteins bound to the plate-bound anti-human IgG antibody in a dose-dependent manner. In contrast, the mCD80-Fc-NKG2a chimeric protein did not bind the plate-bound anti-human IgG antibody. These results demonstrate the presence of a human Fc domain and its accessibility to anti-human IgG antibodies.

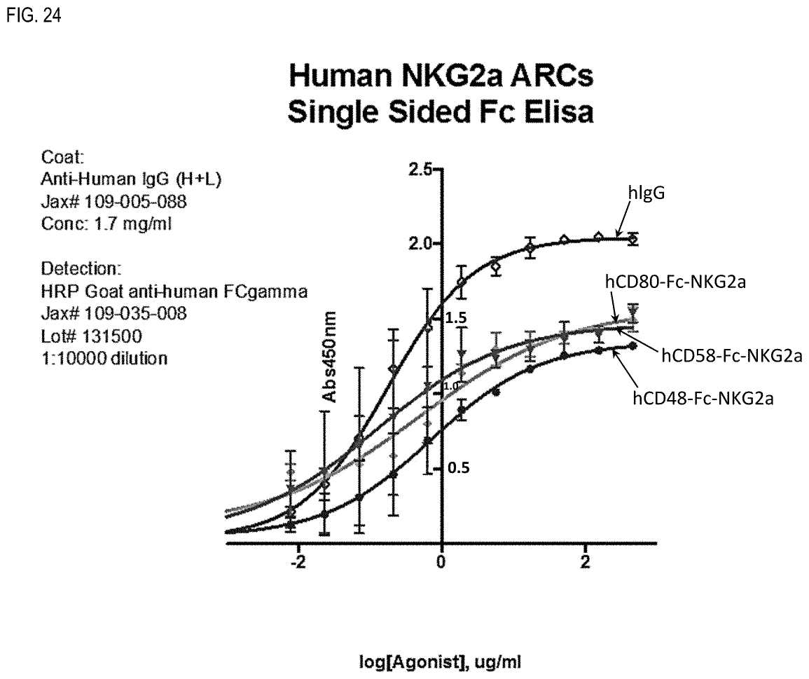

In another set of experiments, an anti-human IgG antibody was coated on plates and increasing amounts of the hCD48-Fc-NKG2A, hCD58-Fc-NKG2A, and hCD80-Fc-NKG2A chimeric proteins, and a human IgG antibody were added to the plates for capture by the plate-bound anti-mFc IgG antibody. The human IgG antibody was used as a positive control and to generate a standard curve. The binding to the anti-human IgG antibody was detected using an anti-human Fcgamma HRP. As shown in FIG. 24, each of the hCD48-Fc-NKG2A, hCD58-Fc-NKG2A, and hCD80-Fc-NKG2A chimeric proteins bound to the plate-bound anti-human IgG antibody in a dose-dependent manner. These results demonstrate the presence of a human Fc domain and its accessibility to anti-human IgG antibodies.

### Example 23: The Ability of the NKG2A Part of the Chimeric Proteins of the Present Disclosure to Bind To Natural Ligand HLA-E

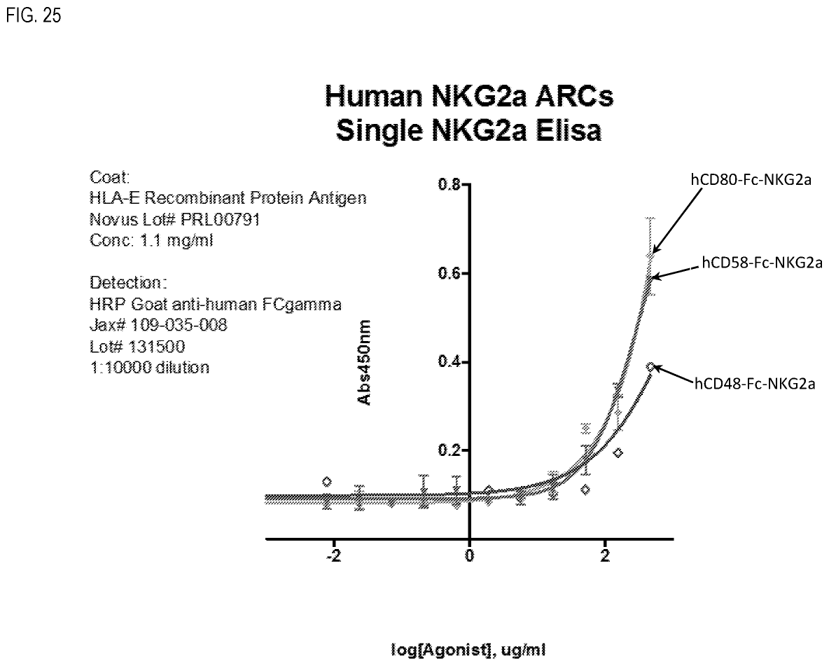

The CD94/NK group 2 member A (NKG2A) heterodimer binds to a nonclassical minimally polymorphic HLA class I molecule (HLA-E), which presents peptides derived from other HLA class I molecules. To understand whether the NKG2A part of the chimeric proteins disclosed herein can bind HLA-E, ELISA-based assays were performed. Briefly, recombinant HLA-E protein was coated on plates and increasing amounts of the hCD48-Fc-NKG2A, hCD58-Fc-NKG2A, and hCD80-Fc-NKG2A chimeric proteins were added to the plates for capture by the plate-bound recombinant HLA-E protein. The chimeric proteins bound to the recombinant HLA-E protein was detected using an anti-human Fcgamma HRP. As shown in FIG. 25, each of the hCD48-Fc-NKG2A, hCD58-Fc-NKG2A, and hCD80-Fc-NKG2A chimeric proteins bound to the plate-bound recombinant HLA-E protein in a dose-dependent manner. These results demonstrate that the hCD48-Fc-NKG2A, hCD58-Fc-NKG2A, and hCD80-Fc-NKG2A chimeric proteins disclosed herein specifically bind the HLA-E protein.

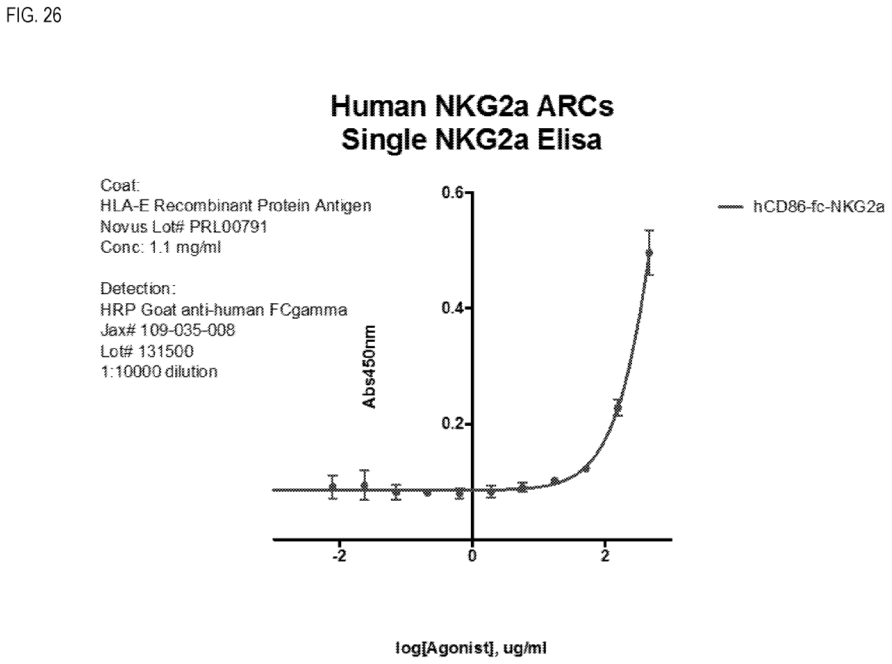

A similar experiment was performed to determine the binding of the hCD80-Fc-NKG2A chimeric protein to the plate-bound HLA-E protein. As shown in FIG. 26, the hCD80-Fc-NKG2A chimeric protein also bound to the plate-bound recombinant HLA-E protein in a dose-dependent manner. These results demonstrate that the hCD80-Fc-NKG2A chimeric protein disclosed herein specifically bind the HLA-E protein.

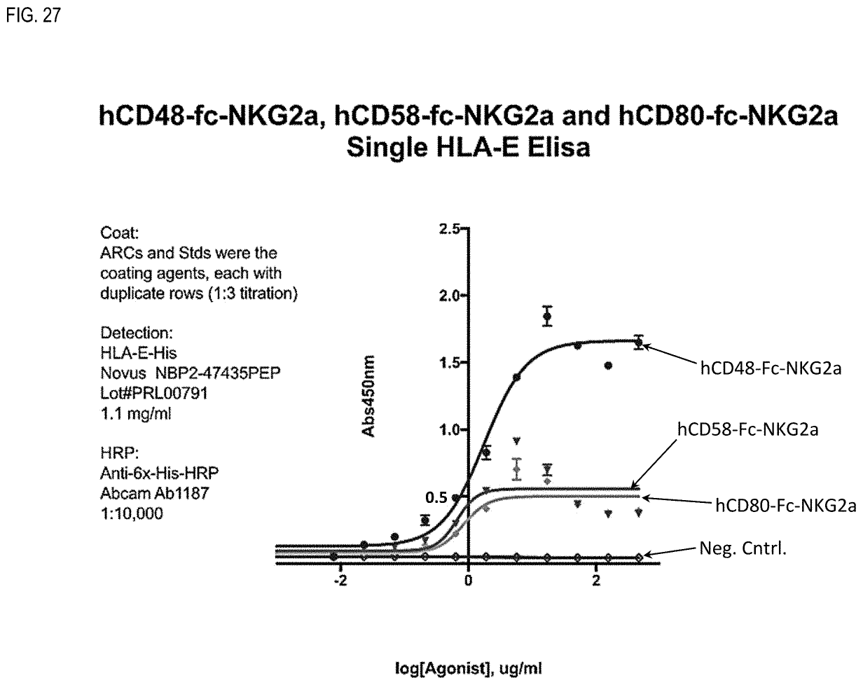

In a complementary experiment, increasing amounts of the hCD48-Fc-NKG2A, hCD58-Fc-NKG2A, and hCD80-Fc-NKG2A chimeric proteins or a negative control protein was coated on plates. Recombinant HLA-E-His protein was added to the plates for capture by the plate-bound chimeric proteins or the negative control protein. The captured HLA-E-His protein was detected using an anti-6X-His-HRP antibody. As shown in FIG. 27, a dose-dependent binding of the hCD48-Fc-NKG2A, hCD58-Fc-NKG2A, and hCD80-Fc-NKG2A chimeric proteins to HLA-E was observed.

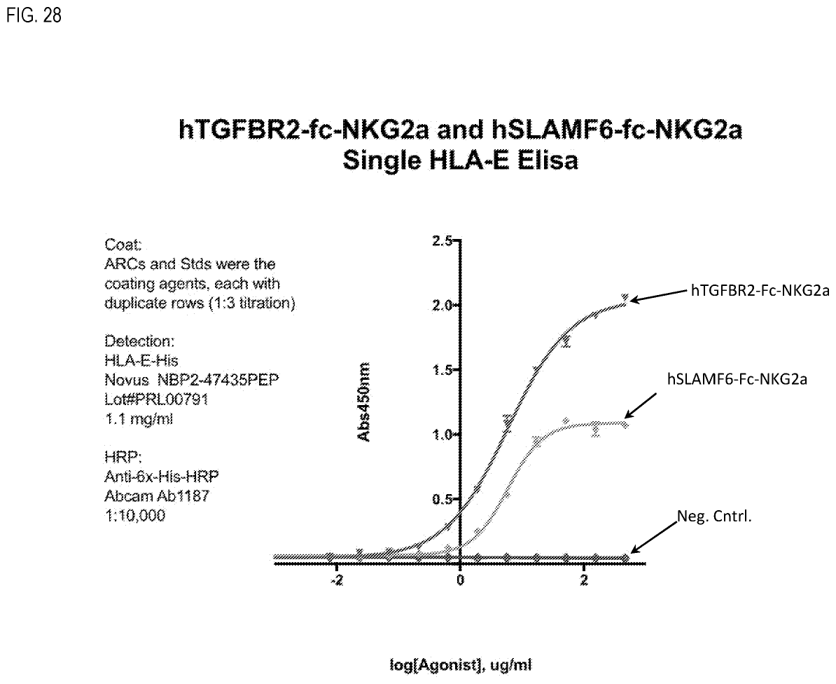

Similarly, increasing amounts of the hTGFBR2-Fc-NKG2A and hSLAMF6-Fc-NKG2A chimeric proteins chimeric proteins or a negative control protein was coated on plates. Recombinant HLA-E-His protein was added to the plates for capture by the plate-bound chimeric proteins or the negative control protein. The captured HLA-E-His protein was detected using an anti-6X-His-HRP antibody. As shown in FIG. 28, a dose-dependent binding of the hTGFBR2-Fc-NKG2A and hSLAMF6-Fc-NKG2A chimeric proteins to HLA-E was observed.

These results demonstrate that NKG2A part of the chimeric proteins disclosed herein specifically bind their natural ligand.

### Example 24: The Ability of the Type I Transmembrane Protein Parts of the NKG2A-Based Chimeric Proteins to Bind Their Natural Ligands

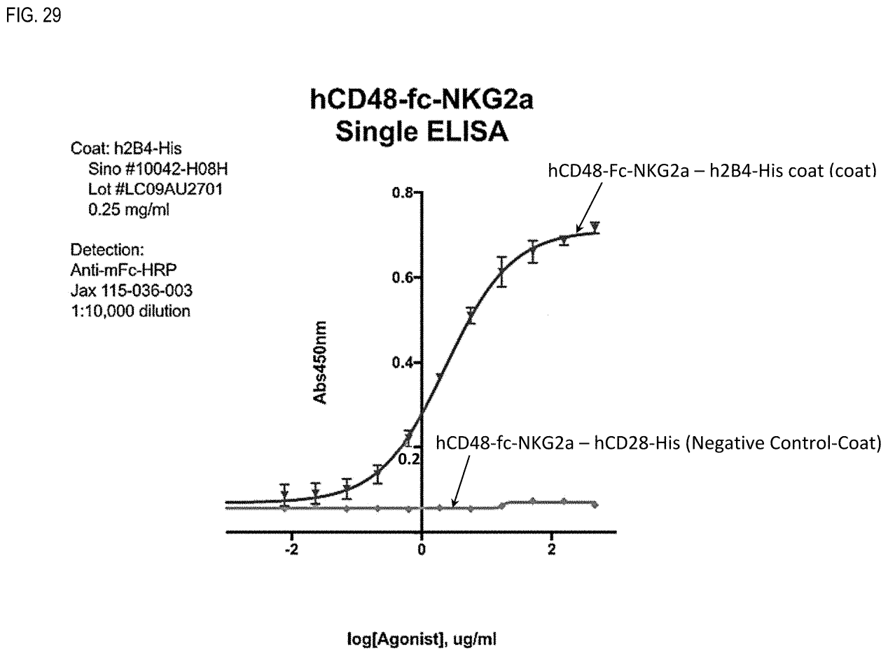

CD48 binds 2B4 (CD244), a member of the signaling lymphocyte-activation molecule (SLAM) family. To understand whether the CD48 part of the hCD48-Fc-NKG2A chimeric protein can bind HLA-E, ELISA-based assays were performed. Briefly, recombinant 2B4-His protein was coated on plates. Human CD28-His protein coated on negative control plates. Increasing amounts of the hCD48-Fc-NKG2A chimeric protein was added to the plates for capture by the plate-bound recombinant 2B4-His protein. The chimeric hCD48-Fc-NKG2A protein bound to the recombinant 2B4-His protein was detected using an anti-human Fcgamma HRP. As shown in FIG. 29, the hCD48-Fc-NKG2A chimeric protein bound to the plate-bound recombinant 2B4-His protein, but not human CD28-His protein, in a dose-dependent manner. These results demonstrate that the hCD48-Fc-NKG2A chimeric protein disclosed herein specifically bind the 2B4 protein.

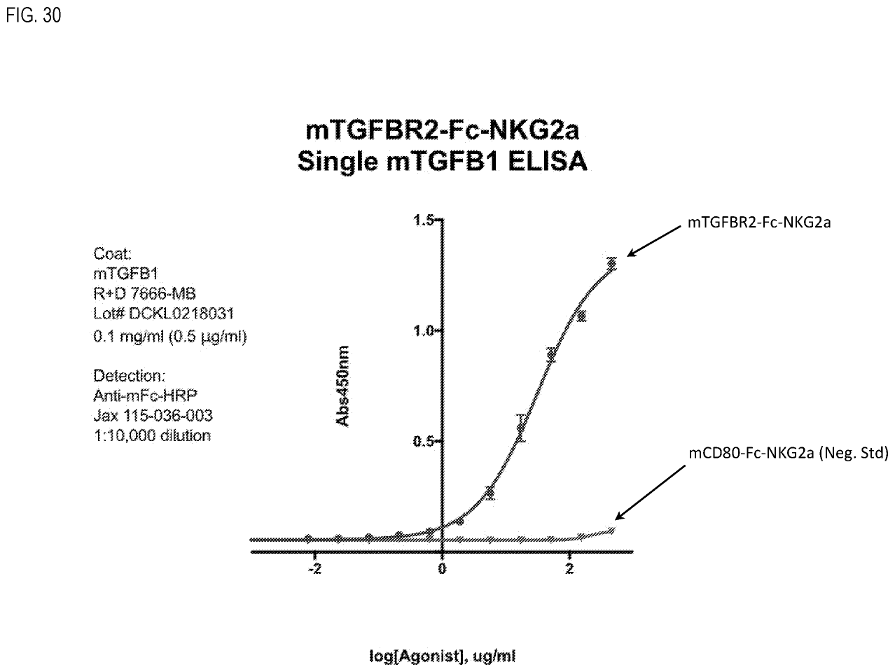

TGFβ1 is a ligand of TGFBR2. To understand whether the TGFBR2 part of the TGFBR2-Fc-NKG2A chimeric protein can bind TGFβ1, ELISA-based assays were performed. Briefly, recombinant TGFβ1 protein was coated on plates and increasing amounts of the TGFBR2-Fc-NKG2A chimeric protein was added to the plates for capture by the plate-bound recombinant TGFβ1 protein. The chimeric protein bound to the recombinant TGFβ1 protein was detected using an anti-mFc HRP. As shown in FIG. 30, each of the TGFBR2-Fc-NKG2A chimeric protein bound to the plate-bound recombinant TGFβ1 protein in a dose-dependent manner. These results demonstrate that the TGFBR2-Fc-NKG2A chimeric protein specifically bind the TGFβ1 protein.

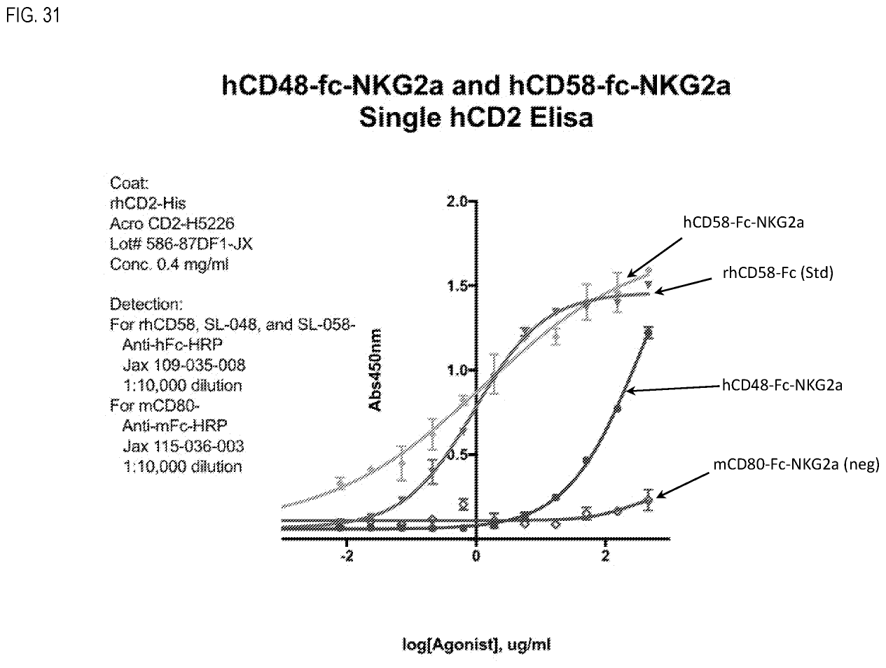

CD2 is a transmembrane protein in T cells that binds to its ligand CD58 on APC in humans and on CD48 in rodents. To understand whether the CD48 and CD58 parts of the hCD48-Fc-NKG2A, and hCD58-Fc-NKG2A chimeric protein can bind CD2, ELISA-based assays were performed. Briefly, recombinant human CD2 protein (rhCD2-His) was coated on plates and increasing amounts of the hCD48-Fc-NKG2A, hCD58-Fc-NKG2A, and mCD80-Fc-NKG2A chimeric proteins and recombinant hCD58-Fc protein was added to the plates for capture by the plate-bound recombinant CD2 protein. The hCD80-Fc-NKG2A chimeric protein served as a negative control and recombinant hCD58-Fc protein served as a positive control. The hCD48-Fc-NKG2A and hCD58-Fc-NKG2A, chimeric proteins and recombinant hCD58-Fc protein bound to the recombinant CD2 protein was detected using an anti-human Fc HRP. The mCD80-Fc-NKG2A chimeric protein bound to the recombinant CD2 protein was detected using an anti-mouse Fc HRP. As shown in FIG. 31, the hCD48-Fc-NKG2A and hCD58-Fc-NKG2A, chimeric proteins bound to the plate-bound recombinant CD2 protein in a dose-dependent manner. Further, the hCD58-Fc-NKG2A and hCD58-Fc exhibited similar kinetics of binding to the plate-bound recombinant CD2 protein. On the other hand, the mCD80-Fc-NKG2A protein did not bind CD2. These results demonstrate that the hCD48-Fc-NKG2A and hCD58-Fc-NKG2A chimeric proteins disclosed herein specifically bind the CD2 protein.

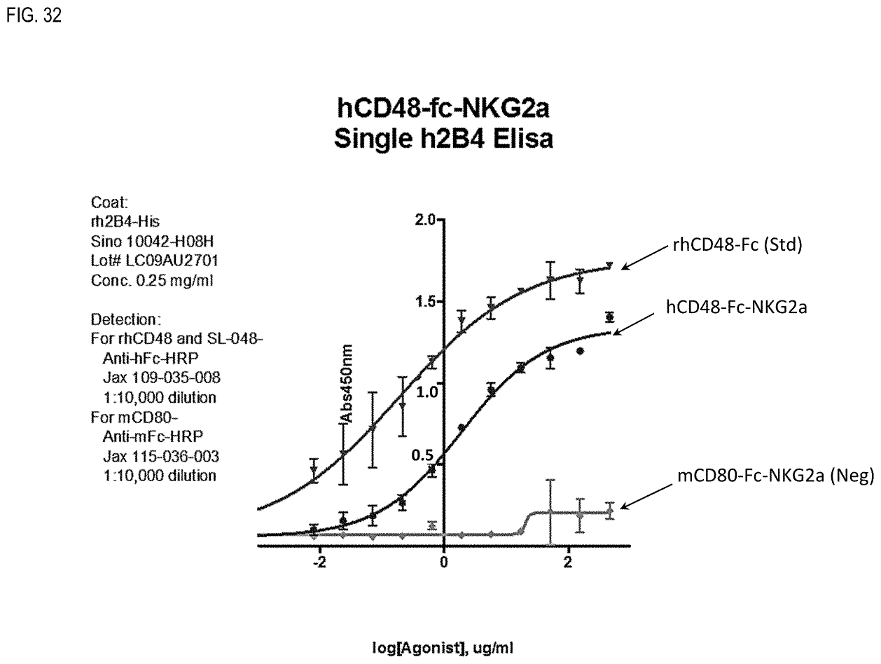

CD48 is the ligand for 2B4. To understand whether the CD48 parts of the hCD48-Fc-NKG2A chimeric protein can bind 2B4, ELISA-based assays were performed. Briefly, recombinant human 2B4 protein (rh2B4-His) was coated on plates and increasing amounts of the hCD48-Fc-NKG2A and mCD80-Fc-NKG2A chimeric proteins and recombinant human CD48-Fc (rhCD48-Fc) protein was added to the plates for capture by the plate-bound recombinant 2B4 protein. The hCD48-Fc-NKG2A chimeric protein and recombinant human CD48-Fc (rhCD48-Fc) protein bound to the recombinant 2B4 protein was detected using an anti-human Fc HRP. The mCD80-Fc-NKG2A chimeric protein, which was used as a negative control, bound to the recombinant 2B4 protein was detected using an anti-mouse Fc HRP. As shown in FIG. 32, the hCD48-Fc-NKG2A, chimeric protein bound to the plate-bound recombinant 2B4 protein in a dose-dependent manner, with a kinetics comparable to that of rhCD48-Fc protein. These results demonstrate that the hCD48-Fc-NKG2A chimeric protein specifically bind the 2B4 protein.

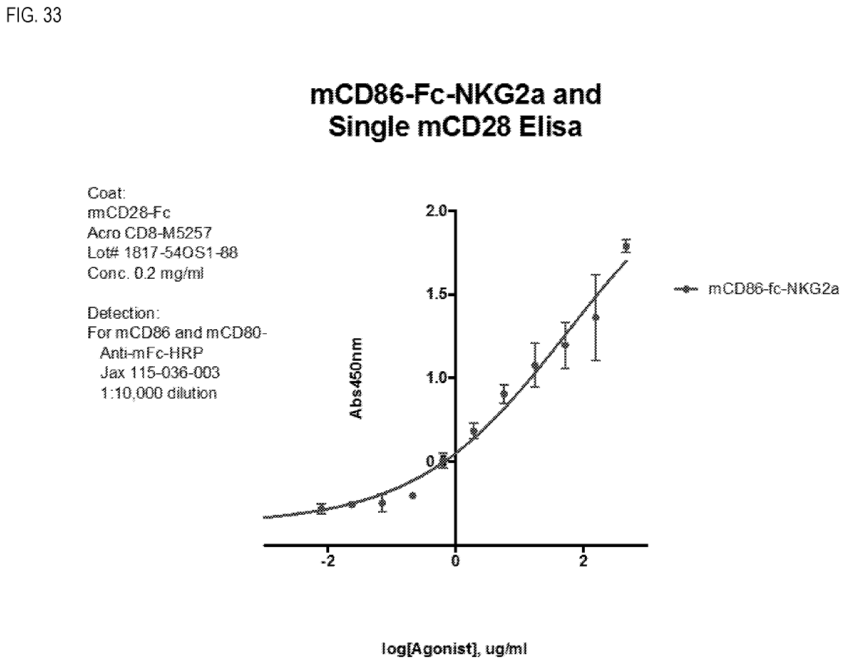

CD28 is stimulated by CD80 and CD86 ligands on cells such as APCs, T cells and NK cells. To understand whether the CD80 part of the mCD80-Fc-NKG2A chimeric protein can bind mouse CD28, ELISA-based assays were performed. Briefly, recombinant mouse CD28 (rmCD29) protein was coated on plates and increasing amounts of the mCD80-Fc-NKG2A chimeric protein was added to the plates for capture by the plate-bound recombinant CD28 protein. The chimeric protein bound to the recombinant CD28 protein was detected using an anti-mouse Fc HRP. As shown in FIG. 33, the mCD80-Fc-NKG2A chimeric protein bound to the plate-bound recombinant CD28 protein in a dose-dependent manner. These results demonstrate that the mCD80-Fc-NKG2A chimeric protein specifically bind CD28.

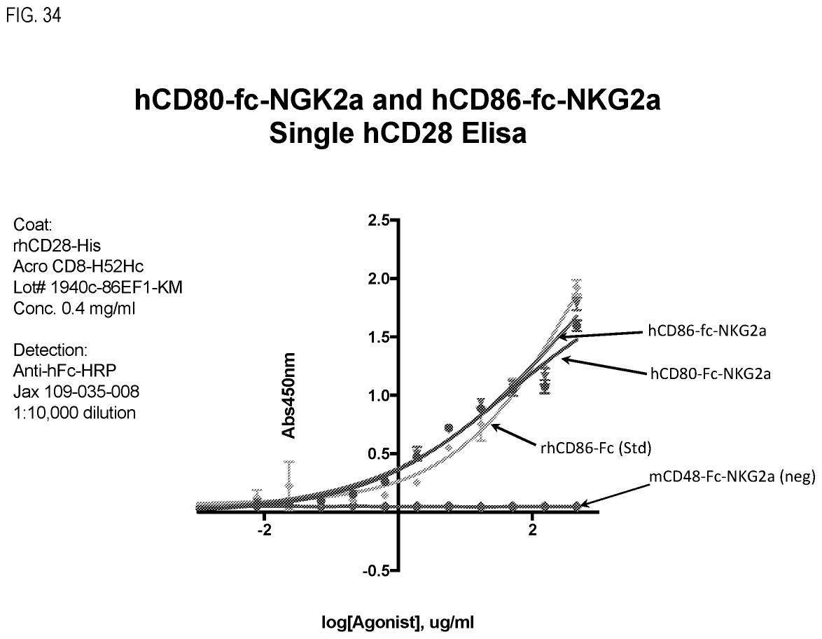

To understand whether the CD80 and CD86 parts of the hCD80-Fc-NKG2A and hCD86-Fc-NKG2A chimeric protein can bind CD28, ELISA-based assays were performed. Briefly, recombinant CD28 protein was coated on plates and increasing amounts of the hCD80-Fc-NKG2A, hCD86-Fc-NKG2A, and mCD48-Fc-NKG2A chimeric proteins, and recombinant human CD86-Fc (rhCD86-Fc) protein was added to the plates for capture by the plate-bound recombinant CD28 protein. The mCD48-Fc-NKG2A chimeric protein, and recombinant human CDrhCD86-Fc protein was used as negative and positive controls, respectively. The chimeric proteins bound to the recombinant CD28 protein was detected using an anti-human Fc HRP. As shown in FIG. 34, the hCD80-Fc-NKG2A and hCD86-Fc-NKG2A chimeric proteins bound to the plate-bound recombinant CD28 protein in a dose-dependent manner, with a binding kinetics comparable to that of the rhCD86-Fc protein. In contrast, the mCD48-Fc-NKG2A chimeric protein did not bind CD28. These results demonstrate that the hCD80-Fc-NKG2A and hCD86-Fc-NKG2A chimeric proteins specifically bind CD28.

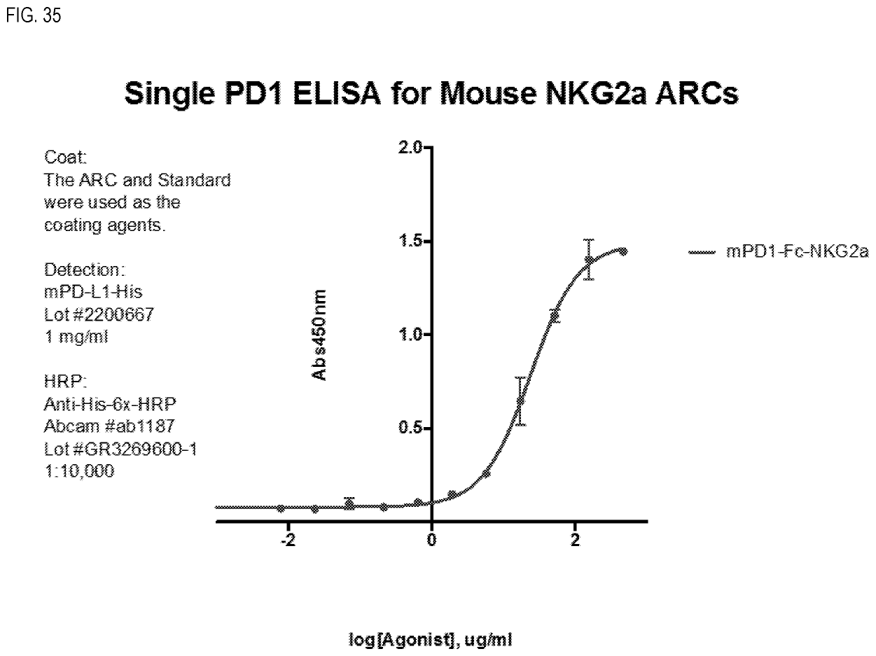

Programmed death-ligand 1 (PD-L1) is a PD-1 ligand. To understand whether the PD-1 parts of the PD-1-Fc-NKG2A chimeric protein can bind PD-L1, ELISA-based assays were performed. Briefly, recombinant PD-L1 protein (mPD-L1-His) was coated on plates and increasing amounts of the PD-1-Fc-NKG2A chimeric protein was added to the plates for capture by the plate-bound recombinant PD-L1 protein. The chimeric protein bound to the recombinant PD-L1 protein was detected using an anti-His-6X HRP. As shown in FIG. 35, each of the PD-1-Fc-NKG2A chimeric protein bound to the plate-bound recombinant PD-L1 protein in a dose-dependent manner. These results demonstrate that the PD-1-Fc-NKG2A chimeric protein disclosed herein specifically bind the PD-L1 protein.

Collectively, these results demonstrate that the type I transmembrane protein part, which is located at or near the N-terminus of the chimeric proteins disclosed herein, efficiently and specifically bind to its natural ligand.

### Example 25: The Ability of the Type I Transmembrane Protein and the NKG2A Part of the Chimeric Proteins Disclosed Herein to Simultaneously Bind Their Ligands

The objective of these experiments was to understand whether the type I transmembrane protein part, which is located at or near the N-terminus of the chimeric proteins, and the NKG2A part, which is located at or near the C-terminus of the chimeric proteins can simulataneously bind their intended targets. To answer these questions, ELISA-based assays were performed.

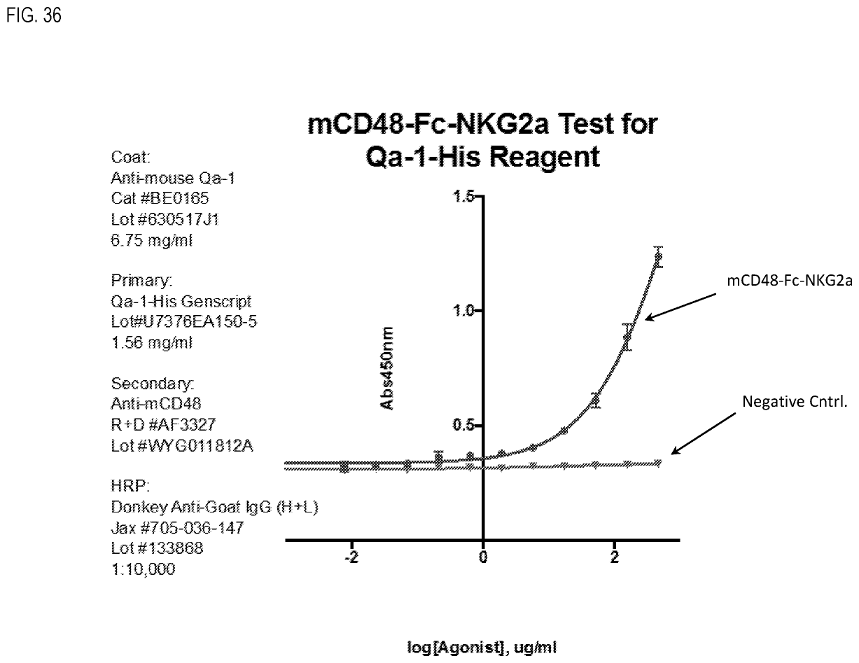

To understand whether the mCD48-Fc-NKG2A chimeric protein can binds simultaneously to both Qa1 and Anti-CD48, the following ELISA experiment was carried out. Briefly, an anti-mouse Qa1 antibody was coated on plates. Recombinant Qa1 protein (Qa1-His) was added for capture by the plate-bound anti-mouse Qa1 antibody. Increasing amounts of the mCD48-Fc-NKG2A chimeric protein was added to the plates for capture by the Qa1-His protein. Further, a goat anti-mCD48 antibody was added to the plate for capture by mCD48-Fc-NKG2A chimeric protein. The binding of the anti-mCD48 antibody was detected using an anti-goat antibody. As shown in FIG. 36, the ELISA assays showed a dose-dependent binding. In contrast, a negative control did not exhibit such binding. Since signal generation requires simultaneous binding of mCD48-Fc-NKG2A to both Qa1 and anti-CD48 antibody, these results demonstrate that mCD48-Fc-NKG2A binds to both Qa1 and Anti-CD48 simultaneously in a dose dependent manner.

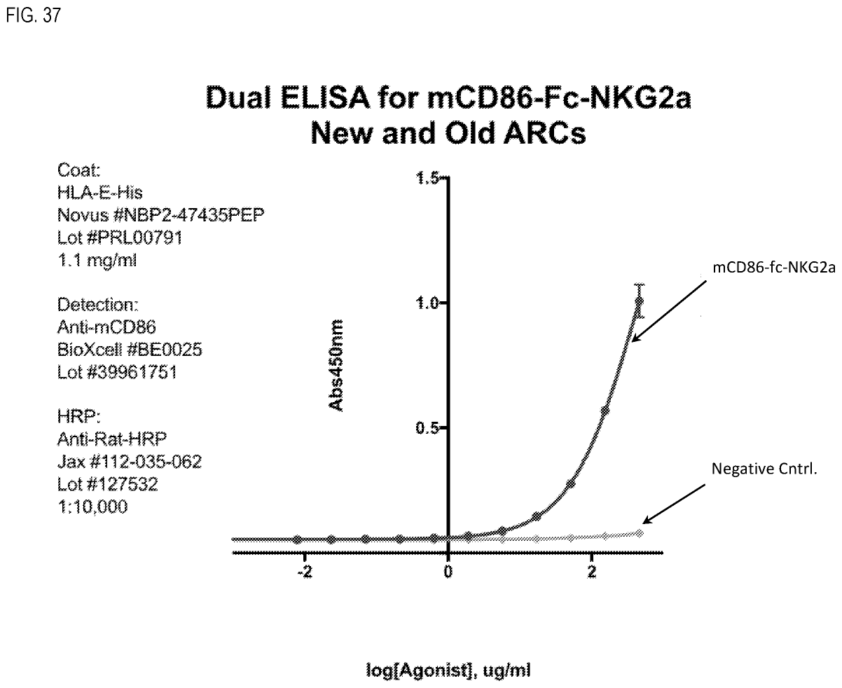

To understand whether the mCD86-Fc-NKG2A chimeric protein can binds simultaneously to both Qa1 and anti-CD86, the following ELISA experiment was carried out. Briefly, recombinant HLA-E (HLA-E-His) protein was coated on plates. Increasing amounts of the mCD86-Fc-NKG2A chimeric protein was added to the plates for capture by the HLA-E-His protein. Further, a rat anti-mCD86 antibody was added to the plate for capture by mCD86-Fc-NKG2A chimeric protein. The binding of the anti-mCD86 antibody was detected using an anti-rat antibody. As shown in FIG. 37, the ELISA assays showed a dose-dependent binding. In contrast, a negative control did not exhibit such binding. Since signal generation requires simultaneous binding of mCD86-Fc-NKG2A to both HLA-E and anti-CD86 antibody, these results demonstrate that mCD86-Fc-NKG2A binds to both HLA-E and Anti-CD86 simultaneously in a dose dependent manner.

To understand whether the mCD86-Fc-NKG2A chimeric protein can binds simultaneously to both HLA-E and anti-CD86, the following ELISA experiment was carried out. Briefly, recombinant HLA-E (HLA-E-His) protein was coated on plates. Increasing amounts of the mCD86-Fc-NKG2A chimeric protein was added to the plates for capture by the HLA-E-His protein. Further, a rat anti-mCD86 antibody was added to the plate for capture by mCD86-Fc-NKG2A chimeric protein. The binding of the anti-mCD86 antibody was detected using an anti-rat antibody. As shown in FIG. 37, the ELISA assays showed a dose-dependent binding. In contrast, a negative control did not exhibit such binding. Since signal generation requires simultaneous binding of mCD86-Fc-NKG2A to both HLA-E and anti-CD86 antibody, these results demonstrate that mCD86-Fc-NKG2A binds to both HLA-E and Anti-CD86 simultaneously in a dose dependent manner.

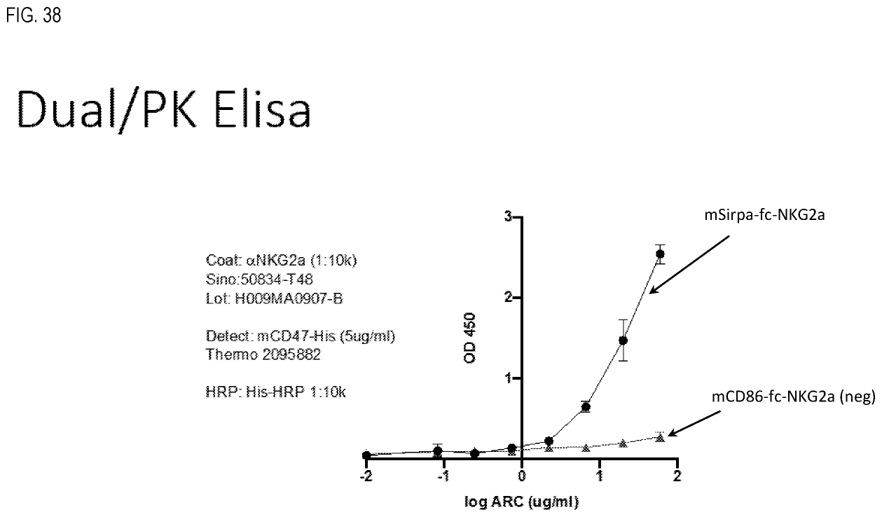

To understand whether the mSIRPα-Fc-NKG2A chimeric protein can binds simultaneously to both anti-NKG2a and CD47, the following ELISA experiment was carried out. Briefly, and anti-NKG2a antibody was coated on plates. Increasing amounts of the mSIRPα-Fc-NKG2A and mCD48-Fc-NKG2A chimeric proteins were added to the plates for capture by the anti-NKG2a antibody. Further, recombinant mCD47 (mCD47-His) protein was added to the plate for capture by mSIRPα-Fc-NKG2A chimeric protein. The binding of the mCD47-His protein was detected using an anti-His HRP antibody. As shown in FIG. 38, the ELISA assays showed a dose-dependent binding by mSIRPα-Fc-NKG2A. In contrast, the mCD48-Fc-NKG2A chimeric protein, which was a negative control, did not exhibit such binding. Since signal generation requires simultaneous binding of mSIRPα-Fc-NKG2A to both anti-NKG2a antibody and mCD47-His protein, these results demonstrate that mSIRPα-Fc-NKG2A binds to both anti-NKG2a antibody and mCD47 simultaneously in a dose dependent manner.

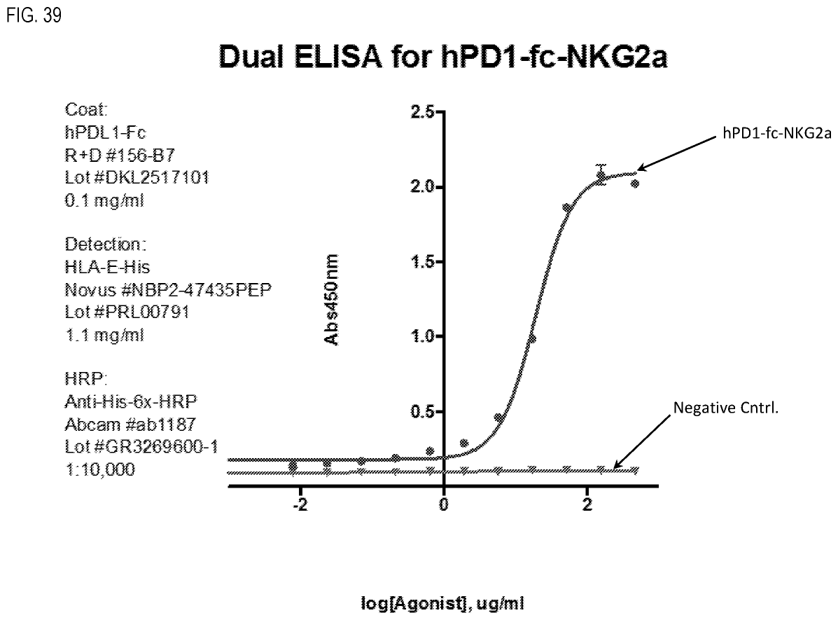

To understand whether the hPD-1-Fc-NKG2A chimeric protein can binds simultaneously to both hPD-L1 and HLA-E, the following ELISA experiment was carried out. Briefly, and recombinant hPD-L1 (hPD-L1-Fc) protein was coated on plates. Increasing amounts of the hPD-1-Fc-NKG2A chimeric protein was added to the plates for capture by the hPD-L1-Fc protein. Further, recombinant HLA-E (HLA-E-His) protein was added to the plate for capture by hPD-1-Fc-NKG2A chimeric protein. The binding of the HLA-E-His protein was detected using an anti-His 6X HRP antibody. As shown in FIG. 39, the ELISA assays showed a dose-dependent binding by hPD-1-Fc-NKG2A. In contrast, a negative control did not exhibit such binding. Since signal generation requires simultaneous binding of hPD-1-Fc-NKG2A to both hPD-L1 protein and HLA-E protein, these results demonstrate that hPD-1-Fc-NKG2A binds to both hPD-L1 protein and HLA-E simultaneously in a dose dependent manner.

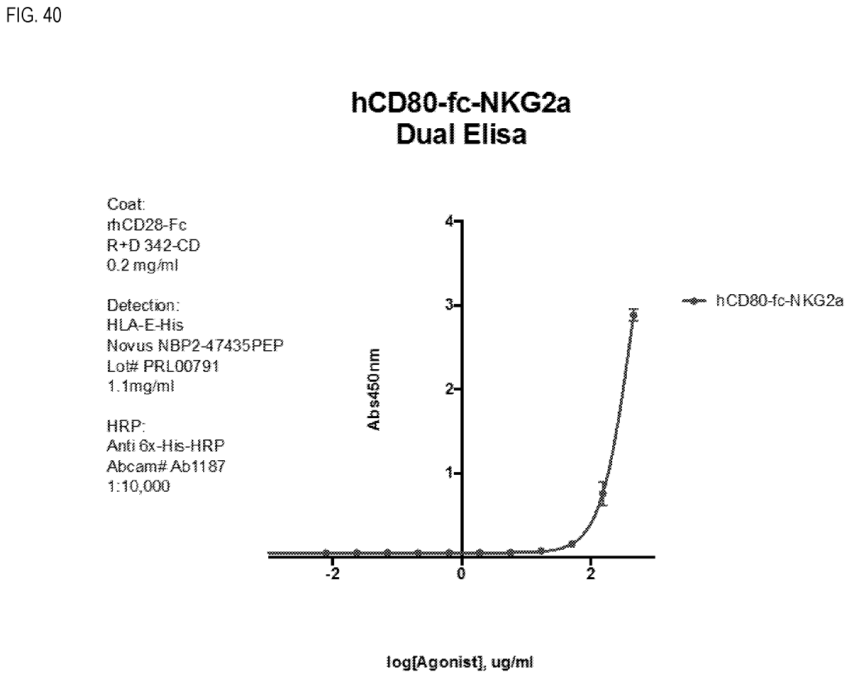

To understand whether the hCD80-Fc-NKG2A chimeric protein can binds simultaneously to both hCD28 and HLA-E, the following ELISA experiment was carried out. Briefly, and recombinant hCD28 (rhCD28-Fc) protein was coated on plates. Increasing amounts of the hCD80-Fc-NKG2A chimeric protein was added to the plates for capture by the recombinant human CD28-Fc (rhCD28-Fc) protein. Further, recombinant HLA-E (HLA-E-His) protein was added to the plate for capture by hCD80-Fc-NKG2A chimeric protein. The binding of the HLA-E-His protein was detected using an anti-His 6X HRP antibody. As shown in FIG. 40, the ELISA assays showed a dose-dependent binding by hCD80-Fc-NKG2A. Since signal generation requires simultaneous binding of hCD80-Fc-NKG2A to both rhCD28 protein and HLA-E protein, these results demonstrate that hCD80-Fc-NKG2A binds to both hCD28 protein and HLA-E simultaneously in a dose dependent manner.

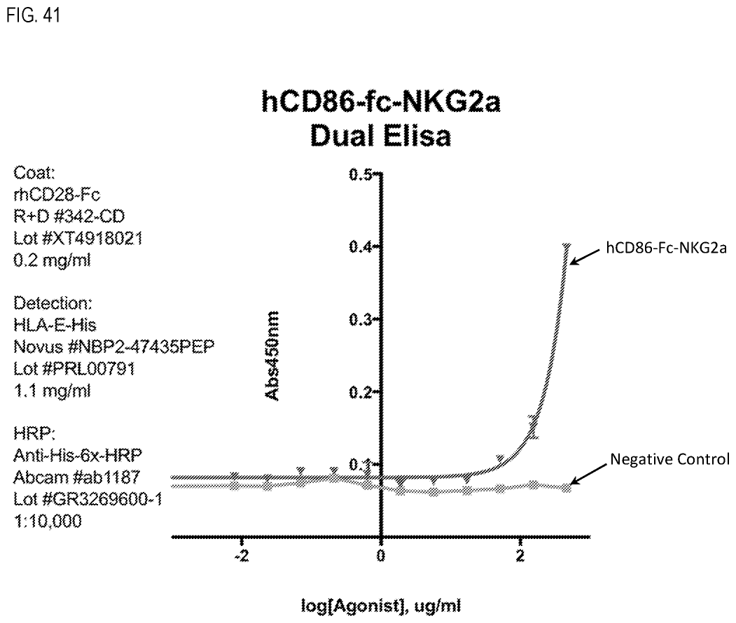

To understand whether the hCD86-Fc-NKG2A chimeric protein can binds simultaneously to both hCD28 and HLA-E, the following ELISA experiment was carried out. Briefly, and recombinant hCD28 (rhCD28-Fc) protein was coated on plates. Increasing amounts of the hCD86-Fc-NKG2A chimeric protein was added to the plates for capture by the rhCD28-Fc protein. Further, recombinant HLA-E (HLA-E-His) protein was added to the plate for capture by hCD86-Fc-NKG2A chimeric protein. The binding of the HLA-E-His protein was detected using an anti-His 6X HRP antibody. As shown in FIG. 41, the ELISA assays showed a dose-dependent binding by hCD86-Fc-NKG2A. In contrast, a negative control did not exhibit such binding. Since signal generation requires simultaneous binding of hCD86-Fc-NKG2A to both rhCD28 protein and HLA-E protein, these results demonstrate that hCD86-Fc-NKG2A binds to both hCD28 protein and HLA-E simultaneously in a dose dependent manner.

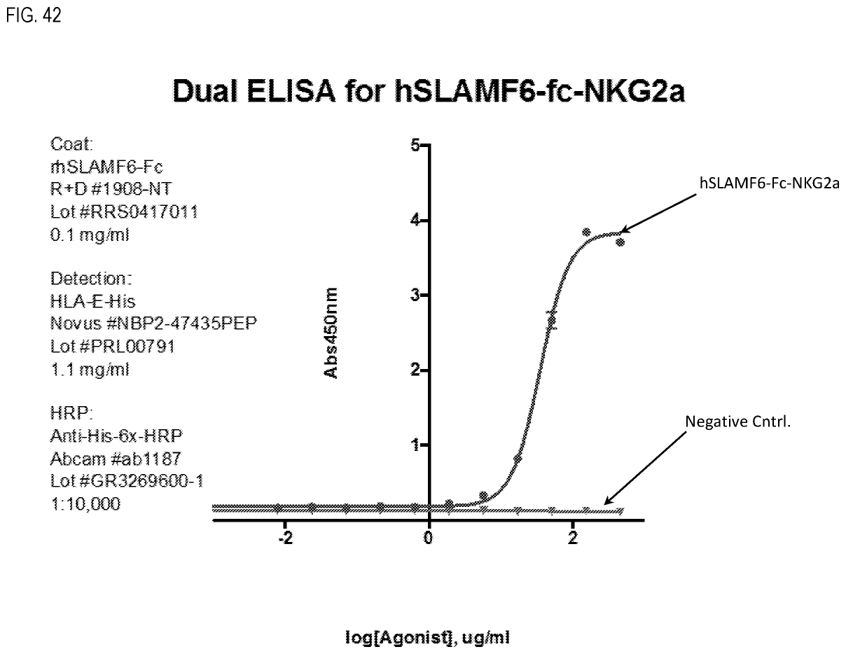

To understand whether the hSLAMF6-Fc-NKG2A chimeric protein can binds simultaneously to both hSLAMF6 and HLA-E, the following ELISA experiment was carried out. Briefly, and recombinant hSLAMF6 (rhSLAMF6-Fc) protein was coated on plates. Increasing amounts of the hSLAMF6-Fc-NKG2A chimeric protein was added to the plates for capture by the recombinant human SLAMF6-Fc (rhSLAMF6-Fc) protein. Further, recombinant HLA-E (HLA-E-His) protein was added to the plate for capture by hSLAMF6-Fc-NKG2A chimeric protein. The binding of the HLA-E-His protein was detected using an anti-His 6X HRP antibody. As shown in FIG. 42, the ELISA assays showed a dose-dependent binding by hSLAMF6-Fc-NKG2A. In contrast, a negative control did not exhibit such binding. Since signal generation requires simultaneous binding of hSLAMF6-Fc-NKG2A to both rhSLAMF6 protein and HLA-E protein, these results demonstrate that hSLAMF6-Fc-NKG2A binds to both hSLAMF6 protein and HLA-E simultaneously in a dose dependent manner.

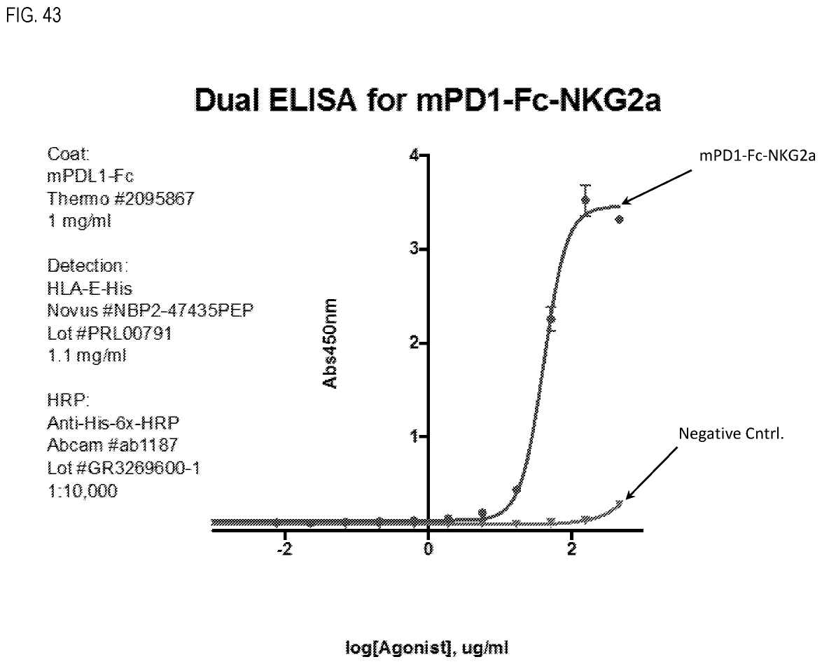

To understand whether the PD-1-Fc-NKG2A chimeric protein can binds simultaneously to both PD-L1 and HLA-E, the following ELISA experiment was carried out. Briefly, and recombinant PD-L1 (rPD-L1-Fc) protein was coated on plates. Increasing amounts of the PD-1-Fc-NKG2A chimeric protein was added to the plates for capture by the rPD-L1-Fc protein. Further, recombinant HLA-E (HLA-E-His) protein was added to the plate for capture by PD-1-Fc-NKG2A chimeric protein. The binding of the HLA-E-His protein was detected using an anti-His 6X HRP antibody. As shown in FIG. 43, the ELISA assays showed a dose-dependent binding by PD-1-Fc-NKG2A. In contrast, a negative control did not exhibit such binding. Since signal generation requires simultaneous binding of PD-1-Fc-NKG2A to both rPD-L1 protein and HLA-E protein, these results demonstrate that PD-1-Fc-NKG2A binds to both PD-L1 protein and HLA-E simultaneously in a dose dependent manner.

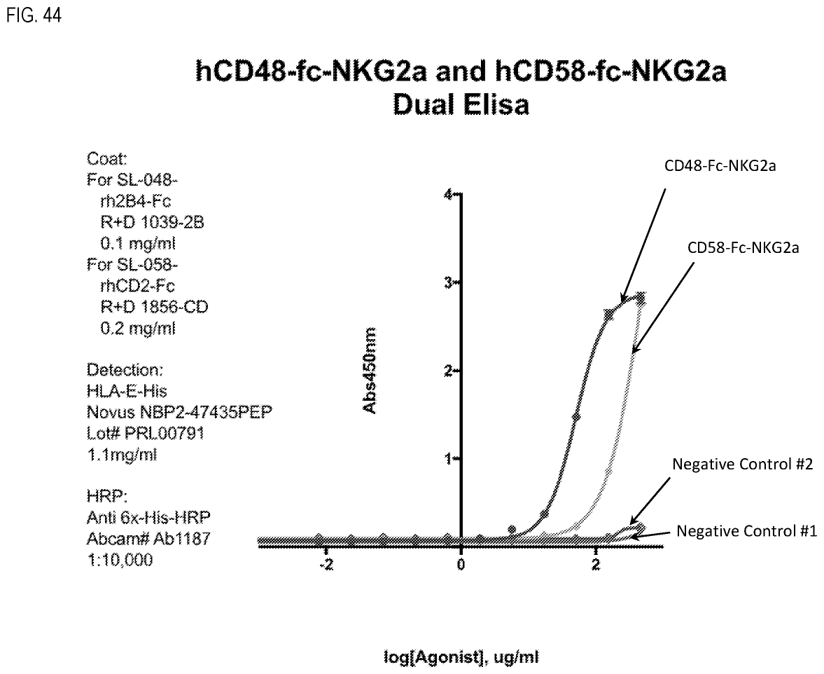

To understand whether the hCD48-Fc-NKG2A chimeric protein can bind simultaneously to both 2B4 and HLA-E, the following ELISA experiment was carried out. Briefly, and recombinant 2B4 (rh2B4-Fc) protein was coated on plates. Increasing amounts of the hCD48-Fc-NKG2A chimeric protein was added to the plates for capture by the recombinant human 2B4-Fc (rh2B4-Fc) protein. Further, recombinant HLA-E (HLA-E-His) protein was added to the plate for capture by hCD48-Fc-NKG2A chimeric proteins. The binding of the HLA-E-His protein was detected using an anti-His 6X HRP antibody. As shown in FIG. 44, the ELISA assays showed a dose-dependent binding by hCD48-Fc-NKG2A. In contrast, a negative control did not exhibit such binding. Since signal generation requires simultaneous binding of hCD48-Fc-NKG2A to both rh2B4 protein and HLA-E protein, these results demonstrate that hCD48-Fc-NKG2A binds to both 2B4 protein and HLA-E simultaneously in a dose dependent manner.

To understand whether the hCD58-Fc-NKG2A chimeric protein can bind simultaneously to both CD2 and HLA-E, the following ELISA experiment was carried out. Briefly, and recombinant CD2 (rhCD2-Fc) protein was coated on plates. Increasing amounts of the hCD58-Fc-NKG2A chimeric protein was added to the plates for capture by the recombinant human CD2-Fc (rhCD2-Fc) protein. Further, recombinant HLA-E (HLA-E-His) protein was added to the plate for capture by hCD58-Fc-NKG2A chimeric proteins. The binding of the HLA-E-His protein was detected using an anti-His 6X HRP antibody. As shown in FIG. 44, the ELISA assays showed a dose-dependent binding by hCD58-Fc-NKG2A. In contrast, a negative control did not exhibit such binding. Since signal generation requires simultaneous binding of hCD58-Fc-NKG2A to both rhCD2 protein and HLA-E protein, these results demonstrate that hCD58-Fc-NKG2A binds to both CD2 protein and HLA-E simultaneously in a dose dependent manner.

Collectively, these results demonstrate that the type I transmembrane protein part, which is located at or near the N-terminus of the chimeric proteins, and the NKG2A part, which is located at or near the C-terminus of the chimeric proteins of the present invention simulataneously bind their intended targets.

### Example 26: Specific Binding of the Human CD48-Fc-NKG2A Chimeric Protein to Cells Expressing h2B4

To understand whether the type I transmembrane protein part, which is located at or near the N-terminus of the chimeric proteins, can specifically bind cells expressing their intended targets, the following series of experiments (Examples 26 to 33) were performed.

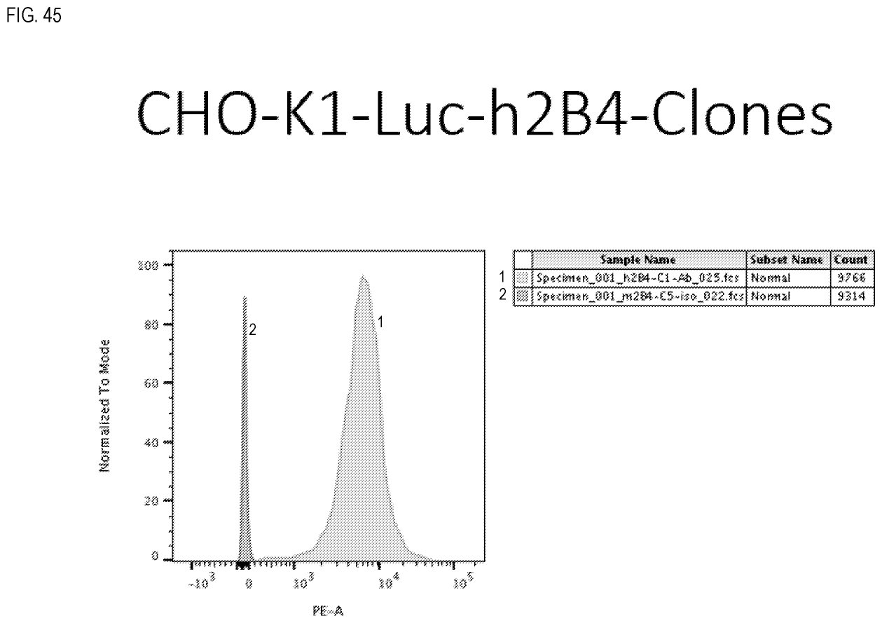

To understand whether the hCD48 part of the hCD48-Fc-NKG2A chimeric protein can specifically bind cells expressing h2B4, clones of CHO-K1 cells expressing h2B4 (the binding partner of CD48) were generated. A positive clone (called CHO-K1/h2B4 cells) was stained with an anti-2B4 antibody or an isotype control, and subjected to flow cytometry analysis. As shown in FIG. 45, staining of the CHO-K1/h2B4 cells by the anti-2B4 antibody but not the isotype control, confirmed the generation of the CHO-K1/h2B4 cells.

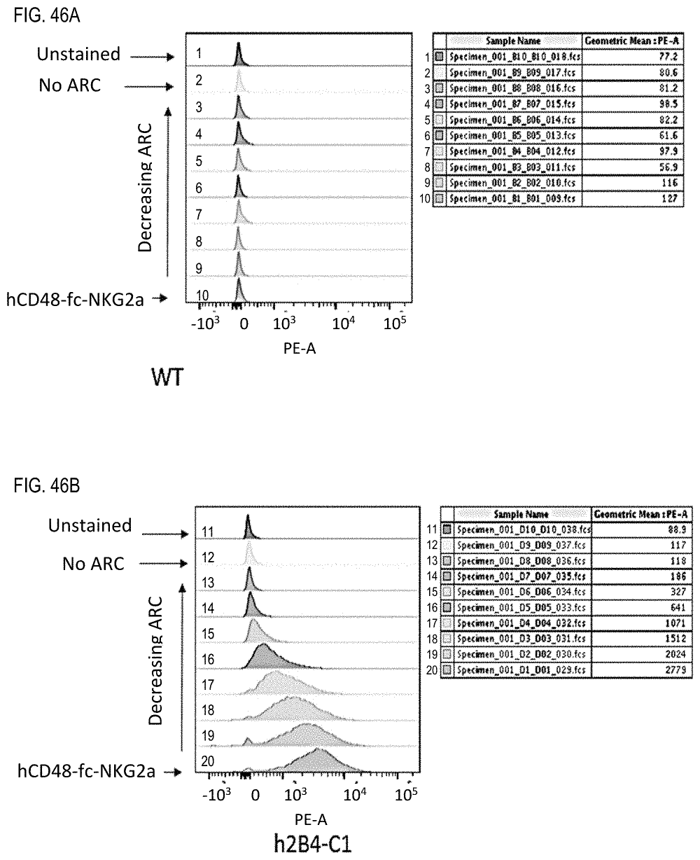

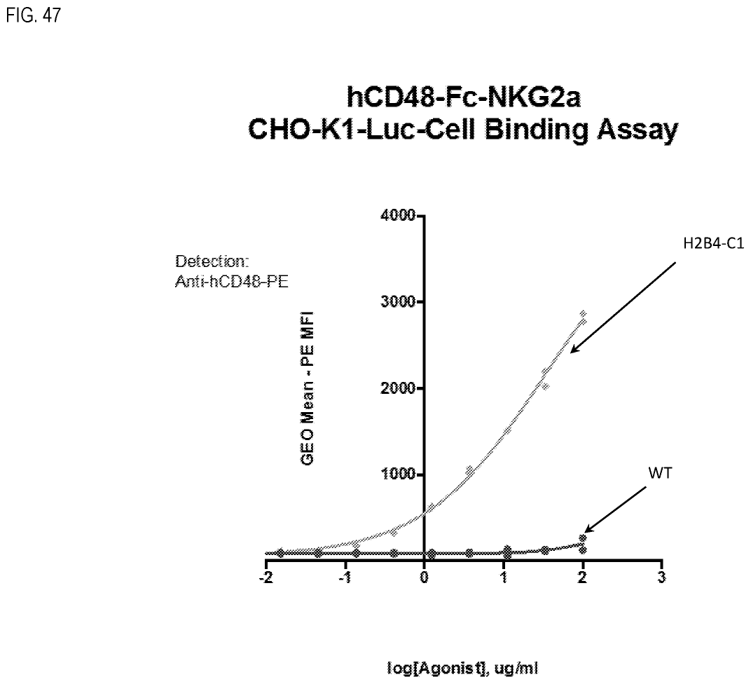

To determine whether the hCD48-Fc-NKG2A chimeric protein can specifically bind the CHO-K1/h2B4 cells, a flow-cytometry-based assay was carried out. As shown in FIG. 46A and FIG. 46B, the hCD48-Fc-NKG2A chimeric protein displayed more binding to CHO-K1/h2B4 cells (FIG. 46B) compared to the WT CHO-K1 cells (FIG. 46A). The dose dependent shifts in the flow cytometry profiles illustrate a dose-dependent binding of the hCD48-Fc-NKG2A chimeric protein to h2B4 expressed by the CHO-K1/h2B4 cells. A quantitation of binding confirmed a dose-dependent binding (FIG. 47). These results demonstrate that the hCD48-Fc-NKG2A chimeric protein specifically binds cells expressing h2B4.

### Example 27: Specific Binding of the mCD48-Fc-NKG2A Chimeric Protein to Cells Expressing m2B4

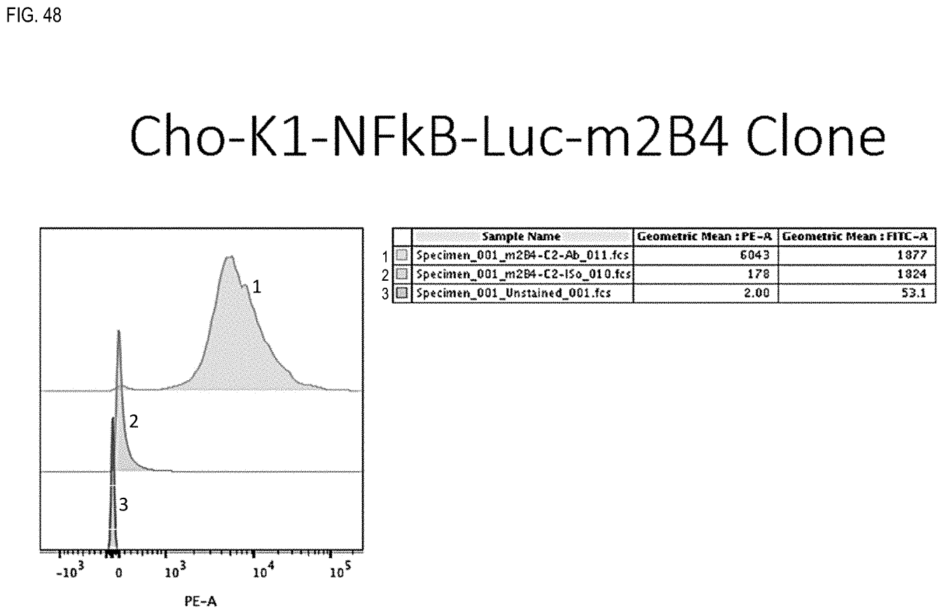

To understand whether the mCD48 part of the mCD48-Fc-NKG2A chimeric protein can specifically bind cells expressing m2B4, clones of CHO-K1 cells expressing m2B4 (the binding partner of CD48) were generated. A positive clone (called CHO-K1/m2B4 cells) was stained with an anti-m2B4 antibody or an isotype control, and subjected to flow cytometry analysis. As shown in FIG. 48, staining of the CHO-K1/m2B4 cells by the anti-m2B4 antibody but not the isotype control, when compared with an unstained cell control, confirmed the generation of the CHO-K1/m2B4 cells.

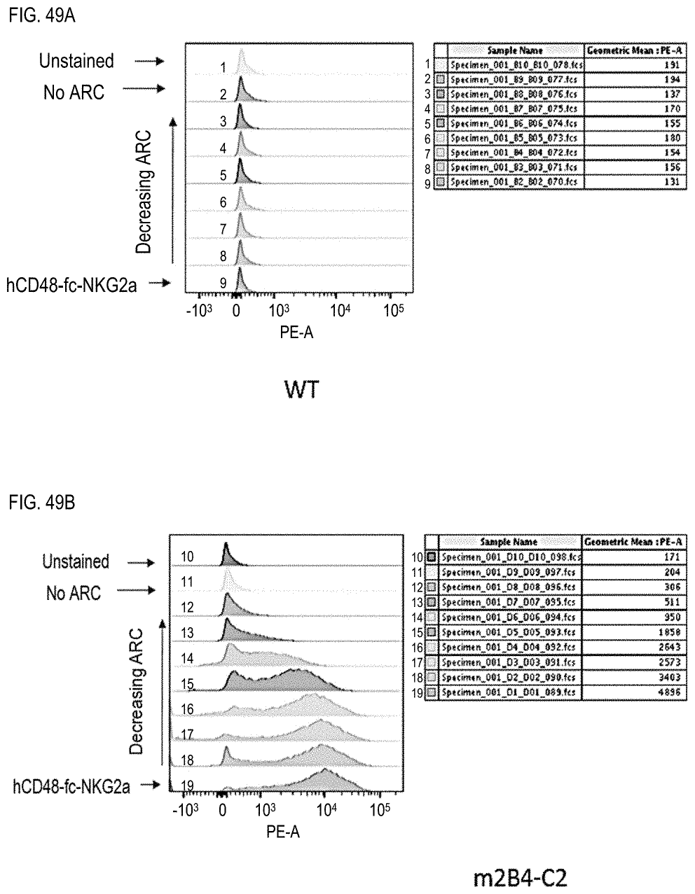

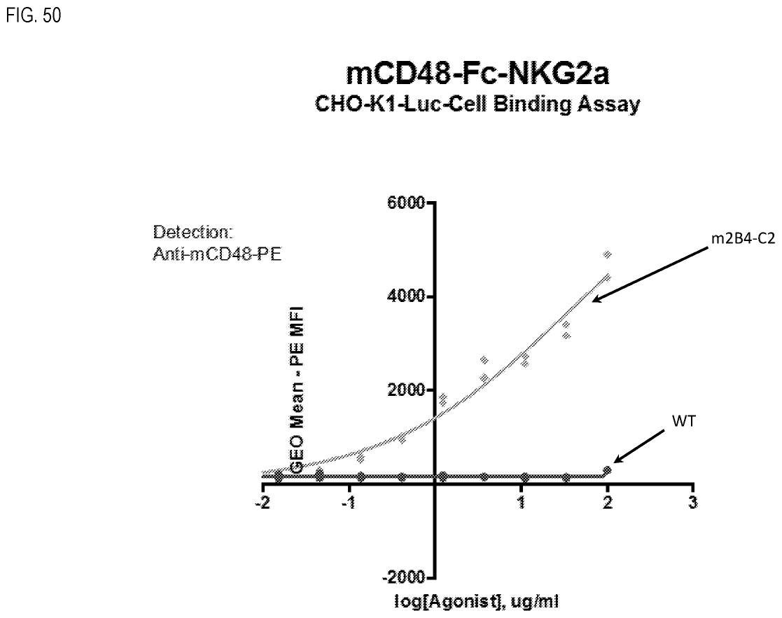

To determine whether the mCD48-Fc-NKG2A chimeric protein can specifically bind the CHO-K1/m2B4 cells, a flow-cytometry-based assay was carried out. As shown in FIG. 49A and FIG. 49B, the mCD48-Fc-NKG2A chimeric protein displayed more binding to CHO-K1/m2B4 cells (FIG. 49B) compared to the WT CHO-K1 cells (FIG. 49A). The dose dependent shifts in the flow cytometry profiles illustrate a dose-dependent binding of the mCD48-Fc-NKG2A chimeric protein to m2B4 expressed by the CHO-K1/m2B4 cells. A quantitation of binding confirmed a dose-dependent binding (FIG. 50). These results demonstrate that the mCD48-Fc-NKG2A chimeric protein specifically binds cells expressing m2B4.

### Example 28: Specific Binding of the Human PD-1-Fc-NKG2A Chimeric Protein to Cells Expressing hPD-L1

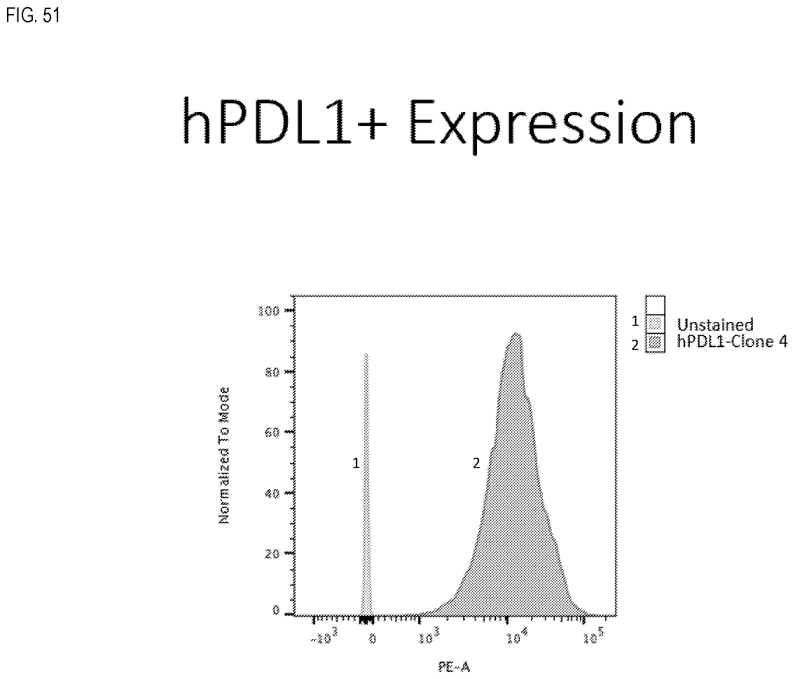

To understand whether the hPD-1 part of the hPD-1-Fc-NKG2A chimeric protein can specifically bind cells expressing hPD-L1, clones of CHO-K1 cells expressing hPD-L1 (the binding partner of PD-1) were generated. A positive clone (called CHO-K1/hPD-L1 cells) was stained with an anti-PD-L1 antibody or an isotype control, and subjected to flow cytometry analysis. As shown in FIG. 51, staining of the CHO-K1/hPD-L1 cells by the anti-PD-L1 antibody but not the isotype control, confirmed the generation of the CHO-K1/hPD-L1 cells.

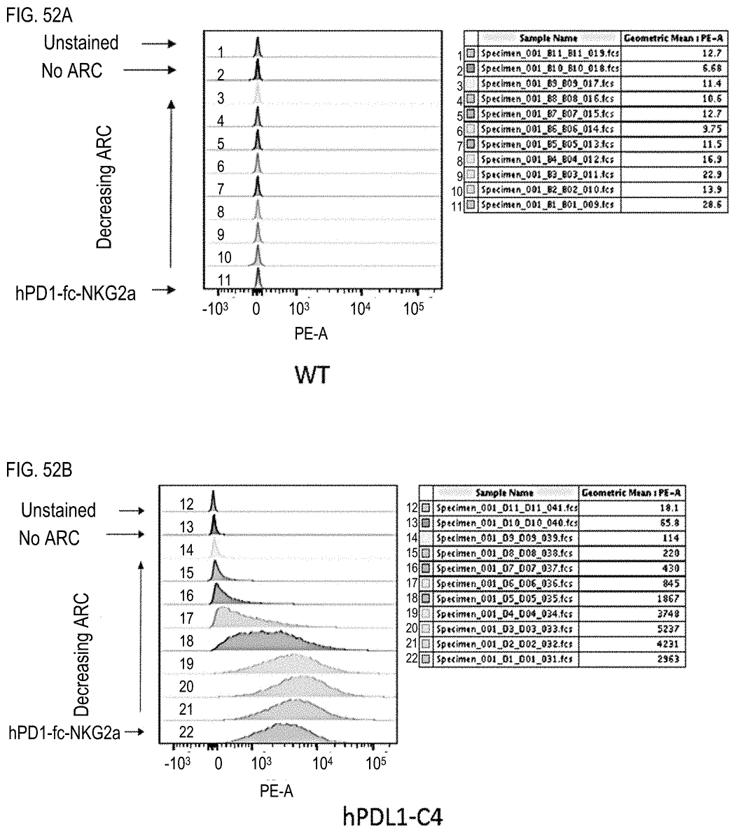

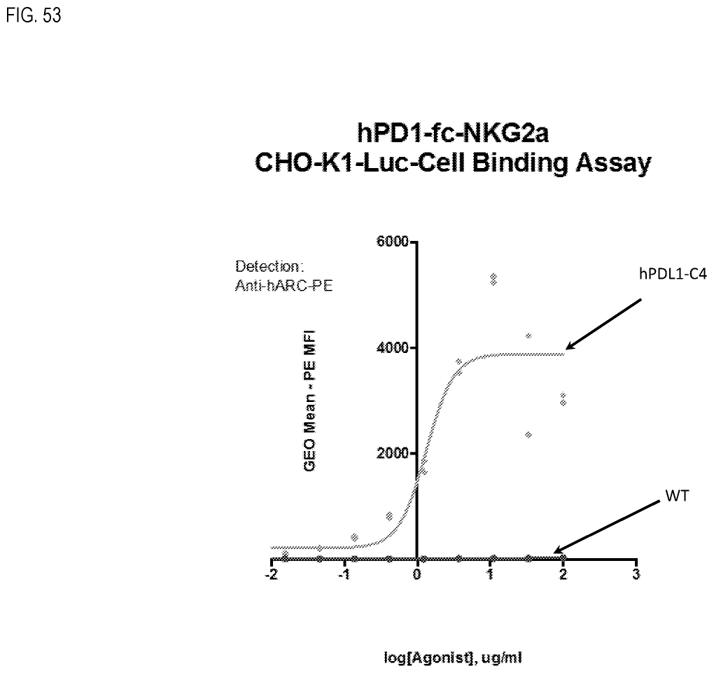

To determine whether the hPD-1-Fc-NKG2A chimeric protein can specifically bind the CHO-K1/hPD-L1 cells, a flow-cytometry-based assay was carried out. As shown in FIG. 52A and FIG. 52B, the hPD-1-Fc-NKG2A chimeric protein displayed more binding to CHO-K1/hPD-L1 cells (FIG. 52B) compared to the WT CHO-K1 cells (FIG. 52A). The dose dependent shifts in the flow cytometry profiles illustrate a dose-dependent binding of the hPD-1-Fc-NKG2A chimeric protein to hPD-L1 expressed by the CHO-K1/hPD-L1 cells. A quantitation of binding confirmed a dose-dependent binding (FIG. 53). These results demonstrate that the hPD-1-Fc-NKG2A chimeric protein specifically binds cells expressing hPD-L1.

### Example 29: Specific Binding of the Mouse PD-1-Fc-NKG2A Chimeric Protein to Cells Expressing mPD-L1

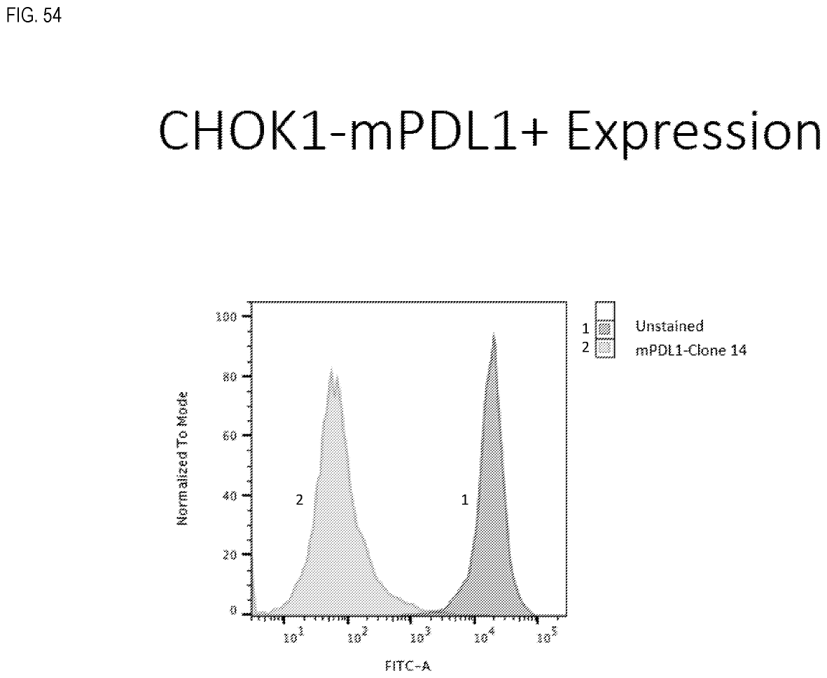

To understand whether the mPD-1 part of the mPD-1-Fc-NKG2A chimeric protein can specifically bind cells expressing mPD-L1, clones of CHO-K1 cells expressing mPD-L1 (the binding partner of PD-1) were generated. A positive clone (called CHO-K1/mPD-L1 cells) was stained with an anti-PD-L1 antibody or an isotype control, and subjected to flow cytometry analysis. As shown in FIG. 54, staining of the CHO-K1/mPD-L1 cells by the anti-PD-L1 antibody but not the isotype control, confirmed the generation of the CHO-K1/mPD-L1 cells.

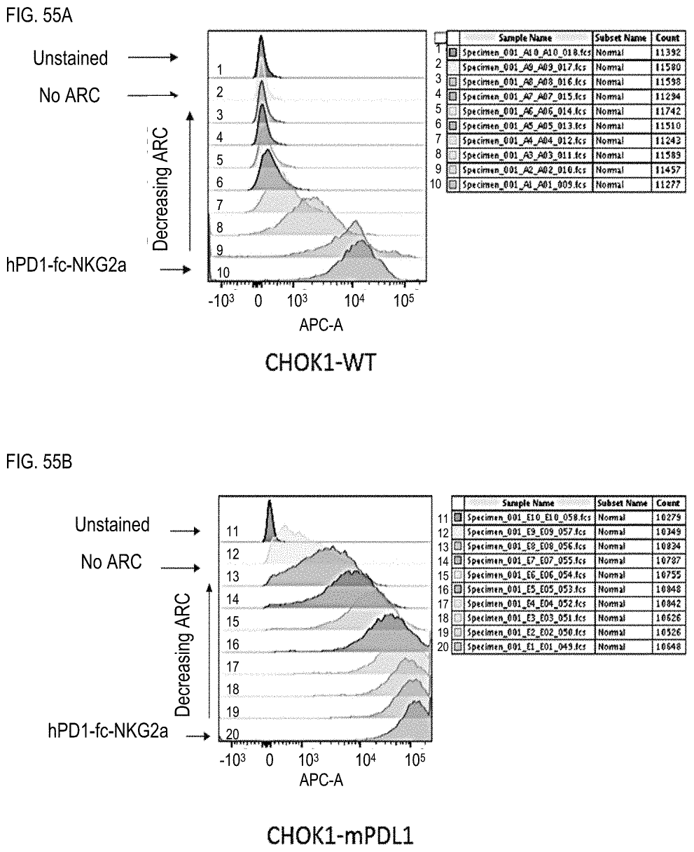

To determine whether the mPD-1-Fc-NKG2A chimeric protein can specifically bind the CHO-K1/mPD-L1 cells, a flow-cytometry-based assay was carried out. As shown in FIG. 55A and FIG. 55B, the mPD-1-Fc-NKG2A chimeric protein displayed more binding to CHO-K1/mPD-L1 cells (FIG. 55B) compared to the WT CHO-K1 cells (FIG. 55A).

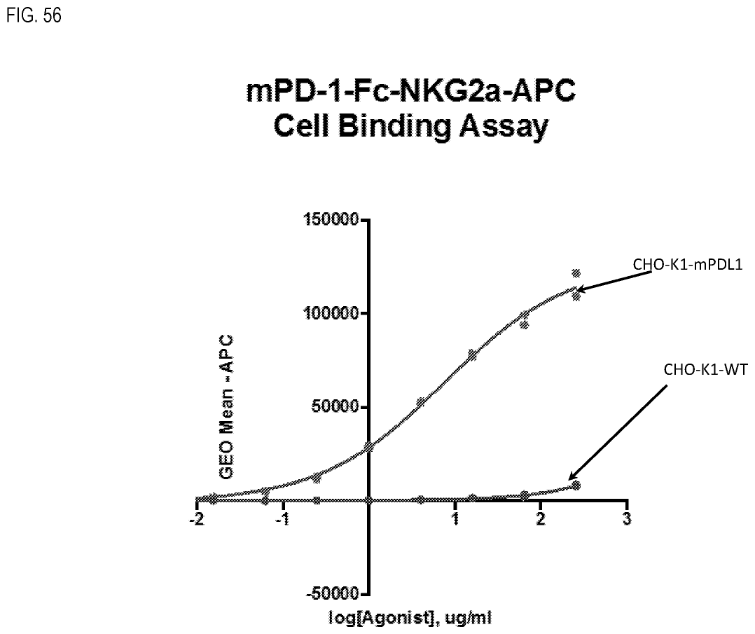

The dose dependent shifts in the flow cytometry profiles illustrate a dose-dependent binding of the mPD-1-Fc-NKG2A chimeric protein to mPD-L1 expressed by the CHO-K1/mPD-L1 cells. A quantitation of binding confirmed a dose-dependent binding (FIG. 56). These results demonstrate that the mPD-1-Fc-NKG2A chimeric protein specifically binds cells expressing mPD-L1.

### Example 30: Specific Binding of the Mouse CD48-Fc-NKG2A Chimeric Protein to Cells Expressing mQa1

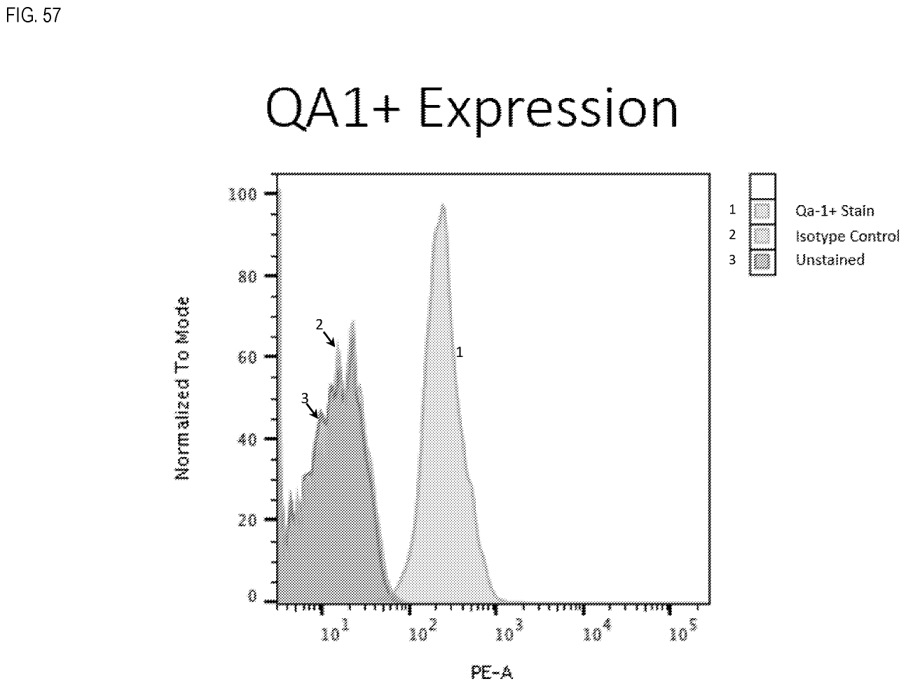

To understand whether the mCD48 part of the mCD48-Fc-NKG2A chimeric protein can specifically bind cells expressing mQa1, clones of CHO-K1 cells expressing mQa1 (the binding partner of mouse NKG2A) were generated. A positive clone (called CHO-K1/mQa1 cells) was stained with an anti-Qa1 antibody or an isotype control, and subjected to flow cytometry analysis. As shown in FIG. 57, staining of the CHO-K1/mQa1 cells by the anti-Qa1 antibody but not the isotype control, compared to an unstained control, confirmed the generation of the CHO-K1/mQa1 cells.

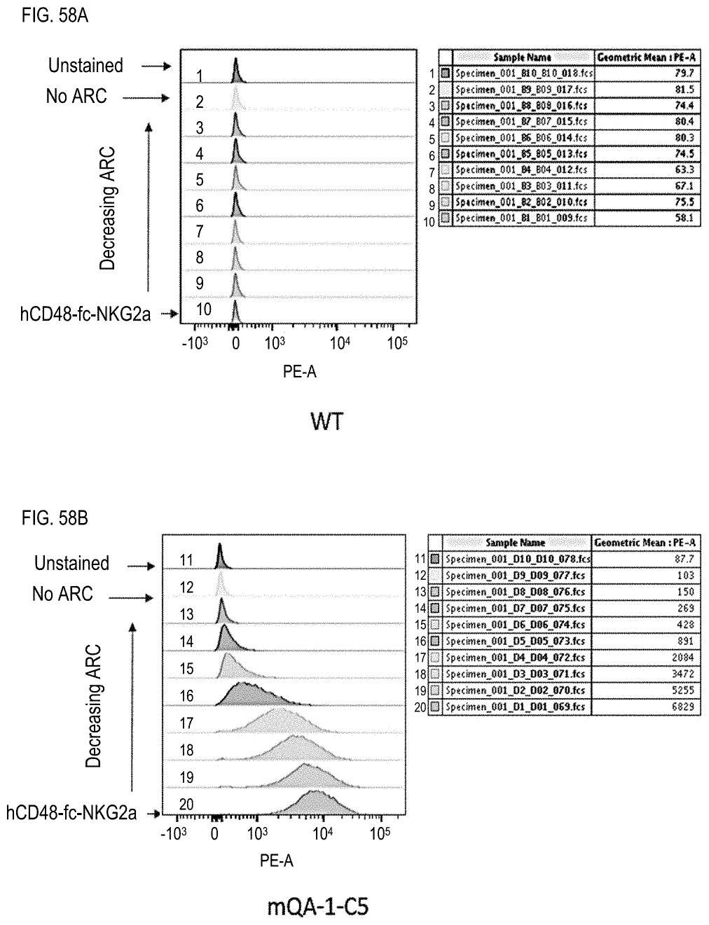

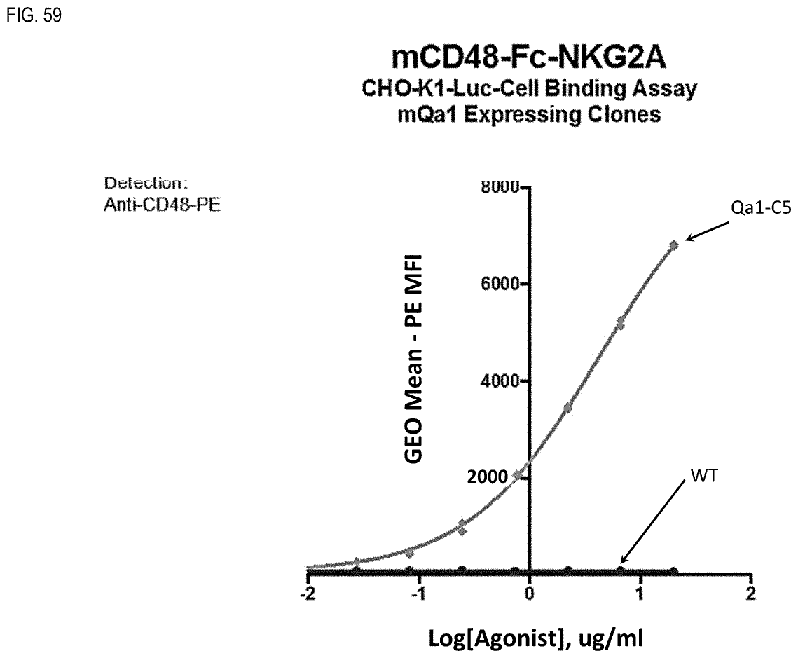

To determine whether the mCD48-Fc-NKG2A chimeric protein can specifically bind the CHO-K1/mQa1 cells, a flow-cytometry-based assay was carried out. As shown in FIG. 58A and FIG. 58B, the mCD48-Fc-NKG2A chimeric protein displayed more binding to CHO-K1/mQa1 cells (FIG. 58B) compared to the WT CHO-K1 cells (FIG. 58A). The dose dependent shifts in the flow cytometry profiles illustrate a dose-dependent binding of the mCD48-Fc-NKG2A chimeric protein to mQa1 expressed by the CHO-K1/mQa1 cells. A quantitation of binding confirmed a dose-dependent binding (FIG. 59). These results demonstrate that the mCD48-Fc-NKG2A chimeric protein specifically binds cells expressing mQa1.

### Example 31: Specific Binding of the Human CD58-Fc-NKG2A Chimeric Protein to Cells Expressing hCD2

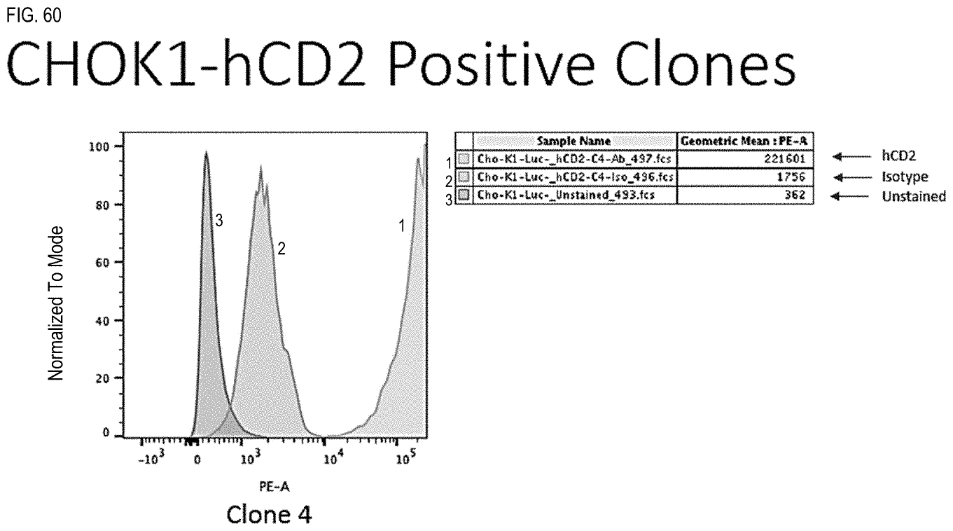

To understand whether the hCD58 part of the hCD58-Fc-NKG2A chimeric protein can specifically bind cells expressing hCD2, clones of CHO-K1 cells expressing hCD2 were generated. A positive clone (called CHO-K1/hCD2 cells) was stained with an anti-CD2 antibody or an isotype control, and subjected to flow cytometry analysis. As shown in FIG. 60, staining of the CHO-K1/hCD2 cells by the anti-CD2 antibody but not the isotype control, compared to an unstained control, confirmed the generation of the CHO-K1/hCD2 cells.

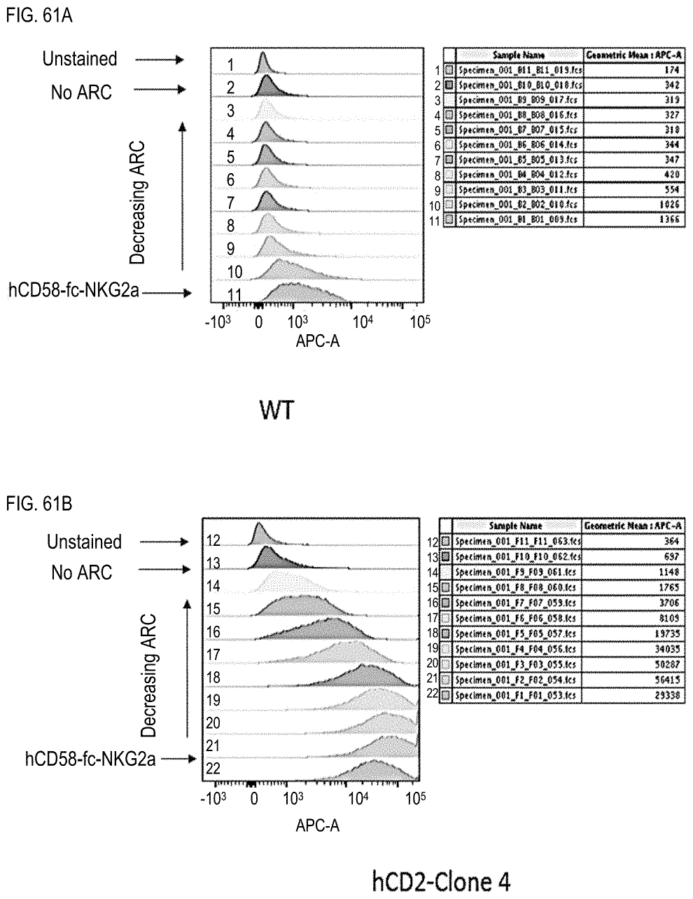

To determine whether the hCD58-Fc-NKG2A chimeric protein can specifically bind the CHO-K1/hCD2 cells, a flow-cytometry-based assay was carried out. As shown in FIG. 61A and FIG. 61B, the hCD58-Fc-NKG2A chimeric protein displayed more binding to CHO-K1/hCD2 cells (FIG. 61B) compared to the WT CHO-K1 cells (FIG. 61A).

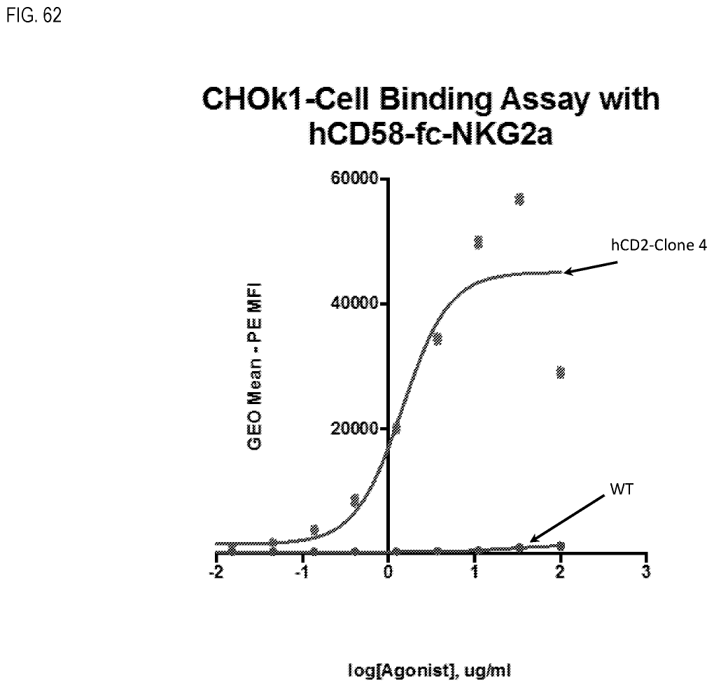

The dose dependent shifts in the flow cytometry profiles illustrate a dose-dependent binding of the hCD58-Fc-NKG2A chimeric protein to hCD2 expressed by the CHO-K1/hCD2 cells. A quantitation of binding confirmed a dose-dependent binding (FIG. 62). These results demonstrate that the hCD58-Fc-NKG2A chimeric protein specifically binds cells expressing hCD2.

### Example 32: Specific Binding of the Human CD86-Fc-NKG2A Chimeric Protein to Cells Expressing hCD28

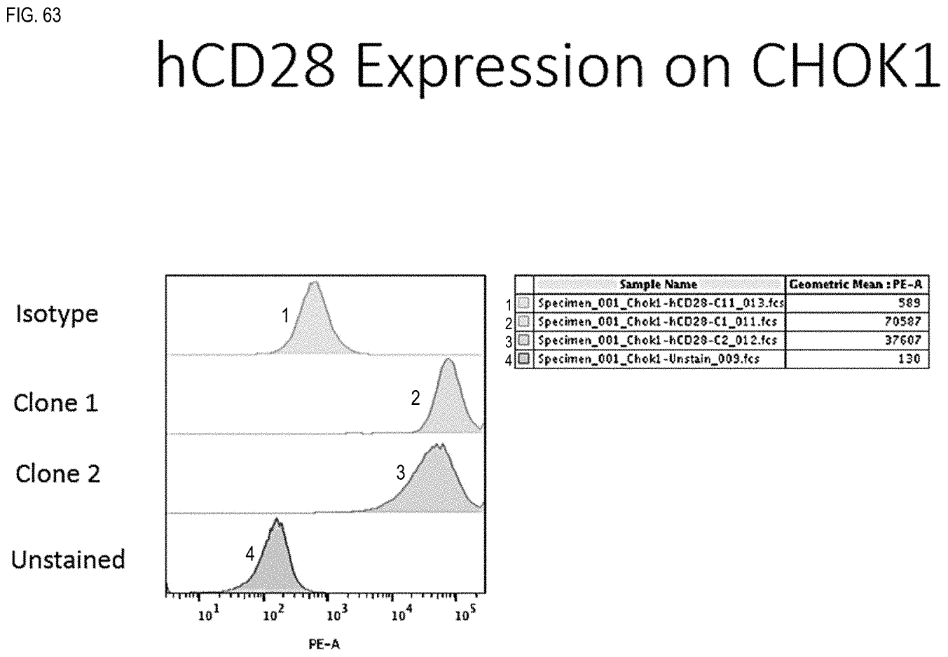

To understand whether the hCD86 part of the hCD86-Fc-NKG2A chimeric protein can specifically bind cells expressing hCD28, clones of CHO-K1 cells expressing hCD28 were generated. Two positive clone (called CHO-K1/hCD28 cell clones) were stained with an anti-CD28 antibody or an isotype control, and subjected to flow cytometry analysis. As shown in FIG. 63, staining of the CHO-K1/hCD28 clones by the anti-CD28 antibody but not the isotype control, compared to an unstained control, confirmed the generation of the CHO-K1/hCD28 clones.

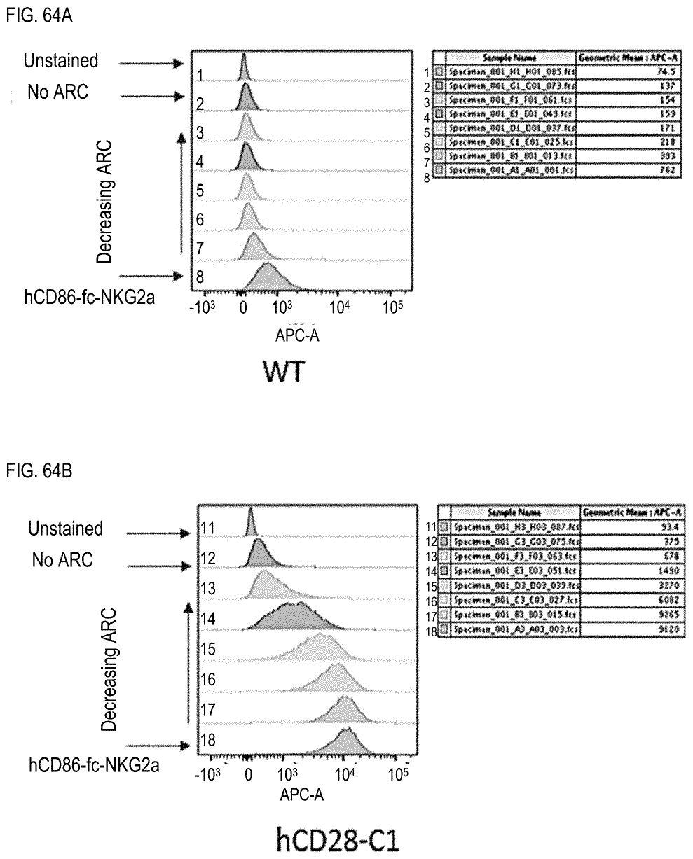

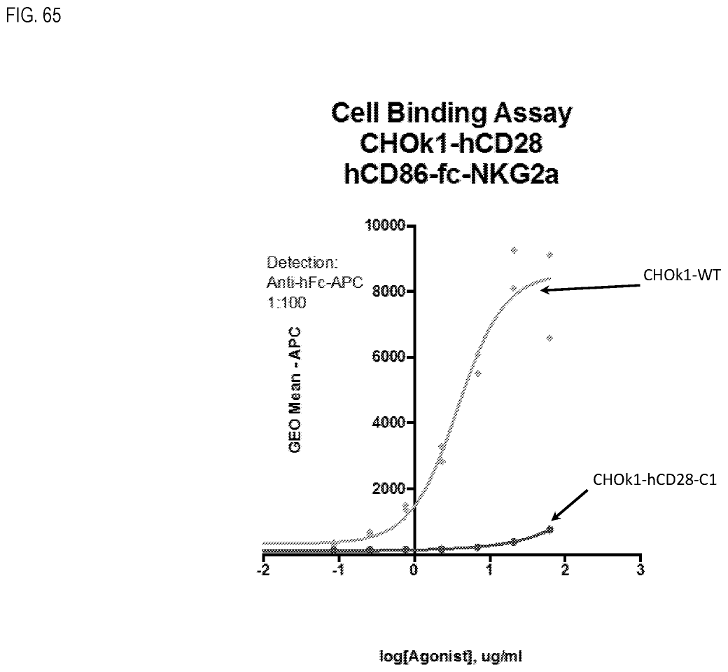

To determine whether the hCD86-Fc-NKG2A chimeric protein can specifically bind the CHO-K1/hCD28 cells, a flow-cytometry-based assay was carried out. As shown in FIG. 64A and FIG. 64B, the hCD86-Fc-NKG2A chimeric protein displayed more binding to CHO-K1/hCD28 cells (FIG. 64B) compared to the WT CHO-K1 cells (FIG. 64A). The dose dependent shifts in the flow cytometry profiles illustrate a dose-dependent binding of the hCD86-Fc-NKG2A chimeric protein to hCD28 expressed by the CHO-K1/hCD28 cells. A quantitation of binding confirmed a dose-dependent binding (FIG. 65). These results demonstrate that the hCD86-Fc-NKG2A chimeric protein specifically binds cells expressing hCD28.

### Example 33: Specific Binding of the Mouse CD80-Fc-NKG2A and CD86-Fc-NKG2A Chimeric Proteins to Cells Expressing mCD28

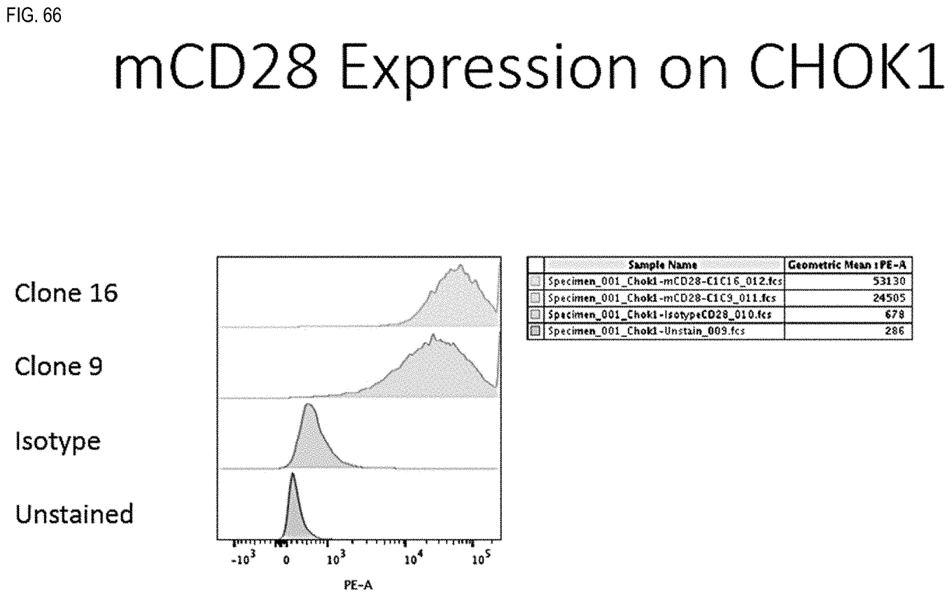

To understand whether the mCD80 and mCD86 part of the mCD80-Fc-NKG2A and mCD86-Fc-NKG2A chimeric protein, respectively, can specifically bind cells expressing mCD28, clones of CHO-K1 cells expressing mCD28 were generated. Two positive clone (called CHO-K1/mCD28 cell clones) were stained with an anti-CD28 antibody or an isotype control, and subjected to flow cytometry analysis. As shown in FIG. 66, staining of the CHO-K1/mCD28 clones by the anti-CD28 antibody but not the isotype control, compared to an unstained control, confirmed the generation of the CHO-K1/mCD28 clones.

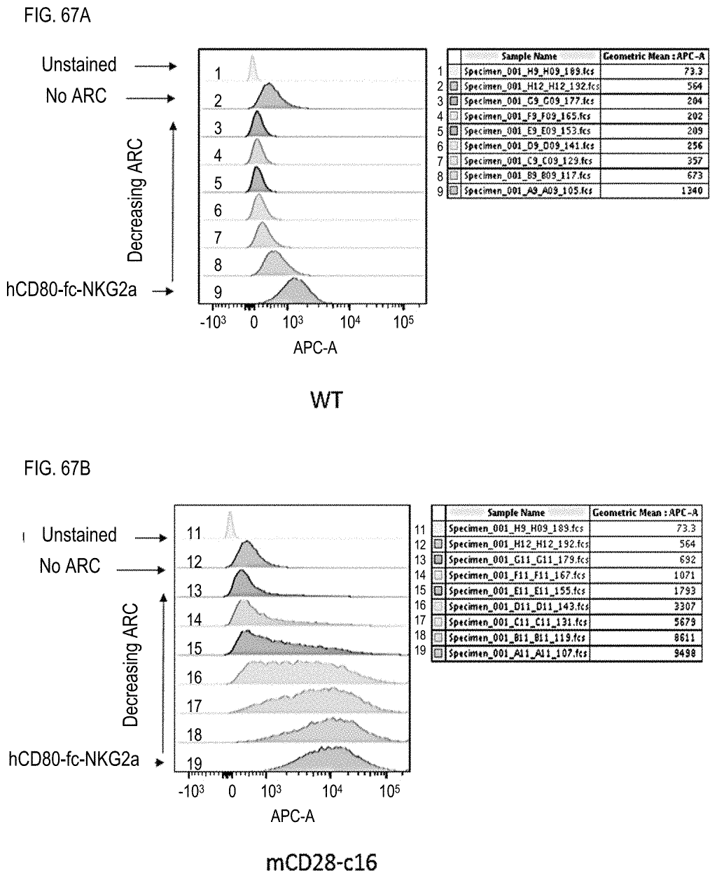

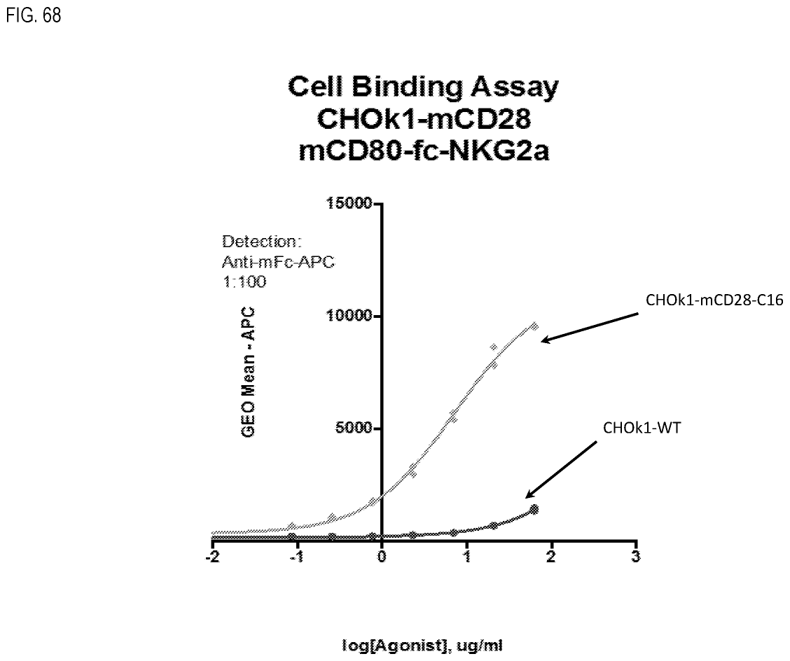

To determine whether the mCD80-Fc-NKG2A chimeric protein can specifically bind the CHO-K1/mCD28 cells, a flow-cytometry-based assay was carried out. As shown in FIG. 67A and FIG. 67B, the mCD80-Fc-NKG2A chimeric protein displayed more binding to CHO-K1/mCD28 cells (FIG. 67B) compared to the WT CHO-K1 cells (FIG. 67A). The dose dependent shifts in the flow cytometry profiles illustrate a dose-dependent binding of the mCD80-Fc-NKG2A chimeric protein to mCD28 expressed by the CHO-K1/mCD28 cells. A quantitation of binding confirmed a dose-dependent binding (FIG. 68). These results demonstrate that the mCD80-Fc-NKG2A chimeric protein specifically binds cells expressing mCD28.

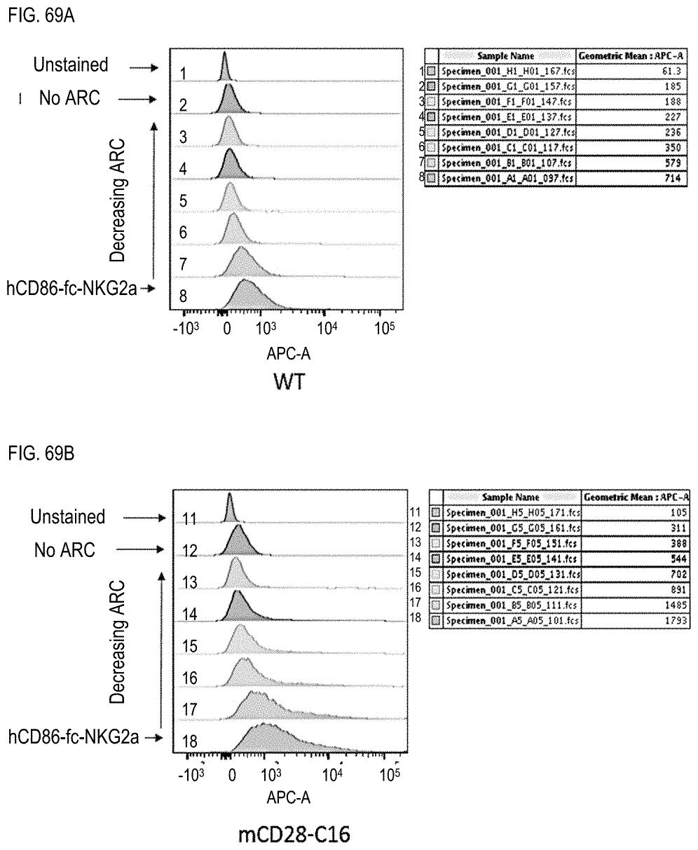

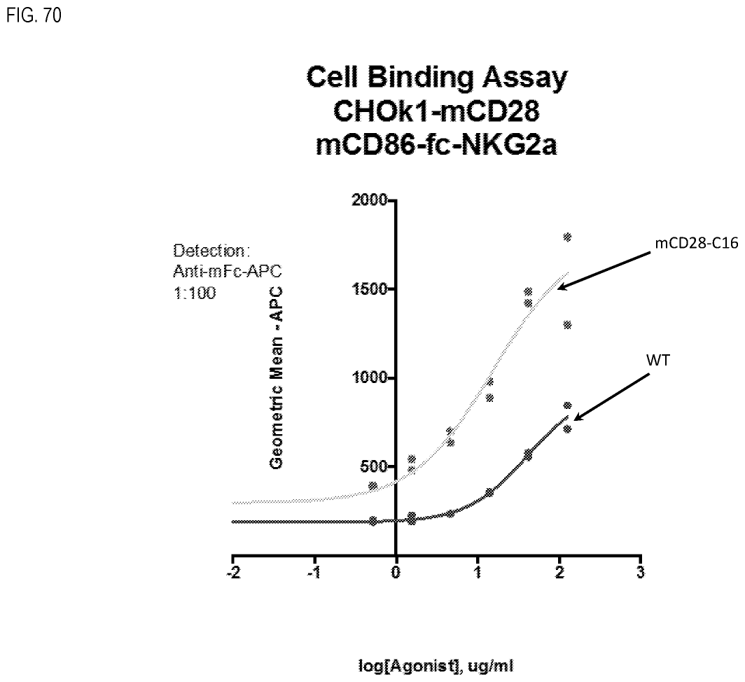

To determine whether the mCD86-Fc-NKG2A chimeric protein can specifically bind the CHO-K1/mCD28 cells, a flow-cytometry-based assay was carried out. As shown in FIG. 69A and FIG. 69B, the mCD80-Fc-NKG2A chimeric protein displayed more binding to CHO-K1/mCD28 cells (FIG. 69B) compared to the WT CHO-K1 cells (FIG. 69A). The dose dependent shifts in the flow cytometry profiles illustrate a dose-dependent binding of the mCD86-Fc-NKG2A chimeric protein to mCD28 expressed by the CHO-K1/mCD28 cells. A quantitation of binding confirmed a dose-dependent binding (FIG. 70). These results demonstrate that the mCD86-Fc-NKG2A chimeric protein specifically binds cells expressing mCD28.

Collectively, the results from Examples 26 to 33 demonstrate that the type I transmembrane protein part, which is located at or near the N-terminus of the chimeric proteins, can specifically bind to cells expressing their natural targets.

### Example 34: The Ability of the Type I Transmembrane Protein and NKG2A Parts of the NKG2A-Based Chimeric Proteins to Initiate Signal Transduction Downstream to Their Ligands

The objectives of these experiments were (i) to understand whether the type I transmembrane protein part, which is located at or near the N-terminus of the chimeric proteins, can activate their targets; and (ii) to understand whether the NKG2A part, which is located at or near the C-terminus of the chimeric proteins, can activate its targets. The CHO-K1 cell clones expressing various ligands that were used in these assays harbor an NFκB-luciferase reporter that is sensitive to the binding of a ligand to the ligands that the CHO-K1 cell clones express.

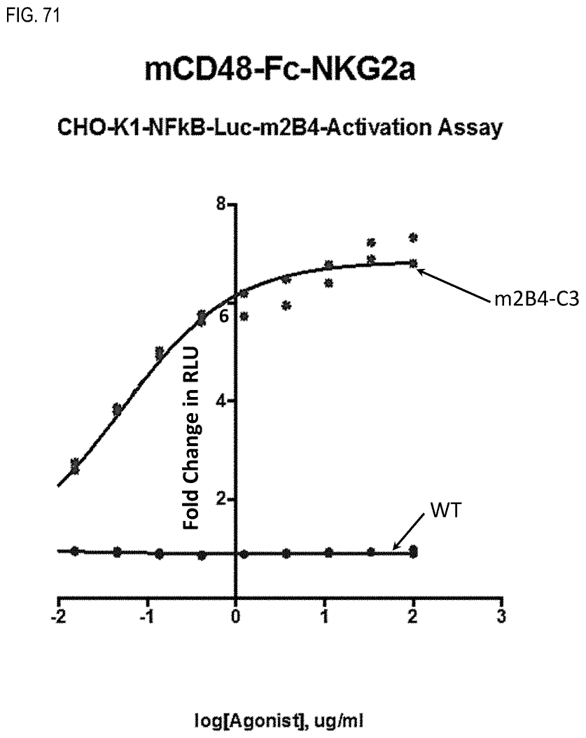

To understand whether the mCD48-Fc-NKG2A chimeric protein can activate 2B4 signaling, the following experiment was performed. Briefly, increasing amounts of the mCD48-Fc-NKG2A chimeric protein was incubated with the CHO-K1/m2B4 cells, or WT CHO-K1 cells, and the activation of the m2B4 was measured by a luciferase assay. As shown in FIG. 71, the mCD48-Fc-NKG2A chimeric protein induced luciferase activity in CHO-K1/m2B4 cells, but not WT CHO-K1 cells, in a dose dependent manner. These data demonstrate that the mCD48-Fc-NKG2A chimeric protein activates m2B4 signaling in a dose dependent manner.

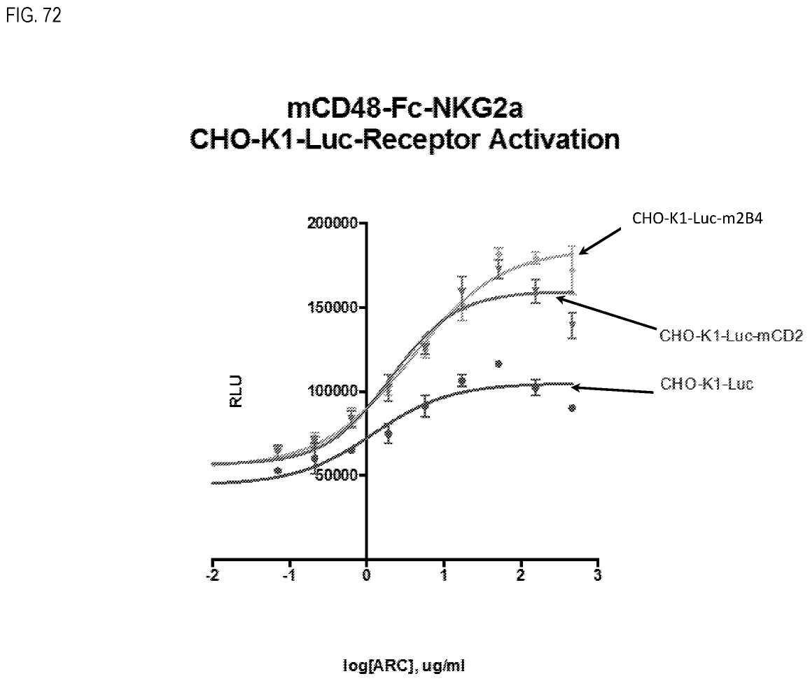

To understand whether the mCD48-Fc-NKG2A chimeric protein can activate 2B4 and mCD2 signaling, the following experiment was performed. Briefly, increasing amounts of the mCD48-Fc-NKG2A chimeric protein was incubated with the CHO-K1/m2B4 cells, the CHO-K1/mCD2 cells, or WT CHO-K1 cells, and the activation of the m2B4 or mCD2 was measured by a luciferase assay. As shown in FIG. 72, the mCD48-Fc-NKG2A chimeric protein induced luciferase activity in the CHO-K1/m2B4 and CHO-K1/mCD2 cells, but not WT CHO-K1 cells, in a dose dependent manner. These data demonstrate that the mCD48-Fc-NKG2A chimeric protein activates m2B4 and mCD2 signaling in a dose dependent manner.

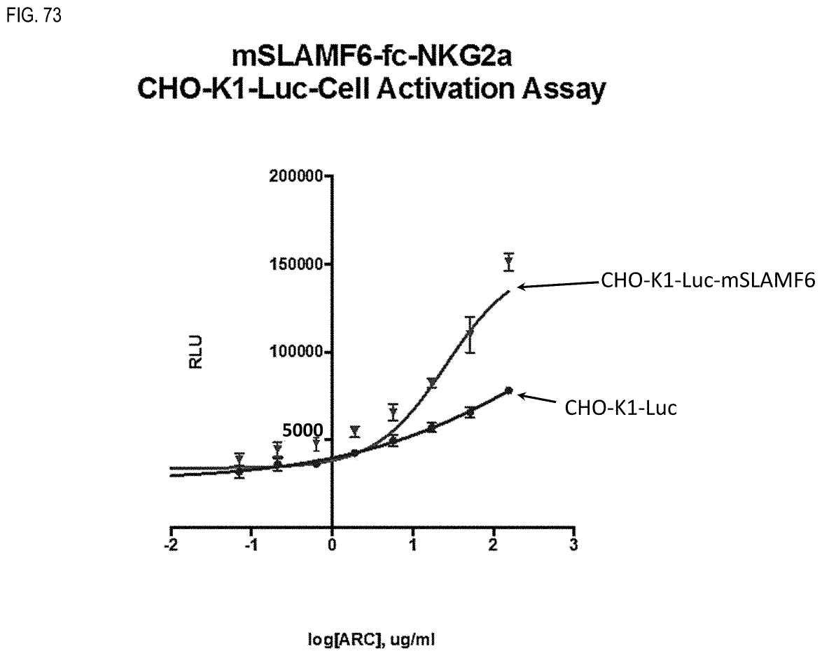

To understand whether the mSLAMF6-Fc-NKG2A chimeric protein can activate mSLAMF6 signaling, the following experiment was performed. Briefly, increasing amounts of the mSLAMF6-Fc-NKG2A chimeric protein was incubated with the CHO-K1/mSLAMF6 cells, or WT CHO-K1 cells, and the activation of the mSLAMF6 was measured by a luciferase assay. As shown in FIG. 73, the mSLAMF6-Fc-NKG2A chimeric protein induced luciferase activity in CHO-K1/mSLAMF6 cells, but not WT CHO-K1 cells, in a dose dependent manner. These data demonstrate that the mSLAMF6-Fc-NKG2A chimeric protein activates mSLAMF6 signaling in a dose dependent manner.

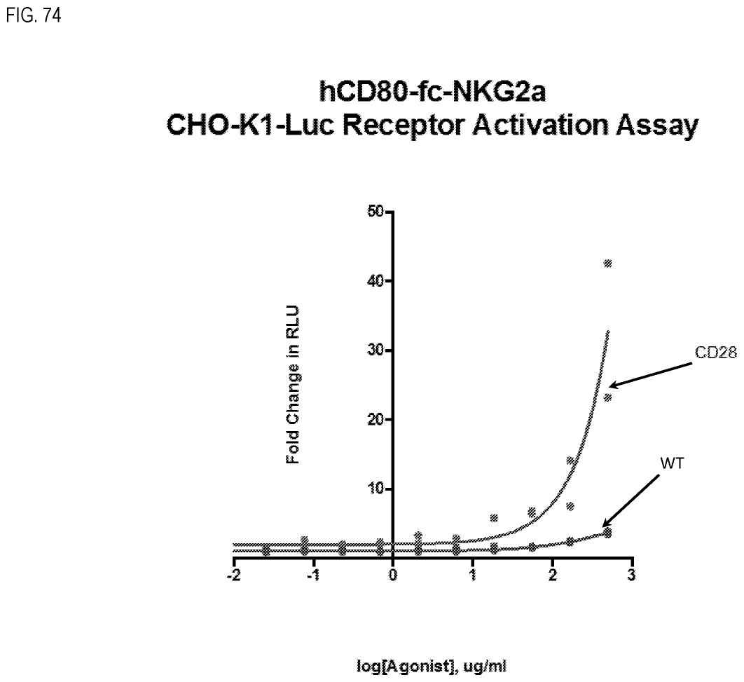

To understand whether the hCD80-Fc-NKG2A chimeric protein can activate human CD28 (hCD28) signaling, the following experiment was performed. Briefly, increasing amounts of the hCD80-Fc-NKG2A chimeric protein was incubated with the CHO-K1/hCD28 cells, or WT CHO-K1 cells, and the activation of the hCD28 was measured by a luciferase assay. As shown in FIG. 74, the hCD80-Fc-NKG2A chimeric protein induced luciferase activity in CHO-K1/hCD28 cells, but not WT CHO-K1 cells, in a dose dependent manner. These data demonstrate that the hCD80-Fc-NKG2A chimeric protein activates hCD28 signaling in a dose dependent manner.

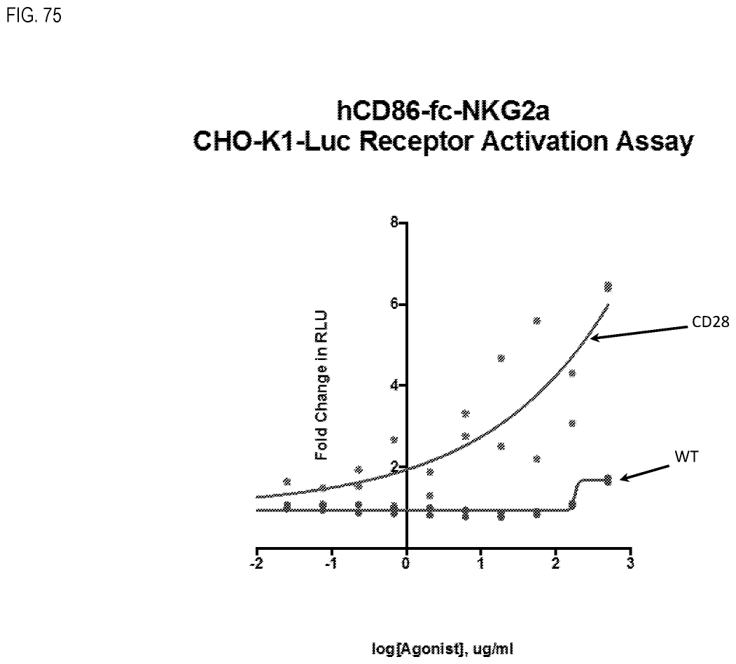

To understand whether the hCD86-Fc-NKG2A chimeric protein can activate human CD28 (hCD28) signaling, the following experiment was performed. Briefly, increasing amounts of the hCD86-Fc-NKG2A chimeric protein was incubated with the CHO-K1/hCD28 cells, or WT CHO-K1 cells, and the activation of the hCD28 was measured by a luciferase assay. As shown in FIG. 75, the hCD86-Fc-NKG2A chimeric protein induced luciferase activity in CHO-K1/hCD28 cells, but not WT CHO-K1 cells, in a dose dependent manner. These data demonstrate that the hCD86-Fc-NKG2A chimeric protein activates hCD28 signaling in a dose dependent manner.

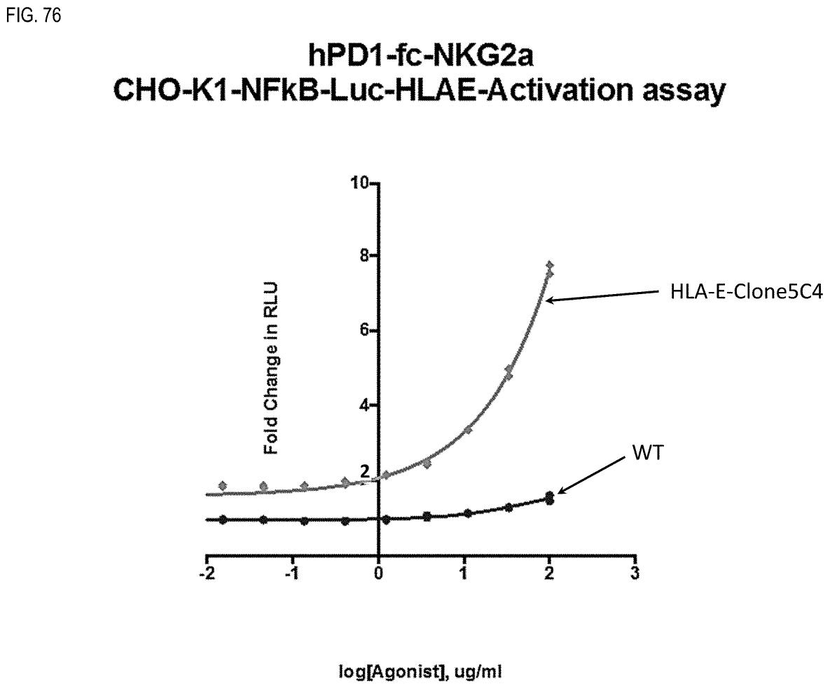

To understand whether the hPD-1-Fc-NKG2A chimeric protein can activate human HLA-E signaling, the following experiment was performed. Briefly, increasing amounts of the hPD-1-Fc-NKG2A chimeric protein was incubated with the CHO-K1/HLA-E cells, or WT CHO-K1 cells, and the activation of the hHLA-E was measured by a luciferase assay. As shown in FIG. 76, the hPD-1-Fc-NKG2A chimeric protein induced luciferase activity in CHO-K1/HLA-E cells, but not WT CHO-K1 cells, in a dose dependent manner. These data demonstrate that the hPD-1-Fc-NKG2A chimeric protein activates HLA-E signaling in a dose dependent manner.

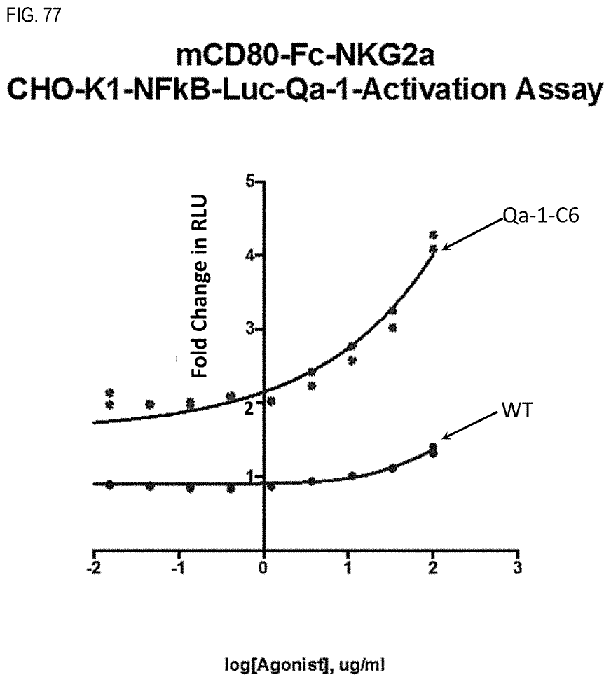

To understand whether the mCD80-Fc-NKG2A chimeric protein can activate mouse Qa1 (mQa1) signaling, the following experiment was performed. Briefly, increasing amounts of the mCD80-Fc-NKG2A chimeric protein was incubated with the CHO-K1/mQa1 cells, or WT CHO-K1 cells, and the activation of the mQa1 was measured by a luciferase assay. As shown in FIG. 77, the mCD80-Fc-NKG2A chimeric protein induced luciferase activity in CHO-K1/mQa1 cells, but not WT CHO-K1 cells, in a dose dependent manner. These data demonstrate that the mCD80-Fc-NKG2A chimeric protein activates mQa1 signaling in a dose dependent manner.

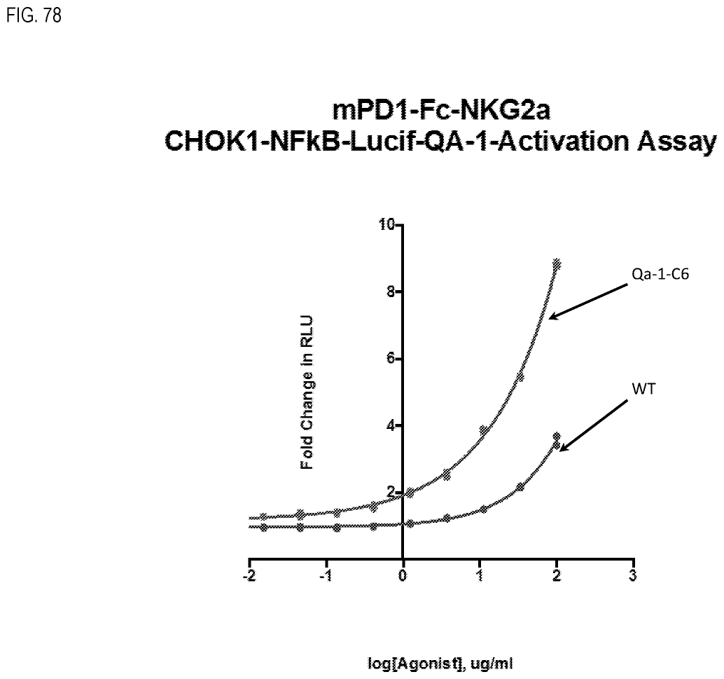

To understand whether the mPD-1-Fc-NKG2A chimeric protein can activate mQa1 signaling, the following experiment was performed. Briefly, increasing amounts of the mPD-1-Fc-NKG2A chimeric protein was incubated with the CHO-K1/mQa1 cells, or WT CHO-K1 cells, and the activation of the mQa1 was measured by a luciferase assay. As shown in FIG. 78, the mPD-1-Fc-NKG2A chimeric protein induced luciferase activity in CHO-K1/mQa1 cells, but not WT CHO-K1 cells, in a dose dependent manner. These data demonstrate that the mPD-1-Fc-NKG2A chimeric protein activates mQa1 signaling in a dose dependent manner.

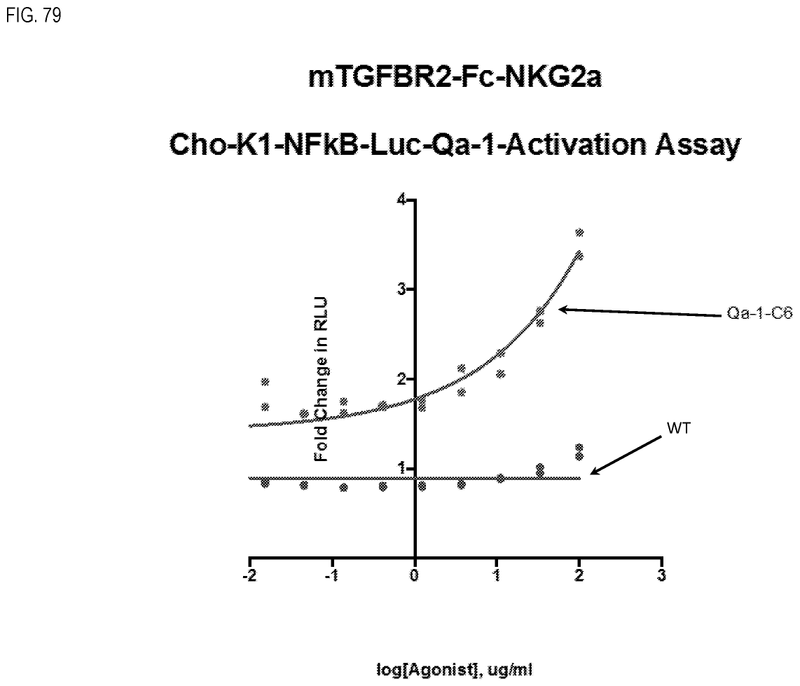

To understand whether the TGFBR2-Fc-NKG2A chimeric protein can activate Qa1 signaling, the following experiment was performed. Briefly, increasing amounts of the TGFBR2-Fc-NKG2A chimeric protein was incubated with the CHO-K1/mQa1 cells, or WT CHO-K1 cells, and the activation of the mQa1 was measured by a luciferase assay. As shown in FIG. 79, the TGFBR2-Fc-NKG2A chimeric protein induced luciferase activity in CHO-K1/mQa1 cells, but not WT CHO-K1 cells, in a dose dependent manner. These data demonstrate that the TGFBR2-Fc-NKG2A chimeric protein activates mQa1 signaling in a dose dependent manner.

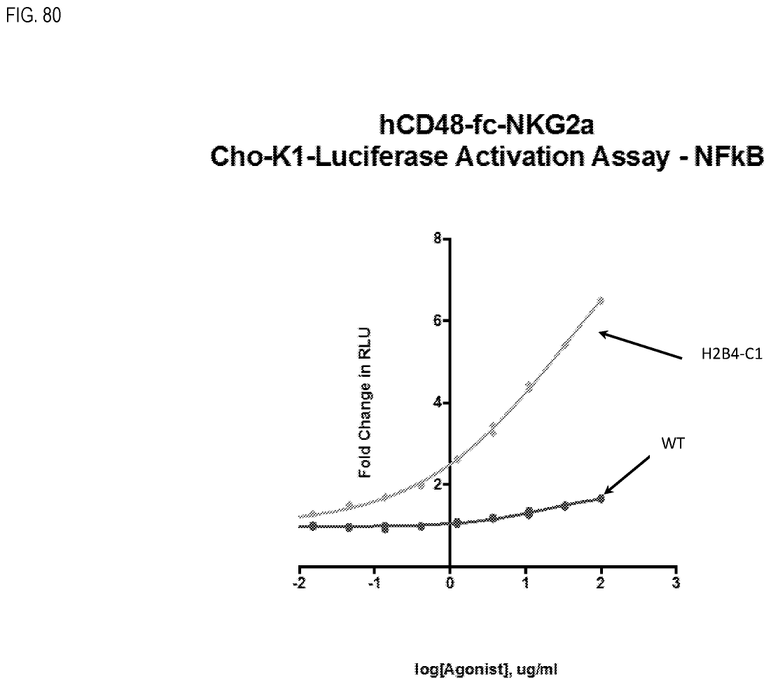

To understand whether the hCD48-Fc-NKG2A chimeric protein can activate human 2B4 (h2b4) signaling, the following experiment was performed. Briefly, increasing amounts of the hCD48-Fc-NKG2A chimeric protein was incubated with the CHO-K1/h2B4 cells, or WT CHO-K1 cells, and the activation of the h2B4 was measured by a luciferase assay. As shown in FIG. 80, the hCD48-Fc-NKG2A chimeric protein induced luciferase activity in CHO-K1/h2B4 cells, but not WT CHO-K1 cells, in a dose dependent manner. These data demonstrate that the hCD48-Fc-NKG2A chimeric protein activates h2B4 signaling in a dose dependent manner.

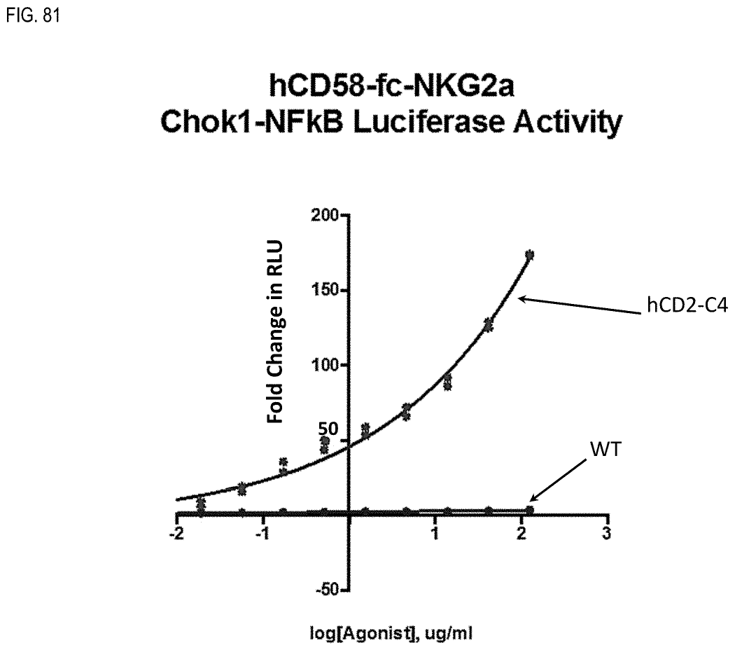

To understand whether the hCD58-Fc-NKG2A chimeric protein can activate human CD2 (h2b4) signaling, the following experiment was performed. Briefly, increasing amounts of the hCD58-Fc-NKG2A chimeric protein was incubated with the CHO-K1/hCD2 cells, or WT CHO-K1 cells, and the activation of the hCD2 was measured by a luciferase assay. As shown in FIG. 81, the hCD58-Fc-NKG2A chimeric protein induced luciferase activity in CHO-K1/hCD2 cells, but not WT CHO-K1 cells, in a dose dependent manner. These data demonstrate that the hCD58-Fc-NKG2A chimeric protein activates hCD2 signaling in a dose dependent manner.

Collectively, these results demonstrate that the type I transmembrane protein part, which is located at or near the N-terminus of the chimeric proteins, as well as the NKG2A part, which is located at or near the N-terminus of the chimeric proteins, can independently activate their natural ligands.

### Example 35: Binding of the Chimeric Proteins of the Present Disclosure to the Human Natural Killer Cell Line NK-92

The objective of this experiment was to understand whether the human chimeric proteins disclosed herein can bind to NK cells. The human NK cell line NK92 CD16V was employed for this purpose.

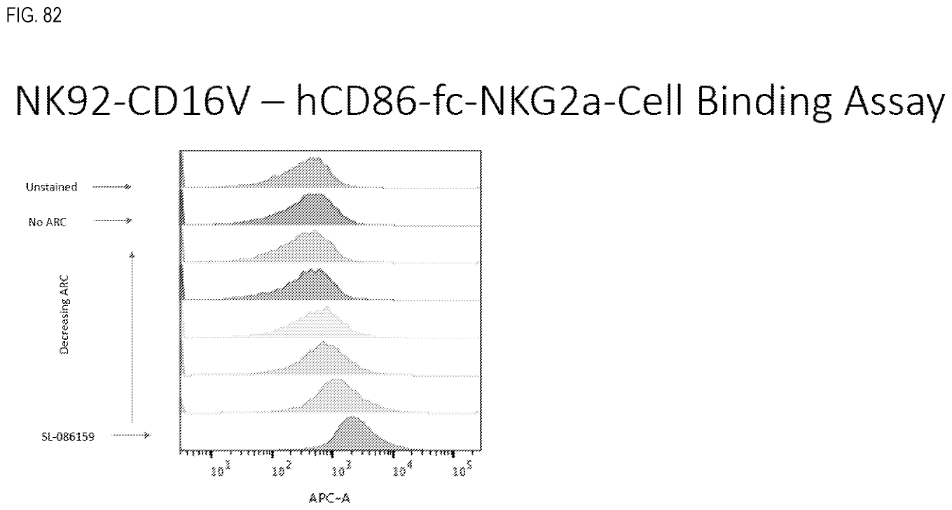

The binding of the hCD86-Fc-NKG2A chimeric protein to NK92-CD16V cells as measured by flow cytometry. As shown in FIG. 82, flow cytometry profiles exhibited a dose-dependent shift to the right hand side. These results demonstrate a dose-dependent binding of the hCD86-Fc-NKG2A chimeric protein to NK92-CD16V cells.

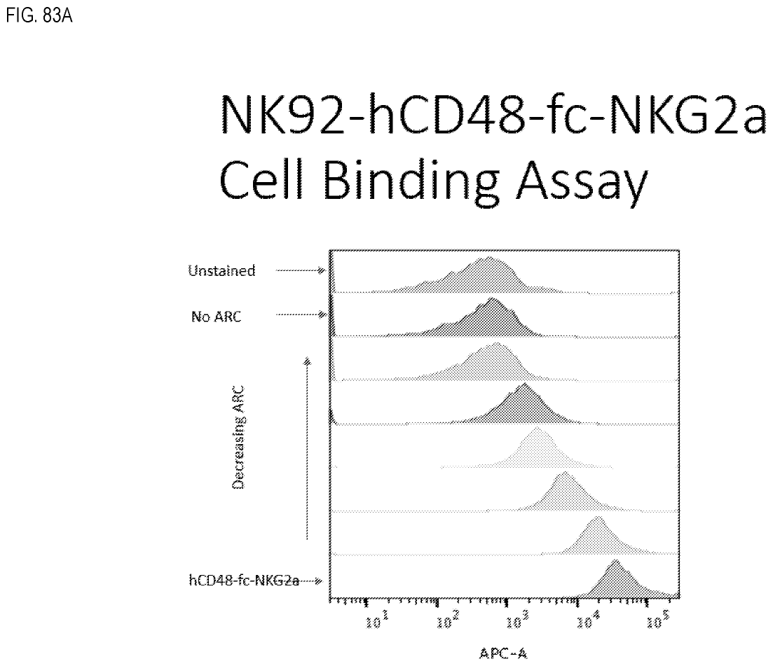

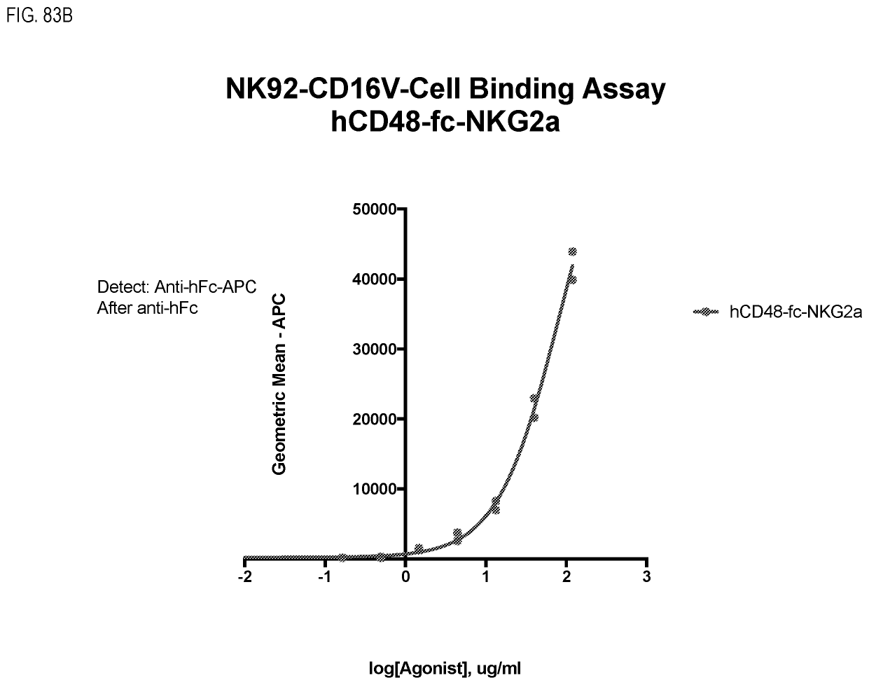

The binding of the hCD48-Fc-NKG2A chimeric protein to NK92-CD16V cells was measured by flow cytometry. As shown in FIG. 83A, flow cytometry profiles exhibited a dose-dependent shift to the right hand side. The geometric mean of the peaks was plotted. As shown in FIG. 83B, the geometric mean of binding showed a dose-dependent increase. These results demonstrate a dose-dependent binding of the hCD48-Fc-NKG2A chimeric protein to NK92-CD16V cells.

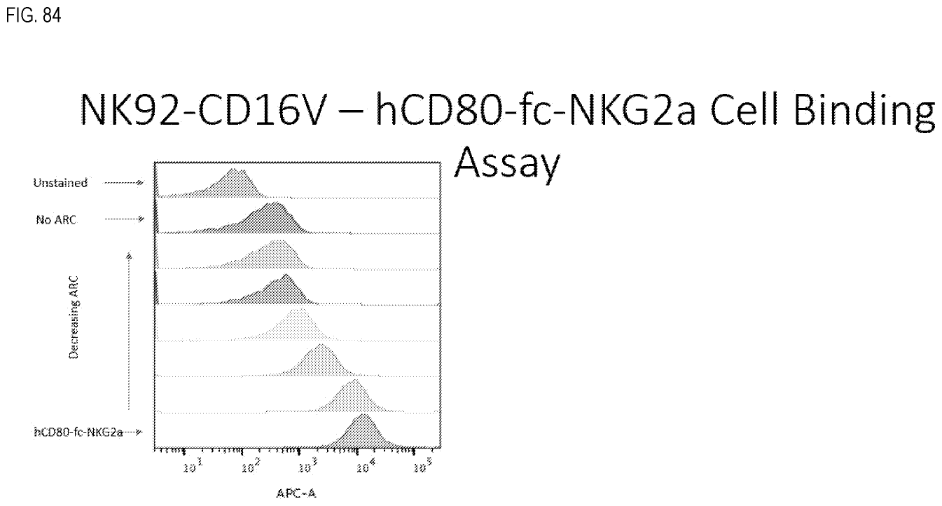

The binding of the hCD80-Fc-NKG2A chimeric protein to NK92-CD16V cells as measured by flow cytometry. As shown in FIG. 84, flow cytometry profiles exhibited a dose-dependent shift to the right hand side. These results demonstrate a dose-dependent binding of the hCD80-Fc-NKG2A chimeric protein to NK92-CD16V cells.

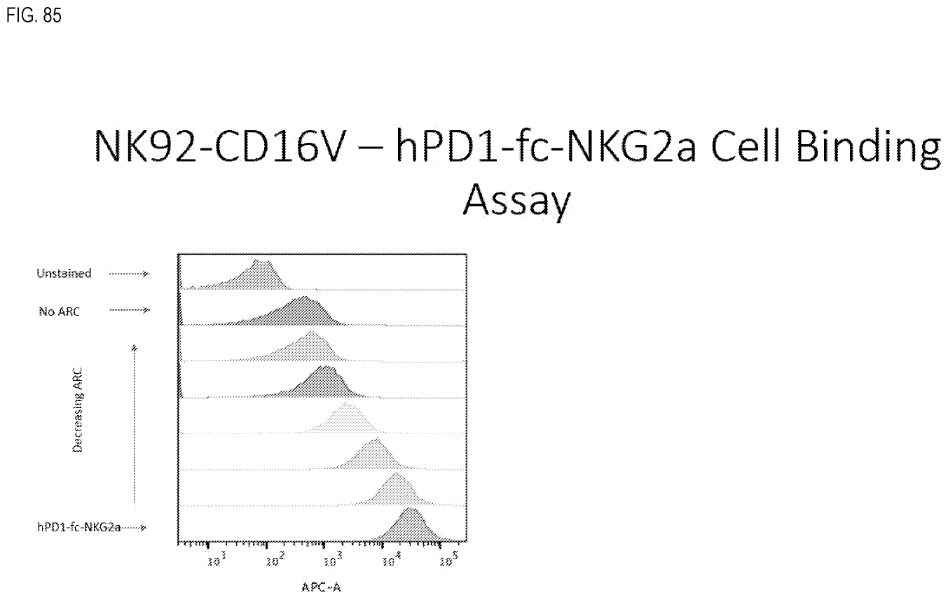

The binding of the hPD-1-Fc-NKG2A chimeric protein to NK92-CD16V cells as measured by flow cytometry. As shown in FIG. 85, flow cytometry profiles exhibited a dose-dependent shift to the right hand side. These results demonstrate a dose-dependent binding of the PD-1-Fc-NKG2A chimeric protein to NK92-CD16V cells.

Collectively, these results demonstrate that the chimeric proteins disclosed herein bind to NK cells in a dose-dependent manner.

### Example 36: Induction by the Chimeric Proteins of the Present Disclosure of Killing of Antigen Positive Cells Mediated by the Activated T Cells

The objective of this experiment was to understand whether the human chimeric proteins disclosed herein can induce killing of antigen positive cells mediated by the antigen activated T cells. An assay to determine if the mouse chimeric proteins can enhance antigen specific anti-tumor response when exposed to antigen activated T cells (OT-1 naïve T cells) was developed. Target cells were antigen positive (OVA+) cells.

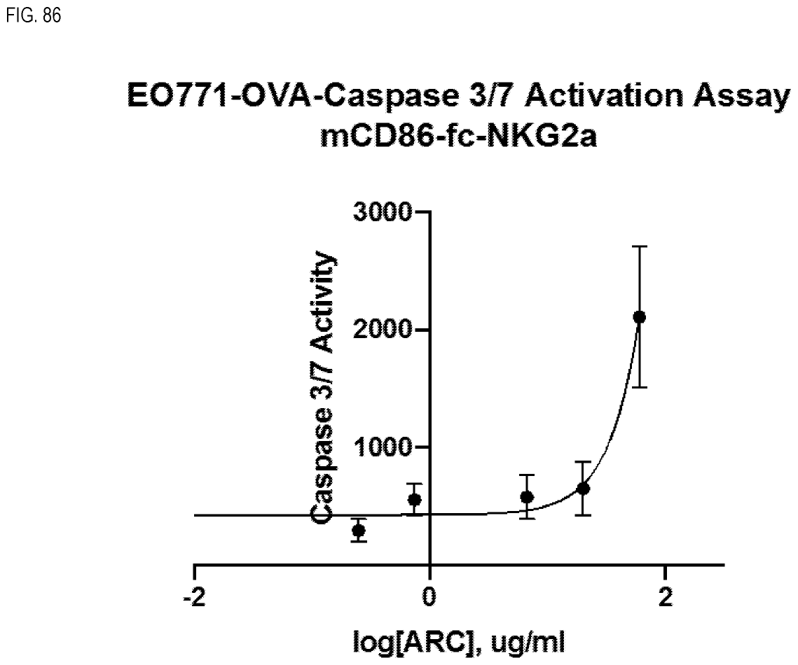

Increasing amounts of the mCD86-Fc-NKG2A chimeric protein was incubated with OT-1 naïve T cells (effector cells) and E0771 OVA+ cells (target cells) at the effector cells: target cell ration of 5:1. Apoptosis was assessed by measuring caspase 3/7 activity. The INCUCYTE system, which allows live-cell imaging and fluorescent signaling of caspase 3/7 (an apoptosis marker), was employed for this assay. As shown in FIG. 86, the mCD86-Fc-NKG2A chimeric protein induced a dose-dependent increase in apoptosis of the target cells mediated by the effector cells. These data demonstrate that the chimeric proteins disclosed herein induce apoptosis of target cells when exposed to effector cells in a dose dependent manner.

### Example 37: Induction by the Chimeric Proteins of the Present Disclosure of NK Cell-Mediated Antibody Dependent Cellular Cytotoxicity (ADCC)

The objective of this experiment was to understand whether the chimeric proteins disclosed herein can enhance the antibody dependent cellular cytotoxicity (ADCC) mediated by NK cells.

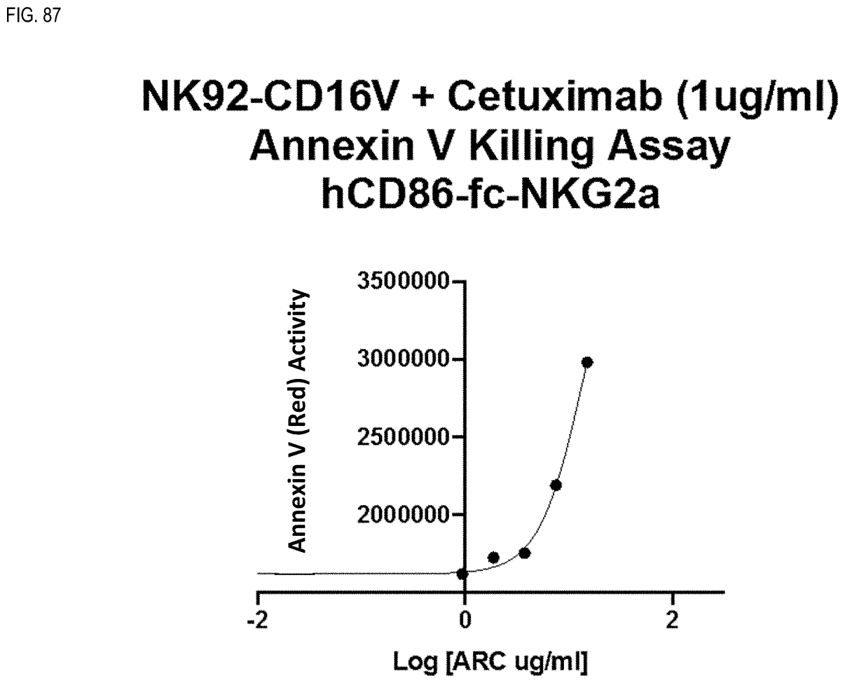

For this assay, the ADCC-capable anti-EGFR antibody Cetuximab and the EGFR-positive human A431 human non-small cell lung cancer (NSCLC) cells were used as target cells. Increasing amounts of the hCD86-Fc-NKG2A chimeric protein and 1 pg/ml Cetuximab and NK92-CD16V cells (effector cells) were mixed with A431 cells (target cells) at the effector cells: target cell ration of 5:1, and incubated. Apoptosis was assessed by measuring annexin V using the INCUCYTE live cell imaging system. As shown in FIG. 87, the hCD86-Fc-NKG2A chimeric protein caused a dose-dependent increase in annexin V positive cells. Thus, the hCD86-Fc-NKG2A chimeric protein enhanced cell death of human A431 cells when exposed to human NK cells and the ADCC capable Cetuximab antibody in a dose-dependent manner. Therefore, the chimeric protein disclosed herein enhanced cell death of human A431 cells when exposed to human NK cells and the ADCC capable Cetuximab antibody in a dose-dependent manner.

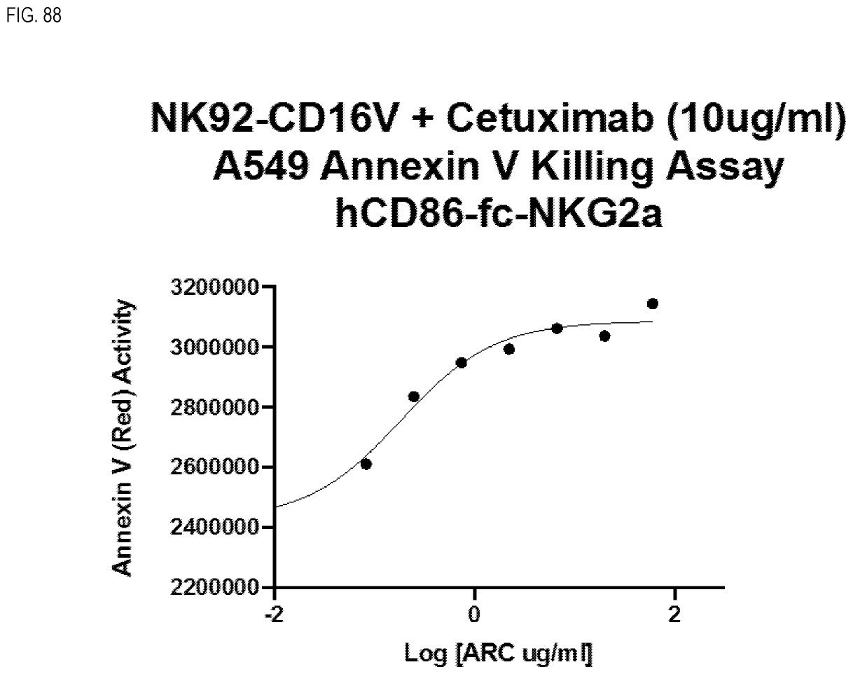

The experiment was performed with different EGFR-positive target cells: the A549 human lung carcinoma cell line. For this assay, and the ADCC-capable anti-EGFR antibody Cetuximab and the EGFR-positive human A549 human non-small cell lung cancer (NSCLC) cells were used as target cells. Increasing amounts of the hCD86-Fc-NKG2A chimeric protein and 10 pg/ml Cetuximab, and NK92-CD16V cells (effector cells) were mixed with A549 cells (target cells) at the effector cells: target cell ration of 5:1, and incubated. Apoptosis was assessed by measuring annexin V using the INCUCYTE live cell imaging system. As shown in FIG. 88, the hCD86-Fc-NKG2A chimeric protein caused a dose-dependent increase in annexin V positive cells. Thus, the hCD86-Fc-NKG2A chimeric protein enhanced cell death of human A549 cells when exposed to human NK cells and the ADCC capable Cetuximab antibody in a dose-dependent manner. Therefore, the chimeric protein disclosed herein enhanced cell death of human A549 cells when exposed to human NK cells and the ADCC capable Cetuximab antibody in a dose-dependent manner.

These data demonstrate that the chimeric proteins disclosed herein enhance the antibody dependent cellular cytotoxicity (ADCC) mediated by NK cells.

### Example 38: Induction by the Chimeric Proteins of the Present Disclosure of Killing of Antigen Positive Cells Mediated by Splenocytes

The objective of this experiment was to understand whether the human chimeric proteins disclosed herein can induce killing of antigen positive cells mediated by the splenocytes that are not antigen-activated.

Non-activated splenocytes, which comprised of multiple different immune effector cells (T cells, NK cells, etc.), were used as effector cells and murine A20 lymphocytes murine A20 lymphocytes were used as target cells.

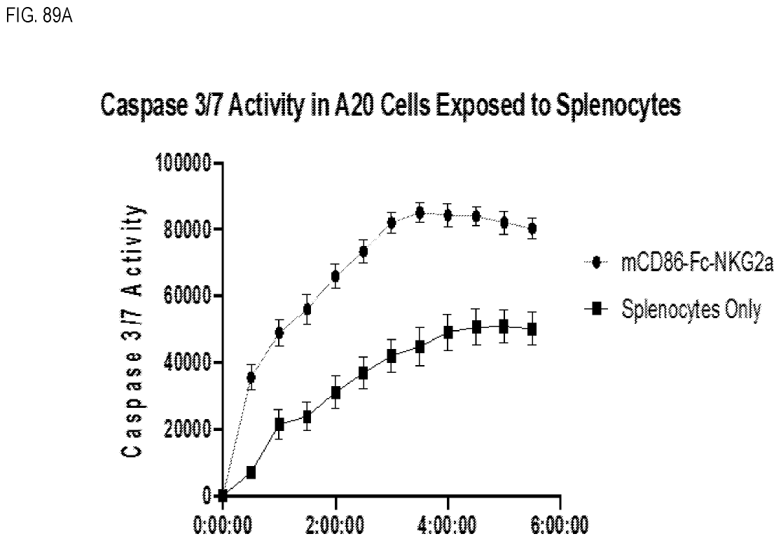

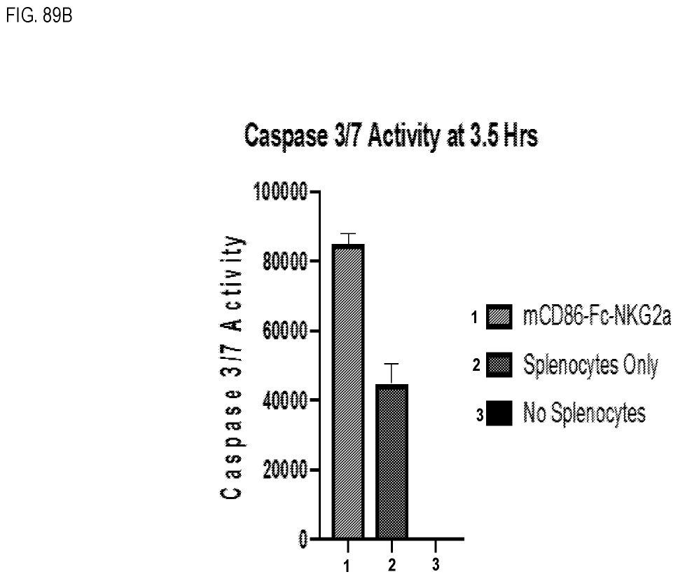

Increasing amounts of the mCD86-Fc-NKG2A chimeric protein was incubated with freshly isolated splenocytes (effector cells) and A20 cells (target cells). Negative controls included splenocytes only (without target cells) and A20 cells only (without effector cells). Apoptosis was assessed by measuring caspase 3/7 activity using the INCUCYTE system. As shown in FIG. 89A, the mCD86-Fc-NKG2A chimeric protein induced a dose-dependent increase in apoptosis of the target cells mediated by the effector cells. FIG. 89B shows a bar graph showing the caspase 3/7 activity at 3.5 hr. As shown in FIG. 89B, no apoptosis was observed without splenocytes (the invisible black bar in FIG. 89B). Splenocytes only (without the target cells) showed apoptosis, indicating that the apoptosis going on in fresh splenocytes. Interestingly, the mCD86-Fc-NKG2A chimeric protein induced significant apoptosis of target cells in the presence of splenocytes. These data demonstrate that the chimeric proteins disclosed herein induce apoptosis of target cells when exposed to effector cells in a dose dependent manner.

### Example 39: In Vivo Efficacy of the Chimeric Proteins of the Present Disclosure Against Colorectal Carcinoma Allografts

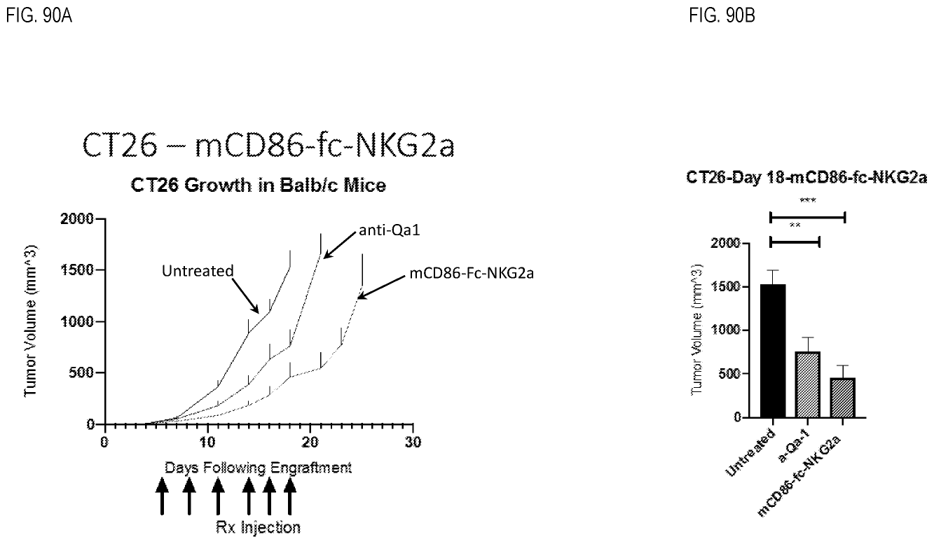

The objective of this experiment was to study the efficacy of the chimeric proteins disclosed against cancer. Efficacy of the CD86-Fc-NKG2A chimeric protein was studied in comparison with an anti-Qa1 antibody in murine colorectal carcinoma cell line CT26 allografts. Balb/c mice were inoculated with CT26 cells. After the tumors were established, the mice were randomly assigned to the following treatment groups: (1) untreated, (2) 100 pg/mouse of an anti-Qa1 antibody (Bioxcell Clone 4C2.4A7.5H11), and (3) 300 pg/mouse of the CD86-Fc-NKG2A chimeric protein. The mice were treated on days 5, 8, 11, 14, 16, and 18 post-inoculation and tumor volumes were measured at indicated times. As shown in FIG. 90A, the treatment with the anti-Qa1 antibody as well as the CD86-Fc-NKG2A chimeric protein significantly retarded the tumor growth compared to the untreated mice. The CD86-Fc-NKG2A chimeric protein caused higher tumor size reduction compared to the anti-Qa1 antibody. Tumor volumes on day 18 are plotted in FIG. 90B. The treatment with the CD86-Fc-NKG2A chimeric protein caused more reduction in tumor volume on day 18 compared to that of the anti-Qa1 antibody (FIG. 90B). These results demonstrate that the mCD86-Fc-NKG2A chimeric protein significantly reduced tumor growth and outperformed the anti-QA-1 antibody.

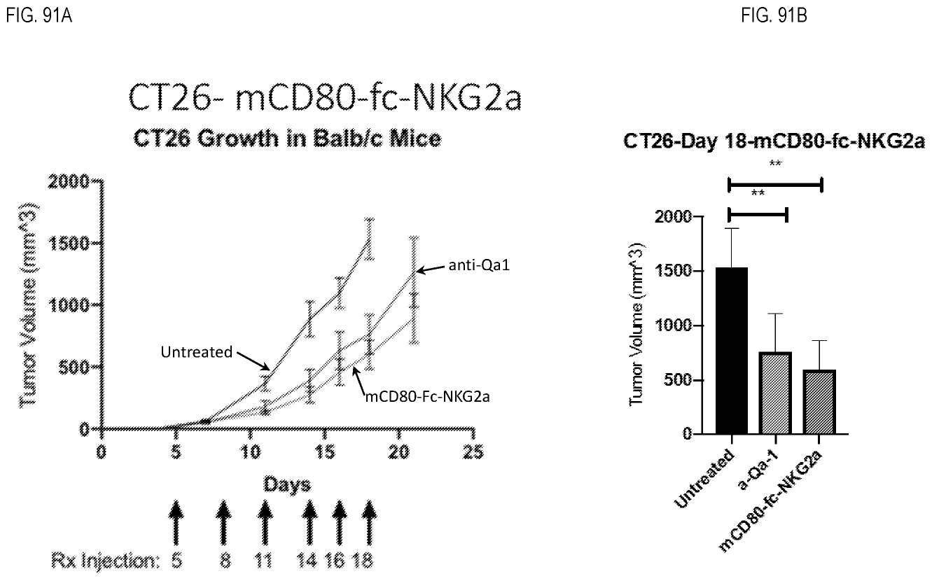

Efficacy of the mCD80-Fc-NKG2A chimeric protein was studied in comparison with an anti-Qa1 antibody in murine colorectal carcinoma cell line CT26 allografts. Balb/c mice were inoculated with CT26 cells. After the tumors were established, the mice were randomly assigned to the following treatment groups: (1) untreated, (2) 100 pg/mouse of an anti-Qa1 antibody (Bioxcell Clone 4C2.4A7.5H11), and (3) 300 pg/mouse of the mCD80-Fc-NKG2A chimeric protein. The mice were treated on days 5, 8, 11, 14, 16, and 18 post-inoculation and tumor volumes were measured at indicated times. As shown in FIG. 91A, the treatment with the anti-Qa1 antibody as well as the mCD80-Fc-NKG2A chimeric protein significantly retarded the tumor growth compared to the untreated mice. Tumor volumes on day 18 are plotted in FIG. 91B. The treatment with the mCD80-Fc-NKG2A chimeric protein caused more reduction in tumor volume on day 18 compared to that of the anti-Qa1 antibody (FIG. 91B). These results demonstrate that the mCD80-Fc-NKG2A chimeric protein significantly reduced tumor growth, with an effect that was similar to that of the anti-QA-1 antibody.

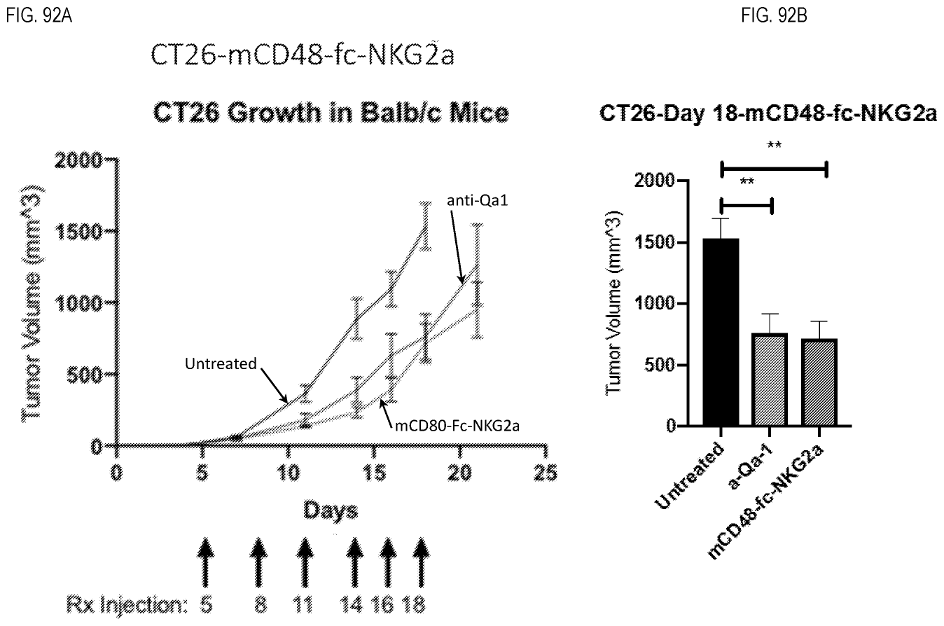

Efficacy of the mCD48-Fc-NKG2A chimeric protein was studied in comparison with an anti-Qa1 antibody in murine colorectal carcinoma cell line CT26 allografts. Balb/c mice were inoculated with CT26 cells. After the tumors were established, the mice were randomly assigned to the following treatment groups: (1) untreated, (2) 100 pg/mouse of an anti-Qa1 antibody (Bioxcell Clone 4C2.4A7.5H11), and (3) 300 pg/mouse of the mCD48-Fc-NKG2A chimeric protein. The mice were treated on days 5, 8, 11, 14, 16, and 18 post-inoculation and tumor volumes were measured at indicated times. As shown in FIG. 92A, the treatment with the anti-Qa1 antibody as well as the mCD48-Fc-NKG2A chimeric protein significantly retarded the tumor growth compared to the untreated mice. Tumor volumes on day 18 are plotted in FIG. 92B. The treatment with the mCD48-Fc-NKG2A chimeric protein caused more reduction in tumor volume on day 18 compared to that of the anti-Qa1 antibody (FIG. 92B). These results demonstrate that the mCD48-Fc-NKG2A chimeric protein significantly reduced tumor growth, with an effect that was similar to that of the anti-QA-1 antibody.

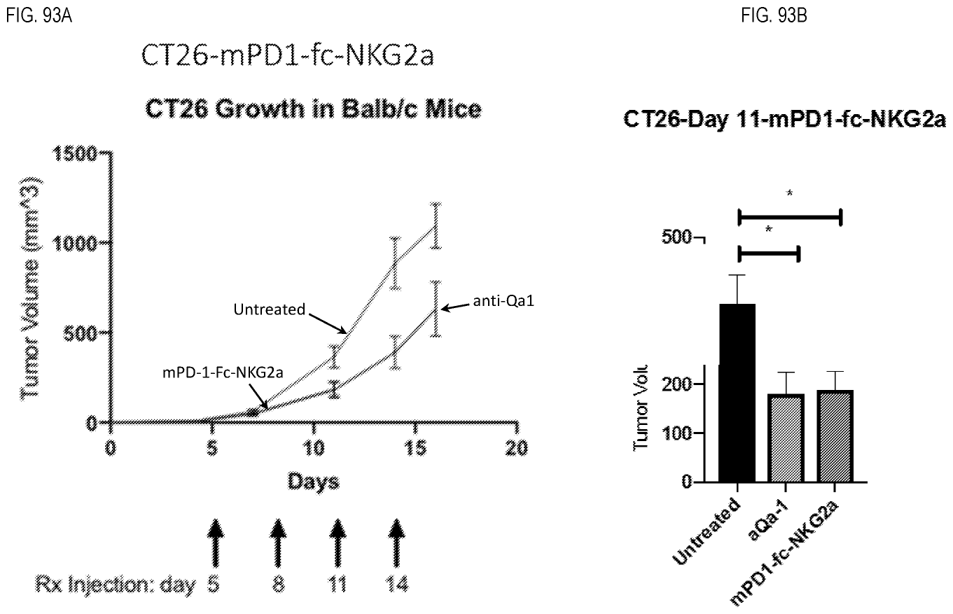

Efficacy of the mPD-1-Fc-NKG2A chimeric protein was studied in comparison with an anti-Qa1 antibody in murine colorectal carcinoma cell line CT26 allografts. Balb/c mice were inoculated with CT26 cells. After the tumors were established, the mice were randomly assigned to the following treatment groups: (1) untreated, (2) 100 pg/mouse of an anti-Qa1 antibody (Bioxcell Clone 4C2.4A7.5H11), and (3) 300 pg/mouse of the mPD-1-Fc-NKG2A chimeric protein. The mice were treated on days 5, 8, 11, and 14 post-inoculation and tumor volumes were measured at indicated times. As shown in FIG. 93A, the treatment with the anti-Qa1 antibody as well as the mPD-1-Fc-NKG2A chimeric protein significantly retarded the tumor growth compared to the untreated mice. Tumor volumes on day 11 are plotted in FIG. 93B. The treatment with the mPD-1-Fc-NKG2A chimeric protein caused more reduction in tumor volume on day 11 compared to that of the anti-Qa1 antibody (FIG. 93B). These results demonstrate that the mPD-1-Fc-NKG2A chimeric protein significantly reduced tumor growth, with an effect that was similar to that of the anti-QA-1 antibody.

Collectively, these results demonstrate that the chimeric proteins disclosed herein are at least as efficacious against cancer as the anti-QA-1 antibody.

### Example 40: In Vivo Efficacy of the Chimeric Proteins of the Present Disclosure Against an Antigen Positive Lymphoma Allograft with the Antigen Activated T Cells

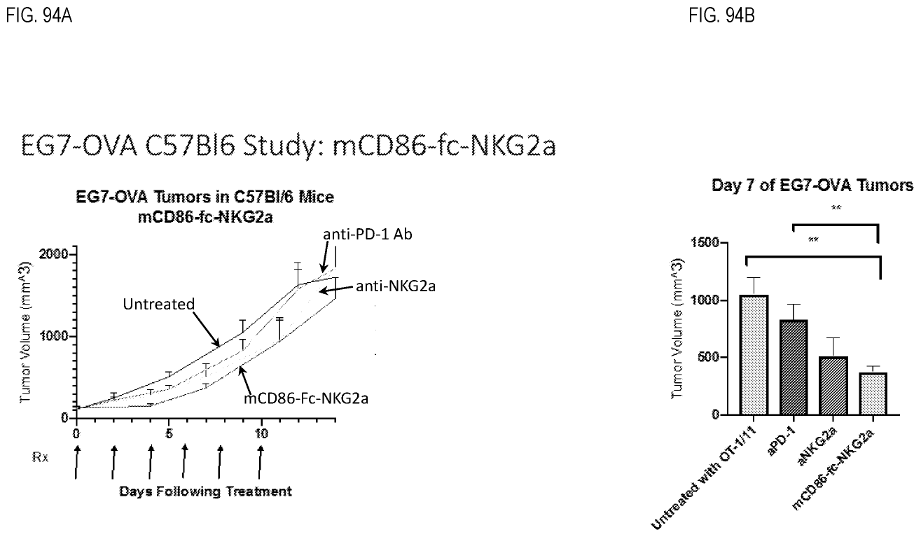

The objective of this experiment was to test the effect of the chimeric proteins of the present disclosure on the growth of cancer positive for an antigen in the presence of antigen-activated T cells. The effect of the mCD86-Fc-NKG2A chimeric protein on the growth of the murine lymphoma cell line EG7 which has been engineered to express the novel antigen OVA (EG7-OVA) was studied. Briefly, mice were inoculated with EG7-OVA cells, and infused with CD4 and CD8 OVA specific T cells. Mice were randomly assigned to one of four treatment groups: (1) untreated, (2) 100 pg/mouse anti-PD1 antibody (Bioxcell clone RMP1-14), (3) 100 pg/mouse anti-NKG2A (BioXcell clone 20D5), and (4) 300 pg/mouse of the CD86-Fc-NKG2A chimeric protein. The mice were treated on days 0, 2, 4, 6, 8 and 10 post-inoculation. The mice were treated on days 0, 2, 4, 6, 8 and 10 post-inoculation. Tumor volumes were measured. As shown in FIG. 94A, each of the treatment with anti-PD1 antibody, anti-NKG2A antibody, or the CD86-Fc-NKG2A chimeric protein retarded the tumor growth compared to the untreated mice. The CD86-Fc-NKG2A chimeric protein caused higher tumor size reduction compared to anti-PD1 antibody or anti-NKG2A antibody. Tumor volumes on day 7 are plotted in FIG. 94B. The treatment with the CD86-Fc-NKG2A chimeric protein caused significantly more reduction in tumor compared to anti-PD-1 antibody. These results demonstrate that the mCD86-Fc-NKG2A chimeric protein significantly reduced tumor growth and outperformed the anti-PD-1 antibody.

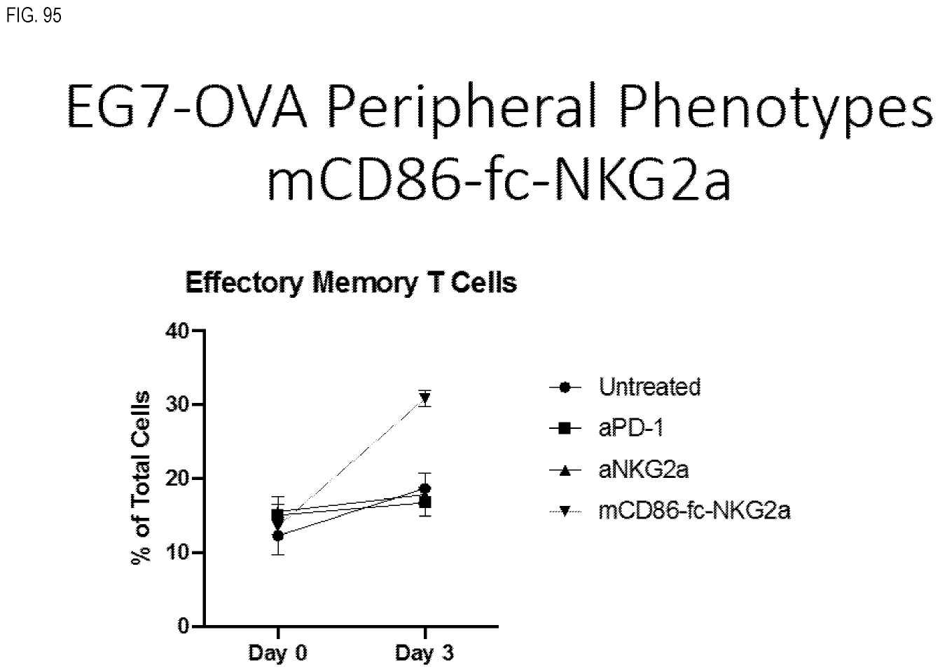

To understand the immune makeup of the mice treated with the chimeric proteins disclosed herein, peripheral phenotyping was performed using following antibodies: CD44:PE/Cy7 (Biolegend: 103209 Clone IM7), CD62L:APC (Biolegend: 104427 Clone MEL-14), CD4:BV605 (Biolegend: 100451 Clone GK1.5), Va2:AF488 (Biolegend: 127819 Clone B20.1), CD8:AF700 (Biolegend: 100729 Clone 53-6/7), and PD1:BV421 (Biolegend: 135217 Clone 29F.1A12). Cells from OT-I:GFP from transgenic to OT-1 mice were used to measure OT-1 cells. The fraction of effector memory T cells (TEM cells) on days 0 and 3 are plotted. As shown in FIG. 95, the effect with the CD86-Fc-NKG2A chimeric protein resulted in increased effector memory T cells (TEM cells) compared to the treatments with anti-PD1 antibody, anti-NKG2A antibody or with untreated mice.

These results indicate that the chimeric proteins disclosed herein significantly reducing tumor growth and boost the effector memory T cell (TEM cell) population.

### Example 41: In Vivo Efficacy of the Chimeric Proteins of the Present Disclosure Against Myelomonocytic Leukemia Allografts

The objective of this experiment was to further study the efficacy of the chimeric proteins disclosed herein against cancer. Efficacy of several chimeric proteins was studied in comparison with an anti-PD-1 antibody and anti-NKG2a antibody in murine myelomonocytic leukemia cell line WEHI-3 allografts.

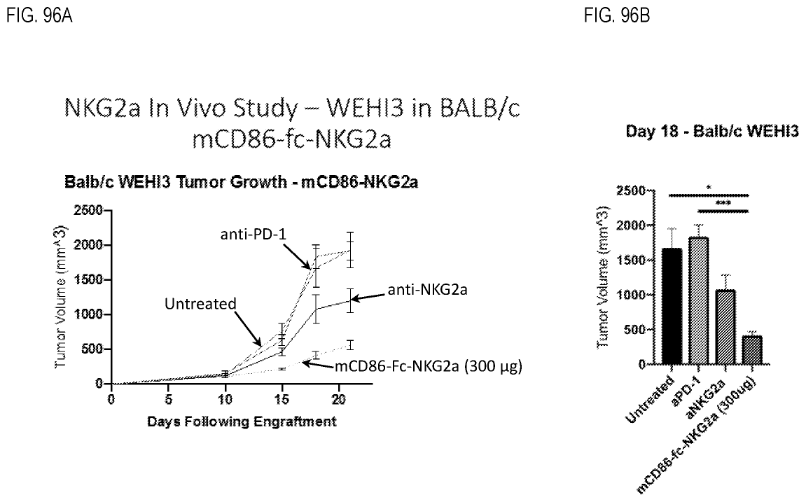

The effect of the CD86-Fc-NKG2A chimeric protein on WEHI-3 allografts was studied in comparison with an anti-PD-1 antibody and anti-NKG2a antibody. Briefly, Balb/c mice were inoculated with WEHI-3 cells. After the tumors were established, the mice were randomly assigned to the following treatment groups: (1) untreated, (2) 100 pg/mouse of an anti-PD1 antibody (Bioxcell clone RMP1-14), (3) 100 pg/mouse of an anti-NKG2A antibody (BioXcell clone 20D5) and (4) 300 pg/mouse of the CD86-Fc-NKG2A chimeric protein. The mice were treated on days 0, 2, 4, 6, 8 and 10 post-inoculation. The mice were treated six times, two days apart. Tumor volumes were measured. As shown in FIG. 96A, each of the treatments with anti-NKG2A antibody or the CD86-Fc-NKG2A chimeric protein retarded the tumor growth compared to the untreated mice. The treatment with anti-PD1 antibody did not have a significant effect. The CD86-Fc-NKG2A chimeric protein caused higher tumor size reduction compared to anti-PD1 antibody or anti-NKG2A antibody. Tumor volumes on day 18 are plotted in FIG. 96B. The treatment with the CD86-Fc-NKG2A chimeric protein significant reduction in tumor volume compared to the tumors in both untreated and anti-PD-1 antibody-treated mice. These results demonstrate that the mCD86-Fc-NKG2A chimeric protein significantly reduced tumor growth and outperformed the anti-PD-1 antibody. These results demonstrate that the CD86-Fc-NKG2A chimeric protein outperformed the anti-PD-1 and anti-NKG2A antibodies in reducing the tumor volume of WEHI3 tumors.

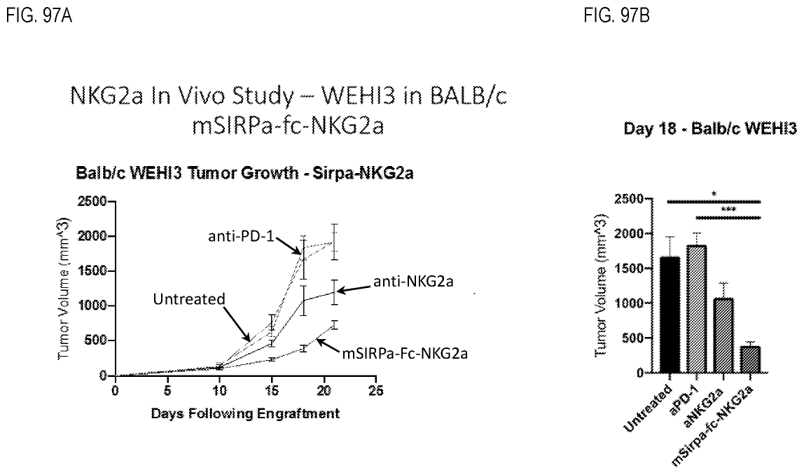

The effect of the SIRPα-Fc-NKG2A chimeric protein on WEHI-3 allografts was studied in comparison with an anti-PD-1 antibody and anti-NKG2a antibody. Briefly, Balb/c mice were inoculated with WEHI-3 cells. After the tumors were established, the mice were randomly assigned to the following treatment groups: (1) untreated, (2) 100 pg/mouse of an anti-PD1 antibody (Bioxcell clone RMP1-14), (3) 100 pg/mouse of an anti-NKG2A antibody (BioXcell clone 20D5) and (4) 300 pg/mouse of the SIRPα-Fc-NKG2A chimeric protein. The mice were treated on days 0, 2, 4, 6, 8 and 10 post-inoculation. The mice were treated six times, two days apart. Tumor volumes were measured. As shown in FIG. 97A, each of the treatments with anti-NKG2A antibody or the SIRPα-Fc-NKG2A chimeric protein retarded the tumor growth compared to the untreated mice. The treatment with anti-PD1 antibody did not have a significant effect. The SIRPα-Fc-NKG2A chimeric protein caused higher tumor size reduction compared to anti-PD1 antibody or anti-NKG2A antibody. Tumor volumes on day 18 are plotted in FIG. 97B. The treatment with the SIRPα-Fc-NKG2A chimeric protein significant reduction in tumor volume compared to the tumors in both untreated and anti-PD-1 antibody-treated mice. These results demonstrate that the mSIRPα-Fc-NKG2A chimeric protein significantly reduced tumor growth and outperformed the anti-PD-1 antibody. These results demonstrate that the SIRPα-Fc-NKG2A chimeric protein outperformed the anti-PD-1 and anti-NKG2A antibodies in reducing the tumor volume of WEHI3 tumors.

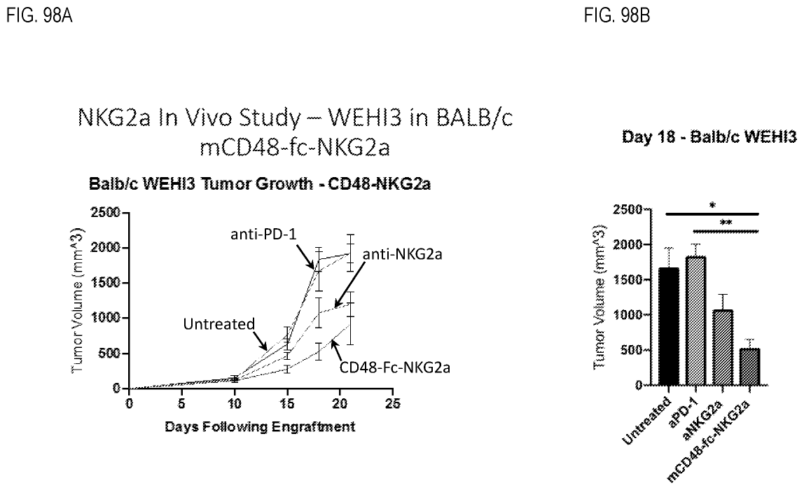

The effect of the CD48-Fc-NKG2A chimeric protein on WEHI-3 allografts was studied in comparison with an anti-PD-1 antibody and anti-NKG2a antibody. Briefly, Balb/c mice were inoculated with WEHI-3 cells. After the tumors were established, the mice were randomly assigned to the following treatment groups: (1) untreated, (2) 100 pg/mouse of an anti-PD1 antibody (Bioxcell clone RMP1-14), (3) 100 pg/mouse of an anti-NKG2A antibody (BioXcell clone 20D5) and (4) 300 pg/mouse of the CD48-Fc-NKG2A chimeric protein. The mice were treated on days 0, 2, 4, 6, 8 and 10 post-inoculation. The mice were treated six times, two days apart. Tumor volumes were measured. As shown in FIG. 98A, each of the treatments with anti-NKG2A antibody or the CD48-Fc-NKG2A chimeric protein retarded the tumor growth compared to the untreated mice. The treatment with anti-PD1 antibody did not have a significant effect. The CD48-Fc-NKG2A chimeric protein caused higher tumor size reduction compared to anti-PD1 antibody or anti-NKG2A antibody. Tumor volumes on day 18 are plotted in FIG. 98B. The treatment with the CD48-Fc-NKG2A chimeric protein significant reduction in tumor volume compared to the tumors in both untreated and anti-PD-1 antibody-treated mice. These results demonstrate that the mCD48-Fc-NKG2A chimeric protein significantly reduced tumor growth and outperformed the anti-PD-1 antibody. These results demonstrate that the CD48-Fc-NKG2A chimeric protein outperformed the anti-PD-1 and anti-NKG2A antibodies in reducing the tumor volume of WEHI3 tumors.

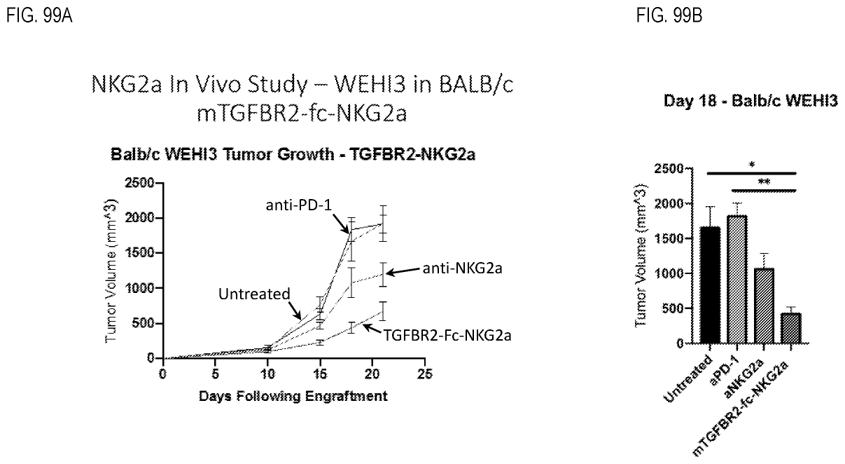

The effect of the TGFBR2-Fc-NKG2A chimeric protein on WEHI-3 allografts was studied in comparison with an anti-PD-1 antibody and anti-NKG2a antibody. Briefly, Balb/c mice were inoculated with WEHI-3 cells. After the tumors were established, the mice were randomly assigned to the following treatment groups: (1) untreated, (2) 100 pg/mouse of an anti-PD1 antibody (Bioxcell clone RMP1-14), (3) 100 pg/mouse of an anti-NKG2A antibody (BioXcell clone 20D5) and (4) 300 pg/mouse of the TGFBR2-Fc-NKG2A chimeric protein. The mice were treated on days 0, 2, 4, 6, 8 and 10 post-inoculation. The mice were treated six times, two days apart. Tumor volumes were measured. As shown in FIG. 99A, each of the treatments with anti-NKG2A antibody or the TGFBR2-Fc-NKG2A chimeric protein retarded the tumor growth compared to the untreated mice. The treatment with anti-PD1 antibody did not have a significant effect. The TGFBR2-Fc-NKG2A chimeric protein caused higher tumor size reduction compared to anti-PD1 antibody or anti-NKG2A antibody. Tumor volumes on day 18 are plotted in FIG. 99B. The treatment with the TGFBR2-Fc-NKG2A chimeric protein significant reduction in tumor volume compared to the tumors in both untreated and anti-PD-1 antibody-treated mice. These results demonstrate that the mTGFBR2-Fc-NKG2A chimeric protein significantly reduced tumor growth and outperformed the anti-PD-1 antibody. These results demonstrate that the TGFBR2-Fc-NKG2A chimeric protein outperformed the anti-PD-1 and anti-NKG2A antibodies in reducing the tumor volume of WEHI3 tumors.

Collectively, these results demonstrate that the chimeric proteins disclosed herein are more efficacious than the anti-PD-1 and anti-NKG2A antibodies against cancer.

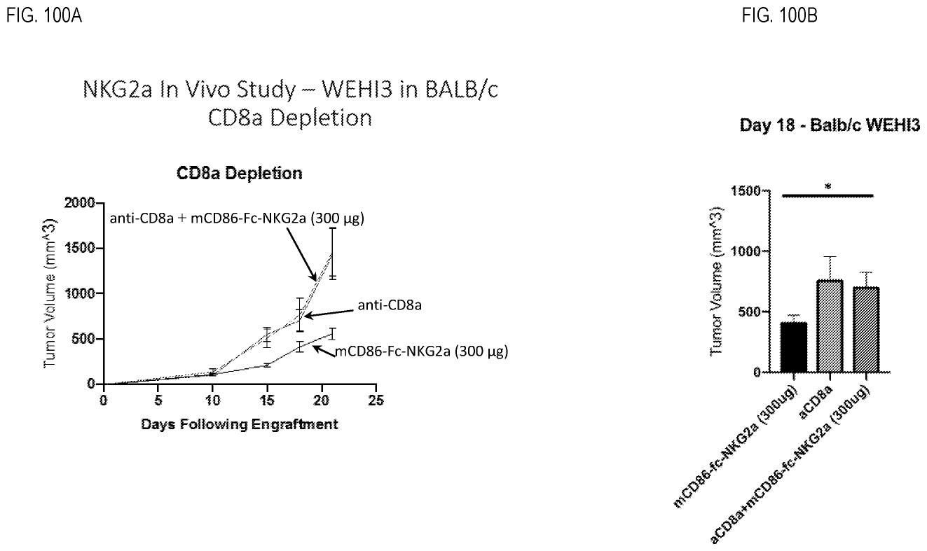

To further understand the mechanism of action of the chimeric proteins disclosed herein, the role of CD8 T cells was probed. CD8 T cells were depleted using an anti-CD8a antibody (Bioxcell clone 2.43) that is known to deplete CD8+ cells, and the ability of the mCD86-Fc-NKG2A chimeric protein to control tumor growth was measured. Briefly, Balb/c mice were inoculated with WEHI-3 cells. After the tumors were established, the mice were randomly assigned to the following treatment groups: (1) 250 pg/mouse of an anti-CD8a antibody (Bioxcell clone 2.43), (2) 300 pg/mouse of the CD86-Fc-NKG2A chimeric protein, and (3) 250 pg/mouse of the anti-CD8a antibody (Bioxcell clone 2.43)+300 pg/mouse of the CD86-Fc-NKG2A chimeric protein. The anti-CD8a antibody (Bioxcell clone 2.43) was administered on days −1, 1, and 3 of the treatment regimen to deplete CD8 cells. The mice from groups 2 and 3 were treated on days 0, 2, 4, 6, 8 and 10 post-inoculation with the CD86-Fc-NKG2A chimeric protein. The mice were treated six times, two days apart. Tumor volumes were measured on indicated days. As shown in FIG. 100A, mice treated with the CD86-Fc-NKG2A chimeric protein showed more antitumor activity compared to the mice treated with the anti-CD8a antibody. Interestingly, combined treatment with the anti-CD8a antibody and the CD86-Fc-NKG2A chimeric protein extinguished the observed antitumor activity. As shown in FIG. 100B, the tumor volumes on day 18 were significantly smaller in mice treated with the CD86-Fc-NKG2A chimeric protein alone compared to the combination of the anti-CD8a antibody and the CD86-Fc-NKG2A chimeric protein. These results demonstrate that CD8 T cells are required for the anti-tumor effect of chimeric proteins disclosed herein.

### Example 42: Infiltration of Immune Cells in Tumor, Spleen, and Lymph Node During the Treatment with the Chimeric Proteins of the Present Disclosure

The effect of the chimeric proteins of the present disclosure on levels of immune cells in tumor, spleen, and lymph node was studied. Briefly, Balb/c mice were inoculated with WEHI-3 cells. After the tumors were established, the mice were randomly assigned to the following treatment groups: (1) untreated, (2) 100 pg/mouse of an anti-NKG2A antibody (BioXcell clone 20D5) and (3) 300 pg/mouse of the CD86-Fc-NKG2A chimeric protein. The mice were treated on days 1, 3, 5, and 7 post-inoculation. At day 8 post treatment of untreated, an anti-NKG2A antibody, and mCD86-Fc-NKG2A mice were sacrificed, and the tumor, lymph node, and spleen were isolated, digested and analyzed for the amounts of the different immune cells in each of these environments using an NK panel and a T cell panel of antibodies. The NK panel comprised NKG2A-PeCy7 (Biolegend 142809 Clone 16A11), CD107-PE (Biolegend: 121611 Clone 1D4B), CD137-APC (Biolegend:106109 Clone 17B5), Granzyme B-Fitc (Biolegend 515402 Clone GB11), NKP46-BV421 (Biolegend 137611 Clone 29A1.4), CD16-BV510 (Biolegend 149531 Clone 9E9), CD27-PerCPCy5.5 (Biolegend 124213 Clone LG.3A10), KLRG1-BV605 (Biolegend 138419 Clone 2F1/KLRG1), CD11b-APC/Cy7 (Biolegend 101225 Clone M1/70), and CD3-AF700 (Biolegend 100215 Clone 17A2). The T Cell panel comprised NKG2A-PeCy7 (Biolegend 142809 Clone 16A11), CD3-Fitc (Biolegend 100306 Clone 145-2C11), CD8a-AF700 (Biolegend 100729 Clone 54-6.7), CD62L-BV421 (Biolegend 104435 Clone MEL-14), CD44-PeCy5 (Biolegend 103009 Clone IM7), Perforin: PE (Biolegend 154305 Clone S16009A), IAIE-BV605 (Biolegend 107639 Clone M5/114.15.2), PD1-BV510 (Biolegend 135241 Clone 29F.1A12), IFN-g-APC/Cy7 (Biolegend 505849 Clone XMG1.2), and CD137-APC (Biolegend 106109 Clone 17B5).

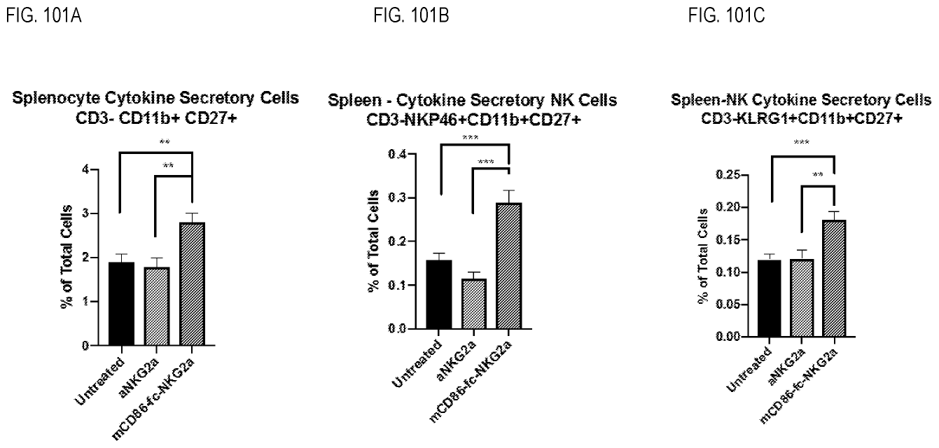

As shown in FIG. 101A, the cytokine secretory CD3- CD11 b+CD27+ splenocytes significantly increased in the mice treated with the CD86-Fc-NKG2A chimeric protein compared to the untreated mice and the mice treated with the anti-NKG2A antibody. Similarly, the CD3- NKP46+CD11b+CD27+ cytokine secretory NK cells in spleen (FIG. 101B), and the CD3- KLRG1+CD11b+CD27+NK cytokine secretory cells in spleen (FIG. 101C) significantly increased in the mice treated with the CD86-Fc-NKG2A chimeric protein compared to the untreated mice and the mice treated with the anti-NKG2A antibody. Therefore, the mCD86-Fc-NKG2A chimeric protein induced the growth of cytokine secretory cells in the spleen compared to both untreated and anti-NKG2A antibody therapy. These results demonstrate that the chimeric proteins of the present disclosure induce the growth of cytokine secretory cells in the spleen compared to both untreated and anti-NKG2A antibody therapy.

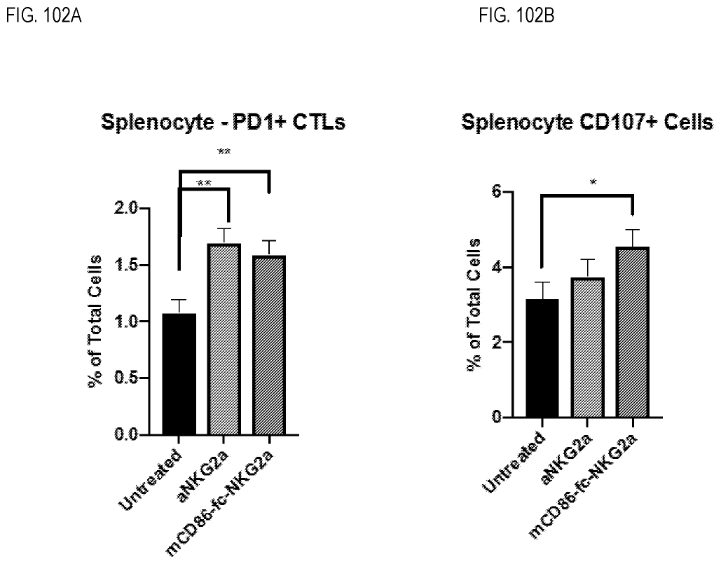

The spleens were isolated, digested and analyzed for the amounts of the different immune cells. As shown in FIG. 102A, the PD-1+ cytotoxic T lymphocytes (CTLs) in the spleens significantly increased in the mice treated with the CD86-Fc-NKG2A chimeric protein and the mice treated with the anti-NKG2A antibody compared to the untreated mice. Further, as shown in FIG. 102B, the CD107+ cells in the spleens of the mice significantly increased in the mice treated with the CD86-Fc-NKG2A chimeric protein compared to the untreated mice. Therefore, the CD86-Fc-NKG2A chimetric protein induced the growth of activated cytotoxic T cells and CD107+ cell (a marker for enhanced cytotoxicity). As shown, the effects of the CD86-Fc-NKG2A chimeric protein and anti-NKG2A antibody were equivalent in this system. These results demonstrate that the chimeric proteins of the present disclosure induce the growth of activated cytotoxic T cells and CD107+cell (a marker for enhanced cytotoxicity).

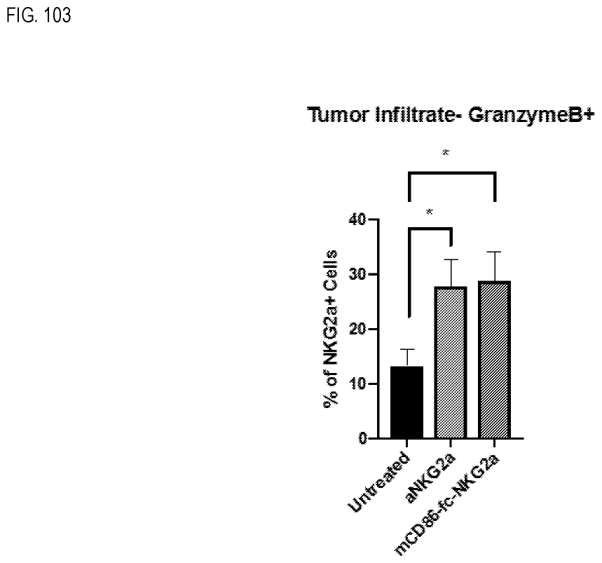

The tumors were isolated, digested and analyzed for the amounts of granzyme B+ cells in the tumors. As shown in FIG. 103, the granzyme B+ cells in the tumors significantly increased in the mice treated with the CD86-Fc-NKG2A chimeric protein and the mice treated with the anti-NKG2A antibody compared to the untreated mice. Therefore, the CD86-Fc-NKG2A chimetric protein induced the enhanced infiltration of immune cells into the tumor with the cytolytic marker Granzyme B. The effects of the CD86-Fc-NKG2A chimeric protein and anti-NKG2A antibody were equivalent in this system. These results demonstrate that the chimeric proteins of the present disclosure induce the enhanced infiltration of immune cells into the tumor with the cytolytic marker Granzyme B.

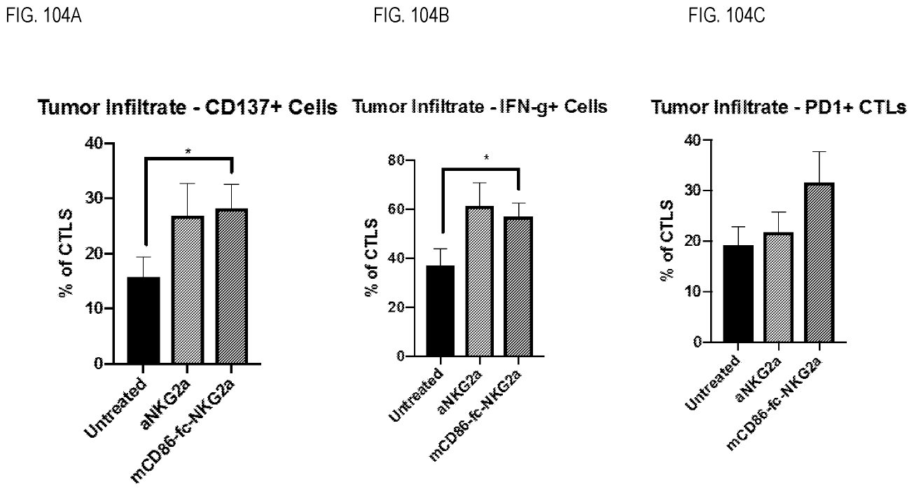

In addition, as shown in FIG. 104A, the CD137+ cells in the tumors significantly increased in mice treated with the CD86-Fc-NKG2A chimeric protein and the mice treated with the anti-NKG2A antibody compared to the untreated mice. Likewise, the IFNγ+ cells in the tumors significantly increased in mice treated with the CD86-Fc-NKG2A chimeric protein and the mice treated with the anti-NKG2A antibody compared to the untreated mice (FIG. 104B). The PD-1+ cytotoxic T lymphocytes (CTLs) in the tumors significantly increased in mice treated with the CD86-Fc-NKG2A chimeric protein compared to the untreated mice (FIG. 104C). Therefore, the CD86-Fc-NKG2A chimeric protein induced enhanced infiltration of immune cells into the tumor that express potent activation markers (CD137, IFN-γ, and PD1). The effects of the CD86-Fc-NKG2A chimeric protein and anti-NKG2A antibody were equivalent in this system. These results demonstrate that the chimeric proteins of the present disclosure induce the enhanced infiltration of immune cells into the tumor that express potent activation markers (e.g. CD137, IFN-γ, and PD1).

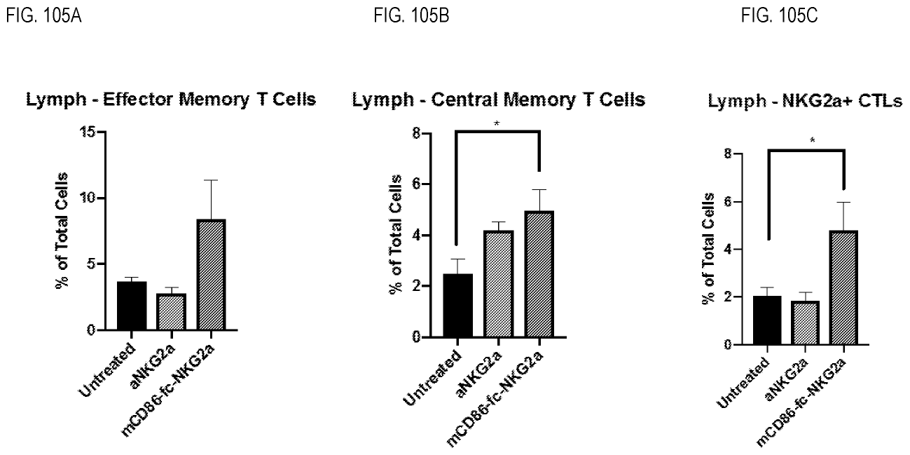

The lymph nodes were isolated, digested and analyzed for the amounts of the different immune cells. As shown in FIG. 105A, the effector memory T cells in the lymph nodes significantly increased in the mice treated with the CD86-Fc-NKG2A chimeric protein compared to the untreated mice and the mice treated with the anti-NKG2A antibody. Likewise, the central memory T cells significantly increased in the lymph nodes of the mice treated with the CD86-Fc-NKG2A chimeric protein compared to the untreated mice (FIG. 105B). Moreover, the NKG2a+ cytotoxic T lymphocytes (CTLs) in the lymph nodes of the mice increased in the mice treated with the CD86-Fc-NKG2A chimeric protein compared to the untreated mice and the mice treated with the anti-NKG2A antibody (FIG. 105C). Therefore, the CD86-Fc-NKG2A chimeric protein induced enhanced infiltration of immune cells in the draining lymph node of effector memory T cells, central memory t cells, and NKG2A+ CD8+ T cells indicating mCD86-Fc-NKG2A was able to induce the infiltration and proliferation of these important effector immune cells.

The effects of the CD86-Fc-NKG2A chimeric protein and anti-NKG2A antibody were equivalent in this system. These results demonstrate that the chimeric proteins of the present disclosure induce enhanced infiltration of immune cells in the draining lymph node of effector memory T cells, central memory t cells, and NKG2A+ CD8+ T cells indicating mCD86-Fc-NKG2A was able to induce the infiltration and proliferation of these important effector immune cells.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present technology is not entitled to antedate such publication by virtue of prior invention.

As used herein, all headings are simply for organization and are not intended to limit the disclosure in any manner. The content of any individual section may be equally applicable to all sections.

### EQUIVALENTS

While the invention has been disclosed in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments disclosed specifically herein. Such equivalents are intended to be encompassed in the scope of the following claims.

1.-93. (canceled)

94. A chimeric protein of a general structure of:
N terminus-(a)-(b)-(c)-C terminus,
wherein:
(a) is a first domain comprising a portion of CD48, the first domain being capable of binding a CD48 ligand/receptor,
(b) is a linker comprising a hinge-CH2-CH3 Fc domain, and
(c) is a second domain comprising an extracellular domain of NKG2A, the second domain being capable of binding an NKG2A ligand/receptor.

95. The chimeric protein of claim 94, wherein the CD48 ligand/receptor is CD2 or 2B4.

96. The chimeric protein of claim 94, wherein the NKG2A ligand/receptor is HLA-E.

97. The chimeric protein of claim 94, wherein binding the NKG2A ligand/receptor blocks transmission of an immune inhibitory signal to an NK cell.

98. The chimeric protein of claim 94, wherein the first domain comprises an amino acid sequence that is at least 95% identical to the amino acid sequence of SEQ ID NO: 81.

99. The chimeric protein of claim 94, wherein the second domain comprises an amino acid sequence that is at least 95% identical to the amino acid sequence of SEQ ID NO: 57.

100. The chimeric protein of claim 94, wherein the hinge-CH2-CH3 Fc domain is derived from an IgG selected from IgG1, IgG2, IgG3, and IgG4.

101. The chimeric protein of claim 100, wherein the IgG is IgG4.

102. The chimeric protein of claim 94, wherein the linker comprises an amino acid sequence that is at least 95% identical to one of the amino acid sequences of SEQ ID NO: 1, SEQ ID NO: 2, or SEQ ID NO: 3.

103. The chimeric protein of claim 102, wherein the linker further comprises one or more joining linkers, the joining linkers being independently selected from SEQ ID NOs: 4-50.

104. The chimeric protein of claim 94, wherein the chimeric protein has an amino acid sequence that is at least 95% identical to the amino acid sequence of SEQ ID NO: 83.

105. The chimeric protein of claim 94, wherein binding of the first domain to its ligand/receptor inhibits an immunosuppressive signal and/or activates another immunosuppressive signal.

106. The chimeric protein of claim 94, wherein the chimeric protein is capable of forming a stable synapse between an NK cell and a tumor cell, or an NK cell and a virus-infected cell.

107. The chimeric protein of claim 106, wherein the stable synapse between cells provides a spatial orientation that favors tumor reduction or killing of virus-infected cells by the NK cell.

108. The chimeric protein of claim 107, wherein the spatial orientation positions the NK cells to attack target cells and/or sterically prevents the target cells from delivering an immune-inhibiting signal to NK cells, wherein the target cells are selected from tumor cells and virus-infected cells.

109. A chimeric protein comprising:
(a) a first domain comprising a portion of CD48 comprising an amino acid sequence that is at least 95% identical to the amino acid sequence of SEQ ID NO: 81,
(b) a second domain comprising a portion of NKG2A comprising an amino acid sequence that is at least 95% identical to the amino acid sequence of SEQ ID NO: 57, and
(c) a linker linking the first domain and the second domain and comprising a hinge-CH2-CH3 Fc domain comprising an amino acid sequence that is at least 95% identical to the amino acid sequence of SEQ ID NO: 1, SEQ ID NO: 2, or SEQ ID NO: 3, and optionally further comprising one, two, or more joining linkers independently selected from SEQ ID NOs: 4-50.

110. The chimeric protein of claim 109, wherein the chimeric protein comprises an amino acid sequence having at least about 95% sequence identity with the amino acid sequence of SEQ ID NO: 83.

111. A nucleic acid encoding the chimeric protein of claim 94.

112. A pharmaceutical composition comprising the chimeric protein of claim 94.

113. A method of treating cancer or an inflammatory disease, comprising administering an effective amount of a pharmaceutical composition of claim 112 to a subject in need thereof.
